(19) United States
(12) Patent Application Publication
(10) Pub. No.: US 2026/0176226 A1
(43) Pub. Date: Jun. 25, 2026

(54) OLIGOMERIC BINAPHTHYL COMPOUNDS AND THERMOPLASTIC RESINS

(71) Applicant: Mitsubishi Gas Chemical Company, Inc., Tokyo (JP)

(72) Inventors: Vasyl ANDRUSHKO, Freiburg (DE); Karl REUTER, Freiburg (DE)

(73) Assignee: Mitsubishi Gas Chemical Company, Inc., Tokyo (JP)

(21) Appl. No.: 19/116,800

(22) PCT Filed: Sep. 28, 2023
(86) PCT No.: PCT/EP2023/076930

§ 371 Date: Mar. 28, 2025

(30) Foreign Application Priority Data
Sep. 30, 2022 (EP) 22199261.3

(51) Int. Cl. C07C 43/295; C08G 63/672

(52) U.S. Cl. CPC C07C 43/295; C08G 63/672

(57) ABSTRACT

The present invention relates to the use of the compound of the formula (I) where the variables are as defined in the claims and the description, or of a mixture thereof, as a monomer for producing a thermoplastic resin selected from polyesters, polycarbonates and polyestercarbonates. The invention relates moreover to compounds (I), except for compounds where A¹ and A² are both unsubstituted 1,4-phenylene, p and q are both 0, and X¹ and X² are both —CH₂OH or —C(O)OH, and except for the compound where A¹ and A² are both unsubstituted 2,3-quinolinylene, p and q are both 0, and X¹ and X² are both —CH₂OH.

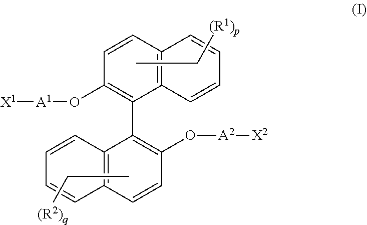

The present invention relates to oligomeric binaphtyl compounds that are suitable as monomers for preparing thermoplastic resins, such as polycarbonate resins, which have beneficial optical and mechanical properties and can be used for producing optical devices.

### BACKGROUND OF INVENTION

Optical devices, such as optical lenses made of optical resin instead of optical glass are advantageous in that they can be produced in large numbers by injection molding. Nowadays, optical resins, in particular, transparent polycarbonate resins, are frequently used for producing camera lenses. In this regard, resins with a higher refractive index are highly desirable, as they allow for reducing the size and weight of final products. In general, when using an optical material with a higher refractive index, a lens element of the same refractive power can be achieved with a surface having less curvature, so that the amount of aberration generated on this surface can be reduced. As a result, it is possible to reduce the number of lenses, to reduce the eccentric sensitivity of lenses and/or to reduce the lens thickness to thereby achieve weight reduction.

U.S. Pat. No. 9,360,593 describes polycarbonate resins having repeating units derived from binaphthyl monomers of the formula (A):

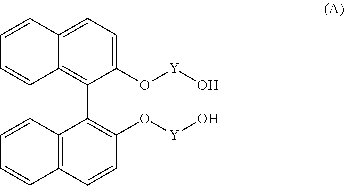

where Y is C₁-C₄-alkandiyl, in particular 1,2-ethandiyl. It is said that the polycarbonate resins have beneficial optical properties in terms of a high refractive index, a low Abbe's number, a high degree of transparency, low birefringence, and a glass transition temperature suitable for injection molding.

Co-Polycarbonates of monomers of the formula (A) with 10,10-bis(4-hydroxyphenyl)anthrone monomers and their use for preparing optical lenses are described in US 2016/0319069.

WO 2019/043060 describes thermoplastic resins for producing optical materials, where the thermoplastic resins comprise a polymerized compound of formula (B)

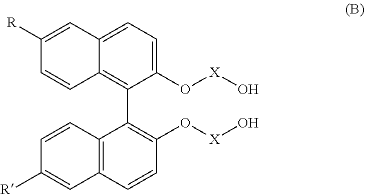

where
X is e.g. C₂-C₄-alkandiyl;
R and R′ are identical or different and selected from optionally substituted mono or polycyclic aryl having from 6 to 36 carbon atoms and optionally substituted monoor polycyclic hetaryl having a total of 5 to 36 atoms.

However, as observed by the inventors of the present application, binaphtyl derived monomers, such as those of formulae A and B above, despite their multiple advantages, suffer from the disadvantage that they form a significant proportion of undesirable cyclic oligomers when used as monomers in the production of thermoplastic resins such as in the production of polyesters and polycarbonates. These cyclic oligomers may aggravate the molecular weight build-up and/or worsen the product properties of the resin, such as reduced mechanical strength, lower glass transition temperature and/or optical properties. Unfortunately those cyclic components can hardly be removed from the resin in an efficient way. To reduce the formation of such cyclic compounds, it is typically necessary to polymerize the binaphthyl-containing monomers with relatively high amounts of co-monomers.

Without being bound to theory it is assumed that the reason for the increased formation of cyclic compounds when using these monomers is in particular related to their flexible and typically short linker units (see moieties —Y—OH and —X—OH in formulae A and B).

The inventors now found that these problems can be alleviated by the compounds of the formula (I) as described below. The use of the compounds of the formula (I) as monomers in the production of thermoplastic resins, in particular polycarbonates, will yield resins having a reduced content of undesirable cyclic oligomers and/or higher molecular weight and higher refractive index and thus have improved optical properties and/or improved mechanical properties.

Therefore, a first aspect of the present invention relates to the use of the compound of the formula (I) or a mixture thereof,

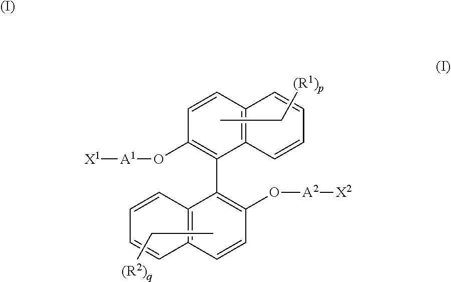

where
X¹ and X² are independently selected from —CH₂OH and —C(O)OR^{x},
   where R^{x} is selected from the group consisting of hydrogen, phenyl, benzyl and C₁-C₄-alkyl;
A¹ and A² are independently selected from the group consisting of a mono- or polycyclic arylene having from 6 to 18 carbon atoms as ring members and a mono- or polycyclic hetarylene having a total of 5 to 18 atoms, which are ring members, where 1, 2, 3 or 4 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1, 2, 3 or 4 radicals R^{Ar};
R¹ and R² are independently selected from the group consisting of halogen, C₂-C₃-alkynyl, CN, R, S(O)ₖR, NHR, OR, CH₅R′₃₋₅, NR₂, C(O)R and CH═CHR″, it being possible that R¹ and R² are identical or different if p+q>1, where s on each occurrence is 0, 1 or 2 and where k on each occurrence is 0, 1 or 2;
p and q are independently 0, 1 or 2;
R is selected from the group consisting of C₁-C₄-alkyl and a radical Q¹;
Q¹ is selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R^{Ar1} is selected from the group consisting of R¹¹, S(O)ₖR¹¹, NHR¹¹, OR¹¹, CN, halogen, CHₜR′₃₋ₜ, NR¹¹₂ and CH═CHR″, where R^{Ar1} may be identical or different if more than one is present on the same (het)aryl or (het)arylene group, where t on each occurrence is 0, 1 or 2 and where k on each occurrence is 0, 1 or 2;
R¹¹ is selected from the group consisting of C₁-C₄-alkyl and a radical Q²;
Q² is selected from the group consisting of mono- or polycyclic aryl having from 6 to 18 carbon atoms as ring members and mono- or polycyclic hetaryl having a total of 5 to 18 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R′ is selected from the group consisting of mono- or polycyclic aryl having from 6 to 18 carbon atoms as ring members and mono- or polycyclic hetaryl having a total of 5 to 18 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R″ is selected from hydrogen, methyl, phenyl and naphthyl, where phenyl and naphthyl are unsubstituted or substituted by 1, 2, 3 or 4 identical or different radicals R′″;
R′″ is selected from the group consisting of phenyl, halogen, OCH₃, CH₃, N(CH₃)₂ and C(O)CH₃;
as a monomer for producing a thermoplastic resin, selected from polyesters, polycarbonates and polyestercarbonates, in particular for producing polycarbonates.

The compounds of the formula (I) are novel, except for compounds of formula (I), where A¹ and A² are both unsubstituted 1,4-phenylene, p and q are both 0, and X¹ and X² are both —CH₂OH or —C(O)OH and further except for the compound of formula (I), where A¹ and A² are both unsubstituted 2,3-quinolinylene, p and q are both 0, and X¹ and X² are both —CH₂OH.

These compounds are known from S. I. Murahashi et al, Advanced Synthesis and Catalysis 346(2) (2004), 195-198 (https://doi.org/10.1002/adsc.200303190);
G. S. Liou et al, J. Polym. Sci. Part A 31(13), (1993), 3265-72 (https://doi.org/10.1002/poIa.1993.080311314); and
P. Rajakumar et al., Bioorganic and Medicinal Chemistry Letters 19(13) (2009), 3466-3470 (https://doi.org/10.1016/j.bmcl.2009.05.019).

Therefore, a second aspect relates to compounds of the formula (I) that are novel. In other words, the second aspect relates to compounds of the formula (I) except for compounds of formula (I), where A¹ and A² are both unsubstituted 1,4-phenylene, p and q are both 0, and X¹ and X² are both —CH₂OH or —C(O)OH and further except for the compound of formula (I), where A¹ and A² are both unsubstituted 2,3-quinolinylene, p and q are both 0, and X¹ and X² are both —CH₂OH.

Using the compounds of the formula (I) in the preparation of thermoplastic polymers, namely polycarbonates, polyesters or polyestercarbonates, will result in thermoplastic polymers bearing polymerized units of the compound of formula (I), i.e. a thermoplastic resin comprising structural units represented by formula (II) below;

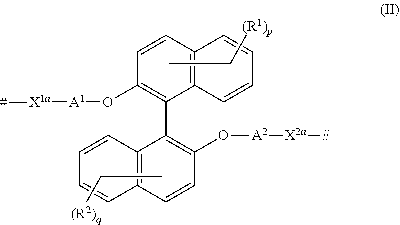

where
#represents a connection point to a neighboring structural unit; and where X^{1a} and X^{2a} are derived from X¹ and X², respectively, by replacing the —OH or —OR^{x} group of X¹ or X² with an oxo (—O—) moiety, i. e. X^{1a} and X^{2a} are O or C(O)—O, respectively, and where A¹, A², R¹, R², p and q are as defined herein above.

Due to the beneficial properties conferred by the monomers of the formula (I), the thermoplastic resins as described herein which have repeating units of the formula (II), in particular the polyesters and especially polycarbonates, are particularly suitable for the production of optical devices.

### DETAILED DESCRIPTION OF INVENTION

The compounds of formula (I) may have axial chirality due to the limited rotation along the bond between the naphthalene units and therefore compounds of the formula (I) may exist in the form of their (S)-enantiomer and their (R)-enantiomer. Consequently, the compounds of formula (I) may exist as a racemic mixture or as non-racemic mixtures or in the form of their pure (S)- and (R)-enantiomers, respectively. The present invention relates to both the racemic and the non-racemic mixtures of the enantiomers of the compounds of formula (I) and also to their pure (S)- and (R)-enantiomers, as far as these enantiomers exist.

In terms of the present invention, the term “C₁-C₄-alkandiyl group” may alternatively also be designated “C₁-C₄-alkylene group” and refers to a bivalent, saturated, aliphatic hydrocarbon radical having 1, 2, 3 or 4 carbon atoms. Examples of C₁-C₄-alkandiyl are in particular the methylene group (CH₂), linear alkandiyl such as 1,2-ethandiyl (CH₂CH₂), 1,3-propandiyl (CH₂CH₂CH₂) and 1,4-butdandiyl (CH₂CH₂CH₂CH₂), but also branched alkandiyl such as 1-methyl-1,2-ethandiyl, 1-methyl-1,2-propandiyl, 2-methyl-1,2-propandiyl, 2-methyl-1,3-propandiyl and 1,3-butandiyl.

In terms of the present invention, the term “monocyclic aryl” refers to a monovalent aromatic monocyclic radical, such as in particular phenyl.

In terms of the present invention, the term “monocyclic hetaryl” refers to a monovalent heteroaromatic monocyclic radical, i.e. a heteroaromatic monocycle linked by a single covalent bond to the remainder of the molecule, where the ring member atoms are part of a conjugate π-electron system, where the heteroaromatic monocycle has 5 or 6 ring atoms, which comprise as heterocyclic ring members 1, 2, 3 or 4 nitrogen atoms or 1 oxygen atom and 0, 1, 2 or 3 nitrogen atoms, or 1 sulphur atom and 0, 1, 2 or 3 nitrogen atoms, where the remaining ring atoms are carbon atoms. Examples include furyl (=furanyl), pyrrolyl (=1H-pyrrolyl), thienyl (=thiophenyl), imidazolyl (=1H-imidazolyl), pyrazolyl (=1H-pyrazolyl), 1,2,3-triazolyl, 1,2,4-triazolyl, tetrazolyl, oxazolyl, thiazolyl, isoxazolyl, isothiazolyl, 1,3,4-oxadiazolyl, 1,3,4-thiadiazolyl, pyridyl (=pyridinyl), pyrazinyl, pyridazinyl, pyrimidinyl and triazinyl.

In terms of the present invention, the term “mono- or polycyclic aryl” refers to a monovalent aromatic monocyclic radical as defined herein or to a monovalent aromatic polycyclic radical, i.e. a polycyclic arene linked by a single covalent bond to the remainder of the molecule, where the polycyclic arene is
(i) an aromatic polycyclic hydrocarbon, i.e. a completely unsaturated polycyclic hydrocarbon, where each of the carbon atoms is part of a conjugate π-electron system,
(ii) a polycyclic hydrocarbon which bears at least 1 phenyl ring which is fused to a saturated or unsaturated 4 to 10-membered mono- or bicyclic hydrocarbon ring,
(iii) a polycyclic hydrocarbon which bears at least 2 phenyl rings which are linked to each other by a covalent bond or which are fused to each other directly and/or which are fused to a saturated or unsaturated 4 to 10-membered mono- or bicyclic hydrocarbon ring.

Mono- or polycyclic aryl has from 6 to 26, e.g. 6, 9, 10, 12, 13, 14, 16, 17, 18, 19, 20, 22 or 24 carbon atoms as ring atoms, often from 6 to 22 carbon atoms, in particular 6 to 18 carbon atoms, especially 6 to 14 carbon atoms, especially 6, 10, 12, 13, 14, 16, 17 or 18 carbon atoms. Polycyclic aryl typically has 9 to 26 carbon atoms as ring atoms, in particular from 10 to 18 carbon atoms, especially 10 to 14 carbon atoms, e.g. 10, 12, 13, 14, 16 or 18 carbon atoms, as ring members.

In this context, polycyclic aryl bearing 2, 3 or 4 phenyl rings which are linked to each other via a single bond include e.g. biphenylyl and terphenylyl. Polycyclic aryl bearing 2, 3 or 4 phenyl rings which are directly fused to each other include e.g. naphthyl, anthracenyl, phenanthrenyl, pyrenyl, triphenylenyl, chrysenyl and benzo[c]phenanthrenyl. Polycyclic aryl bearing 2, 3 or 4 phenyl rings which are fused to a saturated or unsaturated 4- to 10-membered mono- or bicyclic hydrocarbon ring include e.g. 9H-fluorenyl, biphenylenyl, tetraphenylenyl, acenaphthenyl (1,2-dihydroacenaphthylenyl), acenaphthylenyl, 9,10-dihydroanthracen-1-yl, 1,2,3,4-tetrahydrophenanthrenyl, 5,6,7,8-tetrahydrophenanthrenyl, cyclopent[fg]acenaphthylenyl, phenalenyl, fluoranthenyl, benzo[k]fluoranthenyl, perylenyl, 9,10-dihydro-9,10[1′,2′]-benzenoanthracenyl, dibenzo[a,e][8]annulenyl, 9,9′-spirobi[9H-fluoren]yl and spiro[1H-cyclobuta[de]naphthalene-1,9′-[9H]fluoren]yl.

Mono- or polycyclic aryl includes, by way of example phenyl, naphthyl, 9H-fluorenyl, phenanthryl, anthracenyl, pyrenyl, chrysenyl, benzo[c]phenanthrenyl, acenaphthenyl, acenaphthylenyl, 2,3-dihydro-1H-indenyl, 5,6,7,8-tetrahydro-naphthalenyl, cyclopent[fg]acenaphthylenyl, 2,3-dihydrophenalenyl, 9,10-dihydroanthracen-1-yl, 1,2,3,4-tetrahydrophenanthrenyl, 5,6,7,8-tetrahydrophenanthrenyl, fluoranthenyl, benzo[k]fluoranthenyl, biphenylenyl, triphenylenyl, tetraphenylenyl, 1,2-dihydroacenaphthylenyl, dibenzo[a,e][8]annulenyl, perylenyl, biphenylyl, terphenylyl, naphthylenphenyl, phenanthrylphenyl, anthracenylphenyl, pyrenylphenyl, 9H-fluorenyl-phenyl, di(naphthylen)phenyl, naphthylenbiphenyl, tri(phenyl)phenyl, tetra(phenyl)phenyl, pentaphenyl(phenyl), phenylnaphthyl, binaphthyl, phenanthrylnaphthyl, pyrenylnaphthyl, phenylanthracenyl, biphenylanthracenyl, naphthalenylanthracenyl, phenanthrylanthracenyl, dibenzo[a,e][8]annulenyl, 9,10-dihydro-9,10[1′,2′]benzoanthracenyl, 9,9′-spirobi-9H-fluorenyl and spiro[1H-cyclobuta[de]naphthalene-1,9′-[9H]fluoren]yl.

In terms of the present invention, the term “mono- or polycyclic hetaryl” refers to a monovalent heteroaromatic monocyclic radical as defined herein or to a monovalent heteroaromatic polycyclic radical, i.e. a polycyclic hetarene linked by a single covalent bond to the remainder of the molecule, where
(i) the polycyclic hetarene bears a heteroaromatic monocycle as defined above and at least one, e.g. 1, 2, 3, 4 or 5, further aromatic rings selected from phenyl and heteroaromatic monocycles as defined above, where the aromatic rings of the polycyclic hetarene are linked to each other by a covalent bond and/or fused to each other directly and/or fused to a saturated or unsaturated 4 to 10-membered mono- or bicyclic hydrocarbon ring, or
(ii) the polycyclic hetarene bears at least one saturated or partially or fully unsaturated 5-, 6-, 7- or 8-membered heterocyclic ring bearing 1, 2 or 3 heteroatoms selected from oxygen, sulphur and nitrogen as ring atoms, such as 2H-pyran, 4H-pyran, thiopyran, 1,4-dihydropyridin, 4H-1,4-oxazin, 4H-1,4-thiazin, 1,4-dioxin, oxepin, thiepin, dioxin, dithiin, dioxepin, dithiepin, dioxocine, dithiocine and at least one, e.g. 1, 2, 3, 4 or 5, aromatic rings selected from phenyl and heteroaromatic monocycles as defined above, where at least one of the aromatic rings is directly fused to the saturated or partially unsaturated 5- to 8-membered heterocyclic ring and where the aromatic rings of the polycyclic hetarene are linked to each other by a covalent bond or fused to each other directly and/or fused to a saturated or unsaturated 4 to 10-membered mono- or bicyclic hydrocarbon ring.

Mono- or polycyclic hetaryl has from 5 to 26, often from 5 to 22, in particular 5 to 18 and especially 5 to 14 ring atoms, which comprise 1, 2, 3 or 4 atoms, in particular 1 or 2 atoms selected from nitrogen atoms, sulphur atoms and oxygen atoms, where the remainder of the ring atoms are carbon atoms. Polycyclic hetaryl generally has from 9 to 26, often from 9 to 20 ring atoms, in particular 9 to 18 ring atoms, especially 9 to 14 ring atoms, which comprise 1, 2, 3 or 4 atoms, in particular 1 or 2 atoms selected from nitrogen atoms, sulphur atoms and oxygen atoms, where the remainder of the ring atoms are carbon atoms.

Examples of polycyclic hetaryl include, but are not limited to, benzofuryl, benzothienyl, dibenzofuranyl (=dibenzo[b,a]furanyl), dibenzothienyl (=dibenzo[b,a]thienyl), naphtho-furyl, naphthothienyl, furo[3,2-b]furanyl, furo[2,3-b]furanyl, furo[3,4-b]furanyl, thieno[3,2-b]thienyl, thieno[2,3-b]thienyl, thieno[3,4-b]thienyl, oxanthrenyl, thianthrenyl, indolyl (=1H-indolyl), isoindolyl (=2H-isoindolyl), carbazolyl, indolizinyl, benzopyrazolyl, ben-zimidazolyl, benzoxazolyl, benzothiazolyl, benzo[c,a]indolyl, 1H-benzo[g]indolyl, quinolinyl, isoquinolinyl, acridinyl, phenazinyl, quinazolinyl, quinoxalinyl, phenoxazinyl, phen-thiazinyl, benzo[b][1,5]naphthyridinyl, cinnolinyl, 1,5-naphthyridinyl, 1,8-naphthyridinyl, phenylpyrrolyl, naphthylpyrrolyl, dipyridyl, phenylpyridyl, naphthylpyridyl, pyrido[4,3-b]indolyl, pyrido[3,2-b]indolyl, pyrido[3,2-g]quinolinyl, pyrido[2,3-b][1,8]naphthyridinyl, pyrrolo[3,2-b]pyridinyl, pteridinyl, puryl, 9H-xanthenyl, 9H-thioxanthenyl, 2H-chromenyl, 2H-thiochromenyl, phenanthridinyl, phenanthrolinyl, benzo[1,2-b:4,3-b′]difuranyl, benzo[1,2-b:6,5-b′]difuranyl, benzo[1,2-b:5,4-b′]difuranyl, benzo[1,2-b:4,5-b′]difuranyl, naphthofuranyl, benzo[b]naphtho[1,2-d]furanyl, benzo[b]naphtho[2,3-a]furanyl, benzo[b]naphtho[2,1-d]furanyl, tribenzo[b,d,f]oxepinyl, dibenzo[b,d]thienyl, naphtho[1,2-b]thienyl, naphtho[2,3-b]thienyl, naphtho[2,1-b]thienyl, benzo[b]naphtho[1,2-d]thienyl, benzo[b]naphtho[2,3-a]thienyl, benzo[b]naphtho[2,1-d]thienyl, 6H-dibenzo[b,a]thiopyranyl, 5H,9H-[1]benzothiopyrano[5,4,3-c,d,e][2]benzothiopyranyl, 5H,10H-[1]benzothiopyrano[5,4,3-c,d,e][2]benzothiopyranyl, benzo[1,2-6:4,3-b′]bisthienyl, benzo[1,2-b6,5-b′]bisthienyl, benzo[1,2-b:5,4-b′]bisthienyl, benzo[1,2-b:4,5-b′]bisthienyl, 1,4-benzodithiinyl, naphtho[1,2-b][1,4]dithiinyl, naphtho[2,3-b][1,4]dithiinyl, thianthrenyl, benzo[a]thianthrenyl, benzo[b]thianthrenyl, dibenzo[a,c]thianthrenyl, dibenzo[a,h]thianthrenyl, dibenzo[a,i]thianthrenyl, dibenzo[a,j]thianthrenyl, dibenzo[b,i]thianthrenyl, 2H-naphtho[1,8-b,c]thienyl, 5H-phenanthro[4,5-b,c,d]thiopyranyl, 10,11-dihydrodibenzo[b,f]thiepinyl, 6,7-dihydrodibenzo[b,d]thiepinyl, dibenzo[b,f]thiepinyl, dibenzo[b,d]thiepinyl, 6H-dibenzo[d,f][1,3]dithiepinyl, tribenzo[b,d,f]thiepinyl, benzothieno[3,4-c,d]thieno[2,3,4-j,k][2]benzothiepinyl, dinaphtho[1,8-bc-1′,8′-f,g][1,5]dithiocinyl, furo[3,2-g]quinolinyl, furo[2,3-g]quinolinyl, furo[2,3-g]quinoxalinyl, benzo[g]chromenyl, thieno[3,2-f][1]benzothienyl, thieno[2,3-f][1]benzothienyl, thieno[3,2-g]quinolinyl, thieno[2,3-g]quinolinyl, thieno[2,3-g]quinoxalinyl, benzo[g]thiochromenyl, pyrrolo[3,2,1-h,i]indolyl, benzo[g]quinoxalinyl, benzo[f]quinoxalinyl, and benzo[h]isoquinolinyl.

In terms of the present invention, the term “monocyclic arylene” refers to a bivalent aromatic monocyclic radical, such as in particular phenylene.

In terms of the present invention, the term “monocyclic hetarylene” refers to a bivalent heteroaromatic monocyclic radical, i.e. a heteroaromatic monocycle linked by two single covalent bonds to the two remaining parts of the molecule, where the ring member atoms are part of a conjugate π-electron system, where the heteroaromatic monocycle has 5 or 6 ring atoms, which comprise as heterocyclic ring members 1, 2, 3 or 4 nitro-gen atoms or 1 oxygen atom and 0, 1, 2 or 3 nitrogen atoms, or 1 sulphur atom and 0, 1, 2 or 3 nitrogen atoms, where the remaining ring atoms are carbon atoms. Examples include furylene (=furanylene), pyrrolylene (=1H-pyrrolylene), thienylene (=thio-phenylene), imidazolylene (=1H-imidazolylene), pyrazolylene (=1H-pyrazolylene), 1,2,3-triazolylene, 1,2,4-triazolylene, tetrazolylene, oxazolylene, thiazolylene, isoxazol-ylene, isothiazolylene, 1,3,4-oxadiazolylene, 1,3,4-thiadiazolylene, pyridylene (=pyridinylene), pyrazinylene, pyridazinylene, pyrimidinylene and triazinylene.

In terms of the present invention, the term “mono- or polycyclic arylene” refers to a bivalent aromatic monocyclic radical as defined herein or to a bivalent aromatic polycyclic radical, i.e. a polycyclic arene linked by two single covalent bonds to the two remaining parts of the molecule, where the polycyclic arene is
(i) an aromatic polycyclic hydrocarbon, i.e. a completely unsaturated polycyclic hydrocarbon, where each of the carbon atoms is part of a conjugate π-electron system,
(ii) a polycyclic hydrocarbon which bears at least 1 phenyl ring which is fused to a saturated or unsaturated 4 to 10-membered mono- or bicyclic hydrocarbon ring,
(iii) a polycyclic hydrocarbon which bears at least 2 phenyl rings which are fused to each other directly, and/or which are fused to a saturated or unsaturated 4 to 10-membered mono- or bicyclic hydrocarbon ring.

Mono- or polycyclic arylene has from 6 to 18, e.g. 6, 9, 10, 12, 13, 14, 16, 17 or 18 carbon atoms as ring atoms, in particular from 6 to 16 carbon atoms, especially 6 to 14 carbon atoms as ring atoms. Polycyclic arylene typically has 10 to 18 carbon atoms as ring atoms, in particular from 10 to 16 carbon atoms, especially 10 to 14 carbon atoms, e. g. 10, 12, 13 or 14 carbon atoms.

In this context, polycyclic arylene bearing 2 or 3 phenyl rings which are directly fused to each other include e.g. naphthylene, anthracenylene, phenanthrenylene, pyrenylene, triphenylenylene, chrysenylene and benzo[c]phenanthrenylene. Polycyclic arylene bearing 2 or 3 phenyl rings which are fused to a saturated or unsaturated 4- to 10-membered mono- or bicyclic hydrocarbon ring include e.g. 9H-fluorenylene, biphenylenylene, acenaphthenylene (1,2-dihydroacenaphthylenylene), acenaphthylenylene, 9,10-dihydroanthracen-1-ylene, 1,2,3,4-tetrahydrophenanthrenylene, 5,6,7,8-tetrahydrophenanthrenylene, cyclopent[fg]acenaphthylenylene, phenalenylene, fluoranthenylene, benzo[k]fluoranthenylene, perylenylene, 9,10-dihydro-9,10[1′,2′]-benzenoanthracenylene, dibenzo[a,e][8]annulenylene, 9,9′-spirobi[9H-fluoren]ylene and spiro[1H-cyclobuta[de]naphthalene-1,9′-[9H]fluoren]ylene.

Mono- or polycyclic arylene includes, by way of example phenylene, naphthylene, 9H-fluorenylene, phenanthrylene, anthracenylene, pyrenylene, chrysenylene, benzo[c]phenanthrenylene, acenaphthenylene, acenaphthylenylene, 2,3-dihydro-1H-indenylene, 5,6,7,8-tetrahydro-naphthalenylene, cyclopent[fg]acenaphthylenylene, 2,3-dihydrophenalenylene, 9,10-dihydroanthracen-1-ylene, 1,2,3,4-tetrahydrophenanthrenylene, 5,6,7,8-tetrahydrophenanthrenylene, fluoranthenylene, biphenylenylene, triphenylenylene, 1,2-dihydroacenaphthylenylene, dibenzo[a,e][8]annulenylene, naphthylenphenylene, phenanthrylphenylene, anthra-cenylphenylene, pyrenylphenylene, 9H-fluorenylphenylene, dibenzo[a,e][8]annulenylene, 9,10-dihydro-9,10[1′,2′]benzoanthracenylene.

In terms of the present invention, the term “mono- or polycyclic hetarylene” refers to a bivalent heteroaromatic monocyclic radical as defined herein or to a bivalent heteroaromatic polycyclic radical, i.e. a polycyclic hetarene linked by two single covalent bonds to the two remaining parts of the molecule, where
(i) the polycyclic hetarene bears a heteroaromatic monocycle as defined above and at least one, e.g. 1, 2 or 3, further aromatic rings selected from phenyl and heteroaromatic monocycles as defined above, where the aromatic rings of the polycyclic hetarene are linked to each other by a covalent bond and/or fused to each other directly and/or fused to a saturated or unsaturated 4 to 10-membered mono- or bicyclic hydrocarbon ring, or
(ii) the polycyclic hetarene bears at least one saturated or partially or fully unsaturated 5-, 6-, 7- or 8-membered heterocyclic ring bearing 1, 2 or 3 heteroatoms selected from oxygen, sulphur and nitrogen as ring atoms, such as 2H-pyran, 4H-pyran, thiopyran, 1,4-dihydropyridin, 4H-1,4-oxazin, 4H-1,4-thiazin, 1,4-dioxin, oxepin, thiepin, dioxin, dithiin, dioxepin, dithiepin, dioxocine, dithiocine and at least one, e.g. 1, 2, 3, 4 or 5, aromatic rings selected from phenyl and heteroaromatic monocycles as defined above, where at least one of the aromatic rings is directly fused to the saturated or partially unsaturated 5- to 8-membered heterocyclic ring and where the aromatic rings of the polycyclic hetarene are linked to each other by a covalent bond or fused to each other directly and/or fused to a saturated or unsaturated 4 to 10-membered mono- or bicyclic hydrocarbon ring.

Mono- or polycyclic hetarylene has from 5 to 18, often from 5 to 16 ring atoms, in particular 5 to 16 ring atoms, which comprise 1, 2, 3 or 4 atoms, in particular 1 or 2 atoms, selected from nitrogen atoms, sulphur atoms and oxygen atoms, where the remainder of the ring atoms are carbon atoms. Polycyclic hetaryl generally has from 9 to 18, often from 9 to 16 ring atoms, in particular 9 to 14 ring atoms, which comprise 1, 2, 3 or 4 atoms, in particular 1 or 2 atoms, which are selected from nitrogen atoms, sulphur atoms and oxygen atoms, where the remainder of the ring atoms are carbon atoms.

Examples of polycyclic hetarylene include, but are not limited to, benzofurylene, benzothienylene, dibenzofuranylene (=dibenzo[b,a]furanylene), dibenzothienylene (=dibenzo[b,a]thienylene), naphthofurylene, naphthothienylene, furo[3,2-b]furanylene, furo[2,3-b]furanylene, furo[3,4-b]furanylene, thieno[3,2-b]thienylene, thieno[2,3-b]thienylene, thieno[3,4-b]thienylene, oxanthrenylene (=dibenzo[1,4]dioxinylene), thianthrenylene, indolylene (=1H-indolylene), isoindolylene (=2H-isoindolylene), carbazolylene, indolizinylene, benzopyrazolylene, benzimidazolylene, benzoxazolylene, benzothiazolylene, benzo[c,a]indolylene, 1H-benzo[g]indolylene, quinolinylene, isoquinolinylene, acridinylene, phenazinylene, quinazolinylene, quinoxalinylene, phenoxazinylene, phenthiazinylene, benzo[b][1,5]naphthyridinylene, cinnolinylene, 1,5-naphthyridinylene, 1,8-naphthyridinylene, phenylpyrrolylene, naphthylpyrrolylene, di-pyridylene, phenylpyridylene, naphthylpyridylene, pyrido[4,3-b]indolylene, pyrido[3,2-b]indolylene, pyrido[3,2-g]quinolinylene, pyrido[2,3-b][1,8]naphthyridinylene, pyrrolo[3,2-b]pyridinylene, pteridinylene, purylene, 9H-xanthenylene, 9H-thioxanthenylene, 2H-chromenylene, 2H-thiochromenylene, phenanthridinylene, phenanthrolinylene, benzo[1,2-b:4,3-b′]difuranylene, benzo[1,2-b:6,5-b′]difuranylene, benzo[1,2-b:5,4-b′]difuranylene, benzo[1,2-b:4,5-b′]difuranylene, naphthofuranylene, benzo[b]naphtho[1,2-d]furanylene, benzo[b]naphtho[2,3-a]furanylene, benzo[b]naphtho[2,1-d]furanylene, tribenzo[b,d,f]oxepinylene, dibenzo[b,d]thienylene, naphtho[1,2-b]thienylene, naphtho[2,3-b]thienylene, naphtho[2,1-b]thienylene, benzo[b]naphtho[1,2-o]thienylene, 6H-dibenzo[b,d]thiopyranylene, 5H,9H-[1]benzothiopyrano[5,4,3-c,d,e][2]benzothiopyranylene, 5H,10H-[1]benzothiopyrano[5,4,3-c,d,e][2]benzothiopyranylene, benzo[1,2-b:4,3-b′]bisthienylene, benzo[1,2-b-6,5-b′]bisthienylene, benzo[1,2-b-5,4-b′]bisthienylene, benzo[1,2-b:4,5-b′]bisthienylene, 1,4-benzodithiinylene, naphtho[1,2-b][1,4]dithiinylene, naphtho[2,3-b][1,4]dithiinylene, thianthrenylene, benzo[a]thianthrenylene, benzo[b]thianthrenylene, dibenzo[a,c]thianthrenylene, dibenzo[a,h]thianthrenylene, 2H-naphtho[1,8-b,c]thienylene, 5H-phenanthro[4,5-b,c,d]thiopyranylene, 10,11-dihydrodibenzo[b,f]thiepinylene, 6,7-dihydrodibenzo[b,d]thiepinylene, dibenzo[b,f]thiepinylene, dibenzo[b,d]thiepinylene, 6H-dibenzo[d,f][1,3]dithiepinylene, furo[3,2-g]quinolinylene, furo[2,3-g]quinolinylene, furo[2,3-g]quinoxalinylene, benzo[g]chromenylene, thieno[3,2-f][1]benzothienylene, thieno[2,3-f][1]benzothienylene, thieno[3,2-g]quinolinylene, thieno[2,3-g]quinolinylene, thieno[2,3-g]quinoxalinylene, benzo[g]thiochromenylene, pyrrolo[3,2,1-h,i]indolylene, benzo[g]quinoxalinylene, benzo[f]quinoxalinylene, and benzo[h]isoquinolinylene.

In terms of the present invention, the suffix “-ylene” means, as customary in the art, that the respective het(arene) moiety is in the form of its diradical. Accordingly, the suffix “-ylene”, as e.g. in phenylene or 1,4-phenylene, is used here synonymously with the suffix “-diyl”, as e.g. in phendiyl or phen-1,4-diyl.

In terms of the present invention, a “structural unit” is a structural element which is present repeatedly in the polymer backbone of the thermoplastic resin. Therefore, the terms “structural unit” and “repeating unit” are used synonymously.

In terms of the present invention, the term “optical device” refers to a device that is transparent for visible light and manipulates light beams, in particular by refraction.

Optical devices include but are not limited to prisms, lenses, optical films and combinations thereof, especially lenses for cameras and lenses for glasses.

The remarks made below as to preferred embodiments of the variables (substituents) of the compounds of formula (I) and of the structural units of formula (II) are valid on their own as well as preferably in combination with each other.

The remarks made below concerning preferred embodiments of the variables further are valid on their own as well as preferably in combination with each other concerning the compounds of formula (I) and the structural units of formula (II), where applicable, as well as concerning the uses according to the invention.

In formula (I) and likewise in formula (II), the variables X¹, X², A¹, A², R¹, R², p and q on their own or preferably in any combination preferably have the following meanings: Preference is given to those variables X¹ and X² in formula (I) that are independently selected —CH₂OH and —C(O)OR^{x}, where R^{x} is selected from the group consisting of hydrogen and C₁-C₄-alkyl, and accordingly to those variables X^{1a} and X^{2a} in formula (II) that are independently selected from —CH₂O— and —C(O)O—.

In a preferred group (1) of embodiments, the variables X¹ and X² in formula (I) are both —CH₂OH and accordingly the variables X^{1a} and X^{2a} in formula (II) are both —CH₂O—.

In another group (2) of embodiments the variables X¹ and X² in formulae (I) and (II) are independently —C(O)OR^{x} and accordingly the variables X^{1a} and X^{2a} in formula (II) are both —C(O)O—, where R^{x} is selected from the meanings defined herein for R^{x}, and in particular selected from the group consisting of hydrogen, phenyl, benzyl and C₁-C₄-alkyl, preferably hydrogen and C₁-C₄-alkyl, more preferably hydrogen, methyl and ethyl, and in particular hydrogen and methyl.

In a particular subgroup (2′) of embodiments the variables X¹ and X² in formula (I) have the same meaning, which is selected from the meanings defined in group (2) of embodiments for X¹ and X².

In preferred group (3) of embodiments, which is a combination of groups (1) and (2) of embodiments, the variables X¹ and X² in formula (I) are independently selected from —CH₂OH and —C(O)OR^{x}, wherein R^{x} is hydrogen or C₁-C₄-alkyl, in particular independently selected from —CH₂OH, —C(O)OH, —C(O)OCH₃ and —C(O)OCH₂CH₃, and specifically independently selected from —CH₂OH, —C(O)OH and —C(O)OCH₃. Correspondingly, in this preferred group (4) of embodiments the variables X^{1a} and X^{2a} in formula (II) are independently selected from —CH₂O— and —C(O)O—.

In a particular subgroup (3′) of embodiments the variables X¹ and X² in formula (I) have the same meaning, which is selected from the meanings defined herein for X¹ and X², especially those mentioned as preferred, and in particular selected from the meanings defined in groups (3), of embodiments and, likewise, the variables X^{1a} and X^{2a} in formula (II) have the same meaning, which is selected from the meanings defined in groups (3) of embodiments.

In a preferred group (4) of embodiments the variables A¹ and A² in formulae (I) and (II) are independently selected from the group consisting of mono- or polycyclic arylene having from 6 to 16, in particular 6 to 14, carbon atoms as ring members and mono- or polycyclic hetarylene having from 9 to 16 atoms as ring members, where 1, 2, 3 or 4 of these atoms, in particular 1 or 2 of these atoms are nitrogen, oxygen or sulfur atoms, and in particular 1 or 2 of these atoms are oxygen or sulfur atoms, while the remainder of these atoms are carbon atoms. In the preferred group (4) of embodiments, the mono- or polycyclic arylene and mono- or polycyclic hetarylene are unsubstituted or carry 1, 2, 3 or 4, in particular 1 or 2, radicals R^{Ar1}, where R^{Ar1} has one of the meanings defined herein, especially one of the meanings mentioned as preferred.

In a more preferred subgroup (4.1) of group (4) of embodiments, A¹ and A² are independently selected from the group consisting of phenylene, naphthylene, 1,2-dihydroacenaphthylene, biphenylylene, 1,1′-oxydiphenylene, 1,1′-thiodiphenylene, 9H-fluorenylene, 11H-benzo[a]fluorenylene, 11H-benzo[b]fluorenylene, 7H-benzo[c]fluorenylene, anthracylene, phenanthrylene, benzo[c]phenanthrylene, pyrenylene, chrysenylene, picenylene, triphenylenylene, furanylene, benzo[b]furanylene, dibenzo[b,d]furanylene, naphtho[1,2-b]furanylene, naphtho[2,3-b]furanylene, naphtho[2,1-b]furanylene, benzo[b]naphtho[1,2-d]furanylene, benzo[b]naphtho[2,3-d]furanylene, benzo[b]naphtho[2,1-d]furanylene, benzo[1,2-b:4,3-b′]difuranylene, benzo[1,2-b:6,5-b′]difuranylene, benzo[1,2-b:5,4-b′]difuranylene, benzo[1,2-b:4,5-b′]difuranylene, 9H-xanthylene, tribenzo[b,d,f]oxepinylene, dibenzo[1,4]dioxinylene, 2H-naphtho[1,8-d,e][1,3]dioxinylene, phenoxathiinylene, dinaphtho[2,3-b:2′,3′-d]furanylene, oxanthrenylene, benzo[a]oxanthrenylene, benzo[b]oxanthrenylene, thienylene, benzo[b]thienylene, dibenzo[b,d]thienylene, naphtho[1,2-b]thienylene, naphtho[2,3-b]thienylene, naphtho[2,1-b]thienylene, benzo[b]naphtho[1,2-o]thienylene, benzo[b]naphtho[2,3-d]thienylene, benzo[b]naphtho[2,1-d]thienylene, benzo[1,2-b-4,3-b′]dithienylene, benzo[1,2-b-6,5-b′]dithienylene, benzo[1,2-b-5,4-b′]dithienylene, benzo[1,2-b:4,5-b′]dithienylene, 9H-thioxanthylene, 6H-dibenzo[b,d]thiopyranylene, 1,4-benzodithiinylene, naphtho[1,2-b][1,4]dithiinylene, naphtho[2,3-b][1,4]dithiinylene, thianthrenylene, benzo[a]thianthrenylene, benzo[b]thianthrenylene, dibenzo[a,c]thianthrenylene, dibenzo[a,h]thianthrenylene, dibenzo[a,i]thianthrenylene, dibenzo[a,j]thianthrenylene, dibenzo[b,d]thianthrenylene, 2H-naphtho[1,8-b,c]thienylene, dibenzo[b,d]thiepinylene, dibenzo[b,f]thiepinylene, 5H-phenanthro[4,5-b,c,d]thiopyranylene, tribenzo[b,d,f]thiepinylene, 2,5-dihydronaphtho[1,8-b,c.4,5-b′,c′]dithienylene, 2,6-dihydronaphtho[1,8-b,c.5,4-b′,c′]dithienylene, tribenzo[a,c,i]thianthrenylene, benzo[b]naphtho[1,8-e,f][1,4]dithiepinylene, dinaphtho[2,3-b:2′,3′-d]thienylene, 5H-phenanthro[1,10-b,c]thienylene, 7H-phenanthro[1,10-c,b]thienylene, dibenzo[d,d′]benzo[1,2-b-4,5-b′]dithienylene and dibenzo[d,d′]benzo[1,2-b:5,4-b′]dithienylene, where the aforementioned mono- or polycyclic arylene and mono- or polycyclic hetarylene are unsubstituted or carry 1 or 2 radicals R^{Ar1}.

In an especially preferred subgroup (4.2) of group (4) of embodiments, A¹ and A² are independently selected from the group consisting of phenylene, naphthylene, benzo[b]thienylene, benzo[b]furanylene, biphenylylene, 9H-fluorenylene, oxanthrenylene, phenoxathiinylene, thianthrenylene, 9H-xanthylene and 9H-thioxanthylene, where the aforementioned mono- or polycyclic arylene and mono- and polycyclic hetarylene are unsubstituted or carry 1 or 2 radicals R^{Ar1}.

In a particularly preferred subgroup (4.3) of group (4) of embodiments, A¹ and A² are independently selected from the group consisting of phenylene, naphthylene, biphenylylene, benzo[b]furanylene, dibenzo[b,d]furanylene, benzo[b]thienylene, dibenzo[b,d]thienylene, 9H-fluorenylene, and thianthrenylene, where phenylene, naphthylene, biphenylylene, benzo[b]furanylene, dibenzo[b,a]furanylene, benzo[b]thienylene, dibenzo[b,a]thienylene, 9H-fluorenylene, and thianthrenylene are unsubstituted or carry 1 or 2 radicals R^{Ar1}.

In a particularly preferred subgroup (4.4) of group (4) of embodiments, A¹ and A² are independently selected from the group consisting of phenylene, naphthylene, biphenylylene, benzo[b]furanylene, dibenzo[b,a]furanylene, benzo[b]thienylene, dibenzo[b,a]thienylene, 9H-fluorenylene, and thianthrenylene, where phenylene, naphthylene, biphenylylene, benzo[b]furanylene, dibenzo[b,a]furanylene, benzo[b]thienylene, dibenzo[b,a]thienylene, 9H-fluorenylene, and thianthrenylene are unsubstituted or substituted by 1 radical Q².

A preferred subgroup (4.a) of the group (4) of embodiments, relates to compounds of the formula (I), where each of the moieties A¹ and A² comprises a phenylene ring, which may bear one or two fused rings selected from fused benzene rings and fused 5- or 6-membered heteroaromatic rings. The moieties A¹ and A² may be unsubstituted or bear 1 or 2 radicals R^{Ar1}, where R^{Ar1} is in particular a radical Q². Amongst the compounds of group (4.a) of embodiments preference is given to those compounds, wherein the group X¹ or X² and the atom —O— are connected in the para-positions of the phenylene ring of A¹ or A². These compounds are also referred to the para-isomers of group (4.a) of embodiments. Amongst the compounds of group (4.a) of embodiments, particular preference is given to the compounds of formula (I), where A¹ and A² are both 1,4-phenylene or are both 1,4-naphthylene or are both 2,6-naphthylene, where 1,4-phenylene, 1,4-naphthylene and 2,6-naphthylene may be unsubstituted or bear 1 or 2 radicals R^{Ar1}, where R^{Ar1} is in particular a radical Q².

In an especially preferred subgroup (4.5) of group (4) of embodiments, A¹ and A² are independently selected from the group consisting of 1,4-phenylene, 1,3-phenylene, 1,2-phenylene, 1,4-naphthylene, 1,2-naphthylene, 2,1-naphthylene, 2,6-naphthylene, 4,4′-biphenylylene, 3,3′-biphenylylene, 2,2′-biphenylylene, 2,4-dibenzo[b,d]thienylene, 2,6-dibenzo[b,d]thienylene, 4,2-dibenzo[b,d]thienylene, 2,8-dibenzo[b,a]thienylene, 4,6-dibenzo[b,d]thienylene, 6,2-dibenzo[b,d]thienylene, 2,4-dibenzo[b,d]furanylene, 2,6-dibenzo[b,d]furanylene, 4,2-dibenzo[b,d]furanylene, 2,8-dibenzo[b,d]furanylene, 4,6-dibenzo[b,d]furanylene, 6,2-dibenzo[b,d]furanylene, 1,2-thianthrenylene, 2,1-thianthrenylene, 3,2-thianthrenylene, 1,3-thianthrenylene, 1,4-thianthrenylene, 1,6-thianthrenylene, 2,7-thianthrenylene, 2,8-thianthrenylene, 1,9-thianthrenylene, 2,7-9H-fluroenylene, 9,2-9H-fluroenylene, 2,9-9H-fluroenylene, 2-(phenyl)-1,4-phenylene, 3-(phenyl)-1,4-phenylene, 2-(1-naphthyl)-1,4-phenylene, 3-(1-naphthyl)-1,4-phenylene, 2-(2-naphthyl)-1,4-phenylene, 3-(2-naphthyl)-1.4-phenylene, 2-(9-phenanthryl)-1,4-phenylene, 3-(9-phenanthryl)-1,4-phenylene, 2-(dibenzo[b,d]thien-4-yl)-1,4-phenylene, 3-(dibenzo[b,d]thien-4-yl)-1,4-phenylene, 2-(thianthren-1-yl)-1,4-phenylene, 3-(thianthren-1-yl)-1,4-phenylene, 2-phenyl-1,4-naphthylene, 2-(1-naphthyl)-1,4-naphthylene, 2-(2-naphthyl)-1,4-naphthylene, 2-(9-phenanthryl)-1,4-naphthylene, 2-(dibenzo[b,d]thien-4-yl)-1,4-naphthylene, 2-(thianthren-1-yl)-1,4-naphthylene, 8-phenyl-2,6-naphthylene, 8-(1-naphthyl)-2,6-naphthylene, 8-(2-naphthyl)-2,6-naphthylene, 8-(9-phenanthryl)-2,6-naphthylene, 8-(dibenzo[b,d]thien-4-yl)-2,6-naphthylene and 8-(thianthren-1-yl)-2,6-naphthylene.

In a preferred group (4′) of embodiments the variables A¹ and A² in formulae (I) and (II) are independently selected from the group consisting of polycyclic arylene having from 10 to 16, in particular 10 to 14, carbon atoms as ring members and mono- or polycyclic hetarylene having from 9 to 16 atoms as ring members, where 1, 2, 3 or 4 of these atoms, in particular 1 or 2 of these atoms are nitrogen, oxygen or sulfur atoms, and in particular 1 or 2 of these atoms are oxygen or sulfur atoms, while the remainder of these atoms are carbon atoms. In the preferred group (4) of embodiments, the polycyclic arylene and mono- or polycyclic hetarylene are unsubstituted or carry 1, 2, 3 or 4, in particular 1 or 2, radicals R^{A}r, where R^{Ar1} has one of the meanings defined herein, especially one of the meanings mentioned as preferred.

In a more preferred subgroup (4.1′) of group (4′) of embodiments, A¹ and A² are independently selected from the group consisting of naphthylene, 1,2-dihydroacenaphthylene, biphenylylene, 1,1′-oxydiphenylene, 1,1′-thiodiphenylene, 9H-fluorenylene, 11H-benzo[a]fluorenylene, 11H-benzo[b]fluorenylene, 7H-benzo[c]fluorenylene, anthracylene, phenanthrylene, benzo[c]phenanthrylene, pyrenylene, chrysenylene, picenylene, triphenylenylene, furanylene, benzo[b]furanylene, dibenzo[b,d]furanylene, naphtho[1,2-b]furanylene, naphtho[2,3-b]furanylene, naphtho[2,1-b]furanylene, benzo[b]naphtho[1,2-o]furanylene, benzo[b]naphtho[2,3-a]furanylene, benzo[b]naphtho[2,1-d]furanylene, benzo[1,2-b:4,3-b′]difuranylene, benzo[1,2-b:6,5-b′]difuranylene, benzo[1,2-b:5,4-b′]difuranylene, benzo[1,2-b:4,5-b′]difuranylene, 9H-xanthylene, tribenzo[b,d,f]oxepinylene, dibenzo[1,4]dioxinylene, 2H-naphtho[1,8-d,e][1,3]dioxinylene, phenoxathiinylene, dinaphtho[2,3-b:2′,3′-a]furanylene, oxanthrenylene, benzo[a]oxanthrenylene, benzo[b]oxanthrenylene, thienylene, benzo[b]thienylene, dibenzo[b]thienylene, naphtho[1,2-b]thienylene, naphtho[2,3-b]thienylene, naphtho[2,1-b]thienylene, benzo[b]naphtho[1,2-d]thienylene, benzo[b]naphtho[2,3-d]thienylene, benzo[b]naphtho[2,1-d]thienylene, benzo[1,2-b-4,3-b′]dithienylene, benzo[1,2-b-6,5-b′]dithienylene, benzo[1,2-b-5,4-b′]dithienylene, benzo[1,2-b:4,5-b′]dithienylene, 9H-thioxanthylene, 6H-dibenzo[b,d]thiopyranylene, 1,4-benzodithiinylene, naphtho[1,2-b][1,4]dithiinylene, naphtho[2,3-b][1,4]dithiinylene, thianthrenylene, benzo[a]thianthrenylene, benzo[b]thianthrenylene, dibenzo[a,c]thianthrenylene, dibenzo[a,h]thianthrenylene, dibenzo[a,i]thianthrenylene, dibenzo[a,j]thianthrenylene, dibenzo[b,d]thianthrenylene, 2H-naphtho[1,8-b,c]thienylene, dibenzo[b,d]thiepinylene, dibenzo[b,f]thiepinylene, 5H-phenanthro[4,5-b,c,d]thiopyranylene, tribenzo[b,d,f]thiepinylene, 2,5-dihydronaphtho[1,8-b,c.4,5-b′,c′]dithienylene, 2,6-dihydronaphtho[1,8-b,c.5,4-b′,c′]dithienylene, tribenzo[a,c,d]thianthrenylene, benzo[b]naphtho[1,8-e,f][1,4]dithiepinylene, dinaphtho[2,3-b:2′,3′-d]thienylene, 5H-phenanthro[1,10-b,c]thienylene, 7H-phenanthro[1,10-c,b]thienylene, dibenzo[d,d′]benzo[1,2-b-4,5-b′]dithienylene and dibenzo[d,d′]benzo[1,2-b:5,4-b′]dithienylene, where the aforementioned polycyclic arylene and mono- or polycyclic hetarylene are unsubstituted or carry 1 or 2 radicals R^{Ar1}.

In an especially preferred subgroup (4.2′) of group (4′) of embodiments, A¹ and A² are independently selected from the group consisting of naphthylene, benzo[b]thienylene, benzo[b]furanylene, biphenylylene, 9H-fluorenylene, oxanthrenylene, phenoxathiinylene, thianthrenylene, 9H-xanthylene and 9H-thioxanthylene, where the aforementioned polycyclic arylene and mono- and polycyclic hetarylene are unsubstituted or carry 1 or 2 radicals R^{Ar1}.

In a particularly preferred subgroup (4.3′) of group (4′) of embodiments, A¹ and A² are independently selected from the group consisting of naphthylene, biphenylylene, benzo[b]furanylene, dibenzo[b,d]furanylene, benzo[b]thienylene, dibenzo[b,a]thienylene, 9H-fluorenylene, and thianthrenylene, where naphthylene, biphenylylene, benzo[b]furanylene, dibenzo[b,d]furanylene, benzo[b]thienylene, dibenzo[b,a]thienylene, 9H-fluorenylene, and thianthrenylene are unsubstituted or carry 1 or 2 radicals R^{Ar1}.

In a particularly preferred subgroup (4.4′) of group (4′) of embodiments, A¹ and A² are independently selected from the group consisting of naphthylene, biphenylylene, benzo[b]furanylene, dibenzo[b,d]furanylene, benzo[b]thienylene, dibenzo[b,a]thienylene, 9H-fluorenylene, and thianthrenylene, where naphthylene, benzo[b]furanylene, dibenzo[b,d]furanylene, benzo[b]thienylene, dibenzo[b,d]thienylene, 9H-fluorenylene, and thianthrenylene are unsubstituted or substituted by 1 radical Q².

A preferred subgroup (4.a′) of the group (4′) of embodiments, relates to compounds of the formula (I), where each of the moieties A¹ and A² comprises a phenylene ring, which bears one or two fused rings selected from fused benzene rings and fused 5- or 6-membered heteroaromatic rings. The moieties A¹ and A² may be unsubstituted or bear 1 or 2 radicals R^{Ar1}, where R^{Ar1} is in particular a radical Q². Amongst the compounds of group (4.a′) of embodiments preference is given to those compounds, wherein the group X¹ or X² and the atom —O— are connected in the para-positions of the phenylene ring of A¹ or A². These compounds are also referred to the para-isomers of group (4.a′) of embodiments. Amongst the compounds of group (4.a′) of embodiments, particular preference is given to the compounds of formula (I), where A¹ and A² are both 1,4-naphthylene or are both 2,6-naphthylene, where 1,4-naphthylene and 2,6-naphthylene may be unsubstituted or bear 1 or 2 radicals R^{Ar1}, where R^{Ar1} is in particular a radical Q².

In an especially preferred subgroup (4.5′) of group (4′) of embodiments, A¹ and A² are independently selected from the group consisting of 1,4-naphthylene, 1,2-naphthylene, 2,1-naphthylene, 2,6-naphthylene, 4,4′-biphenylylene, 3,3′-biphenylylene, 2,2′-biphenylylene, 2,4-dibenzo[b,a]thienylene, 2,6-dibenzo[b]thienylene, 4,2-dibenzo[b,d]thienylene, 2,8-dibenzo[b,d]thienylene, 4,6-dibenzo[b,a]thienylene, 6,2-dibenzo[b,d]thienylene, 2,4-dibenzo[b,d]furanylene, 2,6-dibenzo[b,d]furanylene, 4,2-dibenzo[b,d]furanylene, 2,8-dibenzo[b,d]furanylene, 4,6-dibenzo[b,d]furanylene, 6,2-dibenzo[b,d]furanylene, 1,2-thianthrenylene, 2,1-thianthrenylene, 3,2-thianthrenylene, 1,3-thianthrenylene, 1,4-thianthrenylene, 1,6-thianthrenylene, 2,7-thianthrenylene, 2,8-thianthrenylene, 1,9-thianthrenylene, 2,7-9H-fluroenylene, 9,2-9H-fluroenylene, 2,9-9H-fluroenylene, 2-(phenyl)-1,4-phenylene, 3-(phenyl)-1,4-phenylene, 2-(1-naphthyl)-1,4-phenylene, 3-(1-naphthyl)-1,4-phenylene, 2-(2-naphthyl)-1,4-phenylene, 3-(2-naphthyl)-1,4-phenylene, 2-(9-phenanthryl)-1,4-phenylene, 3-(9-phenanthryl)-1,4-phenylene, 2-(dibenzo[b,a]thien-4-yl)-1,4-phenylene, 3-(dibenzo[b,d]thien-4-yl)-1,4-phenylene, 2-(thianthren-1-yl)-1,4-phenylene, 3-(thianthren-1-yl)-1,4-phenylene, 2-phenyl-1,4-naphthylene, 2-(1-naphthyl)-1,4-naphthylene, 2-(2-naphthyl)-1,4-naphthylene, 2-(9-phenanthryl)-1,4-naphthylene, 2-(dibenzo[b,d]thien-4-yl)-1,4-naphthylene, 2-(thianthren-1-yl)-1,4-naphthylene, 8-phenyl-2,6-naphthylene, 8-(1-naphthyl)-2,6-naphthylene, 8-(2-naphthyl)-2,6-naphthylene, 8-(9-phenanthryl)-2,6-naphthylene, 8-(dibenzo[b,a]thien-4-yl)-2,6-naphthylene and 8-(thianthren-1-yl)-2,6-naphthylene.

In a particular subgroup (4″) of embodiments the variables A¹ and A² in formulae (I) and (II) have the same meaning, which is selected from the meanings defined herein for A¹ and A², especially those mentioned as preferred, and in particular selected from the meanings defined in groups (4), (4.1), (4.2), (4.3), (4.4), (4.5), (4.a), (4′), (4.1′), (4.2′), (4.3′), (4.4′), (4.5′) and (4.a′) of embodiments.

In a preferred group (5) of embodiments, the variables R¹ and R² in formulae (I) and (II), if present, are independently of one another selected from the group consisting of halogen, C₂-C₃-alkynyl, CN, R, SR, OR and CH₅R′₃₋₅, and more preferably from the group of fluorine, CN, R and OR, where s is 1 or 2, especially 2, and the variable R and R′ each have one of the meanings defined herein, especially the preferred ones.

In a particularly preferred subgroup (5.1) of embodiments, R¹ and R², if present, are independently selected from the group consisting of fluorine, CN, methyl, methoxy and a radical Q¹. Especially, R¹ and R², if present, are a radical Q¹.

In the context of the subgroup (5.1) of group (5) of embodiments, the radical Q¹ is mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms.

Especially Q¹ is selected from the group consisting of phenyl, naphthyl, such as 1-naphthyl or 2-naphthyl, phenanthrenyl, such as 1-phenanthrenyl, 2-phenanthrenyl, 3-phenanthrenyl, 4-phenanthrenyl or 9-phenanthrenyl, thianthrenyl, such as 1-thianthrenyl or 2-thianthrenyl and dibenzo[b,a]thienyl, such as dibenzo[b,a]thien-1-yl, dibenzo[b,d]thien-2-yl, dibenzo[b,a]thien-3-yl or dibenzo[b,a]thien-4-yl, and specifically from the group consisting of phenyl or naphthyl, such as 1-naphthyl or 2-naphthyl.

In a particular subgroup (5′) of embodiments the variables R¹ and R² in formulae (I) and (II) have the same meaning, which is selected from the meanings defined herein for R¹ and R², especially those mentioned as preferred, and in particular selected from the meanings defined in groups (5) and (5.1) of embodiments.

Preference is given to the variables p and q in formulae (I) and (II) that have identical meanings selected from 0, 1 and 2.

In a preferred group (6) of embodiments, the variables p and q in formulae (I) and (II) are both 0, i.e. the binaphthyl moiety in formulae (I) and (II) neither carries a substituent R¹ nor a substituent R².

In a preferred group (7) of embodiments, the variables p and q in formulae (I) and (II) are both 1, i.e. the binaphthyl moiety in formulae (I) and (II) carries one substituent R¹ and one substituent R². Additionally, in this group (7) of embodiments the variables R¹ and R² preferably have the same meaning which is selected from the meanings defined herein, especially those mentioned herein as preferred, and is preferably selected from the meanings defined in group (5) of embodiments, in particular those defined in group (5.1) of embodiments. In group 7 of embodiments, R¹ and R² are especially a radical Q¹.

In a particularly preferred subgroup (7.1) of group (7) of embodiments, the two substituents R¹ and R² are each bound to the corresponding positions of their respective naphthyl units, i.e., if R¹ is, for example, bound to position 5 of the binaphthyl moiety of formulae (I) or (II), then R² is bound to position 5′ of that moiety. In subgroup (7.1) of embodiments, R¹ and R² are especially a radical Q¹.

In a particularly preferred subgroup (7.2) of embodiments, the two substituents R¹ and R² are bound to the positions 6 and 6′, respectively, of the binaphthyl moiety of formulae (I) or (II). In subgroup (7.2) of embodiments, R¹ and R² are especially a radical Q¹.

In a preferred group (8) of embodiments, the variables p and q in formulae (I) and (II) are both 2, i.e. the binaphthyl moiety in formulae (I) and (II) carries two substituents R¹ and two substituents R². Additionally, in this group (8) of embodiments the variables R¹ and R² preferably have the same meaning which is selected from the meanings defined herein, especially those mentioned herein as preferred, and is more preferably selected from the meanings defined in group (5), in particular those defined in group (5.1) of embodiments. Furthermore, in this group (8) of embodiments the two substituents R¹ and R² are preferably bound to the corresponding positions of their respective naphthyl units, i.e., if the two substituents R¹ are, for example, bound to positions 3 and 6 of the binaphthyl moiety of formulae (I) or (II), then the two substituents R² are bound to positions 3′ and 6′ of that moiety. In group (8) of embodiments, R¹ and R² are especially a radical Q¹.

A skilled person will readily appreciate that in the formulae (I) and (II) the meanings of X¹ and X² given in group (1) of embodiments may be combined with the meanings of A¹ and A² according to one or more of groups (4), (4.1), (4.2), (4.3), (4.4), (4.5), (4.a), (4′), (4.1′), (4.2′), (4.3′), (4.4′), (4.5′), (4.a′) and (4″) of embodiments, with the meanings of R¹ and R² according to one or more of groups (5), (5.1) and (5′) of embodiments, with the meaning of p and q according either to group (6) of embodiments, to one or more of groups (7), (7.1) and (7.2) of embodiments or to group (8) of embodiments. A skilled person will also appreciate that in the formulae (I) and (II) the meanings of X¹ and X² given in one of groups (2) and (2′) of embodiments may be combined with the meanings of A¹ and A² according to one or more of groups (4), (4.1), (4.2), (4.3), (4.4), (4.5), (4.a), (4′), (4.1′), (4.2′), (4.3′), (4.4′), (4.5′), (4.a′) and (4″) of embodiments, with the meanings of R¹ and R² according to one or more of groups (5), (5.1) and (5′) of embodiments, with the meaning of p and q according either to group (6) of embodiments, to one or more of groups (7), (7.1) and (7.2) of embodiments or to group (8) of embodiments. A skilled person will also appreciate that in the formulae (I) and (II) the meanings of X¹ and X² given in one of groups (3) and (3′) of embodiments may be combined with the meanings of A¹ and A² according to one or more of groups (4), (4.1), (4.2), (4.3), (4.4), (4.5), (4.a), (4′), (4.1′), (4.2′), (4.3′), (4.4′), (4.5′), (4.a′) and (4″) of embodiments, with the meanings of R¹ and R² according to one or more of groups (5), (5.1) and (5′) of embodiments, with the meaning of p and q according either to group (6) of embodiments, to one or more of groups (7), (7.1) and (7.2) of embodiments or to group (8) of embodiments.

It is also preferred in the use according to the first aspect of the present invention and also with regard to the compounds according to the second aspect of the invention that the compound of the formula (I) is not a compound of formula (I), where A¹ and A² are both unsubstituted 1,4-phenylene and p and q are both 0.

It is preferred with regard to both the first aspect and the second aspect of the present invention that p and q are both 1 or 2, especially 1, if both A¹ and A² are unsubstituted phenylene, such as 1,4-phenylene.

It is also preferred with regard to both the first aspect and the second aspect of the present invention that p and q are both 1 or 2, especially 1, if both A¹ and A² are unsubstituted phenylene or phenylene substituted by 1, 2, 3 or 4 radicals R^{Ar1} In particular, it is preferred with regard to both the first aspect and the second aspect of the present invention that p and q are both 1 or 2, especially 1, and at least one of R¹ and R², especially both R¹ and R², represent a radical Q, if both A¹ and A² are unsubstituted phenylene, such as 1,4-phenylene.

In particular, it is also preferred with regard to both the first aspect and the second aspect of the present invention that p and q are both 1 or 2, especially 1, and at least one of R¹ and R², especially both R¹ and R², represent a radical Q, if both A¹ and A² are unsubstituted phenylene or phenylene substituted by 1, 2, 3 or 4 radicals R^{Ar1} Especially, it is preferred with regard to both the first aspect and the second aspect of the present invention that p and q are both 1, and both R¹ and R² represent a radical Q, located in the 6 and 6′ position of the binaphthyl moiety if both A¹ and A² are unsubstituted phenylene, such as 1,4-phenylene.

In particular it also preferred with regard to both the first aspect and the second aspect of the present invention that both A¹ and A² are independently selected from the group consisting of polycyclic arylene having from 10 to 18 carbon atoms as ring members and a mono- or polycyclic hetarylene having a total of 5 to 18 atoms, which are ring members, where 1, 2, 3 or 4 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1, 2, 3 or 4 radicals R^{Ar1}, if p and q are both 0.

If p and q are both 0, it is particularly preferred, that both A¹ and A² are as disclosed in the groups (4′), (4.1′), (4.2′), (4.3′), (4.4′), (4.5′) and (4.a′) of embodiments.

Apart from that and if not stated otherwise, the variables s, R^{Ar1}, Q¹, Q², R¹¹, R, R′, R″ and R′″ either alone or preferably in combination with each other and with the meanings and preferred meanings of the variables X¹, X¹, A¹, A², R¹, R², p and q described above, have the following meanings.

The variable s is in particular 2.

R^{Ar1} is preferably selected from the group consisting of R¹¹, S(O)ₖR¹¹, OR¹¹, CHₜR′₃₋ₜ, where t is 1 or 2, especially 2, and more preferably from the group of R¹¹, SR¹¹ and OR¹¹, and the variables R¹¹ and R′ each have one of the meanings defined herein, especially a preferred one. In particular, the radical R^{Ar1} is selected from the group consisting of methyl, methoxy, SR¹¹ and a radical Q². Especially, the radical R^{Ar1} is a radical Q².

In this context, R¹¹ is preferably, methyl or a radical Q².

In this context, Q² is preferably mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms. Especially Q² is selected from the group consisting of phenyl, naphthyl, such as 1-naphthyl or 2-naphthyl, phenanthrenyl, such as 1-phenanthrenyl, 2-phenanthrenyl, 3-phenanthrenyl, 4-phenanthrenyl or 9-phenanthrenyl, thianthrenyl, such as 1-thianthrenyl or 2-thianthrenyl and dibenzo[b,d]thienyl, such as dibenzo[b,d]thien-1-yl, dibenzo[b,a]thien-2-yl, dibenzo[b,d]thien-3-yl or dibenzo[b,d]thien-4-yl, and specifically from the group consisting of phenyl or naphthyl, such as 1-naphthyl or 2-naphthyl.

R is preferably selected from the group consisting of methyl, ethyl and a radical Q¹, where Q¹ is unsubstituted or substituted by 1, 2 or 3 identical or different radicals R′″, where R′″, independently of each occurrence, has one of the meanings defined herein, in particular a preferred one. More preferably, R is a radical Q¹ as defined herein.

Irrespective of ist occurrence, Q¹ is preferably mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms. Especially Q¹ is selected from the group consisting of phenyl, naphthyl, such as 1-naphthyl or 2-naphthyl, phenanthrenyl, such as 1-phenanthrenyl, 2-phenanthrenyl, 3-phenanthrenyl, 4-phenanthrenyl or 9-phenanthrenyl, thianthrenyl, such as 1-thianthrenyl or 2-thianthrenyl and dibenzo[b,d]thienyl, such as dibenzo[b,d]thien-1-yl, dibenzo[b,a]thien-2-yl, dibenzo[b,d]thien-3-yl or dibenzo[b,d]thien-4-yl, and specifically from the group consisting of phenyl or naphthyl, such as 1-naphthyl or 2-naphthyl.

R′ is preferably selected from the group consisting of phenyl, naphthyl, phenanthrenyl and triphenylenyl, which are unsubstituted or substituted by 1, 2 or 3 identical or different radicals R′″, where R′″, independently of each occurrence, has one of the meanings defined herein, in particular a preferred one. More preferably, R′ is selected from the group consisting of phenyl, naphthyl, such as 1-naphthyl or 2-naphthyl, and phenanthrenyl, such as 1-phenanthrenyl, 2-phenanthrenyl, 3-phenanthrenyl, 4-phenanthrenyl or 9-phenanthrenyl, which are unsubstituted.

R″ is preferably selected from the group consisting of hydrogen, methyl, phenyl and naphthyl, where phenyl and naphthyl are unsubstituted or substituted by 1, 2 or 3, especially 1 or 2, identical or different radicals R′″, where R′″, independently of each occurrence, has one of the meanings defined herein, in particular a preferred one. More preferably, R″ is unsubstituted phenyl or unsubstituted naphthyl, such as 1-naphthyl or 2-naphthyl.

R″ is preferably selected from the group consisting of phenyl, OCH₃ and CH₃.

In a particular subgroup (6/7) of groups (6) and (7) of embodiments, the compound of formula (I) is a compound of the formula (Ia). Formula (Ia) corresponds to formula (I) where the variables p and q are either both 0 or both 1, the groups X¹ and X² are represented by the groups X which have the same meaning, the groups A¹ and A² are represented by the groups A and have the same meaning and R⁰ are hydrogen or represent the radicals R¹ and R², which have the same meaning:

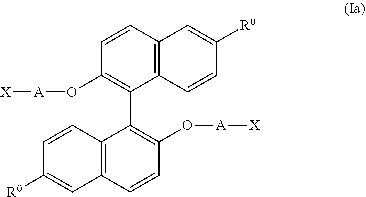

Thus, In formula (Ia) the variable X represents the identical groups X¹ and X², A represents the identical groups A¹ and A², and R⁰ is H or a radical R¹/R², in particular H, fluorine or a radical R, especially H or a radical Q¹, and where X¹, X², Q¹, A¹ and A² have the meanings defined herein, in particular the meanings mentioned herein as preferred, namely those given in groups (3), (3′), (4), (4.1), (4.2), (4.3), (4.4), (4.5), (4′), (4.1′), (4.2′), (4.3′), (4.4′), (4.5′) and (5.1) of embodiments.

In this subgroup (6/7) of groups (6) and (7) of embodiments the structural unit of the formula (II) is a structural unit of the formula (IIa),

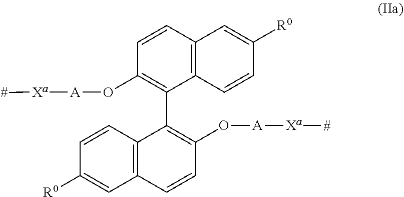

where #represents a connection point to a neighboring structural unit, where X^{a} represents the identical groups X^{1a} and X^{2a}, where A represents the identical groups A¹ and A², where R⁰ is H, fluorine or a radical R, in particular H or a radical Q¹, and where the variables X^{1a}, X^{2a}, Q¹, A¹ and A² have the meanings defined herein, in particular the meanings mentioned as preferred, namely those given in groups (3), (3′), (4), (4.1), (4.2), (4.3), (4.4), (4.5), (4′), (4.1′), (4.2′), (4.3′), (4.4′), (4.5′) and (5.1) of embodiments.

Preferably, the moieties X in formula (Ia) as well as the moieties X^{a} in formula (IIa) are defined either as in group (1) of the embodiments, in group (2) of the embodiments or in group (3) of the embodiments. Thus, the moieties X in formula (Ia) are here in particular selected from the group consisting of —CH₂OH (i.e. hydroxymethyl) and —C(O)OR^{x}, wherein R^{X} is hydrogen or C₁-C₄-alkyl, especially selected from —CH₂OH, —C(O)OH, —C(O)OCH₃ and —C(O)OCH₂CH₃, and specifically selected from —CH₂OH, —C(O)OH and —C(O)OCH₃. Accordingly, the moieties X^{a} in formula (IIa) are here selected from the group consisting of —CH₂O— and —C(O)O—.

Preference is also given to compounds of the formula (Ia) and to structural units of the formula (IIa), where the moieties A are defined as in one of groups (4), (4.1), (4.2), (4.3) and (4.4) of embodiments and where R⁰ is hydrogen or has one of the meanings given for R¹ and R², respectively, and where R⁰ is in particular hydrogen or has one of the meanings given in group (5.1) of embodiments. More preferably, the moiety A in formula (Ia) as well as in formula (IIa) is defined as in group (4.5) of the embodiments.

Especially, the moiety A in formula (Ia) as well as in formula (IIa) is defined as in group (4.5) of the embodiments and R⁰ is in particular hydrogen or has one of the meanings given in group (5.1) of embodiments.

Thus, the moieties A in formulae (Ia) and (IIa) are here in particular selected from the group consisting of A¹ and A² are independently selected from the group consisting of of 1,4-phenylene, 1,3-phenylene, 1,2-phenylene, 1,4-naphthylene, 1,2-naphthylene, 2,1-naphthylene, 2,6-naphthylene, 4,4′-biphenylylene, 3,3′-biphenylylene, 2,2′-biphenylylene, 2,4-dibenzo[b,a]thienylene, 2,6-dibenzo[b]thienylene, 4,2-dibenzo[b,d]thienylene, 2,8-dibenzo[b,d]thienylene, 4,6-dibenzo[b,a]thienylene, 6,2-dibenzo[b,d]thienylene, 2,4-dibenzo[b,d]furanylene, 2,6-dibenzo[b,d]furanylene, 4,2-dibenzo[b,d]furanylene, 2,8-dibenzo[b,d]furanylene, 4,6-dibenzo[b,d]furanylene, 6,2-dibenzo[b,d]furanylene, 1,2-thianthrenylene, 2,1-thianthrenylene, 3,2-thianthrenylene, 1,3-thianthrenylene, 1,4-thianthrenylene, 1,6-thianthrenylene, 2,7-thianthrenylene, 2,8-thianthrenylene, 1,9-thianthrenylene, 2,7-9H-fluroenylene, 9,2-9H-fluroenylene, 2,9-9H-fluroenylene, 2-(phenyl)-1,4-phenylene, 3-(phenyl)-1,4-phenylene, 2-(1-naphthyl)-1,4-phenylene, 3-(1-naphthyl)-1,4-phenylene, 2-(2-naphthyl)-1,4-phenylene, 3-(2-naphthyl)-1,4-phenylene, 2-(9-phenanthryl)-1,4-phenylene, 3-(9-phenanthryl)-1,4-phenylene, 2-(dibenzo[b,d]thien-4-yl)-1,4-phenylene, 3-(dibenzo[b,d]thien-4-yl)-1,4-phenylene, 2-(thianthren-1-yl)-1,4-phenylene, 3-(thianthren-1-yl)-1,4-phenylene, 2-phenyl-1,4-naphthylene, 2-(1-naphthyl)-1,4-naphthylene, 2-(2-naphthyl)-1,4-naphthylene, 2-(9-phenanthryl)-1,4-naphthylene, 2-(dibenzo[b,d]thien-4-yl)-1,4-naphthylene, 2-(thianthren-1-yl)-1,4-naphthylene, 8-phenyl-2,6-naphthylene, 8-(1-naphthyl)-2,6-naphthylene, 8-(2-naphthyl)-2,6-naphthylene, 8-(9-phenanthryl)-2,6-naphthylene, 8-(dibenzo[b,a]thien-4-yl)-2,6-naphthylene and 8-(thianthren-1-yl)-2,6-naphthylene and the radicals R⁰ are in particular hydrogen or Q¹, where Q¹ is mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms and where Q¹ is more preferably selected from the group consisting of phenyl, naphthyl, such as 1-naphthyl or 2-naphthyl, phenanthrenyl, such as 1-phenanthrenyl, 2-phenanthrenyl, 3-phenanthrenyl, 4-phenanthrenyl or 9-phenanthrenyl, thianthrenyl, such as 1-thianthrenyl or 2-thianthrenyl and dibenzo[b,a]thienyl, such as dibenzo[b,a]thien-1-yl, dibenzo[b,a]thien-2-yl, dibenzo[b,a]thien-3-yl or dibenzo[b,a]thien-4-yl, and specifically from the group consisting of phenyl or naphthyl, such as 1-naphthyl or 2-naphthyl.

Examples of the particular subgroup (6/7) are the compounds of the formula (Ia) and the structural units of formula (IIa), in which the combination of the moieties X or moieties X^{a}, respectively, and the moieties A is as defined in any one of the lines 1 to 243 in table A below, where X^{a} in each case is derived from X in formula (Ia) by replacing the —OH or —OR^{x} group of X with an oxo (—O—) unit.

TABLE A

| # | X | A *) | R⁰ |
|---|---|---|---|
| 1 | —CH₂OH | 1,4-phenylene | H |
| 2 | —CH₂OH | 1,3-phenylene | H |
| 3 | —CH₂OH | 1,2-phenylene | H |
| 4 | —CH₂OH | 1,4-naphthylene | H |
| 5 | —CH₂OH | 2,1-naphthylene | H |
| 6 | —CH₂OH | 2,6-naphthylene | H |
| 7 | —CH₂OH | 4,4′-biphenylylene | H |
| 8 | —CH₂OH | 3,3′-biphenylylene | H |
| 9 | —CH₂OH | 2,2′-biphenylylene | H |
| 10 | —CH₂OH | 2,7-9H-fluorenylene | H |
| 11 | —CH₂OH | 2,9-9H-fluorenylene | H |
| 12 | —CH₂OH | 9,2-9H-fluorenylene | H |
| 13 | —CH₂OH | 2,4-dibenzo[b,d]thienylene | H |
| 14 | —CH₂OH | 2,6-dibenzo[b,d]thienylene | H |
| 15 | —CH₂OH | 4,2-dibenzo[b,d]thienylene | H |
| 16 | —CH₂OH | 6,2-dibenzo[b,d]thienylene | H |
| 17 | —CH₂OH | 4,6-dibenzo[b,d]thienylene | H |
| 18 | —CH₂OH | 2,8-dibenzo[b,d]thienylene | H |
| 19 | —CH₂OH | 2,4-dibenzo[b,d]furanylene | H |
| 20 | —CH₂OH | 2,6-dibenzo[b,d]furanylene | H |
| 21 | —CH₂OH | 4,2-dibenzo[b,d]furanylene | H |
| 22 | —CH₂OH | 6,2-dibenzo[b,d]furanylene | H |
| 23 | —CH₂OH | 4,6-dibenzo[b,d]furanylene | H |
| 24 | —CH₂OH | 2,8-dibenzo[b,d]furanylene | H |
| 25 | —CH₂OH | 1,2-thianthrenylene | H |
| 26 | —CH₂OH | 2,1-thianthrenylene | H |
| 27 | —CH₂OH | 1,3-thianthrenylene | H |
| 28 | —CH₂OH | 2,3-thianthrenylene | H |
| 29 | —CH₂OH | 1,4-thianthrenylene | H |
| 30 | —CH₂OH | 1,6-thianthrenylene | H |
| 31 | —CH₂OH | 2,7-thianthrenylene | H |
| 32 | —CH₂OH | 2,8-thianthrenylene | H |
| 33 | —CH₂OH | 1,9-thianthrenylene | H |
| 34 | —C(O)OH | 1,4-phenylene | H |
| 35 | —C(O)OH | 1,3-phenylene | H |
| 36 | —C(O)OH | 1,2-phenylene | H |
| 37 | —C(O)OH | 1,4-naphthylene | H |
| 38 | —C(O)OH | 2,1-naphthylene | H |
| 39 | —C(O)OH | 2,6-naphthylene | H |
| 40 | —C(O)OH | 4,4′-biphenylylene | H |
| 41 | —C(O)OH | 3,3′-biphenylylene | H |
| 42 | —C(O)OH | 2,2′-biphenylylene | H |
| 43 | —C(O)OH | 2,7-9H-fluorenylene | H |
| 44 | —C(O)OH | 2,9-9H-fluorenylene | H |
| 45 | —C(O)OH | 9,2-9H-fluorenylene | H |
| 46 | —C(O)OH | 2,4-dibenzo[b,d]thienylene | H |
| 47 | —C(O)OH | 2,6-dibenzo[b,d]thienylene | H |
| 48 | —C(O)OH | 4,2-dibenzo[b,d]thienylene | H |
| 49 | —C(O)OH | 6,2-dibenzo[b,d]thienylene | H |
| 50 | —C(O)OH | 4,6-dibenzo[b,d]thienylene | H |
| 51 | —C(O)OH | 2,8-dibenzo[b,d]thienylene | H |
| 52 | —C(O)OH | 2,4-dibenzo[b,d]furanylene | H |
| 53 | —C(O)OH | 2,6-dibenzo[b,d]furanylene | H |
| 54 | —C(O)OH | 4,2-dibenzo[b,d]furanylene | H |
| 55 | —C(O)OH | 6,2-dibenzo[b,d]furanylene | H |
| 56 | —C(O)OH | 4,6-dibenzo[b,d]furanylene | H |
| 57 | —C(O)OH | 2,8-dibenzo[b,d]furanylene | H |
| 58 | —C(O)OH | 1,2-thianthrenylene | H |
| 59 | —C(O)OH | 2,1-thianthrenylene | H |
| 60 | —C(O)OH | 1,3-thianthrenylene | H |
| 61 | —C(O)OH | 2,3-thianthrenylene | H |
| 62 | —C(O)OH | 1,4-thianthrenylene | H |
| 63 | —C(O)OH | 1,6-thianthrenylene | H |
| 64 | —C(O)OH | 2,7-thianthrenylene | H |
| 65 | —C(O)OH | 2,8-thianthrenylene | H |
| 66 | —C(O)OH | 1,9-thianthrenylene | H |
| 67 | —C(O)OCH₃ | 1,4-phenylene | H |
| 68 | —C(O)OCH₃ | 1,3-phenylene | H |
| 69 | —C(O)OCH₃ | 1,2-phenylene | H |
| 70 | —C(O)OCH₃ | 1,4-naphthylene | H |
| 71 | —C(O)OCH₃ | 2,1-naphthylene | H |
| 72 | —C(O)OCH₃ | 2,6-naphthylene | H |
| 73 | —C(O)OCH₃ | 4,4′-biphenylylene | H |
| 74 | —C(O)OCH₃ | 3,3′-biphenylylene | H |
| 75 | —C(O)OCH₃ | 2,2′-biphenylylene | H |
| 76 | —C(O)OCH₃ | 2,7-9H-fluorenylene | H |
| 77 | —C(O)OCH₃ | 2,9-9H-fluorenylene | H |
| 78 | —C(O)OCH₃ | 9,2-9H-fluorenylene | H |
| 79 | —C(O)OCH₃ | 2,4-dibenzo[b,d]thienylene | H |
| 80 | —C(O)OCH₃ | 2,6-dibenzo[b,d]thienylene | H |
| 81 | —C(O)OCH₃ | 4,2-dibenzo[b,d]thienylene | H |
| 82 | —C(O)OCH₃ | 6,2-dibenzo[b,d]thienylene | H |
| 83 | —C(O)OCH₃ | 4,6-dibenzo[b,d]thienylene | H |
| 84 | —C(O)OCH₃ | 2,8-dibenzo[b,d]thienylene | H |
| 85 | —C(O)OCH₃ | 2,4-dibenzo[b,d]furanylene | H |
| 86 | —C(O)OCH₃ | 2,6-dibenzo[b,d]furanylene | H |
| 87 | —C(O)OCH₃ | 4,2-dibenzo[b,d]furanylene | H |
| 88 | —C(O)OCH₃ | 6,2-dibenzo[b,d]furanylene | H |
| 89 | —C(O)OCH₃ | 4,6-dibenzo[b,d]furanylene | H |
| 90 | —C(O)OCH₃ | 2,8-dibenzo[b,d]furanylene | H |
| 91 | —C(O)OCH₃ | 1,2-thianthrenylene | H |
| 92 | —C(O)OCH₃ | 2,1-thianthrenylene | H |
| 93 | —C(O)OCH₃ | 1,3-thianthrenylene | H |
| 94 | —C(O)OCH₃ | 2,3-thianthrenylene | H |
| 95 | —C(O)OCH₃ | 1,4-thianthrenylene | H |
| 96 | —C(O)OCH₃ | 1,6-thianthrenylene | H |
| 97 | —C(O)OCH₃ | 2,7-thianthrenylene | H |
| 98 | —C(O)OCH₃ | 2,8-thianthrenylene | H |
| 99 | —C(O)OCH₃ | 1,9-thianthrenylene | H |
| 100 | —CH₂OH | 1,4-phenylene | phenyl |
| 101 | —CH₂OH | 1,2-phenylene | phenyl |
| 102 | —CH₂OH | 1,4-naphthylene | phenyl |
| 103 | —CH₂OH | 2,1-naphthylene | phenyl |
| 104 | —CH₂OH | 2,6-naphthylene | phenyl |
| 105 | —CH₂OH | 4,4′-biphenylylene | phenyl |
| 106 | —CH₂OH | 2,2′-biphenylylene | phenyl |
| 107 | —CH₂OH | 2,7-9H-fluorenylene | phenyl |
| 108 | —C(O)OH | 1,4-phenylene | phenyl |
| 109 | —C(O)OH | 1,2-phenylene | phenyl |
| 110 | —C(O)OH | 1,4-naphthylene | phenyl |
| 111 | —C(O)OH | 2,1-naphthylene | phenyl |
| 112 | —C(O)OH | 2,6-naphthylene | phenyl |
| 113 | —C(O)OH | 4,4′-biphenylylene | phenyl |
| 114 | —C(O)OH | 2,2′-biphenylylene | phenyl |
| 115 | —C(O)OH | 2,7-9H-fluorenylene | phenyl |
| 116 | —C(O)OCH₃ | 1,4-phenylene | phenyl |
| 117 | —C(O)OCH₃ | 1,2-phenylene | phenyl |
| 118 | —C(O)OCH₃ | 1,4-naphthylene | phenyl |
| 119 | —C(O)OCH₃ | 2,1-naphthylene | phenyl |
| 120 | —C(O)OCH₃ | 2,6-naphthylene | phenyl |
| 121 | —C(O)OCH₃ | 4,4′-biphenylylene | phenyl |
| 122 | —C(O)OCH₃ | 2,2′-biphenylylene | phenyl |
| 123 | —C(O)OCH₃ | 2,7-9H-fluorenylene | phenyl |
| 124 | —CH₂OH | 1,4-phenylene | 1-naphthyl |
| 125 | —CH₂OH | 1,2-phenylene | 1-naphthyl |
| 126 | —CH₂OH | 1,4-naphthylene | 1-naphthyl |
| 127 | —CH₂OH | 2,1-naphthylene | 1-naphthyl |
| 128 | —CH₂OH | 2,6-naphthylene | 1-naphthyl |
| 129 | —CH₂OH | 4,4′-biphenylylene | 1-naphthyl |
| 130 | —CH₂OH | 2,2′-biphenylylene | 1-naphthyl |
| 131 | —CH₂OH | 2,7-9H-fluorenylene | 1-naphthyl |
| 132 | —C(O)OH | 1,4-phenylene | 1-naphthyl |
| 133 | —C(O)OH | 1,2-phenylene | 1-naphthyl |
| 134 | —C(O)OH | 1,4-naphthylene | 1-naphthyl |
| 135 | —C(O)OH | 2,1-naphthylene | 1-naphthyl |
| 136 | —C(O)OH | 2,6-naphthylene | 1-naphthyl |
| 137 | —C(O)OH | 4,4′-biphenylylene | 1-naphthyl |
| 138 | —C(O)OH | 2,2′-biphenylylene | 1-naphthyl |
| 139 | —C(O)OH | 2,7-9H-fluorenylene | 1-naphthyl |
| 140 | —C(O)OCH₃ | 1,4-phenylene | 1-naphthyl |
| 141 | —C(O)OCH₃ | 1,2-phenylene | 1-naphthyl |
| 142 | —C(O)OCH₃ | 1,4-naphthylene | 1-naphthyl |
| 143 | —C(O)OCH₃ | 2,1-naphthylene | 1-naphthyl |
| 144 | —C(O)OCH₃ | 2,6-naphthylene | 1-naphthyl |
| 145 | —C(O)OCH₃ | 4,4′-biphenylylene | 1-naphthyl |
| 146 | —C(O)OCH₃ | 2,2′-biphenylylene | 1-naphthyl |
| 147 | —C(O)OCH₃ | 2,7-9H-fluorenylene | 1-naphthyl |
| 148 | —CH₂OH | 1,4-phenylene | 2-naphthyl |
| 149 | —CH₂OH | 1,2-phenylene | 2-naphthyl |
| 150 | —CH₂OH | 1,4-naphthylene | 2-naphthyl |
| 151 | —CH₂OH | 2,1-naphthylene | 2-naphthyl |
| 152 | —CH₂OH | 2,6-naphthylene | 2-naphthyl |
| 153 | —CH₂OH | 4,4′-biphenylylene | 2-naphthyl |
| 154 | —CH₂OH | 2,2′-biphenylylene | 2-naphthyl |
| 155 | —CH₂OH | 2,7-9H-fluorenylene | 2-naphthyl |
| 156 | —C(O)OH | 1,4-phenylene | 2-naphthyl |
| 157 | —C(O)OH | 1,2-phenylene | 2-naphthyl |
| 158 | —C(O)OH | 1,4-naphthylene | 2-naphthyl |
| 159 | —C(O)OH | 2,1-naphthylene | 2-naphthyl |
| 160 | —C(O)OH | 2,6-naphthylene | 2-naphthyl |
| 161 | —C(O)OH | 4,4′-biphenylylene | 2-naphthyl |
| 162 | —C(O)OH | 2,2′-biphenylylene | 2-naphthyl |
| 163 | —C(O)OH | 2,7-9H-fluorenylene | 2-naphthyl |
| 164 | —C(O)OCH₃ | 1,4-phenylene | 2-naphthyl |
| 165 | —C(O)OCH₃ | 1,2-phenylene | 2-naphthyl |
| 166 | —C(O)OCH₃ | 1,4-naphthylene | 2-naphthyl |
| 167 | —C(O)OCH₃ | 2,1-naphthylene | 2-naphthyl |
| 168 | —C(O)OCH₃ | 2,6-naphthylene | 2-naphthyl |
| 169 | —C(O)OCH₃ | 4,4′-biphenylylene | 2-naphthyl |
| 170 | —C(O)OCH₃ | 2,2′-biphenylylene | 2-naphthyl |
| 171 | —C(O)OCH₃ | 2,7-9H-fluorenylene | 2-naphthyl |
| 172 | —CH₂OH | 1,4-phenylene | 9-phenanthryl |
| 173 | —CH₂OH | 1,2-phenylene | 9-phenanthryl |
| 174 | —CH₂OH | 1,4-naphthylene | 9-phenanthryl |
| 175 | —CH₂OH | 2,1-naphthylene | 9-phenanthryl |
| 176 | —CH₂OH | 2,6-naphthylene | 9-phenanthryl |
| 177 | —CH₂OH | 4,4′-biphenylylene | 9-phenanthryl |
| 178 | —CH₂OH | 2,2′-biphenylylene | 9-phenanthryl |
| 179 | —CH₂OH | 2,7-9H-fluorenylene | 9-phenanthryl |
| 180 | —C(O)OH | 1,4-phenylene | 9-phenanthryl |
| 181 | —C(O)OH | 1,2-phenylene | 9-phenanthryl |
| 182 | —C(O)OH | 1,4-naphthylene | 9-phenanthryl |
| 183 | —C(O)OH | 2,1-naphthylene | 9-phenanthryl |
| 184 | —C(O)OH | 2,6-naphthylene | 9-phenanthryl |
| 185 | —C(O)OH | 4,4′-biphenylylene | 9-phenanthryl |
| 186 | —C(O)OH | 2,2′-biphenylylene | 9-phenanthryl |
| 187 | —C(O)OH | 2,7-9H-fluorenylene | 9-phenanthryl |
| 188 | —C(O)OCH₃ | 1,4-phenylene | 9-phenanthryl |
| 189 | —C(O)OCH₃ | 1,2-phenylene | 9-phenanthryl |
| 190 | —C(O)OCH₃ | 1,4-naphthylene | 9-phenanthryl |
| 191 | —C(O)OCH₃ | 2,1-naphthylene | 9-phenanthryl |
| 192 | —C(O)OCH₃ | 2,6-naphthylene | 9-phenanthryl |
| 193 | —C(O)OCH₃ | 4,4′-biphenylylene | 9-phenanthryl |
| 194 | —C(O)OCH₃ | 2,2′-biphenylylene | 9-phenanthryl |
| 195 | —C(O)OCH₃ | 2,7-9H-fluorenylene | 9-phenanthryl |
| 196 | —CH₂OH | 1,4-phenylene | dibenzo[b,d]thien-4-yl |
| 197 | —CH₂OH | 1,2-phenylene | dibenzo[b,d]thien-4-yl |
| 198 | —CH₂OH | 1,4-naphthylene | dibenzo[b,d]thien-4-yl |
| 199 | —CH₂OH | 2,1-naphthylene | dibenzo[b,d]thien-4-yl |
| 200 | —CH₂OH | 2,6-naphthylene | dibenzo[b,d]thien-4-yl |
| 201 | —CH₂OH | 4,4′-biphenylylene | dibenzo[b,d]thien-4-yl |
| 202 | —CH₂OH | 2,2′-biphenylylene | dibenzo[b,d]thien-4-yl |
| 203 | —CH₂OH | 2,7-9H-fluorenylene | dibenzo[b,d]thien-4-yl |
| 204 | —C(O)OH | 1,4-phenylene | dibenzo[b,d]thien-4-yl |
| 205 | —C(O)OH | 1,2-phenylene | dibenzo[b,d]thien-4-yl |
| 206 | —C(O)OH | 1,4-naphthylene | dibenzo[b,d]thien-4-yl |
| 207 | —C(O)OH | 2,1-naphthylene | dibenzo[b,d]thien-4-yl |
| 208 | —C(O)OH | 2,6-naphthylene | dibenzo[b,d]thien-4-yl |
| 209 | —C(O)OH | 4,4′-biphenylylene | dibenzo[b,d]thien-4-yl |
| 210 | —C(O)OH | 2,2′-biphenylylene | dibenzo[b,d]thien-4-yl |
| 211 | —C(O)OH | 2,7-9H-fluorenylene | dibenzo[b,d]thien-4-yl |
| 212 | —C(O)OCH₃ | 1,4-phenylene | dibenzo[b,d]thien-4-yl |
| 213 | —C(O)OCH₃ | 1,2-phenylene | dibenzo[b,d]thien-4-yl |
| 214 | —C(O)OCH₃ | 1,4-naphthylene | dibenzo[b,d]thien-4-yl |
| 215 | —C(O)OCH₃ | 2,1-naphthylene | dibenzo[b,d]thien-4-yl |
| 216 | —C(O)OCH₃ | 2,6-naphthylene | dibenzo[b,d]thien-4-yl |
| 217 | —C(O)OCH₃ | 4,4′-biphenylylene | dibenzo[b,d]thien-4-yl |
| 218 | —C(O)OCH₃ | 2,2′-biphenylylene | dibenzo[b,d]thien-4-yl |
| 219 | —C(O)OCH₃ | 2,7-9H-fluorenylene | dibenzo[b,d]thien-4-yl |
| 220 | —CH₂OH | 1,4-phenylene | thianthren-1-yl |
| 221 | —CH₂OH | 1,2-phenylene | thianthren-1-yl |
| 222 | —CH₂OH | 1,4-naphthylene | thianthren-1-yl |
| 223 | —CH₂OH | 2,1-naphthylene | thianthren-1-yl |
| 224 | —CH₂OH | 2,6-naphthylene | thianthren-1-yl |
| 225 | —CH₂OH | 4,4′-biphenylylene | thianthren-1-yl |
| 226 | —CH₂OH | 2,2′-biphenylylene | thianthren-1-yl |
| 227 | —CH₂OH | 2,7-9H-fluorenylene | thianthren-1-yl |
| 228 | —C(O)OH | 1,4-phenylene | thianthren-1-yl |
| 229 | —C(O)OH | 1,2-phenylene | thianthren-1-yl |
| 230 | —C(O)OH | 1,4-naphthylene | thianthren-1-yl |
| 231 | —C(O)OH | 2,1-naphthylene | thianthren-1-yl |
| 232 | —C(O)OH | 2,6-naphthylene | thianthren-1-yl |
| 233 | —C(O)OH | 4,4′-biphenylylene | thianthren-1-yl |
| 234 | —C(O)OH | 2,2′-biphenylylene | thianthren-1-yl |
| 235 | —C(O)OH | 2,7-9H-fluorenylene | thianthren-1-yl |
| 236 | —C(O)OCH₃ | 1,4-phenylene | thianthren-1-yl |
| 237 | —C(O)OCH₃ | 1,2-phenylene | thianthren-1-yl |
| 238 | —C(O)OCH₃ | 1,4-naphthylene | thianthren-1-yl |
| 239 | —C(O)OCH₃ | 2,1-naphthylene | thianthren-1-yl |
| 240 | —C(O)OCH₃ | 2,6-naphthylene | thianthren-1-yl |
| 241 | —C(O)OCH₃ | 4,4′-biphenylylene | thianthren-1-yl |
| 242 | —C(O)OCH₃ | 2,2′-biphenylylene | thianthren-1-yl |
| 243 | —C(O)OCH₃ | 2,7-9H-fluorenylene | thianthren-1-yl |

*) the linkage positions “n,m-” included in the names of the moieties A are to be understood such that the first one, i.e. n, indicates the position of the carbon atom linked to X, and the second one, i.e. m, indicates the position of the carbon atom linked to the O atom.

Amongst the compounds of formula (Ia) recited in table A, particular preference is given to the following compounds:
{[1,1′-binaphthalene]-2,2′-diylbis(oxy-4,1-phenylene)}dimethanol
{[1,1′-binaphthalene]-2,2′-diylbis(oxy-3,1-phenylene)}dimethanol
{[1,1′-binaphthalene]-2,2′-diylbis(oxy-2,1-phenylene)}dimethanol
{[1,1′-binaphthalene]-2,2′-diylbis(oxynaphthalene-4,1-diyl)}dimethanol
{[1,1′-binaphthalene]-2,2′-diylbis(oxynaphthalene-6,2-diyl)}dimethanol
{[1,1′-binaphthalene]-2,2′-diylbis(oxynaphthalene-1,2-diyl)}dimethanol
{[1,1′-binaphthalene]-2,2′-diylbis(oxy[1,1′-biphenyl]-4′,4-diyl)}dimethanol
{[1,1′-binaphthalene]-2,2′-diylbis(oxy[1,1′-biphenyl]-3′,3-diyl)}dimethanol
{[1,1′-binaphthalene]-2,2′-diylbis(oxy[1,1′-biphenyl]-2′,2-diyl)}dimethanol
{[1,1′-binaphthalene]-2,2′-diylbis(oxy-9H-fluorene-7,2-diyl)}dimethanol
{[1,1′-binaphthalene]-2,2′-diylbis(oxy-9H-fluorene-2,9-diyl)}dimethanol
{[1,1′-binaphthalene]-2,2′-diylbis(oxy-9H-fluorene-9,2-diyl)}dimethanol
{[1,1′-binaphthalene]-2,2′-diylbis(oxydibenzo[b,a]thiene-6,4-diyl)}dimethanol
{[1,1′-binaphthalene]-2,2′-diylbis(oxydibenzo[b,a]thiene-6,2-diyl)}dimethanol
{[1,1′-binaphthalene]-2,2′-diylbis(oxydibenzo[b,d]thiene-4,2-diyl)}dimethanol
{[1,1′-binaphthalene]-2,2′-diylbis(oxydibenzo[b,a]thiene-8,2-diyl)}dimethanol
{[1,1′-binaphthalene]-2,2′-diylbis(oxydibenzo[b,a]thiene-2,6-diyl)}dimethanol
{[1,1′-binaphthalene]-2,2′-diylbis(oxydibenzo[b,a]thiene-2,4-diyl)}dimethanol
{[1,1′-binaphthalene]-2,2′-diylbis(oxydibenzo[b,]furan-6,4-diyl)}dimethanol
{[1,1′-binaphthalene]-2,2′-diylbis(oxydibenzo[b,d]furan-6,2-diyl)}dimethanol
{[1,1′-binaphthalene]-2,2′-diylbis(oxydibenzo[b,a]furan-4,2-diyl)}dimethanol
{[1,1′-binaphthalene]-2,2′-diylbis(oxydibenzo[b,a]furan-8,2-diyl)}dimethanol
{[1,1′-binaphthalene]-2,2′-diylbis(oxydibenzo[b,a]furan-2,6-diyl)}dimethanol
{[1,1′-binaphthalene]-2,2′-diylbis(oxydibenzo[b,a]furan-2,4-diyl)}dimethanol
{[1,1′-binaphthalene]-2,2′-diylbis(oxythianthrene-9,1-diyl)}dimethanol
{[1,1′-binaphthalene]-2,2′-diylbis(oxythianthrene-6,1-diyl)}dimethanol
{[1,1′-binaphthalene]-2,2′-diylbis(oxythianthrene-1,2-diyl)}dimethanol
{[1,1′-binaphthalene]-2,2′-diylbis(oxythianthrene-1,3-diyl)}dimethanol
{[1,1′-binaphthalene]-2,2′-diylbis(oxythianthrene-7,2-diyl)}dimethanol
{[1,1′-binaphthalene]-2,2′-diylbis(oxythianthrene-8,2-diyl)}dimethanol
{[1,1′-binaphthalene]-2,2′-diylbis(oxythianthrene-2,1-diyl)}dimethanol
{[1,1′-binaphthalene]-2,2′-diylbis(oxythianthrene-3,2-diyl)}dimethanol
4,4′-{[1,1′-binaphthalene]-2,2′-diylbis(oxy)}dibenzoic acid
3,3′-{[1,1′-binaphthalene]-2,2′-diylbis(oxy)}dibenzoic acid
2,2′-{[1,1′-binaphthalene]-2,2′-diylbis(oxy)}dibenzoic acid
4,4′-[[1,1′-binaphthalene]-2,2′-diylbis(oxy)]di(naphthalene-1-carboxylic acid)
6,6′-[[1,1′-binaphthalene]-2,2′-diylbis(oxy)]di(naphthalene-2-carboxylic acid)
1,1′-[[1,1′-binaphthalene]-2,2′-diylbis(oxy)]di(naphthalene-2-carboxylic acid)
7,7′-[[1,1′-binaphthalene]-2,2′-diylbis(oxy)]di(9H-fluorene-2-carboxylic acid)
2,2′-[[1,1′-binaphthalene]-2,2′-diylbis(oxy)]di(9H-fluorene-9-carboxylic acid)
9,9′-[[1,1′-binaphthalene]-2,2′-diylbis(oxy)]di(9H-fluorene-2-carboxylic acid)
6,6′-[[1,1′-binaphthalene]-2,2′-diylbis(oxy)]di(dibenzo[b,a]thiophene-4-carboxylic acid)
6,6′-[[1,1′-binaphthalene]-2,2′-diylbis(oxy)]di(dibenzo[b,a]thiophene-2-carboxylic acid)
4,4′-[[1,1′-binaphthalene]-2,2′-diylbis(oxy)]di(dibenzo[b,a]thiophene-2-carboxylic acid)
8,8′-[[1,1′-binaphthalene]-2,2′-diylbis(oxy)]di(dibenzo[b,d]thiophene-2-carboxylic acid)
8,8′-[[1,1′-binaphthalene]-2,2′-diylbis(oxy)]di(dibenzo[b,a]thiophene-4-carboxylic acid)
2,2′-[[1,1′-binaphthalene]-2,2′-diylbis(oxy)]di(dibenzo[b,a]thiophene-4-carboxylic acid)
6,6′-[[1.1′-binaphthalene]-2,2′-diylbis(oxy)]di(dibenzo[b,d]furan-4-carboxylic acid)
9,9′-[[1,1′-binaphthalene]-2,2′-diylbis(oxy)]di(thianthrene-1-carboxylic acid)
dimethyl 4,4′-{[1,1′-binaphthalene]-2,2′-diylbis(oxy)}dibenzoate
dimethyl 2,2′-{[1,1′-binaphthalene]-2,2′-diylbis(oxy)}dibenzoate
dimethyl 4,4′-[[1,1′-binaphthalene]-2,2′-diylbis(oxy)]di(naphthalene-1-carboxylate)
dimethyl 6,6′-[[1,1′-binaphthalene]-2,2′-diylbis(oxy)]di(naphthalene-2-carboxylate)
{(6,6′-diphenyl[1,1′-binaphthalene]-2,2′-diyl)bis(oxy-4,1-phenylene)}dimethanol
{(6,6′-diphenyl[1,1′-binaphthalene]-2,2′-diyl)bis(oxy-2,1-phenylene)}dimethanol
[(6,6′-diphenyl[1,1′-binaphthalene]-2,2′-diyl)bis(oxynaphthalene-4,1-diyl)]dimethanol
4,4′-[(6,6′-diphenyl[1,1′-binaphthalene]-2,2′-diyl)bis(oxy)]dibenzoic acid
2,2′-[(6,6′-diphenyl[1,1′-binaphthalene]-2,2′-diyl)bis(oxy)]dibenzoic acid
4,4′-[(6,6′-diphenyl[1,1′-binaphthalene]-2,2′-diyl)bis(oxy)]di(naphthalene-1-carboxylic acid)
dimethyl 4,4′-[(6,6′-diphenyl[1,1′-binaphthalene]-2,2′-diyl)bis(oxy)]dibenzoate
dimethyl 2,2′-[(6,6′-diphenyl[1,1′-binaphthalene]-2,2′-diyl)bis(oxy)]dibenzoate
dimethyl 4,4′-[(6,6′-diphenyl[1,1′-binaphthalene]-2,2′-diyl)bis(oxy)]di(naphthalene-1-carboxylate)
{(6,6′-bis(naphthalen-1-yl)[1,1′-binaphthalene]-2,2′-diyl)bis(oxy-4,1-phenylene)}dimethanol
{(6,6′-bis(naphthalen-1-yl)[1,1′-binaphthalene]-2,2′-diyl)bis(oxy-2,1-phenylene)}dimethanol
[(6,6′-bis(naphthalen-1-yl)[1,1′-binaphthalene]-2,2′-diyl)bis(oxynaphthalene-4,1-diyl)]dimethanol
4,4′-{(6,6′-bis(naphthalen-1-yl)[1,1′-binaphthalene]-2,2′-diyl)bis(oxy)}dibenzoic acid
4,4′-[(6,6′-bis(naphthalen-1-yl)[1,1′-binaphthalene]-2,2′-diyl)bis(oxy)]di(naphthalene-1-carboxylic acid)
dimethyl 4,4′-{(6,6′-bis(naphthalen-1-yl)[1,1′-binaphthalene]-2,2′-diyl)bis(oxy)}dibenzoate
dimethyl 4,4′-[(bis(naphthalen-1-yl)[1,1′-binaphthalene]-2,2′-diyl)bis(oxy)]di(naphthalene-1-carboxylate)
{(6,6′-bis(naphthalen-2-yl)[1,1′-binaphthalene]-2,2′-diyl)bis(oxy-4,1-phenylene)}dimethanol
[(bis(naphthalen-2-yl)[1,1′-binaphthalene]-2,2′-diyl)bis(oxynaphthalene-4,1-diyl)]dimethanol
4,4′-{(6,6′-bis(naphthalen-2-yl)[1,1′-binaphthalene]-2,2′-diyl)bis(oxy)}dibenzoic acid
4,4′-[(bis(naphthalen-2-yl)[1,1′-binaphthalene]-2,2′-diyl)bis(oxy)]di(naphthalene-1-carboxylic acid)
dimethyl 4,4′-{(6,6′-bis(naphthalen-2-yl)[1,1′-binaphthalene]-2,2′-diyl)bis(oxy)}dibenzoate
dimethyl 4,4′-[(bis(naphthalen-2-yl)[1,1′-binaphthalene]-2,2′-diyl)bis(oxy)]di(naphthalene-1-carboxylate)
{[6,6′-di(phenanthren-9-yl)[1,1′-binaphthalene]-2,2′-diyl]bis(oxy-4,1-phenylene)}dimethanol
[(6,6′-di(phenanthren-9-yl)[1,1′-binaphthalene]-2,2′-diyl)bis(oxynaphthalene-4,1-diyl)]dimethanol
4,4′-{[6,6′-di(phenanthren-9-yl)[1,1′-binaphthalene]-2,2′-diyl]bis(oxy)}dibenzoic acid
4,4′-[(6,6′-di(phenanthren-9-yl)[1,1′-binaphthalene]-2,2′-diyl)bis(oxy)]di(naphthalene-1-carboxylic acid)
dimethyl 4,4′-{[6,6′-di(phenanthren-9-yl)[1,1′-binaphthalene]-2,2′-diyl]bis(oxy)}dibenzoate
dimethyl 4,4′-[(di(phenanthren-9-yl)[1,1′-binaphthalene]-2,2′-diyl)bis(oxy)]di(naphthalene-1-carboxylate)
{[6,6′-bis(dibenzo[b,d]thiophen-4-yl)[1,1′-binaphthalene]-2,2′-diyl]bis(oxy-4,1-phenylene)}dimethanol
{[6,6′-bis(dibenzo[b,a]thiophen-4-yl)[1,1′-binaphthalene]-2,2′-diyl]bis(oxynaphthalene-4,1-diyl)}dimethanol 2,2′-{[6,6′-bis(dibenzo[b,d]thiophen-4-yl)[1,1′-binaphthalene]-2,2′-diyl]bis(oxy)}dibenzoic acid
4,4′-{[6,6′-bis(dibenzo[b,]thiophen-4-yl)[1,1′-binaphthalene]-2,2′-diyl]bis(oxy)}di(naphthalene-1-carboxylic acid)
dimethyl 4,4′-{[6,6′-bis(dibenzo[b,d]thiophen-4-yl)[1,1′-binaphthalene]-2,2′-diyl]bis(oxy)}dibenzoate
dimethyl 4,4′-{[6,6′-bis(dibenzo[b,d]thiophen-4-yl)[1,1′-binaphthalene]-2,2′-diyl]bis(oxy)}di(naphthalene-1-carboxylate)
{[6,6′-di(thianthren-1-yl)[1,1′-binaphthalene]-2,2′-diyl]bis(oxy-4,1-phenylene)}dimethanol
{[6,6′-di(thianthren-1-yl)[1,1′-binaphthalene]-2,2′-diyl]bis(oxynaphthalene-4,1-diyl)}dimethanol
4,4-{[6,6′-di(thianthren-1-yl)[1,1′-binaphthalene]-2,2′-diyl]bis(oxy)}dibenzoic acid
4,4′-{[6,6′-di(thianthren-1-yl)[1,1′-binaphthalene]-2,2′-diyl]bis(oxy)}di(naphthalene-1-carboxylic acid)
dimethyl 4,4′-{[6,6′-di(thianthren-1-yl)[1,1′-binaphthalene]-2,2′-diyl]bis(oxy)}dibenzoate
dimethyl 4,4′-{[6,6′-di(thianthren-1-yl)[1,1′-binaphthalene]-2,2′-diyl]bis(oxy)}di(naphthalene-1-carboxylate).

In a particular subgroup (6/7-1) of group (6/7) of embodiments, the compound of formula (I) is a compound of the formula (Ia.1). Formula (Ia.1) corresponds to formula (I) where the variables p and q are either both 0 or both 1, the groups X¹ and X² are represented by the groups X which have the same meaning, the groups A¹ and A² are represented by 1,4-phenylene optionally substituted and the radicals Q⁰ are hydrogen or a radical Q² and the radicals R⁰ are hydrogen or represent the radicals R¹ and R², which have the same meaning:

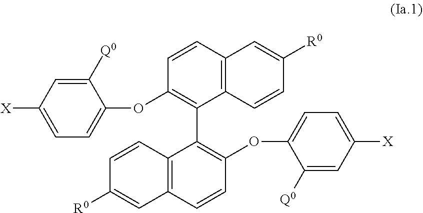

Thus, in formula (Ia.1) the variable X thus represents the identical groups X¹ and X², and R⁰ is H or a radical R¹/R², H, in particular fluorine or a radical R, especially H or a radical Q¹, and where Q⁰ is hydrogen or a radical Q² and where X¹, X², Q¹, Q² have the meanings defined herein, in particular the meanings mentioned herein as preferred.

In another particular subgroup (6/7-2) of group (6/7) of embodiments, the compound of formula (I) is a compound of the formula (Ia.2). Formula (Ia.2) corresponds to formula (I) where the variables p and q are either both 0 or both 1, the groups X¹ and X² are represented by the groups X which have the same meaning, the groups A¹ and A² are represented by 1,4-phenylene optionally substituted and the radicals Q⁰ are hydrogen or a radical Q² and the radicals R^{a} are hydrogen or represent the radicals R¹ and R², which have the same meaning:

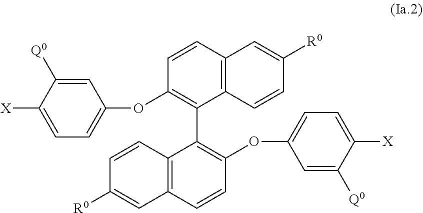

Thus, in formula (Ia.2) the variable X thus represents the identical groups X¹ and X², and R⁰ is H or a radical R¹/R², H, in particular fluorine or a radical R, especially H or a radical Q¹, and where Q⁰ is hydrogen or a radical Q² and where X¹, X², Q¹, Q² have the meanings defined herein, in particular the meanings mentioned herein as preferred.

In yet another particular subgroup (6/7-3) of group (6/7) of embodiments, the compound of formula (I) is a compound of the formula (Ia.3). Formula (Ia.3) corresponds to formula (I) where the variables p and q are either both 0 or both 1, the groups X¹ and X² are represented by the groups X which have the same meaning, the groups A¹ and A² are represented by 2,6-naphthylene optionally substituted and the radicals Q⁰ are hydrogen or a radical Q² and the radicals R⁰ are hydrogen or represent the radicals R¹ and R², which have the same meaning:

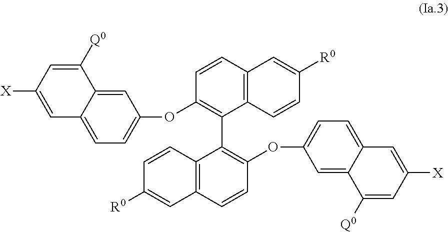

Thus, in formula (Ia.3) the variable X thus represents the identical groups X¹ and X², and R⁰ is H or a radical R¹/R², H, in particular fluorine or a radical R, especially H or a radical Q¹, and where Q⁰ is hydrogen or a radical Q² and where X¹, X², Q¹, Q² have the meanings defined herein, in particular the meanings mentioned herein as preferred.

In a particular subgroup (6/7-4) of group (6/7) of embodiments, the compound of formula (I) is a compound of the formula (Ia.4). Formula (Ia.4) corresponds to formula (I) where the variables p and q are either both 0 or both 1, the groups X¹ and X² are represented by the groups X which have the same meaning, the groups A¹ and A² are represented by 1,4-naphthylene optionally substituted and the radicals Q⁰ are hydrogen or a radical Q² and the radicals R⁰ are hydrogen or represent the radicals R¹ and R², which have the same meaning:

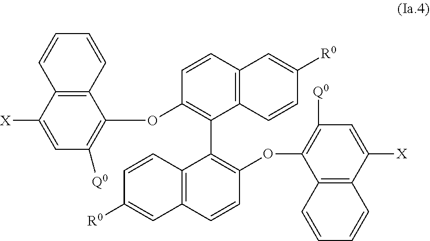

Thus, in formula (Ia.4) the variable X thus represents the identical groups X¹ and X², and R⁰ is H or a radical R¹/R², H, in particular fluorine or a radical R, especially H or a radical Q¹, and where Q⁰ is hydrogen or a radical Q² and where X¹, X², Q¹, Q² have the meanings defined herein, in particular the meanings mentioned herein as preferred.

Preferably, the moieties X in formulae (Ia.1), (Ia.2), (Ia.3) and (Ia.4) are defined either as in group (1) of the embodiments, in group (2) of the embodiments or in group (3) of the embodiments. Thus, the moieties X in formula (Ia) are here in particular selected from the group consisting of —CH₂OH (i.e. hydroxymethyl) and —C(O)OR^{x}, wherein R^{x} is hydrogen or C₁-C₄-alkyl, especially selected from —CH₂OH, —C(O)OH, —C(O)OCH₃ and —C(O)OCH₂CH₃, and specifically selected from —CH₂OH, —C(O)OH and —C(O)OCH₃. Accordingly, the moieties X^{a} in formula (IIa) are here selected from the group consisting of —CH₂O— and —C(O)O—.

Preference is also given to compounds of the formulae (Ia.1), (Ia.2), (Ia.3) and (Ia.4) and to structural units of the formula (IIb), where the groups R⁰ are defined as in one or more of groups (5), (5.1) and (5′) of embodiments. More preferably, the groups R⁰ in formulae (Ia.1), (Ia.2), (Ia.3) and (Ia.4) are defined as in group (5.1) of the embodiments. Thus, the groups R^{a} in formulae (Ib) and (IIb) are here in particular selected from the group consisting of fluorine, CN, methyl, methoxy and a radical Q¹.

In the context of formulae (Ia.1), (Ia.2), (Ia.3) and (Ia.4), R⁰ is in particular hydrogen or Q¹, where Q¹ is preferably mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms. Especially Q¹ is selected from the group consisting of phenyl, naphthyl, such as 1-naphthyl or 2-naphthyl, phenanthrenyl, such as 1-phenanthrenyl, 2-phenanthrenyl, 3-phenanthrenyl, 4-phenanthrenyl or 9-phenanthrenyl, thianthrenyl, such as 1-thianthrenyl or 2-thianthrenyl and dibenzo[b,d]thienyl, such as dibenzo[b,d]thien-1-yl, dibenzo[b,a]thien-2-yl, dibenzo[b,d]thien-3-yl or dibenzo[b,d]thien-4-yl, and specifically from the group consisting of phenyl or naphthyl, such as 1-naphthyl or 2-naphthyl.

In the context of formulae (Ia.1), (Ia.2), (Ia.3) and (Ia.4), Q⁰ is in particular hydrogen or Q², where Q² is preferably mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms. Especially Q² is selected from the group consisting of phenyl, naphthyl, such as 1-naphthyl or 2-naphthyl, phenanthrenyl, such as 1-phenanthrenyl, 2-phenanthrenyl, 3-phenanthrenyl, 4-phenanthrenyl or 9-phenanthrenyl, thianthrenyl, such as 1-thianthrenyl or 2-thianthrenyl and dibenzo[b,d]thienyl, such as dibenzo[b,d]thien-1-yl, dibenzo[b,a]thien-2-yl, dibenzo[b,d]thien-3-yl or dibenzo[b,d]thien-4-yl, and specifically from the group consisting of phenyl or naphthyl, such as 1-naphthyl or 2-naphthyl.

Examples of the particular subgroups (6/7-1), (6/7-2), (6/7-3) and (6/7-4) are the compounds of the formulae (Ia.1), (Ia.2), (Ia.3) and (Ia.4), in which the combination of the moieties X, the groups R⁰ and the groups Q⁰ are as defined in any one of the lines 1 to 504 in table B below:

TABLE B

| # | Formula | X | Q⁰ | R⁰ |
|---|---|---|---|---|
| 1 | Ia.1 | —CH₂OH | phenyl | H |
| 2 | Ia.1 | —C(O)OH | phenyl | H |
| 3 | Ia.1 | —C(O)OCH₃ | phenyl | H |
| 4 | Ia.1 | —CH₂OH | 1-naphthyl | H |
| 5 | Ia.1 | —C(O)OH | 1-naphthyl | H |
| 6 | Ia.1 | —C(O)OCH₃ | 1-naphthyl | H |
| 7 | Ia.1 | —CH₂OH | 2-naphthyl | H |
| 8 | Ia.1 | —C(O)OH | 2-naphthyl | H |
| 9 | Ia.1 | —C(O)OCH₃ | 2-naphthyl | H |
| 10 | Ia.1 | —CH₂OH | 9-phenanthryl | H |
| 11 | Ia.1 | —C(O)OH | 9-phenanthryl | H |
| 12 | Ia.1 | —C(O)OCH₃ | 9-phenanthryl | H |
| 13 | Ia.1 | —CH₂OH | dibenzo[b,d]thien-4-yl | H |
| 14 | Ia.1 | —C(O)OH | dibenzo[b,d]thien-4-yl | H |
| 15 | Ia.1 | —C(O)OCH₃ | dibenzo[b,d]thien-4-yl | H |
| 16 | Ia.1 | —CH₂OH | thianthren-1-yl | H |
| 17 | Ia.1 | —C(O)OH | thianthren-1-yl | H |
| 18 | Ia.1 | —C(O)OCH₃ | thianthren-1-yl | H |
| 19 | Ia.2 | —CH₂OH | phenyl | H |
| 20 | Ia.2 | —C(O)OH | phenyl | H |
| 21 | Ia.2 | —C(O)OCH₃ | phenyl | H |
| 22 | Ia.2 | —CH₂OH | 1-naphthyl | H |
| 23 | Ia.2 | —C(O)OH | 1-naphthyl | H |
| 24 | Ia.2 | —C(O)OCH₃ | 1-naphthyl | H |
| 25 | Ia.2 | —CH₂OH | 2-naphthyl | H |
| 26 | Ia.2 | —C(O)OH | 2-naphthyl | H |
| 27 | Ia.2 | —C(O)OCH₃ | 2-naphthyl | H |
| 28 | Ia.2 | —CH₂OH | 9-phenanthryl | H |
| 29 | Ia.2 | —C(O)OH | 9-phenanthryl | H |
| 30 | Ia.2 | —C(O)OCH₃ | 9-phenanthryl | H |
| 31 | Ia.2 | —CH₂OH | dibenzo[b,d]thien-4-yl | H |
| 32 | Ia.2 | —C(O)OH | dibenzo[b,d]thien-4-yl | H |
| 33 | Ia.2 | —C(O)OCH₃ | dibenzo[b,d]thien-4-yl | H |
| 34 | Ia.2 | —CH₂OH | thianthren-1-yl | H |
| 35 | Ia.2 | —C(O)OH | thianthren-1-yl | H |
| 36 | Ia.2 | —C(O)OCH₃ | thianthren-1-yl | H |
| 37 | Ia.3 | —CH₂OH | phenyl | H |
| 38 | Ia.3 | —C(O)OH | phenyl | H |
| 39 | Ia.3 | —C(O)OCH₃ | phenyl | H |
| 40 | Ia.3 | —CH₂OH | 1-naphthyl | H |
| 41 | Ia.3 | —C(O)OH | 1-naphthyl | H |
| 42 | Ia.3 | —C(O)OCH₃ | 1-naphthyl | H |
| 43 | Ia.3 | —CH₂OH | 2-naphthyl | H |
| 44 | Ia.3 | —C(O)OH | 2-naphthyl | H |
| 45 | Ia.3 | —C(O)OCH₃ | 2-naphthyl | H |
| 46 | Ia.3 | —CH₂OH | 9-phenanthryl | H |
| 47 | Ia.3 | —C(O)OH | 9-phenanthryl | H |
| 48 | Ia.3 | —C(O)OCH₃ | 9-phenanthryl | H |
| 49 | Ia.3 | —CH₂OH | dibenzo[b,d]thien-4-yl | H |
| 50 | Ia.3 | —C(O)OH | dibenzo[b,d]thien-4-yl | H |
| 51 | Ia.3 | —C(O)OCH₃ | dibenzo[b,d]thien-4-yl | H |
| 52 | Ia.3 | —CH₂OH | thianthren-1-yl | H |
| 53 | Ia.3 | —C(O)OH | thianthren-1-yl | H |
| 54 | Ia.3 | —C(O)OCH₃ | thianthren-1-yl | H |
| 55 | Ia.4 | —CH₂OH | phenyl | H |
| 56 | Ia.4 | —C(O)OH | phenyl | H |
| 57 | Ia.4 | —C(O)OCH₃ | phenyl | H |
| 58 | Ia.4 | —CH₂OH | 1-naphthyl | H |
| 59 | Ia.4 | —C(O)OH | 1-naphthyl | H |
| 60 | Ia.4 | —C(O)OCH₃ | 1-naphthyl | H |
| 61 | Ia.4 | —CH₂OH | 2-naphthyl | H |
| 62 | Ia.4 | —C(O)OH | 2-naphthyl | H |
| 63 | Ia.4 | —C(O)OCH₃ | 2-naphthyl | H |
| 64 | Ia.4 | —CH₂OH | 9-phenanthryl | H |
| 65 | Ia.4 | —C(O)OH | 9-phenanthryl | H |
| 66 | Ia.4 | —C(O)OCH₃ | 9-phenanthryl | H |
| 67 | Ia.4 | —CH₂OH | dibenzo[b,d]thien-4-yl | H |
| 68 | Ia.4 | —C(O)OH | dibenzo[b,d]thien-4-yl | H |
| 69 | Ia.4 | —C(O)OCH₃ | dibenzo[b,d]thien-4-yl | H |
| 70 | Ia.4 | —CH₂OH | thianthren-1-yl | H |
| 71 | Ia.4 | —C(O)OH | thianthren-1-yl | H |
| 72 | Ia.4 | —C(O)OCH₃ | thianthren-1-yl | H |
| 73 | Ia.1 | —CH₂OH | phenyl | phenyl |
| 74 | Ia.1 | —C(O)OH | phenyl | phenyl |
| 75 | Ia.1 | —C(O)OCH₃ | phenyl | phenyl |
| 76 | Ia.1 | —CH₂OH | 1-naphthyl | phenyl |
| 77 | Ia.1 | —C(O)OH | 1-naphthyl | phenyl |
| 78 | Ia.1 | —C(O)OCH₃ | 1-naphthyl | phenyl |
| 79 | Ia.1 | —CH₂OH | 2-naphthyl | phenyl |
| 80 | Ia.1 | —C(O)OH | 2-naphthyl | phenyl |
| 81 | Ia.1 | —C(O)OCH₃ | 2-naphthyl | phenyl |
| 82 | Ia.1 | —CH₂OH | 9-phenanthryl | phenyl |
| 83 | Ia.1 | —C(O)OH | 9-phenanthryl | phenyl |
| 84 | Ia.1 | —C(O)OCH₃ | 9-phenanthryl | phenyl |
| 85 | Ia.1 | —CH₂OH | dibenzo[b,d]thien-4-yl | phenyl |
| 86 | Ia.1 | —C(O)OH | dibenzo[b,d]thien-4-yl | phenyl |
| 87 | Ia.1 | —C(O)OCH₃ | dibenzo[b,d]thien-4-yl | phenyl |
| 88 | Ia.1 | —CH₂OH | thianthren-1-yl | phenyl |
| 89 | Ia.1 | —C(O)OH | thianthren-1-yl | phenyl |
| 90 | Ia.1 | —C(O)OCH₃ | thianthren-1-yl | phenyl |
| 91 | Ia.1 | —CH₂OH | phenyl | 1-naphthyl |
| 92 | Ia.1 | —C(O)OH | phenyl | 1-naphthyl |
| 93 | Ia.1 | —C(O)OCH₃ | phenyl | 1-naphthyl |
| 94 | Ia.1 | —CH₂OH | 1-naphthyl | 1-naphthyl |
| 95 | Ia.1 | —C(O)OH | 1-naphthyl | 1-naphthyl |
| 96 | Ia.1 | —C(O)OCH₃ | 1-naphthyl | 1-naphthyl |
| 97 | Ia.1 | —CH₂OH | 2-naphthyl | 1-naphthyl |
| 98 | Ia.1 | —C(O)OH | 2-naphthyl | 1-naphthyl |
| 99 | Ia.1 | —C(O)OCH₃ | 2-naphthyl | 1-naphthyl |
| 100 | Ia.1 | —CH₂OH | 9-phenanthryl | 1-naphthyl |
| 101 | Ia.1 | —C(O)OH | 9-phenanthryl | 1-naphthyl |
| 102 | Ia.1 | —C(O)OCH₃ | 9-phenanthryl | 1-naphthyl |
| 103 | Ia.1 | —CH₂OH | dibenzo[b,d]thien-4-yl | 1-naphthyl |
| 104 | Ia.1 | —C(O)OH | dibenzo[b,d]thien-4-yl | 1-naphthyl |
| 105 | Ia.1 | —C(O)OCH₃ | dibenzo[b,d]thien-4-yl | 1-naphthyl |
| 106 | Ia.1 | —CH₂OH | thianthren-1-yl | 1-naphthyl |
| 107 | Ia.1 | —C(O)OH | thianthren-1-yl | 1-naphthyl |
| 108 | Ia.1 | —C(O)OCH₃ | thianthren-1-yl | 1-naphthyl |
| 109 | Ia.1 | —CH₂OH | phenyl | 2-naphthyl |
| 110 | Ia.1 | —C(O)OH | phenyl | 2-naphthyl |
| 111 | Ia.1 | —C(O)OCH₃ | phenyl | 2-naphthyl |
| 112 | Ia.1 | —CH₂OH | 1-naphthyl | 2-naphthyl |
| 113 | Ia.1 | —C(O)OH | 1-naphthyl | 2-naphthyl |
| 114 | Ia.1 | —C(O)OCH₃ | 1-naphthyl | 2-naphthyl |
| 115 | Ia.1 | —CH₂OH | 2-naphthyl | 2-naphthyl |
| 116 | Ia.1 | —C(O)OH | 2-naphthyl | 2-naphthyl |
| 117 | Ia.1 | —C(O)OCH₃ | 2-naphthyl | 2-naphthyl |
| 118 | Ia.1 | —CH₂OH | 9-phenanthryl | 2-naphthyl |
| 119 | Ia.1 | —C(O)OH | 9-phenanthryl | 2-naphthyl |
| 120 | Ia.1 | —C(O)OCH₃ | 9-phenanthryl | 2-naphthyl |
| 121 | Ia.1 | —CH₂OH | dibenzo[b,d]thien-4-yl | 2-naphthyl |
| 122 | Ia.1 | —C(O)OH | dibenzo[b,d]thien-4-yl | 2-naphthyl |
| 123 | Ia.1 | —C(O)OCH₃ | dibenzo[b,d]thien-4-yl | 2-naphthyl |
| 124 | Ia.1 | —CH₂OH | thianthren-1-yl | 2-naphthyl |
| 125 | Ia.1 | —C(O)OH | thianthren-1-yl | 2-naphthyl |
| 126 | Ia.1 | —C(O)OCH₃ | thianthren-1-yl | 2-naphthyl |
| 127 | Ia.1 | —CH₂OH | phenyl | 9-phenanthryl |
| 128 | Ia.1 | —C(O)OH | phenyl | 9-phenanthryl |
| 129 | Ia.1 | —C(O)OCH₃ | phenyl | 9-phenanthryl |
| 130 | Ia.1 | —CH₂OH | 1-naphthyl | 9-phenanthryl |
| 131 | Ia.1 | —C(O)OH | 1-naphthyl | 9-phenanthryl |
| 132 | Ia.1 | —C(O)OCH₃ | 1-naphthyl | 9-phenanthryl |
| 133 | Ia.1 | —CH₂OH | 2-naphthyl | 9-phenanthryl |
| 134 | Ia.1 | —C(O)OH | 2-naphthyl | 9-phenanthryl |
| 135 | Ia.1 | —C(O)OCH₃ | 2-naphthyl | 9-phenanthryl |
| 136 | Ia.1 | —CH₂OH | 9-phenanthryl | 9-phenanthryl |
| 137 | Ia.1 | —C(O)OH | 9-phenanthryl | 9-phenanthryl |
| 138 | Ia.1 | —C(O)OCH₃ | 9-phenanthryl | 9-phenanthryl |
| 139 | Ia.1 | —CH₂OH | dibenzo[b,d]thien-4-yl | 9-phenanthryl |
| 140 | Ia.1 | —C(O)OH | dibenzo[b,d]thien-4-yl | 9-phenanthryl |
| 141 | Ia.1 | —C(O)OCH₃ | dibenzo[b,d]thien-4-yl | 9-phenanthryl |
| 142 | Ia.1 | —CH₂OH | thianthren-1-yl | 9-phenanthryl |
| 143 | Ia.1 | —C(O)OH | thianthren-1-yl | 9-phenanthryl |
| 144 | Ia.1 | —C(O)OCH₃ | thianthren-1-yl | 9-phenanthryl |
| 145 | Ia.1 | —CH₂OH | phenyl | dibenzo[b,d]thien-4-yl |
| 146 | Ia.1 | —C(O)OH | phenyl | dibenzo[b,d]thien-4-yl |
| 147 | Ia.1 | —C(O)OCH₃ | phenyl | dibenzo[b,d]thien-4-yl |
| 148 | Ia.1 | —CH₂OH | 1-naphthyl | dibenzo[b,d]thien-4-yl |
| 149 | Ia.1 | —C(O)OH | 1-naphthyl | dibenzo[b,d]thien-4-yl |
| 150 | Ia.1 | —C(O)OCH₃ | 1-naphthyl | dibenzo[b,d]thien-4-yl |
| 151 | Ia.1 | —CH₂OH | 2-naphthyl | dibenzo[b,d]thien-4-yl |
| 152 | Ia.1 | —C(O)OH | 2-naphthyl | dibenzo[b,d]thien-4-yl |
| 153 | Ia.1 | —C(O)OCH₃ | 2-naphthyl | dibenzo[b,d]thien-4-yl |
| 154 | Ia.1 | —CH₂OH | 9-phenanthryl | dibenzo[b,d]thien-4-yl |
| 155 | Ia.1 | —C(O)OH | 9-phenanthryl | dibenzo[b,d]thien-4-yl |
| 156 | Ia.1 | —C(O)OCH₃ | 9-phenanthryl | dibenzo[b,d]thien-4-yl |
| 157 | Ia.1 | —CH₂OH | dibenzo[b,d]thien-4-yl | dibenzo[b,d]thien-4-yl |
| 158 | Ia.1 | —C(O)OH | dibenzo[b,d]thien-4-yl | dibenzo[b,d]thien-4-yl |
| 159 | Ia.1 | —C(O)OCH₃ | dibenzo[b,d]thien-4-yl | dibenzo[b,d]thien-4-yl |
| 160 | Ia.1 | —CH₂OH | thianthren-1-yl | dibenzo[b,d]thien-4-yl |
| 161 | Ia.1 | —C(O)OH | thianthren-1-yl | dibenzo[b,d]thien-4-yl |
| 162 | Ia.1 | —C(O)OCH₃ | thianthren-1-yl | dibenzo[b,d]thien-4-yl |
| 163 | Ia.1 | —CH₂OH | phenyl | thianthren-1-yl |
| 164 | Ia.1 | —C(O)OH | phenyl | thianthren-1-yl |
| 165 | Ia.1 | —C(O)OCH₃ | phenyl | thianthren-1-yl |
| 166 | Ia.1 | —CH₂OH | 1-naphthyl | thianthren-1-yl |
| 167 | Ia.1 | —C(O)OH | 1-naphthyl | thianthren-1-yl |
| 168 | Ia.1 | —C(O)OCH₃ | 1-naphthyl | thianthren-1-yl |
| 169 | Ia.1 | —CH₂OH | 2-naphthyl | thianthren-1-yl |
| 170 | Ia.1 | —C(O)OH | 2-naphthyl | thianthren-1-yl |
| 171 | Ia.1 | —C(O)OCH₃ | 2-naphthyl | thianthren-1-yl |
| 172 | Ia.1 | —CH₂OH | 9-phenanthryl | thianthren-1-yl |
| 173 | Ia.1 | —C(O)OH | 9-phenanthryl | thianthren-1-yl |
| 174 | Ia.1 | —C(O)OCH₃ | 9-phenanthryl | thianthren-1-yl |
| 175 | Ia.1 | —CH₂OH | dibenzo[b,d]thien-4-yl | thianthren-1-yl |
| 176 | Ia.1 | —C(O)OH | dibenzo[b,d]thien-4-yl | thianthren-1-yl |
| 177 | Ia.1 | —C(O)OCH₃ | dibenzo[b,d]thien-4-yl | thianthren-1-yl |
| 178 | Ia.1 | —CH₂OH | thianthren-1-yl | thianthren-1-yl |
| 179 | Ia.1 | —C(O)OH | thianthren-1-yl | thianthren-1-yl |
| 180 | Ia.1 | —C(O)OCH₃ | thianthren-1-yl | thianthren-1-yl |
| 181 | Ia.2 | —CH₂OH | phenyl | phenyl |
| 182 | Ia.2 | —C(O)OH | phenyl | phenyl |
| 183 | Ia.2 | —C(O)OCH₃ | phenyl | phenyl |
| 184 | Ia.2 | —CH₂OH | 1-naphthyl | phenyl |
| 185 | Ia.2 | —C(O)OH | 1-naphthyl | phenyl |
| 186 | Ia.2 | —C(O)OCH₃ | 1-naphthyl | phenyl |
| 187 | Ia.2 | —CH₂OH | 2-naphthyl | phenyl |
| 188 | Ia.2 | —C(O)OH | 2-naphthyl | phenyl |
| 189 | Ia.2 | —C(O)OCH₃ | 2-naphthyl | phenyl |
| 190 | Ia.2 | —CH₂OH | 9-phenanthryl | phenyl |
| 191 | Ia.2 | —C(O)OH | 9-phenanthryl | phenyl |
| 192 | Ia.2 | —C(O)OCH₃ | 9-phenanthryl | phenyl |
| 193 | Ia.2 | —CH₂OH | dibenzo[b,d]thien-4-yl | phenyl |
| 194 | Ia.2 | —C(O)OH | dibenzo[b,d]thien-4-yl | phenyl |
| 195 | Ia.2 | —C(O)OCH₃ | dibenzo[b,d]thien-4-yl | phenyl |
| 196 | Ia.2 | —CH₂OH | thianthren-1-yl | phenyl |
| 197 | Ia.2 | —C(O)OH | thianthren-1-yl | phenyl |
| 198 | Ia.2 | —C(O)OCH₃ | thianthren-1-yl | phenyl |
| 199 | Ia.2 | —CH₂OH | phenyl | 1-naphthyl |
| 200 | Ia.2 | —C(O)OH | phenyl | 1-naphthyl |
| 201 | Ia.2 | —C(O)OCH₃ | phenyl | 1-naphthyl |
| 202 | Ia.2 | —CH₂OH | 1-naphthyl | 1-naphthyl |
| 203 | Ia.2 | —C(O)OH | 1-naphthyl | 1-naphthyl |
| 204 | Ia.2 | —C(O)OCH₃ | 1-naphthyl | 1-naphthyl |
| 205 | Ia.2 | —CH₂OH | 2-naphthyl | 1-naphthyl |
| 206 | Ia.2 | —C(O)OH | 2-naphthyl | 1-naphthyl |
| 207 | Ia.2 | —C(O)OCH₃ | 2-naphthyl | 1-naphthyl |
| 208 | Ia.2 | —CH₂OH | 9-phenanthryl | 1-naphthyl |
| 209 | Ia.2 | —C(O)OH | 9-phenanthryl | 1-naphthyl |
| 210 | Ia.2 | —C(O)OCH₃ | 9-phenanthryl | 1-naphthyl |
| 211 | Ia.2 | —CH₂OH | dibenzo[b,d]thien-4-yl | 1-naphthyl |
| 212 | Ia.2 | —C(O)OH | dibenzo[b,d]thien-4-yl | 1-naphthyl |
| 213 | Ia.2 | —C(O)OCH₃ | dibenzo[b,d]thien-4-yl | 1-naphthyl |
| 214 | Ia.2 | —CH₂OH | thianthren-1-yl | 1-naphthyl |
| 215 | Ia.2 | —C(O)OH | thianthren-1-yl | 1-naphthyl |
| 216 | Ia.2 | —C(O)OCH₃ | thianthren-1-yl | 1-naphthyl |
| 217 | Ia.2 | —CH₂OH | phenyl | 2-naphthyl |
| 218 | Ia.2 | —C(O)OH | phenyl | 2-naphthyl |
| 219 | Ia.2 | —C(O)OCH₃ | phenyl | 2-naphthyl |
| 220 | Ia.2 | —CH₂OH | 1-naphthyl | 2-naphthyl |
| 221 | Ia.2 | —C(O)OH | 1-naphthyl | 2-naphthyl |
| 222 | Ia.2 | —C(O)OCH₃ | 1-naphthyl | 2-naphthyl |
| 223 | Ia.2 | —CH₂OH | 2-naphthyl | 2-naphthyl |
| 224 | Ia.2 | —C(O)OH | 2-naphthyl | 2-naphthyl |
| 225 | Ia.2 | —C(O)OCH₃ | 2-naphthyl | 2-naphthyl |
| 226 | Ia.2 | —CH₂OH | 9-phenanthryl | 2-naphthyl |
| 227 | Ia.2 | —C(O)OH | 9-phenanthryl | 2-naphthyl |
| 228 | Ia.2 | —C(O)OCH₃ | 9-phenanthryl | 2-naphthyl |
| 229 | Ia.2 | —CH₂OH | dibenzo[b,d]thien-4-yl | 2-naphthyl |
| 230 | Ia.2 | —C(O)OH | dibenzo[b,d]thien-4-yl | 2-naphthyl |
| 231 | Ia.2 | —C(O)OCH₃ | dibenzo[b,d]thien-4-yl | 2-naphthyl |
| 232 | Ia.2 | —CH₂OH | thianthren-1-yl | 2-naphthyl |
| 233 | Ia.2 | —C(O)OH | thianthren-1-yl | 2-naphthyl |
| 234 | Ia.2 | —C(O)OCH₃ | thianthren-1-yl | 2-naphthyl |
| 235 | Ia.2 | —CH₂OH | phenyl | 9-phenanthryl |
| 236 | Ia.2 | —C(O)OH | phenyl | 9-phenanthryl |
| 237 | Ia.2 | —C(O)OCH₃ | phenyl | 9-phenanthryl |
| 238 | Ia.2 | —CH₂OH | 1-naphthyl | 9-phenanthryl |
| 239 | Ia.2 | —C(O)OH | 1-naphthyl | 9-phenanthryl |
| 240 | Ia.2 | —C(O)OCH₃ | 1-naphthyl | 9-phenanthryl |
| 241 | Ia.2 | —CH₂OH | 2-naphthyl | 9-phenanthryl |
| 242 | Ia.2 | —C(O)OH | 2-naphthyl | 9-phenanthryl |
| 243 | Ia.2 | —C(O)OCH₃ | 2-naphthyl | 9-phenanthryl |
| 244 | Ia.2 | —CH₂OH | 9-phenanthryl | 9-phenanthryl |
| 245 | Ia.2 | —C(O)OH | 9-phenanthryl | 9-phenanthryl |
| 246 | Ia.2 | —C(O)OCH₃ | 9-phenanthryl | 9-phenanthryl |
| 247 | Ia.2 | —CH₂OH | dibenzo[b,d]thien-4-yl | 9-phenanthryl |
| 248 | Ia.2 | —C(O)OH | dibenzo[b,d]thien-4-yl | 9-phenanthryl |
| 249 | Ia.2 | —C(O)OCH₃ | dibenzo[b,d]thien-4-yl | 9-phenanthryl |
| 250 | Ia.2 | —CH₂OH | thianthren-1-yl | 9-phenanthryl |
| 251 | Ia.2 | —C(O)OH | thianthren-1-yl | 9-phenanthryl |
| 252 | Ia.2 | —C(O)OCH₃ | thianthren-1-yl | 9-phenanthryl |
| 253 | Ia.2 | —CH₂OH | phenyl | dibenzo[b,d]thien-4-yl |
| 254 | Ia.2 | —C(O)OH | phenyl | dibenzo[b,d]thien-4-yl |
| 255 | Ia.2 | —C(O)OCH₃ | phenyl | dibenzo[b,d]thien-4-yl |
| 256 | Ia.2 | —CH₂OH | 1-naphthyl | dibenzo[b,d]thien-4-yl |
| 257 | Ia.2 | —C(O)OH | 1-naphthyl | dibenzo[b,d]thien-4-yl |
| 258 | Ia.2 | —C(O)OCH₃ | 1-naphthyl | dibenzo[b,d]thien-4-yl |
| 259 | Ia.2 | —CH₂OH | 2-naphthyl | dibenzo[b,d]thien-4-yl |
| 260 | Ia.2 | —C(O)OH | 2-naphthyl | dibenzo[b,d]thien-4-yl |
| 261 | Ia.2 | —C(O)OCH₃ | 2-naphthyl | dibenzo[b,d]thien-4-yl |
| 262 | Ia.2 | —CH₂OH | 9-phenanthryl | dibenzo[b,d]thien-4-yl |
| 263 | Ia.2 | —C(O)OH | 9-phenanthryl | dibenzo[b,d]thien-4-yl |
| 264 | Ia.2 | —C(O)OCH₃ | 9-phenanthryl | dibenzo[b,d]thien-4-yl |
| 265 | Ia.2 | —CH₂OH | dibenzo[b,d]thien-4-yl | dibenzo[b,d]thien-4-yl |
| 266 | Ia.2 | —C(O)OH | dibenzo[b,d]thien-4-yl | dibenzo[b,d]thien-4-yl |
| 267 | Ia.2 | —C(O)OCH₃ | dibenzo[b,d]thien-4-yl | dibenzo[b,d]thien-4-yl |
| 268 | Ia.2 | —CH₂OH | thianthren-1-yl | dibenzo[b,d]thien-4-yl |
| 269 | Ia.2 | —C(O)OH | thianthren-1-yl | dibenzo[b,d]thien-4-yl |
| 270 | Ia.2 | —C(O)OCH₃ | thianthren-1-yl | dibenzo[b,d]thien-4-yl |
| 271 | Ia.2 | —CH₂OH | phenyl | thianthren-1-yl |
| 272 | Ia.2 | —C(O)OH | phenyl | thianthren-1-yl |
| 273 | Ia.2 | —C(O)OCH₃ | phenyl | thianthren-1-yl |
| 274 | Ia.2 | —CH₂OH | 1-naphthyl | thianthren-1-yl |
| 275 | Ia.2 | —C(O)OH | 1-naphthyl | thianthren-1-yl |
| 276 | Ia.2 | —C(O)OCH₃ | 1-naphthyl | thianthren-1-yl |
| 277 | Ia.2 | —CH₂OH | 2-naphthyl | thianthren-1-yl |
| 278 | Ia.2 | —C(O)OH | 2-naphthyl | thianthren-1-yl |
| 279 | Ia.2 | —C(O)OCH₃ | 2-naphthyl | thianthren-1-yl |
| 280 | Ia.2 | —CH₂OH | 9-phenanthryl | thianthren-1-yl |
| 281 | Ia.2 | —C(O)OH | 9-phenanthryl | thianthren-1-yl |
| 282 | Ia.2 | —C(O)OCH₃ | 9-phenanthryl | thianthren-1-yl |
| 283 | Ia.2 | —CH₂OH | dibenzo[b,d]thien-4-yl | thianthren-1-yl |
| 284 | Ia.2 | —C(O)OH | dibenzo[b,d]thien-4-yl | thianthren-1-yl |
| 285 | Ia.2 | —C(O)OCH₃ | dibenzo[b,d]thien-4-yl | thianthren-1-yl |
| 286 | Ia.2 | —CH₂OH | thianthren-1-yl | thianthren-1-yl |
| 287 | Ia.2 | —C(O)OH | thianthren-1-yl | thianthren-1-yl |
| 288 | Ia.2 | —C(O)OCH₃ | thianthren-1-yl | thianthren-1-yl |
| 289 | Ia.3 | —CH₂OH | phenyl | phenyl |
| 290 | Ia.3 | —C(O)OH | phenyl | phenyl |
| 291 | Ia.3 | —C(O)OCH₃ | phenyl | phenyl |
| 292 | Ia.3 | —CH₂OH | 1-naphthyl | phenyl |
| 293 | Ia.3 | —C(O)OH | 1-naphthyl | phenyl |
| 294 | Ia.3 | —C(O)OCH₃ | 1-naphthyl | phenyl |
| 295 | Ia.3 | —CH₂OH | 2-naphthyl | phenyl |
| 296 | Ia.3 | —C(O)OH | 2-naphthyl | phenyl |
| 297 | Ia.3 | —C(O)OCH₃ | 2-naphthyl | phenyl |
| 298 | Ia.3 | —CH₂OH | 9-phenanthryl | phenyl |
| 299 | Ia.3 | —C(O)OH | 9-phenanthryl | phenyl |
| 300 | Ia.3 | —C(O)OCH₃ | 9-phenanthryl | phenyl |
| 301 | Ia.3 | —CH₂OH | dibenzo[b,d]thien-4-yl | phenyl |
| 302 | Ia.3 | —C(O)OH | dibenzo[b,d]thien-4-yl | phenyl |
| 303 | Ia.3 | —C(O)OCH₃ | dibenzo[b,d]thien-4-yl | phenyl |
| 304 | Ia.3 | —CH₂OH | thianthren-1-yl | phenyl |
| 305 | Ia.3 | —C(O)OH | thianthren-1-yl | phenyl |
| 306 | Ia.3 | —C(O)OCH₃ | thianthren-1-yl | phenyl |
| 307 | Ia.3 | —CH₂OH | phenyl | 1-naphthyl |
| 308 | Ia.3 | —C(O)OH | phenyl | 1-naphthyl |
| 309 | Ia.3 | —C(O)OCH₃ | phenyl | 1-naphthyl |
| 310 | Ia.3 | —CH₂OH | 1-naphthyl | 1-naphthyl |
| 311 | Ia.3 | —C(O)OH | 1-naphthyl | 1-naphthyl |
| 312 | Ia.3 | —C(O)OCH₃ | 1-naphthyl | 1-naphthyl |
| 313 | Ia.3 | —CH₂OH | 2-naphthyl | 1-naphthyl |
| 314 | Ia.3 | —C(O)OH | 2-naphthyl | 1-naphthyl |
| 315 | Ia.3 | —C(O)OCH₃ | 2-naphthyl | 1-naphthyl |
| 316 | Ia.3 | —CH₂OH | 9-phenanthryl | 1-naphthyl |
| 317 | Ia.3 | —C(O)OH | 9-phenanthryl | 1-naphthyl |
| 318 | Ia.3 | —C(O)OCH₃ | 9-phenanthryl | 1-naphthyl |
| 319 | Ia.3 | —CH₂OH | dibenzo[b,d]thien-4-yl | 1-naphthyl |
| 320 | Ia.3 | —C(O)OH | dibenzo[b,d]thien-4-yl | 1-naphthyl |
| 321 | Ia.3 | —C(O)OCH₃ | dibenzo[b,d]thien-4-yl | 1-naphthyl |
| 322 | Ia.3 | —CH₂OH | thianthren-1-yl | 1-naphthyl |
| 323 | Ia.3 | —C(O)OH | thianthren-1-yl | 1-naphthyl |
| 324 | Ia.3 | —C(O)OCH₃ | thianthren-1-yl | 1-naphthyl |
| 325 | Ia.3 | —CH₂OH | phenyl | 2-naphthyl |
| 326 | Ia.3 | —C(O)OH | phenyl | 2-naphthyl |
| 327 | Ia.3 | —C(O)OCH₃ | phenyl | 2-naphthyl |
| 328 | Ia.3 | —CH₂OH | 1-naphthyl | 2-naphthyl |
| 329 | Ia.3 | —C(O)OH | 1-naphthyl | 2-naphthyl |
| 330 | Ia.3 | —C(O)OCH₃ | 1-naphthyl | 2-naphthyl |
| 331 | Ia.3 | —CH₂OH | 2-naphthyl | 2-naphthyl |
| 332 | Ia.3 | —C(O)OH | 2-naphthyl | 2-naphthyl |
| 333 | Ia.3 | —C(O)OCH₃ | 2-naphthyl | 2-naphthyl |
| 334 | Ia.3 | —CH₂OH | 9-phenanthryl | 2-naphthyl |
| 335 | Ia.3 | —C(O)OH | 9-phenanthryl | 2-naphthyl |
| 336 | Ia.3 | —C(O)OCH₃ | 9-phenanthryl | 2-naphthyl |
| 337 | Ia.3 | —CH₂OH | dibenzo[b,d]thien-4-yl | 2-naphthyl |
| 338 | Ia.3 | —C(O)OH | dibenzo[b,d]thien-4-yl | 2-naphthyl |
| 339 | Ia.3 | —C(O)OCH₃ | dibenzo[b,d]thien-4-yl | 2-naphthyl |
| 340 | Ia.3 | —CH₂OH | thianthren-1-yl | 2-naphthyl |
| 341 | Ia.3 | —C(O)OH | thianthren-1-yl | 2-naphthyl |
| 342 | Ia.3 | —C(O)OCH₃ | thianthren-1-yl | 2-naphthyl |
| 343 | Ia.3 | —CH₂OH | phenyl | 9-phenanthryl |
| 344 | Ia.3 | —C(O)OH | phenyl | 9-phenanthryl |
| 345 | Ia.3 | —C(O)OCH₃ | phenyl | 9-phenanthryl |
| 346 | Ia.3 | —CH₂OH | 1-naphthyl | 9-phenanthryl |
| 347 | Ia.3 | —C(O)OH | 1-naphthyl | 9-phenanthryl |
| 348 | Ia.3 | —C(O)OCH₃ | 1-naphthyl | 9-phenanthryl |
| 349 | Ia.3 | —CH₂OH | 2-naphthyl | 9-phenanthryl |
| 350 | Ia.3 | —C(O)OH | 2-naphthyl | 9-phenanthryl |
| 351 | Ia.3 | —C(O)OCH₃ | 2-naphthyl | 9-phenanthryl |
| 352 | Ia.3 | —CH₂OH | 9-phenanthryl | 9-phenanthryl |
| 353 | Ia.3 | —C(O)OH | 9-phenanthryl | 9-phenanthryl |
| 354 | Ia.3 | —C(O)OCH₃ | 9-phenanthryl | 9-phenanthryl |
| 355 | Ia.3 | —CH₂OH | dibenzo[b,d]thien-4-yl | 9-phenanthryl |
| 356 | Ia.3 | —C(O)OH | dibenzo[b,d]thien-4-yl | 9-phenanthryl |
| 357 | Ia.3 | —C(O)OCH₃ | dibenzo[b,d]thien-4-yl | 9-phenanthryl |
| 358 | Ia.3 | —CH₂OH | thianthren-1-yl | 9-phenanthryl |
| 359 | Ia.3 | —C(O)OH | thianthren-1-yl | 9-phenanthryl |
| 360 | Ia.3 | —C(O)OCH₃ | thianthren-1-yl | 9-phenanthryl |
| 361 | Ia.3 | —CH₂OH | phenyl | dibenzo[b,d]thien-4-yl |
| 362 | Ia.3 | —C(O)OH | phenyl | dibenzo[b,d]thien-4-yl |
| 363 | Ia.3 | —C(O)OCH₃ | phenyl | dibenzo[b,d]thien-4-yl |
| 364 | Ia.3 | —CH₂OH | 1-naphthyl | dibenzo[b,d]thien-4-yl |
| 365 | Ia.3 | —C(O)OH | 1-naphthyl | dibenzo[b,d]thien-4-yl |
| 366 | Ia.3 | —C(O)OCH₃ | 1-naphthyl | dibenzo[b,d]thien-4-yl |
| 367 | Ia.3 | —CH₂OH | 2-naphthyl | dibenzo[b,d]thien-4-yl |
| 368 | Ia.3 | —C(O)OH | 2-naphthyl | dibenzo[b,d]thien-4-yl |
| 369 | Ia.3 | —C(O)OCH₃ | 2-naphthyl | dibenzo[b,d]thien-4-yl |
| 370 | Ia.3 | —CH₂OH | 9-phenanthryl | dibenzo[b,d]thien-4-yl |
| 371 | Ia.3 | —C(O)OH | 9-phenanthryl | dibenzo[b,d]thien-4-yl |
| 372 | Ia.3 | —C(O)OCH₃ | 9-phenanthryl | dibenzo[b,d]thien-4-yl |
| 373 | Ia.3 | —CH₂OH | dibenzo[b,d]thien-4-yl | dibenzo[b,d]thien-4-yl |
| 374 | Ia.3 | —C(O)OH | dibenzo[b,d]thien-4-yl | dibenzo[b,d]thien-4-yl |
| 375 | Ia.3 | —C(O)OCH₃ | dibenzo[b,d]thien-4-yl | dibenzo[b,d]thien-4-yl |
| 376 | Ia.3 | —CH₂OH | thianthren-1-yl | dibenzo[b,d]thien-4-yl |
| 377 | Ia.3 | —C(O)OH | thianthren-1-yl | dibenzo[b,d]thien-4-yl |
| 378 | Ia.3 | —C(O)OCH₃ | thianthren-1-yl | dibenzo[b,d]thien-4-yl |
| 379 | Ia.3 | —CH₂OH | phenyl | thianthren-1-yl |
| 380 | Ia.3 | —C(O)OH | phenyl | thianthren-1-yl |
| 381 | Ia.3 | —C(O)OCH₃ | phenyl | thianthren-1-yl |
| 382 | Ia.3 | —CH₂OH | 1-naphthyl | thianthren-1-yl |
| 383 | Ia.3 | —C(O)OH | 1-naphthyl | thianthren-1-yl |
| 384 | Ia.3 | —C(O)OCH₃ | 1-naphthyl | thianthren-1-yl |
| 385 | Ia.3 | —CH₂OH | 2-naphthyl | thianthren-1-yl |
| 386 | Ia.3 | —C(O)OH | 2-naphthyl | thianthren-1-yl |
| 387 | Ia.3 | —C(O)OCH₃ | 2-naphthyl | thianthren-1-yl |
| 388 | Ia.3 | —CH₂OH | 9-phenanthryl | thianthren-1-yl |
| 389 | Ia.3 | —C(O)OH | 9-phenanthryl | thianthren-1-yl |
| 390 | Ia.3 | —C(O)OCH₃ | 9-phenanthryl | thianthren-1-yl |
| 391 | Ia.3 | —CH₂OH | dibenzo[b,d]thien-4-yl | thianthren-1-yl |
| 392 | Ia.3 | —C(O)OH | dibenzo[b,d]thien-4-yl | thianthren-1-yl |
| 393 | Ia.3 | —C(O)OCH₃ | dibenzo[b,d]thien-4-yl | thianthren-1-yl |
| 394 | Ia.3 | —CH₂OH | thianthren-1-yl | thianthren-1-yl |
| 395 | Ia.3 | —C(O)OH | thianthren-1-yl | thianthren-1-yl |
| 396 | Ia.3 | —C(O)OCH₃ | thianthren-1-yl | thianthren-1-yl |
| 397 | Ia.4 | —CH₂OH | phenyl | phenyl |
| 398 | Ia.4 | —C(O)OH | phenyl | phenyl |
| 399 | Ia.4 | —C(O)OCH₃ | phenyl | phenyl |
| 400 | Ia.4 | —CH₂OH | 1-naphthyl | phenyl |
| 401 | Ia.4 | —C(O)OH | 1-naphthyl | phenyl |
| 402 | Ia.4 | —C(O)OCH₃ | 1-naphthyl | phenyl |
| 403 | Ia.4 | —CH₂OH | 2-naphthyl | phenyl |
| 404 | Ia.4 | —C(O)OH | 2-naphthyl | phenyl |
| 405 | Ia.4 | —C(O)OCH₃ | 2-naphthyl | phenyl |
| 406 | Ia.4 | —CH₂OH | 9-phenanthryl | phenyl |
| 407 | Ia.4 | —C(O)OH | 9-phenanthryl | phenyl |
| 408 | Ia.4 | —C(O)OCH₃ | 9-phenanthryl | phenyl |
| 409 | Ia.4 | —CH₂OH | dibenzo[b,d]thien-4-yl | phenyl |
| 410 | Ia.4 | —C(O)OH | dibenzo[b,d]thien-4-yl | phenyl |
| 411 | Ia.4 | —C(O)OCH₃ | dibenzo[b,d]thien-4-yl | phenyl |
| 412 | Ia.4 | —CH₂OH | thianthren-1-yl | phenyl |
| 413 | Ia.4 | —C(O)OH | thianthren-1-yl | phenyl |
| 414 | Ia.4 | —C(O)OCH₃ | thianthren-1-yl | phenyl |
| 415 | Ia.4 | —CH₂OH | phenyl | 1-naphthyl |
| 416 | Ia.4 | —C(O)OH | phenyl | 1-naphthyl |
| 417 | Ia.4 | —C(O)OCH₃ | phenyl | 1-naphthyl |
| 418 | Ia.4 | —CH₂OH | 1-naphthyl | 1-naphthyl |
| 419 | Ia.4 | —C(O)OH | 1-naphthyl | 1-naphthyl |
| 420 | Ia.4 | —C(O)OCH₃ | 1-naphthyl | 1-naphthyl |
| 421 | Ia.4 | —CH₂OH | 2-naphthyl | 1-naphthyl |
| 422 | Ia.4 | —C(O)OH | 2-naphthyl | 1-naphthyl |
| 423 | Ia.4 | —C(O)OCH₃ | 2-naphthyl | 1-naphthyl |
| 424 | Ia.4 | —CH₂OH | 9-phenanthryl | 1-naphthyl |
| 425 | Ia.4 | —C(O)OH | 9-phenanthryl | 1-naphthyl |
| 426 | Ia.4 | —C(O)OCH₃ | 9-phenanthryl | 1-naphthyl |
| 427 | Ia.4 | —CH₂OH | dibenzo[b,d]thien-4-yl | 1-naphthyl |
| 428 | Ia.4 | —C(O)OH | dibenzo[b,d]thien-4-yl | 1-naphthyl |
| 429 | Ia.4 | —C(O)OCH₃ | dibenzo[b,d]thien-4-yl | 1-naphthyl |
| 430 | Ia.4 | —CH₂OH | thianthren-1-yl | 1-naphthyl |
| 431 | Ia.4 | —C(O)OH | thianthren-1-yl | 1-naphthyl |
| 432 | Ia.4 | —C(O)OCH₃ | thianthren-1-yl | 1-naphthyl |
| 433 | Ia.4 | —CH₂OH | phenyl | 2-naphthyl |
| 434 | Ia.4 | —C(O)OH | phenyl | 2-naphthyl |
| 435 | Ia.4 | —C(O)OCH₃ | phenyl | 2-naphthyl |
| 436 | Ia.4 | —CH₂OH | 1-naphthyl | 2-naphthyl |
| 437 | Ia.4 | —C(O)OH | 1-naphthyl | 2-naphthyl |
| 438 | Ia.4 | —C(O)OCH₃ | 1-naphthyl | 2-naphthyl |
| 439 | Ia.4 | —CH₂OH | 2-naphthyl | 2-naphthyl |
| 440 | Ia.4 | —C(O)OH | 2-naphthyl | 2-naphthyl |
| 441 | Ia.4 | —C(O)OCH₃ | 2-naphthyl | 2-naphthyl |
| 442 | Ia.4 | —CH₂OH | 9-phenanthryl | 2-naphthyl |
| 443 | Ia.4 | —C(O)OH | 9-phenanthryl | 2-naphthyl |
| 444 | Ia.4 | —C(O)OCH₃ | 9-phenanthryl | 2-naphthyl |
| 445 | Ia.4 | —CH₂OH | dibenzo[b,d]thien-4-yl | 2-naphthyl |
| 446 | Ia.4 | —C(O)OH | dibenzo[b,d]thien-4-yl | 2-naphthyl |
| 447 | Ia.4 | —C(O)OCH₃ | dibenzo[b,d]thien-4-yl | 2-naphthyl |
| 448 | Ia.4 | —CH₂OH | thianthren-1-yl | 2-naphthyl |
| 449 | Ia.4 | —C(O)OH | thianthren-1-yl | 2-naphthyl |
| 450 | Ia.4 | —C(O)OCH₃ | thianthren-1-yl | 2-naphthyl |
| 451 | Ia.4 | —CH₂OH | phenyl | 9-phenanthryl |
| 452 | Ia.4 | —C(O)OH | phenyl | 9-phenanthryl |
| 453 | Ia.4 | —C(O)OCH₃ | phenyl | 9-phenanthryl |
| 454 | Ia.4 | —CH₂OH | 1-naphthyl | 9-phenanthryl |
| 455 | Ia.4 | —C(O)OH | 1-naphthyl | 9-phenanthryl |
| 456 | Ia.4 | —C(O)OCH₃ | 1-naphthyl | 9-phenanthryl |
| 457 | Ia.4 | —CH₂OH | 2-naphthyl | 9-phenanthryl |
| 458 | Ia.4 | —C(O)OH | 2-naphthyl | 9-phenanthryl |
| 459 | Ia.4 | —C(O)OCH₃ | 2-naphthyl | 9-phenanthryl |
| 460 | Ia.4 | —CH₂OH | 9-phenanthryl | 9-phenanthryl |
| 461 | Ia.4 | —C(O)OH | 9-phenanthryl | 9-phenanthryl |
| 462 | Ia.4 | —C(O)OCH₃ | 9-phenanthryl | 9-phenanthryl |
| 463 | Ia.4 | —CH₂OH | dibenzo[b,d]thien-4-yl | 9-phenanthryl |
| 464 | Ia.4 | —C(O)OH | dibenzo[b,d]thien-4-yl | 9-phenanthryl |
| 465 | Ia.4 | —C(O)OCH₃ | dibenzo[b,d]thien-4-yl | 9-phenanthryl |
| 466 | Ia.4 | —CH₂OH | thianthren-1-yl | 9-phenanthryl |
| 467 | Ia.4 | —C(O)OH | thianthren-1-yl | 9-phenanthryl |
| 468 | Ia.4 | —C(O)OCH₃ | thianthren-1-yl | 9-phenanthryl |
| 469 | Ia.4 | —CH₂OH | phenyl | dibenzo[b,d]thien-4-yl |
| 470 | Ia.4 | —C(O)OH | phenyl | dibenzo[b,d]thien-4-yl |
| 471 | Ia.4 | —C(O)OCH₃ | phenyl | dibenzo[b,d]thien-4-yl |
| 472 | Ia.4 | —CH₂OH | 1-naphthyl | dibenzo[b,d]thien-4-yl |
| 473 | Ia.4 | —C(O)OH | 1-naphthyl | dibenzo[b,d]thien-4-yl |
| 474 | Ia.4 | —C(O)OCH₃ | 1-naphthyl | dibenzo[b,d]thien-4-yl |
| 475 | Ia.4 | —CH₂OH | 2-naphthyl | dibenzo[b,d]thien-4-yl |
| 476 | Ia.4 | —C(O)OH | 2-naphthyl | dibenzo[b,d]thien-4-yl |
| 477 | Ia.4 | —C(O)OCH₃ | 2-naphthyl | dibenzo[b,d]thien-4-yl |
| 478 | Ia.4 | —CH₂OH | 9-phenanthryl | dibenzo[b,d]thien-4-yl |
| 479 | Ia.4 | —C(O)OH | 9-phenanthryl | dibenzo[b,d]thien-4-yl |
| 480 | Ia.4 | —C(O)OCH₃ | 9-phenanthryl | dibenzo[b,d]thien-4-yl |
| 481 | Ia.4 | —CH₂OH | dibenzo[b,d]thien-4-yl | dibenzo[b,d]thien-4-yl |
| 482 | Ia.4 | —C(O)OH | dibenzo[b,d]thien-4-yl | dibenzo[b,d]thien-4-yl |
| 483 | Ia.4 | —C(O)OCH₃ | dibenzo[b,d]thien-4-yl | dibenzo[b,d]thien-4-yl |
| 484 | Ia.4 | —CH₂OH | thianthren-1-yl | dibenzo[b,d]thien-4-yl |
| 485 | Ia.4 | —C(O)OH | thianthren-1-yl | dibenzo[b,d]thien-4-yl |
| 486 | Ia.4 | —C(O)OCH₃ | thianthren-1-yl | dibenzo[b,d]thien-4-yl |
| 487 | Ia.4 | —CH₂OH | phenyl | thianthren-1-yl |
| 488 | Ia.4 | —C(O)OH | phenyl | thianthren-1-yl |
| 489 | Ia.4 | —C(O)OCH₃ | phenyl | thianthren-1-yl |
| 490 | Ia.4 | —CH₂OH | 1-naphthyl | thianthren-1-yl |
| 491 | Ia.4 | —C(O)OH | 1-naphthyl | thianthren-1-yl |
| 492 | Ia.4 | —C(O)OCH₃ | 1-naphthyl | thianthren-1-yl |
| 493 | Ia.4 | —CH₂OH | 2-naphthyl | thianthren-1-yl |
| 494 | Ia.4 | —C(O)OH | 2-naphthyl | thianthren-1-yl |
| 495 | Ia.4 | —C(O)OCH₃ | 2-naphthyl | thianthren-1-yl |
| 496 | Ia.4 | —CH₂OH | 9-phenanthryl | thianthren-1-yl |
| 497 | Ia.4 | —C(O)OH | 9-phenanthryl | thianthren-1-yl |
| 498 | Ia.4 | —C(O)OCH₃ | 9-phenanthryl | thianthren-1-yl |
| 499 | Ia.4 | —CH₂OH | dibenzo[b,d]thien-4-yl | thianthren-1-yl |
| 500 | Ia.4 | —C(O)OH | dibenzo[b,d]thien-4-yl | thianthren-1-yl |
| 501 | Ia.4 | —C(O)OCH₃ | dibenzo[b,d]thien-4-yl | thianthren-1-yl |
| 502 | Ia.4 | —CH₂OH | thianthren-1-yl | thianthren-1-yl |
| 503 | Ia.4 | —C(O)OH | thianthren-1-yl | thianthren-1-yl |
| 504 | Ia.4 | —C(O)OCH₃ | thianthren-1-yl | thianthren-1-yl |

Amongst the compounds of formulae (Ia.1), (Ia.2), (Ia.3) and (Ia.4) recited in table B, particular preference is given to the following compounds:
[[1,1′-binaphthalene]-2,2′-diylbis(oxy[1,1′-biphenyl]-2,5-diyl)]dimethanol
6,6′-[[1,1′-binaphthalene]-2,2′-diylbis(oxy)]di([1,1′-biphenyl]-3-carboxylic acid)
dimethyl 6,6′-[[1,1′-binaphthalene]-2,2′-diylbis(oxy)]di([1,1′-biphenyl]-3-carboxylate)
([1,1′-binaphthalene]-2,2′-diylbis{oxy[3-(naphthalen-1-yl)-4,1-phenylene]})dimethanol
4,4′-[[1,1′-binaphthalene]-2,2′-diylbis(oxy)]bis[3-(naphthalen-1-yl)benzoic acid]
dimethyl 4,4′-[[1,1′-binaphthalene]-2,2′-diylbis(oxy)]bis[3-(naphthalen-1-yl)benzoate]
([1,1′-binaphthalene]-2,2′-diylbis{oxy[3-(naphthalen-2-yl)-4,1-phenylene]})dimethanol
4,4′-[[1,1′-binaphthalene]-2,2′-diylbis(oxy)]bis[3-(naphthalen-2-yl)benzoic acid]
dimethyl 4,4′-[[1,1′-binaphthalene]-2,2′-diylbis(oxy)]bis[3-(naphthalen-2-yl)benzoate]
([1,1′-binaphthalene]-2,2′-diylbis{oxy[3-(phenanthren-9-yl)-4,1-phenylene]})dimethanol
4,4′-[[1,1′-binaphthalene]-2,2′-diylbis(oxy)]bis[3-(phenanthren-9-yl)benzoic acid]
([1,1′-binaphthalene]-2,2′-diylbis{oxy[3-(dibenzo[b,d]thiophen-4-yl)-4,1-phenylene]})dimethanol
4,4′-[[1,1′-binaphthalene]-2,2′-diylbis(oxy)]bis[3-(dibenzo[b,d]thiophen-4-yl)benzoic acid]
([1,1′-binaphthalene]-2,2′-diylbis{oxy[3-(thianthren-1-yl)-4,1-phenylene]})dimethanol
4,4′-[[1,1′-binaphthalene]-2,2′-diylbis(oxy)]bis[3-(thianthren-1-yl)benzoic acid]
[[1,1′-binaphthalene]-2,2′-diylbis(oxy[1,1′-biphenyl]-2,5-diyl)]dimethanol
6,6′-[[1,1′-binaphthalene]-2,2′-diylbis(oxy)]di([1,1′-biphenyl]-3-carboxylic acid)
dimethyl 6,6′-[[1,1′-binaphthalene]-2,2′-diylbis(oxy)]di([1,1′-biphenyl]-3-carboxylate)
([1,1′-binaphthalene]-2,2′-diylbis{oxy[3-(naphthalen-1-yl)-4,1-phenylene]})dimethanol
4,4′-[[1,1′-binaphthalene]-2,2′-diylbis(oxy)]bis[3-(naphthalen-1-yl)benzoic acid]
([1,1′-binaphthalene]-2,2′-diylbis{oxy[3-(naphthalen-2-yl)-4,1-phenylene]})dimethanol
4,4′-[[1,1′-binaphthalene]-2,2′-diylbis(oxy)]bis[3-(naphthalen-2-yl)benzoic acid]
([1,1′-binaphthalene]-2,2′-diylbis{oxy[3-(phenanthren-9-yl)-4,1-phenylene]})dimethanol
([1,1′-binaphthalene]-2,2′-diylbis{oxy[3-(dibenzo[b,d]thiophen-4-yl)-4,1-phenylene]})dimethanol
4,4′-[[1,1′-binaphthalene]-2,2′-diylbis(oxy)]bis[3-(dibenzo[b,d]thiophen-4-yl)benzoic acid]
([1,1′-binaphthalene]-2,2′-diylbis{oxy[3-(thianthren-1-yl)-4,1-phenylene]})dimethanol
4,4′-[[1,1′-binaphthalene]-2,2′-diylbis(oxy)]bis[3-(thianthren-1-yl)benzoic acid]
{[1,1′-binaphthalene]-2,2′-diylbis[oxy(4-phenylnaphthalene-6,2-diyl)]}dimethanol
6,6′-[[1,1′-binaphthalene]-2,2′-diylbis(oxy)]bis(4-phenylnaphthalene-2-carboxylic acid)
[[1,1′-binaphthalene]-2,2′-diylbis(oxy[1,1′-binaphthalene]-7,3-diyl)]dimethanol
7,7′-[[1,1′-binaphthalene]-2,2′-diylbis(oxy)]di([1,1′-binaphthalene]-3-carboxylic acid)
[[1,1′-binaphthalene]-2,2′-diylbis(oxy[1,2′-binaphthalene]-7,3-diyl)]dimethanol
([1,1′-binaphthalene]-2,2′-diylbis{oxy[4-(dibenzo[b,a]thiophen-4-yl)naphthalene-6,2-diyl]})dimethanol
([1,1′-binaphthalene]-2,2′-diylbis{oxy[4-(thianthren-1-yl)naphthalene-6,2-diyl]})dimethanol
{[1,1′-binaphthalene]-2,2′-diylbis[oxy(3-phenylnaphthalene-4,1-diyl)]}dimethanol
4,4′-[[1,1′-binaphthalene]-2,2′-diylbis(oxy)]bis(3-phenylnaphthalene-1-carboxylic acid)
dimethyl 4,4′-[[1,1′-binaphthalene]-2,2′-diylbis(oxy)]bis(3-phenylnaphthalene-1-carboxylate)
[[1,1′-binaphthalene]-2,2′-diylbis(oxy[1,2′-binaphthalene]-1′,4′-diyl)]dimethanol
1′,1″-[[1,1′-binaphthalene]-2,2′-diylbis(oxy)]di([1,2′-binaphthalene]-4′-carboxylic acid)
[[1,1′-binaphthalene]-2,2′-diylbis(oxy[2,2′-binaphthalene]-1,4-diyl)]dimethanol
1,1′-[[1,1′-binaphthalene]-2,2′-diylbis(oxy)]di([2,2′-binaphthalene]-4-carboxylic acid)
dimethyl 1,1′-[[1,1′-binaphthalene]-2,2′-diylbis(oxy)]di([2,2′-binaphthalene]-4-carboxylate
([1,1′-binaphthalene]-2,2′-diylbis{oxy[3-(phenanthren-9-yl)naphthalene-4,1-diyl]})dimethanol
([1,1′-binaphthalene]-2,2′-diylbis{oxy[3-(dibenzo[b,d]thiophen-4-yl)naphthalene-4,1-diyl]})dimethanol
([1,1′-binaphthalene]-2,2′-diylbis{oxy[3-(thianthren-1-yl)naphthalene-4,1-diyl]})dimethanol
[(6,6′-diphenyl[1,1′-binaphthalene]-2,2′-diyl)bis(oxy[1,1′-biphenyl]-2,5-diyl)]dimethanol
6,6′-[(6,6′-diphenyl[1,1′-binaphthalene]-2,2′-diyl)bis(oxy)]di([1,1′-biphenyl]-3-carboxylic acid)
dimethyl 6,6′-[(6,6′-diphenyl[1,1′-binaphthalene]-2,2′-diyl)bis(oxy)]di([1,1′-biphenyl]-3-carboxylate)
[(6,6′-diphenyl[1,1′-binaphthalene]-2,2′-diyl)bis{oxy[3-(naphthalen-1-yl)-4,1-phenylene]}]dimethanol
4,4′-[(6,6′-diphenyl[1,1′-binaphthalene]-2,2′-diyl)bis(oxy)]bis[3-(naphthalen-1-yl)benzoic acid]
[(6,6′-diphenyl[1,1′-binaphthalene]-2,2′-diyl)bis{oxy[3-(naphthalen-2-yl)-4,1-phenylene]}]dimethanol
4,4′-[(6,6′-diphenyl[1,1′-binaphthalene]-2,2′-diyl)bis(oxy)]bis[3-(naphthalen-2-yl)benzoic acid]
[(6,6′-diphenyl[1,1′-binaphthalene]-2,2′-diyl)bis{oxy[3-(dibenzo[b,a]thiophen-4-yl)-4,1-phenylene]}]dimethanol
[(6,6′-diphenyl[1,1′-binaphthalene]-2,2′-diyl)bis{oxy[3-(thianthren-1-yl)-4,1-phenylene]}]dimethanol
[(6,6′-bis(naphthalen-1-yl)[1,1′-binaphthalene]-2,2′-diyl)bis(oxy[1,1′-biphenyl]-2,5-diyl)]dimethanol
[(6,6′-bis(naphthalen-1-yl)[1,1′-binaphthalene]-2,2′-diyl)bis{oxy[3-(naphthalen-1-yl)-4,1-phenylene]}]dimethanol
4,4′-[(6,6′-bis(naphthalen-1-yl)[1,1′-binaphthalene]-2,2′-diyl)bis(oxy)]bis[3-(naphthalen-1-yl)benzoic acid]
dimethyl 4,4′-[(6,6′-bis(naphthalen-1-yl)[1,1′-binaphthalene]-2,2′-diyl)bis(oxy)]bis[3-(naphthalen-1-yl)benzoate]
[(6,6′-bis(naphthalen-1-yl)[1,1′-binaphthalene]-2,2′-diyl)bis{oxy[3-(naphthalen-2-yl)-4,1-phenylene]}]dimethanol
4,4′-[(6,6′-bis(naphthalen-1-yl)[1,1′-binaphthalene]-2,2′-diyl)bis(oxy)]bis[3-(naphthalen-2-yl)benzoic acid]
[(6,6′-bis(naphthalen-1-yl)[1,1′-binaphthalene]-2,2′-diyl)bis{oxy[3-(dibenzo[b,d]thiophen-4-yl)-4,1-phenylene]}]dimethanol
[(6,6′-bis(naphthalen-1-yl)[1,1′-binaphthalene]-2,2′-diyl)bis{oxy[3-(thianthren-1-yl)-4,1-phenylene]}]dimethanol
[(6,6′-bis(naphthalen-2-yl)[1,1′-binaphthalene]-2,2′-diyl)bis(oxy[1,1′-biphenyl]-2,5-diyl)]dimethanol
[(6,6′-bis(naphthalen-2-yl)[1,1′-binaphthalene]-2,2′-diyl)bis{oxy[3-(naphthalen-1-yl)-4,1-phenylene]}]dimethanol
4,4′-[(6,6′-bis(naphthalen-2-yl)[1,1′-binaphthalene]-2,2′-diyl)bis(oxy)]bis[3-(naphthalen-1-yl)benzoic acid]
[(6,6′-bis(naphthalen-2-yl)[1,1′-binaphthalene]-2,2′-diyl)bis{oxy[3-(naphthalen-2-yl)-4,1-phenylene]}]dimethanol
4,4′-[(6,6′-bis(naphthalen-2-yl)[1,1′-binaphthalene]-2,2′-diyl)bis(oxy)]bis[3-(naphthalen-2-yl)benzoic acid]
dimethyl 4,4′-[(6,6′-bis(naphthalen-2-yl)[1,1′-binaphthalene]-2,2′-diyl)bis(oxy)]bis[3-(naphthalen-2-yl)benzoate]
[(6,6′-bis(naphthalen-2-yl)[1,1′-binaphthalene]-2,2′-diyl)bis{oxy[3-(dibenzo[b,a]thiophen-4-yl)-4,1-phenylene]}]dimethanol
[(6,6′-bis(naphthalen-2-yl)[1,1′-binaphthalene]-2,2′-diyl)bis{oxy[3-(thianthren-1-yl)-4,1-phenylene]}]dimethanol
[(6,6′-di(phenanthren-9-yl)[1,1′-binaphthalene]-2,2′-diyl)bis{oxy[3-(phenanthren-9-yl)-4,1-phenylene]}]dimethanol
[(6,6′-bis(dibenzo[b,d]thiophen-4-yl)[1,1′-binaphthalene]-2,2′-diyl)bis{oxy[3-(dibenzo[b,d]thiophen-4-yl)-4,1-phenylene]}]dimethanol
[(6,6′-di(thianthren-1-yl)[1,1′-binaphthalene]-2,2′-diyl)bis{oxy[3-(thianthren-1-yl)-4,1-phenylene]}]dimethanol
[(6,6′-diphenyl[1,1′-binaphthalene]-2,2′-diyl)bis(oxy[1,1′-biphenyl]-5,2-diyl)]dimethanol
5,5′-[(6,6′-diphenyl[1,1′-binaphthalene]-2,2′-diyl)bis(oxy)]di([1,1′-biphenyl]-2-carboxylic acid)
[(6,6′-diphenyl[1,1′-binaphthalene]-2,2′-diyl)bis{oxy[2-(naphthalen-1-yl)-4,1-phenylene]}]dimethanol
4,4′-[(6,6′-diphenyl[1,1′-binaphthalene]-2,2′-diyl)bis(oxy)]bis[2-(naphthalen-1-yl)benzoic acid]
[(6,6′-diphenyl[1,1′-binaphthalene]-2,2′-diyl)bis{oxy[2-(naphthalen-2-yl)-4,1-phenylene]}]dimethanol
4,4′-[(6,6′-diphenyl[1,1′-binaphthalene]-2,2′-diyl)bis(oxy)]bis[2-(naphthalen-2-yl)benzoic acid]
[(6,6′-bis(naphthalen-1-yl)[1,1′-binaphthalene]-2,2′-diyl)bis(oxy[1,1′-biphenyl]-5,2-diyl)]dimethanol
[(6,6′-bis(naphthalen-1-yl)[1,1′-binaphthalene]-2,2′-diyl)bis{oxy[2-(naphthalen-1-yl)-4,1-phenylene]}]dimethanol
4,4′-[(6,6′-bis(naphthalen-1-yl)[1,1′-binaphthalene]-2,2′-diyl)bis(oxy)]bis[2-(naphthalen-1-yl)benzoic acid]
dimethyl 4,4′-[(6,6′-bis(naphthalen-1-yl)[1,1′-binaphthalene]-2,2′-diyl)bis(oxy)]bis[2-(naphthalen-1-yl)benzoate]
[(6,6′-bis(naphthalen-1-yl)[1,1′-binaphthalene]-2,2′-diyl)bis{oxy[2-(naphthalen-2-yl)-4,1-phenylene]}]dimethanol
[(6,6′-bis(naphthalen-2-yl)[1,1′-binaphthalene]-2,2′-diyl)bis(oxy[1,1′-biphenyl]-5,2-diyl)]dimethanol
[(6,6′-bis(naphthalen-2-yl)[1,1′-binaphthalene]-2,2′-diyl)bis{oxy[2-(naphthalen-1-yl)-4,1-phenylene]}]dimethanol
[(6,6′-bis(naphthalen-2-yl)[1,1′-binaphthalene]-2,2′-diyl)bis{oxy[2-(naphthalen-2-yl)-4,1-phenylene]}]dimethanol
4,4′-[(6,6′-bis(naphthalen-2-yl)[1,1′-binaphthalene]-2,2′-diyl)bis(oxy)]bis[2-(naphthalen-2-yl)benzoic acid]
dimethyl 4,4′-[(6,6′-bis(naphthalen-2-yl)[1,1′-binaphthalene]-2,2′-diyl)bis(oxy)]bis[2-(naphthalen-2-yl)benzoate]
[(6,6′-di(phenanthren-9-yl)[1,1′-binaphthalene]-2,2′-diyl)bis{oxy[2-(phenanthren-9-yl)-4,1-phenylene]}]dimethanol
[(6,6′-bis(dibenzo[b,d]thiophen-4-yl)[1,1′-binaphthalene]-2,2′-diyl)bis(oxy[1,1′-biphenyl]-5,2-diyl)]dimethanol
[(6,6′-bis(dibenzo[b,d]thiophen-4-yl)[1,1′-binaphthalene]-2,2′-diyl)bis{oxy[2-(dibenzo[b,d]thiophen-4-yl)-4,1-phenylene]}]dimethanol
[(6,6′-di(thianthren-1-yl)[1,1′-binaphthalene]-2,2′-diyl)bis(oxy[1,1′-biphenyl]-5,2-diyl)]dimethanol
[(6,6′-di(thianthren-1-yl)[1,1′-binaphthalene]-2,2′-diyl)bis{oxy[2-(thianthren-1-yl)-4,1-phenylene]}]dimethanol
{(6,6′-diphenyl[1,1′-binaphthalene]-2,2′-diyl)bis[oxy(4-phenylnaphthalene-6,2-diyl)]}dimethanol
[(6,6′-diphenyl[1,1′-binaphthalene]-2,2′-diyl)bis(oxy[1,1′-binaphthalene]-7,3-diyl)]dimethanol
[(6,6′-diphenyl[1,1′-binaphthalene]-2,2′-diyl)bis(oxy[1,2′-binaphthalene]-7,3-diyl)]dimethanol
{(6,6′-bis(naphthalen-1-yl)[1,1′-binaphthalene]-2,2′-diyl)bis[oxy(4-phenylnaphthalene-6,2-diyl)]}dimethanol
[(6,6′-bis(naphthalen-1-yl)[1,1′-binaphthalene]-2,2′-diyl)bis(oxy[1,1′-binaphthalene]-7,3-diyl)]dimethanol
7,7′-[(6,6′-bis(naphthalen-1-yl)[1,1′-binaphthalene]-2,2′-diyl)bis(oxy)]di([1,1′-binaphthalene]-3-carboxylic acid)
[(6,6′-bis(naphthalen-1-yl)[1,1′-binaphthalene]-2,2′-diyl)bis(oxy[1,2′-binaphthalene]-7,3-diyl)]dimethanol
[(6,6′-bis(naphthalen-2-yl)[1,1′-binaphthalene]-2,2′-diyl)bis(oxy[1,1′-binaphthalene]-7,3-diyl)]dimethanol
[(6,6′-bis(naphthalen-2-yl)[1,1′-binaphthalene]-2,2′-diyl)bis(oxy[1,2′-binaphthalene]-7,3-diyl)]dimethanol
{(6,6′-diphenyl[1,1′-binaphthalene]-2,2′-diyl)bis[oxy(3-phenylnaphthalene-4,1-diyl)]}dimethanol
4,4′-[(6,6′-diphenyl[1,1′-binaphthalene]-2,2′-diyl)bis(oxy)]bis(3-phenylnaphthalene-1-carboxylic acid)
dimethyl 4,4′-[(6,6′-diphenyl[1,1′-binaphthalene]-2,2′-diyl)bis(oxy)]bis(3-phenylnaphthalene-1-carboxylate)
[(6,6′-diphenyl[1,1′-binaphthalene]-2,2′-diyl)bis(oxy[1,2′-binaphthalene]-1′,4′-diyl)]dimethanol
[(6,6′-diphenyl[1,1′-binaphthalene]-2,2′-diyl)bis(oxy[2,2′-binaphthalene]-1,4-diyl)]dimethanol
{(6,6′-bis(naphthalen-1-yl)[1,1′-binaphthalene]-2,2′-diyl)bis[oxy(3-phenylnaphthalene-4,1-diyl)]}dimethanol
[(6,6′-bis(naphthalen-1-yl)[1,1′-binaphthalene]-2,2′-diyl)bis(oxy[1,2′-binaphthalene]-1′,4′-diyl)]dimethanol
1′,1″-[(6,6′-bis(naphthalen-1-yl)[1,1′-binaphthalene]-2,2′-diyl)bis(oxy)]di([1,2′-binaphthalene]-4′-carboxylic acid)
dimethyl 1′,1″-[(6,6′-bis(naphthalen-1-yl)[1,1′-binaphthalene]-2,2′-diyl)bis(oxy)]di([1,2′-binaphthalene]-4′-carboxylate)
[(6,6′-bis(naphthalen-1-yl)[1,1′-binaphthalene]-2,2′-diyl)bis(oxy[2,2′-binaphthalene]-1,4-diyl)]dimethanol
{(6,6′-bis(naphthalen-2-yl)[1,1′-binaphthalene]-2,2′-diyl)bis[oxy(3-phenylnaphthalene-4,1-diyl)]}dimethanol
[(6,6′-bis(naphthalen-2-yl)[1,1′-binaphthalene]-2,2′-diyl)bis(oxy[1,2′-binaphthalene]-1′,4′-diyl)]dimethanol
[(6,6′-bis(naphthalen-2-yl)[1,1′-binaphthalene]-2,2′-diyl)bis(oxy[2,2′-binaphthalene]-1,4-diyl)]dimethanol
1,1′-[(6,6′-bis(naphthalen-2-yl)[1,1′-binaphthalene]-2,2′-diyl)bis(oxy)]di([2,2′-binaphthalene]-4-carboxylic acid)
dimethyl 1,1′-[(6,6′-bis(naphthalen-2-yl)[1,1′-binaphthalene]-2,2′-diyl)bis(oxy)]di([2,2′-binaphthalene]-4-carboxylate)
[(6,6′-bis(dibenzo[b,d]thiophen-4-yl)[1,1′-binaphthalene]-2,2′-diyl)bis(oxy[2,2′-binaphthalene]-1,4-diyl)]dimethanol
[(6,6′-bis(dibenzo[b,d]thiophen-4-yl)[1,1′-binaphthalene]-2,2′-diyl)bis{oxy[3-(dibenzo[b,d]thiophen-4-yl)naphthalene-4,1-diyl]}]dimethanol
[(6,6′-di(thianthren-1-yl)[1,1′-binaphthalene]-2,2′-diyl)bis{oxy[3-(thianthren-1-yl)naphthalene-4,1-diyl]}]dimethanol.

The compounds of the formula (I), where p=q=0 can be prepared in accordance with the process shown in the following reaction scheme 1, where X and A each have one of the meanings defined herein above for X¹ and X² or A¹ and A², respectively. In particular, X is —CH₂OH or —C(O)OR^{x}, wherein R^{x} typically is C₁-C₄-alkyl, and A is mono- or polycyclic (het)arylene.

The compounds of the formula (I) can be prepared in accordance with processes known in the art and as shown in the following reaction schemes.

Scheme 1 shows the synthesis of compounds (I) in which X¹ and X² have the same meaning (and are termed X below) and A¹ and A² have the same meaning (and are termed A below). Such compounds are termed (I′) in the following. R^{1′} has the same meaning as R¹ or is a precursor thereof, i.e. a substituent which can be converted into the desired group R¹. Analogously, R^{2′} has the same meaning as R² or is a precursor thereof, i.e. a substituent which can be converted into the desired group R². A¹ is A² or a precursor thereof, the precursor being a mono- or polycyclic arylene or hetarylene A which carries however a substituent which can be converted into the desired radical R^{Ar1}. X′ is —CHO, —CN or —C(O)OR^{x′}, where R^{x′} is phenyl, benzyl or C₁-C₄-alkyl, and more typically methyl.

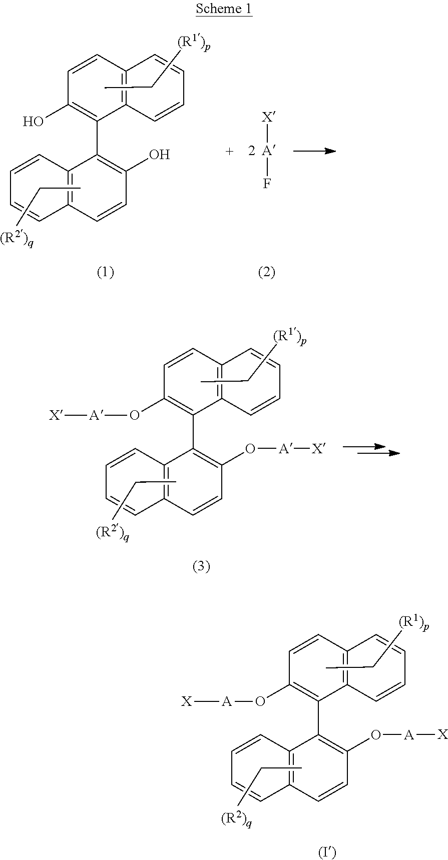

The 1,1′-bis-2-naphthol compound of formula (1) is reacted with about 2 to 2.5 molar equivalents of a compound of formula (2) in the presence of a base, e.g. an oxo base, such as an alkali metal carbonate, or an alkali metal hydride, especially in the presence of an alkali metal carbonate, such as potassium carbonate, to yield the compound of formula (Ia). Suitable solvents for this reaction are polar aprotic organic solvents, such as e.g. dimethyl formamide.

Instead of the compound (2) containing a fluoride leaving group, analogous chlorine or bromine compounds (X′-A′-Cl or X′-A′-Br) can be used. However, fluoride is the most reactive and thus the preferred leaving group.

If in compounds (3) R^{1′}, R^{2′} and A′ are R¹, R² and A, respectively, and X′ has the desired meaning, i.e. is —C(O)OR^{x′}, where R^{x′} has the meaning desired for R^{x}, compound (3) corresponds to the desired compound (I′), and no further conversion is necessary. Compounds (3) wherein X is —CHO are converted into compounds (1′) wherein X is —CH₂OH by reduction of the formyl group with a suitable reduction agent, such as sodium borohydride.

Compounds (I′) wherein X is —CH₂OH can alternatively be prepared from compounds (3) wherein X′ is —C(O)OR^{x′}, where R^{x′} is C₁-C₄-alkyl, in particular methyl, by reduction of the ester group with a suitable reduction agent, such as lithiumaluminium hydride (LAH) or DIBAL-H (diisobutylaluminium hydride).

Compounds (I′) wherein X is —C(O)OH can be prepared from compounds (3) wherein X′ is —C(O)OR^{x′} by hydrolysis, e.g. under basic conditions.

Compounds (I′) wherein X is —C(O)OH can alternatively be prepared from compounds (3) wherein X′ is —CN by hydrolysis, e.g. under basic conditions.

Compounds (I′) wherein X is —C(O)OR^{x} can be prepared from compounds (3) wherein X′ is —C(O)OH by esterification using established methods, e.g. by initially converting the diacid of formula (3) into the corresponding diacid dichloride, for instance via conversion with thionyl chloride, followed by the reaction with the respective alcohol R^{x}—OH, such as methanol, under basic conditions.

Compounds (I′) wherein X is —C(O)OR^{x} can alternatively be prepared from compounds (3) wherein X′ is —CHO by oxidation to the corresponding diacid, followed by esterification with an alcohol according to established methods. These two steps can, for instance, be carried out in a one-pot process by reacting the compound (3), wherein X′ is —CHO, with the oxidizing agent Oxone® (2KHSO₅·KHSO₄·K₂SO₄) in the presence of the respective alcohol R^{x}—OH, such as methanol.

If R^{1′} are R^{2′} precursor groups, they can be converted into the desired groups R¹ and R² by methods generally known in the art. Just to name a few examples, if radicals R¹ and/or R² is/are for instance to be R and R is to be a (het)aryl ring Q¹, the latter can be introduced via a Suzuki reaction of compounds (3) wherein R^{1′} and/or R^{2′} are halogen or a sulfonate group, such as triflate or tosylate, and are in particular Br, with a suitable boronic acid derivative, such as Q¹-B(OH)₂, or with an ester or anhydride thereof, under the application of typical Suzuki conditions, such as Pd catalysis (further details of the Suzuki reaction are given in context with scheme 2). This reaction can be carried out before or after the conversion of X′ into X (if this is at all necessary).

If for instance radicals R¹ and/or R² is/are to be an olefinic group —CH═CHR″, the latter can be introduced via a Heck reaction of compounds (3) wherein R^{1′} and/or R^{2′} are halogen or a sulfonate group, such as triflate or tosylate, and are in particular Br, with the corresponding olefin CH₂=CHR″ under the application of typical Heck conditions, such as Pd catalysis. This reaction, too, can be carried out before or after the conversion of X′ into X (if this is at all necessary).

Similarly, if A′ is a precursor of A, the precursor being a mono- or polycyclic arylene or hetarylene A which carries however a substituent which can be converted into the desired radical R^{Ar1}, the conversion can be carried out by methods generally known in the art. Just to name a few examples, if A is to be a mono- or polycyclic arylene or hetarylene A substituted by a radical R^{Ar1} and R^{Ar1} is to be Q², the latter can be introduced via a Suzuki reaction of compounds (3) wherein A′ is a mono- or polycyclic arylene or hetarylene ring A substituted by halogen or a sulfonate group, such as triflate or tosylate, and in particular by Br, with a suitable boronic acid derivative, such as Q²-B(OH)₂, or with an ester or anhydride thereof, under the application of typical Suzuki conditions, such as Pd catalysis (further details of the Suzuki reaction are given in context with scheme 2). This reaction can be carried out before or after the conversion of X into X (if this is at all necessary).

If A is to be a mono- or polycyclic arylene or hetarylene A substituted by a radical R^{Ar1} and R^{Ar1} is to be —CH═CR″, the latter can be introduced via a Heck reaction of compounds (3) wherein A′ is a mono- or polycyclic arylene or hetarylene ring A substituted by halogen or a sulfonate group, such as triflate or tosylate, and in particular by Br, with the corresponding olefin CH₂=CHR″ under the application of typical Heck conditions, such as Pd catalysis. This reaction, too, can be carried out before or after the conversion of X′ into X (if this is at all necessary). 1,1′-Bis-2-naphthol compounds (1) are either commercially available or can be prepared by methods known in the art. Just by way of the example, scheme 2 illustrates the bromination of 1,1′-bis-2-naphthol (1′) to 6,6′-dibromo-1,1′-bis-2-naphthol (1″) and the further conversion of the latter to 6,6′-(het)aryl-substituted 1,1′-bis-2-naphthol (1′″).

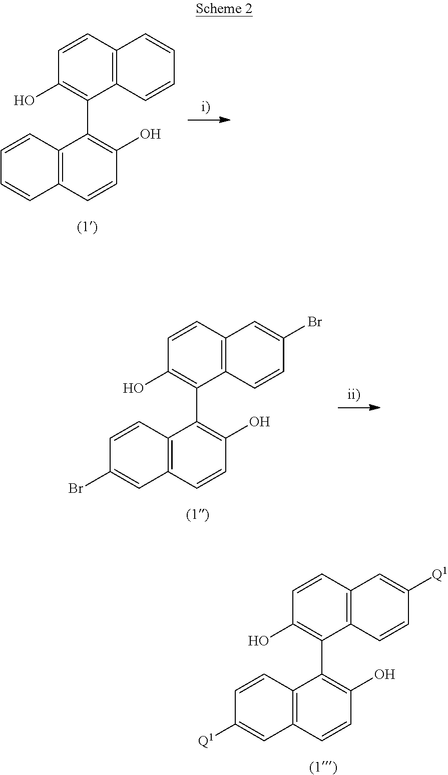

In step i) of the process according to scheme 2, 1,1′-bis-2-naphthol (1′) is brominated to selectively yield the 6,6′-dibromo-1,1′-bi-2-naphthol of formula (1″). Bromination can be simply achieved by mixing 1,1′-bis-2-naphthol (1′) at low temperatures with a suitable brominating reagent in a polar aprotic solvent which is inert against bromination.

Suitable brominating agents are in particular elemental bromine. Suitable polar aprotic solvents for step i) include aliphatic halogenated hydrocarbon compounds, such as dichloromethane or dichloroethane, esters, such as isopropyl acetate or ethyl acetate, and mixtures thereof. Suitable reaction temperatures for bromination of 1,1′-bis-2-naphthol (1′) with bromine are typically in the range from −100 to 10° C., in particular in the range from −100 to −30° C. or, alternatively, in the range from −10 to 10° C. Further details can be taken from Bunzen et al. J. Am. Chem. Soc., 2009, 131(10), 3621-3630.

As an alternative, N-bromosuccinimide can be used as a bromination agent. In this case, reaction temperatures are usually higher than for the bromination with elemental bromine, e.g. from 0 to 50° C. Suitable solvents may then, in addition to aliphatic halogenated hydrocarbons, also include aliphatic ketones having from 3 to 6 carbon atoms, such as acetone or methyl ethyl ketone, ethers having from 4 to 6 carbon atoms, such as tetrahydrofuran, dioxan, diethyl ether, cyclopentyl methyl ether, and other solvents like acetonitrile, dimethylformamide, chloroform, methylene chloride, dichloroethane, as well as mixtures thereof with aliphatic halogenated hydrocarbons.

As a further alternative 6,6′-dibromo-1,1′-bis-2-naphthol of formula (1″) can also be synthesized by copper(II)-catalyzed oxidative coupling of 6-bromo-2-naphthol, e.g. in accordance with the procedure described in H. Egami et al., J. Am. Chem. Soc. 2009, 13 (17), 6082-83.

In step ii) of scheme 2 the compound of formula (1″) is reacted with an (het)arylboronic compound of the formula Q¹-B(OH)₂, where Q¹ is as defined above, or with an ester or anhydride of Q¹-B(OH)₂, in particular a C₁-C₄-alkyl ester thereof, in the presence of a transition metal catalyst, in particular in the presence of a palladium catalyst. Frequently, step ii) is performed under Suzuki coupling conditions (see e.g. A. Suzuki et al., Chem. Rev. 1995, 95, 2457-2483; N. Zhe et al., J. Med. Chem. 2005, 48 (5), 1569-1609; Young et al., J. Med. Chem. 2004, 47 (6), 1547-1552; C. Slee et al., Bioorg. Med. Chem. Lett. 2001, 9, 3243-3253; T. Zhang et al., Tetrahedron Lett., 52 (2011), 311-313, S. Bourrain et al., Synlett. 5 (2004), 795-798, B. Li et al., Europ. J.

Org. Chem. 2011 3932-3937). Suitable transition metal catalysts are in particular palladium compounds, which bear at least one palladium atom and at least one tri-substituted phosphine ligand. Examples of palladium catalysts are tetrakis(triphenylphosphine) palladium, tetrakis(tritolylphosphine) palladium and [1,1-bis(diphenylphosphino)ferrocene]dichloropalladium(II) (PdCl₂(dppf)). Frequently, the palladium catalysts are prepared in situ from a suitable palladium precursor and a suitable phosphine ligand. Suitable palladium precursors are palladium compounds such as tris-(dibenzylideneacetone)dipalladium(0) (Pd₂(dba)₃) or palladium(II) acetate (Pd(OAc)₂). Suitable phosphine ligands are in particular tri(substituted)phosphines, e.g. a triarylphosphines such as triphenylphosphine, tritolylphosphine or 2,2′-bis(diphenyl-phosphino)-1,1′-binaphthalene (BINAP), tri(cyclo)alkylphosphine, such as tris-n-butylphosphine, tris(tert-butyl)phosphine or tris(cyclohexylphosphine), or dicyclohexyl-(2′,4′,6′-tri-isopropyl-1,1′-biphenyl-2-yl)-phosphane (X-Phos). Usually, the reaction is performed in the presence of a base, in particular an oxo base, such as an alkali metal alkoxide, alkali metal hydroxide, alkali metal carbonate or earth alkaline carbonate, e.g. sodium ethoxide, sodium tert-butoxide, lithium hydroxide, sodium carbonate or potassium carbonate; or a phosphate base, e.g. tripotassium phosphate. Frequently, the reaction according to step ii) of scheme 2 is performed in an organic solvent or in a mixture thereof with water. If the reaction is performed in a mixture of an organic solvent and water, the reaction mixture may be monophasic or biphasic. Suitable organic solvents include but are not limited to aromatic hydrocarbons, such as toluene, anisole or xylene, acyclic and cyclic ethers, such as methyl tert.-butyl ether, diisopropylether, dioxane or tetrahydrofurane, and aliphatic alcohols having 1 to 4 carbon atoms, such as methanol, ethanol or isopropanol, as well as mixtures thereof. The reaction according to step ii) of scheme 2 is usually performed at temperatures in the range from 50 to 150° C.

Compounds (2) are either commercially available or can be prepared by methods known in the art. Just by way of example, scheme 3 illustrates conversion of bromine-substituted (2′) into (het)aryl-substituted (2″). This conversion can be carried out under the Suzuki conditions described in context with scheme 2.

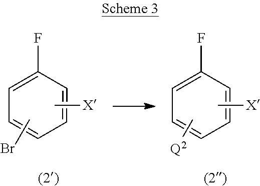

Compounds (I) in which X¹ and X² and/or A¹ and A² have different meanings (termed compounds (I″) in the following) can be prepared by stepwise reaction of the two hydroxyl groups of compounds (1), as illustrated in scheme 4. The process according to scheme 4, however, is particularly suitable for preparing compounds of formula (I) where p and q are both 0, 1 or 2 and the substituents R¹ and R², if present, have the same meaning and are bound to the corresponding positions of their respective naphthyl units.

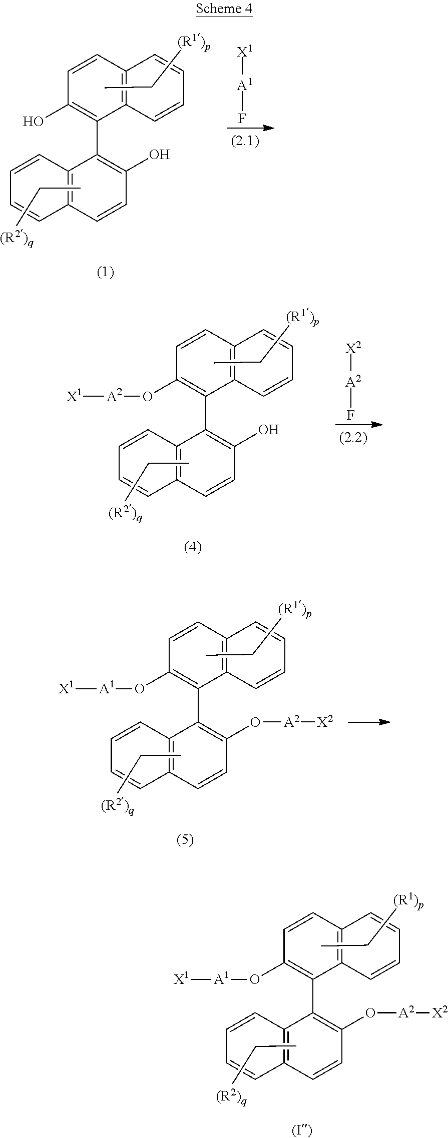

In the first step of the process according to scheme 4, the 1,1′-bis-2-naphthol compound (1) is reacted with about 0.7 to 1.1 molar equivalents of the compound of formula (2.1). The mono-etherified product of formula (4) is then subjected to a further etherification with about 1.0 to 1.5 molar equivalents of the compound of formula (2.2) to afford the compound (51. Both etherification steps can be carried out under reaction conditions that are substantially analogous to those described above in connection with Scheme 1 (reaction of (1) and (2) to (3)).

If in compounds (5) R^{1′} and R^{2′} are R¹ and R², respectively, compound (5) corresponds to the desired compound (I″), and no further conversion is necessary. Otherwise, R^{1′} and R^{2′} are converted into the desired radicals R¹ and R², as described above in context with scheme 1.

If desired or appropriate, compounds of formula (I) in which A¹ and A² are identical or different biphenylylene moieties can be prepared in two, three or four steps by analogy with the process shown in the reaction scheme 5 below. The process according to scheme 5 and analogous ones are particularly suitable for preparing compounds (I) where p and q are both 0, 1 or 2 and the substituents R¹ and R², if present, have the same meaning and are bound to the corresponding positions of their respective naphthyl units. Reaction scheme 5 exemplifies the preparation of a compound (I) where A¹ is 3,4′-biphenylylene and A² is 3,3′-biphenylylene.

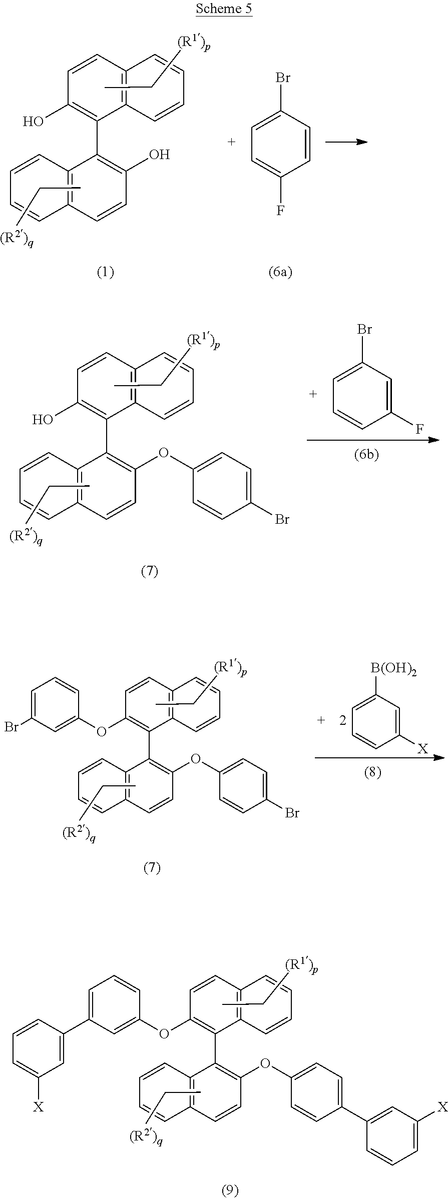

In the first reaction step of scheme 5, 1,1′-bis-2-naphthol (1) is reacted with about 0.7 to 1.1 molar equivalents of the bromide of formula (6a). In the second step, the mono-etherified product of formula (7) is then subjected to a further etherification with about 1.0 to 1.5 molar equivalents of the compound of formula (6b) to give the dibromide of formula (7). The first and second reaction steps can be carried out under reaction conditions that are substantially analogous to those described above in connection with Scheme 1. The dibromide (7) can then be reacted with about two molar equivalents of the phenylboronic compound of the formula (8) in analogy to the coupling step described above in connection with the process of scheme 2 to yield compound (9).

If in compounds (9) R^{1′} and R^{2′} are R¹ and R², respectively, compound (9) corresponds to the desired biphenylyl compound (I), and no further conversion is necessary. Otherwise, R^{1′} and R^{2′} are converted into the desired radicals R¹ and R², as described above in context with scheme 1.

A compound of the formula (9′), which differs from the compound (9) prepared in scheme 5 above only in that A¹ and A² have the same meaning, e.g. 3,4′-biphenylylene, can be prepared via a modified first step of scheme 5 in which 1,1′-bis-2-naphthol (1) is reacted with about 2 molar equivalents of the bromide of formula (6a), followed by the third reaction step of scheme 5.

A compound of the formula (9″), which differs from the compound (9) prepared in scheme 5 above only in that X¹ and X² have different meanings, can also be prepared using a variation of the process according to scheme 5. Specifically, the compound (7) obtained in the first step of the process of scheme 5 is reacted in analogy to the third step with about one molar equivalent of a compound (8′), which is a compound (8) with another X group. The intermediate product obtained this way is then reacted with a compound (6b) in accordance to the second step of scheme 5. The obtained bromide is finally reacted in analogy to the third step iii) with about one molar equivalent of a compound (8).

Compounds of formula (I) comprising as A¹ and A² identical or different bi(het)arylene groups that are not biphenylylene, and wherein the variables p, q, R¹, R², X¹ and X² have the meanings defined herein, can also be prepared in typically two, three or four reaction steps by analogy with the processes described in the context of the reaction scheme 5 above, provided that the bonds between the two (het)arylene moieties of A¹ as well as of A² are C—C bonds.

For an easier understanding, the following schemes illustrate the reaction of scheme 1 on the basis of more specific examples.

Scheme 6 illustrates the reaction of scheme 1 for the synthesis of compounds (I) wherein p and q are 0, A¹ and A² are 1,4-phenylene substituted by a radical Q² and X¹ and X² are CH₂OH starting from compounds (2) which already carry the desired substituent Q².

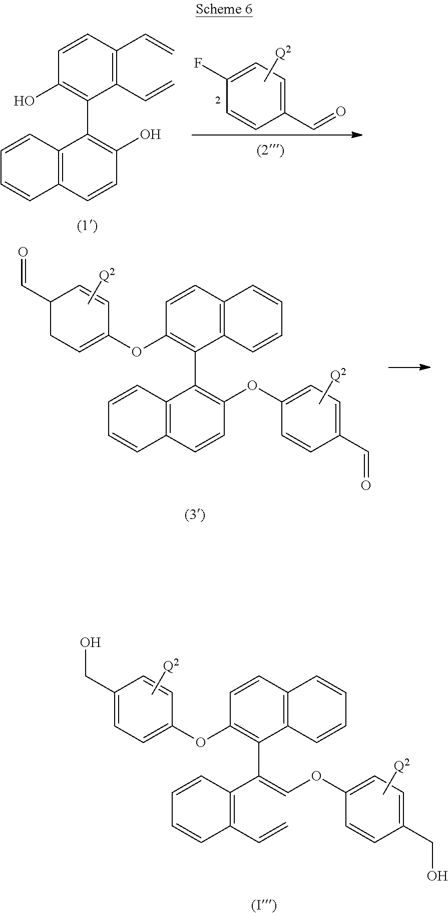

Scheme 7 illustrates the reaction of scheme 1 for the synthesis of compounds (1) wherein p and q are 0, A¹ and A² are 1,4-phenylene substituted by a radical Q² and X¹ and X² are CH₂OH starting from compounds (2) which carries a precursor (Br) of the desired substituent Q². Q² is introduced after the etherification step (reaction of (3′″) to (3^{iv})) or after reduction of the formyl groups (reaction of (3′″) to (I′″)).

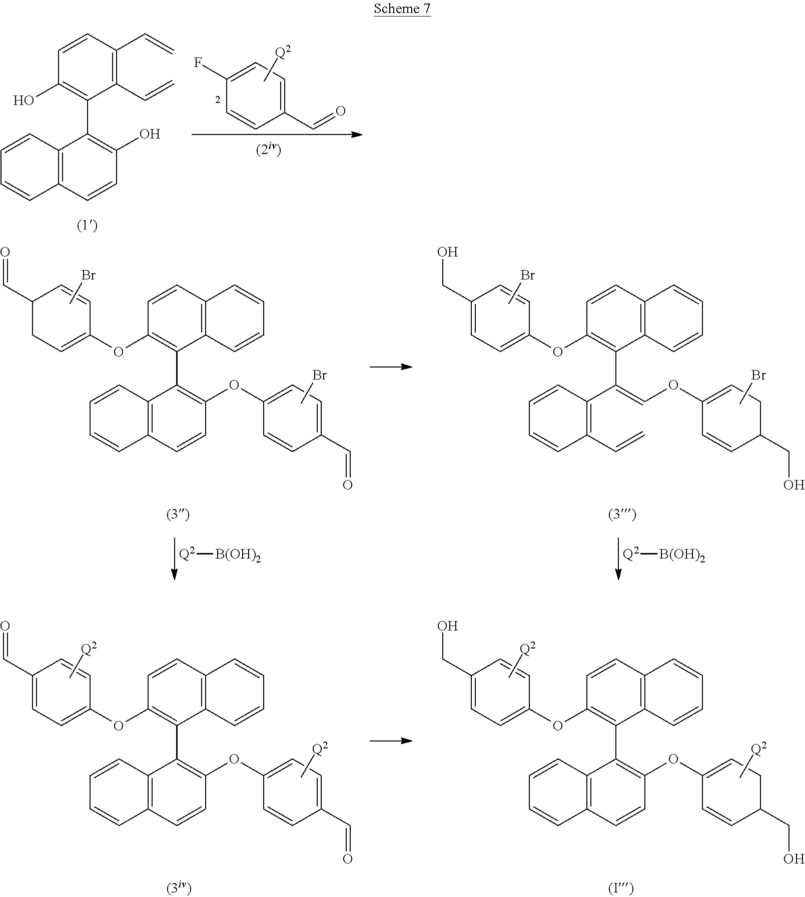

Scheme 8 illustrates the reaction of scheme 1 for the synthesis of compounds (I) wherein p and q are 1, R¹ and R² are Q¹, A¹ and A² are 1,4-phenylene substituted by a radical Q² and X¹ and X² are CH₂OH starting from compound (1″) which carries a precursor (Br) of the desired substituent Q¹ and compound (2′″) which already carries the desired substituent Q². Q¹ is introduced after the etherification step (reaction of (3^{v}) to (3^{vii})) or after reduction of the formyl groups (reaction of (3^{vi}) to (1^{iv})).

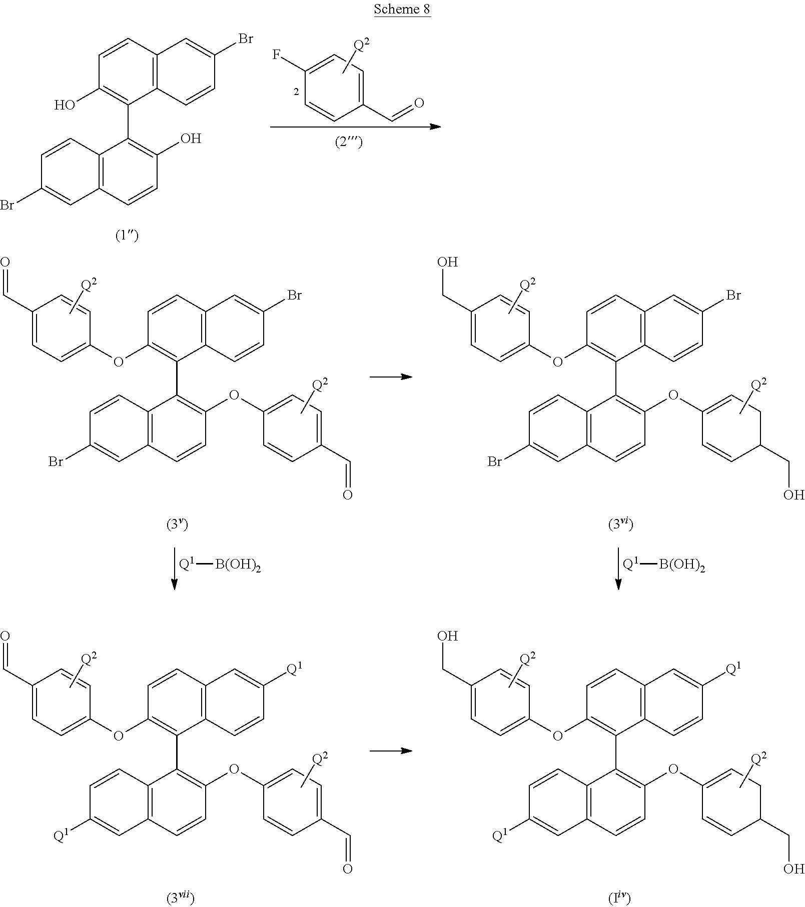

Alternatively, Q¹ can be introduced after the etherification step (reaction of 1″) to (1′″), as shown in scheme 2 above).

Scheme 9 illustrates the reaction of scheme 1 for the synthesis of compounds (Iv) wherein Q¹ and Q² are identical (termed Q below).

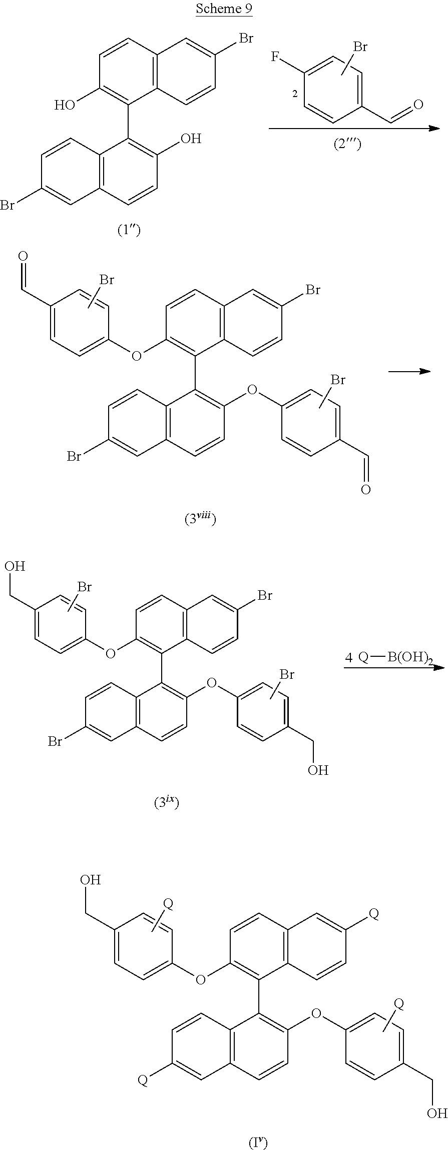

The conversions shown in schemes 1 to 9 can be accomplished by the reactions described above in the context with these schemes or by apparent variations of these reactions, or, alternatively, by procedures well-established in preparative organic chemistry, or combinations thereof.

Further compounds of formula (I) can be prepared by employing apparent variations of the reactions described above and combinations thereof with procedures well-established in preparative organic chemistry.

The reaction mixtures obtained in the individual steps of the syntheses for preparing the compounds described in reaction schemes 1 to 9 above are usually worked up in a conventional way, e.g. by mixing with water, separating the phases and, where appropriate, purifying the crude products by washing, treatment with an adsorbent, such as activated charcoal, chromatography or crystallization. The intermediates in some cases result in the form of colourless or pale brownish, viscous oils, which are freed of volatiles or purified under reduced pressure and at moderately elevated temperature. If the intermediates are obtained as solids, the purification can be achieved by recrystallization or washing procedures, such as slurry washing.

The starting compounds used in the syntheses shown in schemes 1 to 9 above to prepare compounds of formula (I) are commercially available or can be prepared by methods known from the art.

As stated above, the compounds of the present invention can be obtained in high purity, which means that a product is obtained, which does not contain significant amounts of organic impurities different from the compound of formula (I), except for volatiles.

Usually, the purity of compounds of formula (I) is at least 95%, in particular at least 98% and especially at least 99%, based on the non-volatile organic matter, i.e. the product contains at most 5%, in particular at most 2% and especially at most 1% of non-volatile impurities different from the compound of formula (1).

It should be mentioned in this context that mixtures of different compounds of formula (I) are also useful as they may serve as monomer compostions for preparing beneficial thermoplastic resins, such as polycarbonate resins, that include different structural units of formula (II) derived from said different monomers of formula (I). Therefore, mixtures of different compounds of formula (I) as well as corresponding thermoplastic resins including different structural units of formula (II) are also part of the present invention.

The term “volatiles” refers to organic compounds, which have a boiling point of less than 200° C. at standard pressure (105 Pa). Consequently, non-volatile organic matter is understood to mean compounds having a boiling point, which exceeds 200° C. at standard pressure.

It is a particular benefit of the invention that the compounds of formula (I) and likewise their solvates, can often be obtained in crystalline form. In the crystalline form the compound of formula (I) may be present in pure form or in the form of a solvate with water or an organic solvent. Therefore, a particular aspect of the invention relates to the compounds of formula (I), which are essentially present in crystalline form. In particular, the invention relates to crystalline forms, where the compound of formula (I) is present without solvent and to the crystalline solvates of the compounds of formula (I), where the crystals contain a solvent incorporated.

It is a particular benefit of the invention that the compounds of the formula (I) and likewise their solvates, can often be easily crystallized from conventional organic solvents.

This allows for an efficient purification of the compounds of formula (I). Suitable organic solvents for crystallizing the compounds of the formula (I) or their solvates, include but are not limited to aromatic hydrocarbons such as toluene or xylene, aliphatic ketones in particular ketones having from 3 to 6 carbon atoms, such as acetone, methyl ethyl ketone, methyl isopropyl ketone or diethyl ketone, aliphatic and alicyclic ethers, such as diethyl ether, dipropyl ether, methyl isobutyl ether, methyl tert-butyl ether, ethyl tert-butyl ether, dioxane or tetrahydrofuran, aliphatic-aromatic ethers, such as anisole, aliphatic alcohols having 1 to 4 carbon atoms, such as methanol, ethanol or isopropanol, and aliphatic esters, such as ethyl acetate, as well as mixtures thereof. It may be beneficial to subject a dissolved crude preparation of a compound of formula (I) to filtration, e.g. over cellite, prior to the crystallization step, in order to remove solid components that may be present in a crude preparation.

Furthermore, impurities, especially color forming impurities and heavy metals, that may be present in a crude preparation of a compound of formula (I) can be removed at any stage of the purification process, e.g. before a filtration step or a crystallization step, by standard procedures, such as treatment with an adsorbent, e.g. activated charcoal.

Alternatively, the compounds of the formula (I) and likewise their solvates, can be obtained in purified form by employing other simple and efficient methods for purifying the raw products of these compounds, such as in particular slurry washing the raw solids obtained directly after the conversion to prepare the compounds of formula (I). Slurry washing is typically conducted at ambient temperature or elevated temperatures of usually about 30 to 90° C., in particular 40 to 80° C. Suitable organic solvents here are in principle the same as those listed above as being suitable for crystallizing the compounds of formula (I), such as in particular the mentioned aromatic hydrocarbons, aliphatic ketones and aliphatic ethers, e.g. toluene, methyl ethyl ketone and methyl tert-butyl ether.

Accordingly, the compounds of formula (I) used for the preparation of the thermoplastic polymers, in particular the polycarbonates, as defined herein, can be easily prepared and obtained in high yield and high purity. In particular, compounds of formula (I) can be obtained in crystalline form, which allows for an efficient purification to the degree required in the preparation of optical resins. In particular, these compounds can be obtained in a purity which provides for high refractive indices and also low haze, which is particularly important for the use in the preparation of optical resins of which the optical devise is made of. In conclusion, the compounds of formula (I) are particularly useful as monomers in the preparation of the optical resins.

A skilled person will readily appreciate that the formula (I) of the monomer used corresponds to the formula (II) of the structural unit comprised in the thermoplastic resin.

Likewise, the formula (Ia) of the monomer used corresponds to the formula (IIa), respectively, of the structural unit comprised in the thermoplastic resin. Apparently, the formulae (Ia.1), (Ia.2), (Ia.3) and (Ia.4) of the monomer used corresponds to the formulae (IIa.1), (IIa.2), (IIa.3) and (IIa.4), respectively, of the structural unit comprised in the thermoplastic resin, where #, X, R⁰ and Q⁰ are as defined herein.

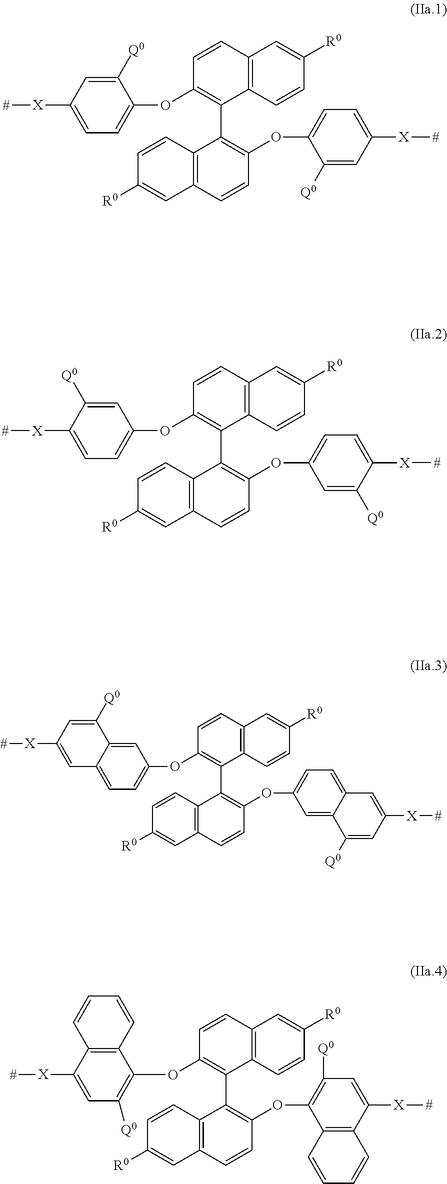

A skilled person will also appreciate that the structural units of the formulae (II), (IIa), (IIa.1), (IIa.2), (IIa.3) and (IIa.4), respectively, are repeating units within the polymer chains of the thermoplastic resin. In addition to the structural units of the formulae (II), (IIa), (IIa.1), (IIa.2), (IIa.3) and (IIa.4), respectively, the thermoplastic resin may have structural units different therefrom. In a preferred embodiment, these further structural units are derived from aromatic monomers of the formula (IV) resulting in structural units of the formula (V):

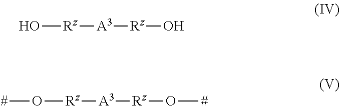

where
#represents a connection point to a neighboring structural unit;
A³ is a polycyclic radical bearing at least 2 benzene rings, wherein the benzene rings may be connected by W and/or directly fused to each other and/or fused by a non-benzene carbocycle and/or fused by two non-benzene carbocycles that are linked via a linker L, where A³ is unsubstituted or substituted by 1, 2 or 3 radicals R^{aa}, which are selected from the group consisting of halogen, C₁-C₆-alkyl, C₅-C₆-cycloalkyl, phenyl, naphthyl, 1,2-dihydroacenaphthylenyl, phenanthrenyl, pyrenyl, triphenylenyl, benzo[b]furanyl, dibenzo[b,d]furanyl, benzo[b]thienyl, dibenzo[b,d]thienyl and thianthrenyl;
W is selected from the group consisting of a single bond, O, C═O, S, S(O), SO₂, CH₂, CH—Ar, CAr₂, CH(CH₃), C(CH₃)₂ and a radical of the formula (A′)

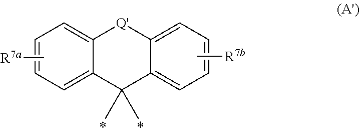

where
Q′ represents a single bond, O, C═O or CH₂;
R^{7a}, R^{7b}, independently of each other are selected from the group consisting of hydrogen, fluorine, CN, R, OR, CHᵥR′₃₋ᵥ, NR₂, C(O)R and C(O)NH₂, where R and R′ are as defined herein above and v is 0, 1 or 2; and
* represents a connection point to a benzene ring;
L is selected from a single bond, C₁-C₄-alkylene, C₄-C₇-cycloalkylene, C₄-C₇-cycloalkylenedimethylene, phenylenedimethylene, where L is unsubstituted or substituted by 1 or 2 radicals R^{L}, which are selected from the group consisting of C₁-C₄-alkyl, halogen, C₁-C₄-haloalkyl, C₄-C₇-cycloalkyl and phenyl,
Ar is selected from the group consisting of mono- or polycyclic aryl having from 6 to 26 carbon atoms as ring atoms and mono- or polycyclic hetaryl having a total of 5 to 26 atoms, which are ring members, where 1, 2, 3 or 4 of these ring member atoms of hetaryl are selected from nitrogen, sulphur and oxygen, while the remainder of these ring member atoms of hetaryl are carbon atoms, where Ar is unsubstituted or substituted by 1, 2 or 3 radicals R^{ab}, which are selected from the group consisting of halogen, phenyl and C₁-C₄-alkyl;
R^{z} is a single bond, Alk³, O-Alk⁴-, 0-Alk⁴-[O-Alk⁴-]_{w}- or O-Alk⁵-C(O)— where O is bound to A³, and where
   w is an integer from 1 to 10;
   Alk³ is C₁-C₄-alkandiyl;
   Alk⁴ is C₂-C₄-alkandiyl; and
   Alk⁵ is C₁-C₄-alkandiyl.

If R^{z} in formula (IV) is O-Alk⁵-C(O), the esters, in particular the C₁-C₄-alkyl esters, of the monomers of formula (IV) may be used instead.

In the context of formulae (IV) and (V), A³ is in particular either a polycyclic radical bearing at least 2 benzene or naphthaline rings, wherein the benzene rings are connected by W or fused by two non-benzene carbocycles that are linked via a linker L, where W is in particular selected from the group consisting of a single bond, S, S(O), SO₂, C(CH₃)₂, and a radical A′ and where L is a single bond or C₁-C₄-alkylene.

In the context of formulae (IV) and (V), R^{z} is in particular O-Alk⁴-, where Alk⁴ is in particular linear alkandiyl having 2 to 4 carbon atoms and especially O—CH₂CH₂.

Amongst the monomers of formula (IV) preference is given to monomers of the general formulae (IV-1) to (IV-8)

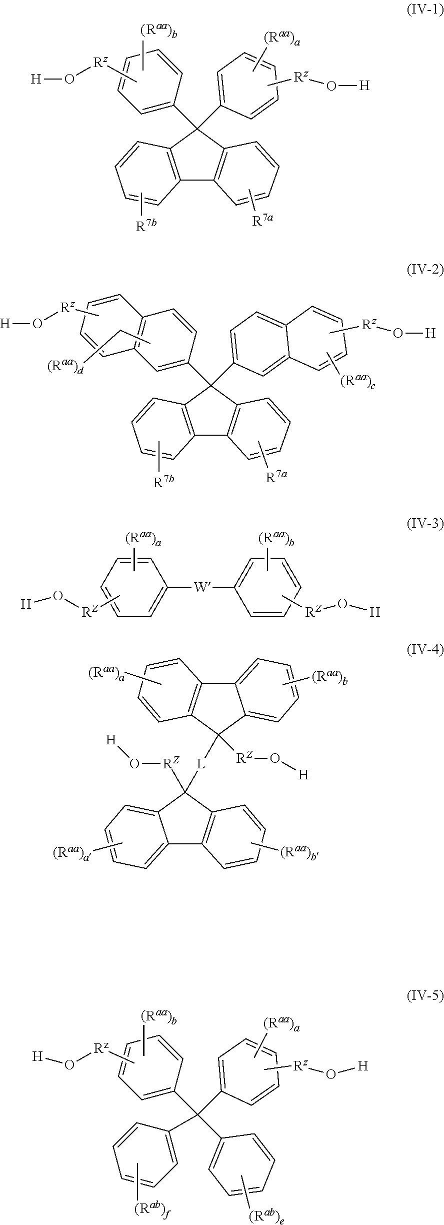

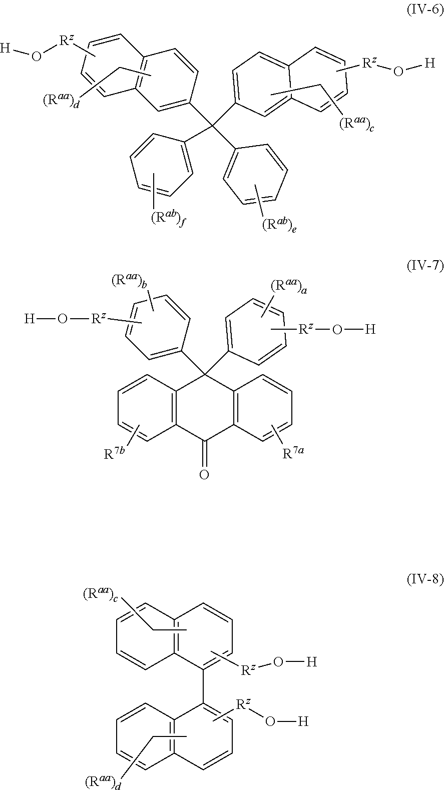

where
a and b are 0, 1, 2 or 3, in particular 0 or 1;
a′ and b′ are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1; W′ is S, S(O), SO₂, O, single bond, CH₂, CH(CH₃), C(CH₃)₂, in particular S, S(O), SO₂ or C(CH₃)₂;
and where R^{z}, R^{aa}, R^{ab}, R^{7a}, R^{7b} and L are as defined for formula (IV) and where R^{z} is in particular selected from a single bond, CH₂ and OCH₂CH₂,

Amongst the monomers of formula (IV) particular preference is given to monomers of the general formulae (IV-11) to (IV-22), where R^{z} and R^{aa} are as defined herein and R^{z} is in particular selected from a single bond, CH₂ and O—CH₂CH₂, and especially is O—CH₂CH₂:

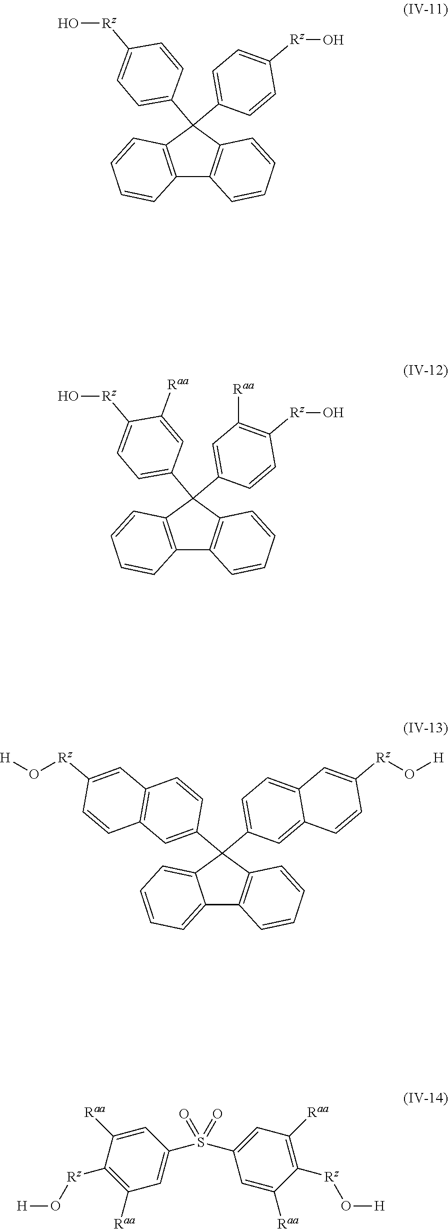

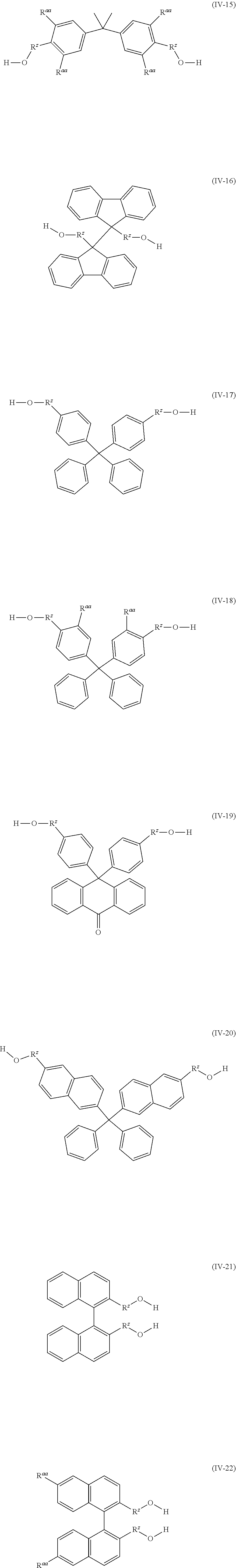

Examples of compounds of the formulae (IV-11) to (IV-22) are 9,9-bis(4-hydroxyphenyl)fluorene, 9,9-bis(4-hydroxy-3-methylphenyl)fluorene, 9,9-bis(4-hydroxy-3-isopropylphenyl)fluorene, 9,9-bis(4-hydroxy-3-tert.-butylphenyl)fluorene, 9,9-bis(4-hydroxy-3-cyclohexylphenyl)fluorene, 9,9-bis(4-hydroxy-3-phenylphenyl)fluorene, 9,9-bis(4-(2-hydroxyethoxy)phenyl)fluorene (BPEF), 9,9-bis(4-(2-hydroxyethoxy)-3-methylphenyl)fluorene, 9,9-bis(4-(2-hydroxyethoxy)-3-isopropylphenyl)fluorene, 9,9-bis(4-(2-hydroxyethoxy)-3-tert.-butylphenyl)fluorene, 9,9-bis(4-(2-hydroxyethoxy)-3-cyclohexylphenyl)fluorene, 9,9-bis(4-(2-hydroxyethoxy)-3-phenylphenyl)fluorene (BPPEF), 9,9-bis(6-hydroxy-2-naphthyl)fluorene, 9,9-bis(6-(2-hydroxyethoxy)-2-naphthyl)fluorene also termed 9,9-bis(6-(2-hydroxyethoxy)naphthalene-2-yl)fluorene (BNEF) or 6,6′-(9-fluorenylidene)bis(2-naphthyloxyethanol) (NOLE), 10,10-bis(4-hydroxyphenyl)anthracen-9-on, 10,10-bis(4-(2-hydroxyethoxy)phenyl)anthracen-9-on, 4,4′-dihydroxytetraphenylmethane, 4,4′-di-(2-hydroxyethoxy)-tetraphenylmethane, 3,3′-diphenyl-4,4′-dihydroxy-tetraphenylmethane, di-(6-hydroxy-2-naphthyl)-diphenylmethane, 2-[4-[1-[4-(2-hydroxyethoxy)-3,5-diphenyl-phenyl]-1-methyl-ethyl]-2,6-diphenyl-phenoxy]ethanol, 2-[4-[1-[4-(2-hydroxyethoxy)-3-phenyl-phenyl]-1-methyl-ethyl]-2,6-diphenyl-phenoxy]ethanol, 9,9′-dihydroxymethyl-9,9′-difluorene, 2,2′-[1,1′-binaphthalene-2,2′-diylbis(oxy)]diethanol also termed 2,2′-bis(2-hydroxyethoxy)-1,1′-binaphtyl or 2,2′-bis(2-hydroxyethoxy)-1,1′-binaphthalene (BNE), 2,2′-bis(1-hydroxymethoxy)-1,1′-binaphtyl, 2,2′-bis(3-hydroxypropyloxy)-1,1′-binaphtyl, 2,2′-bis(4-hydroxybutoxy)-1,1′-binaphtyl, 2,2′-bis(2-hydroxyethoxy)-6,6′-diphenyl-1,1′-binaphthalene, 2,2′-bis(2-hydroxyethoxy)-6,6′-di(naphthalene-1-yl)-1,1′-binaphthalene, 2,2′-bis(2-hydroxymethoxy)-6,6′-diphenyl-1,1′-binaphthalene, 2,2′-bis(2-hydroxy-methoxy)-6,6′-di(naphthalene-1-yl)-1,1′-binaphthalene, 2,2′-bis(2-hydroxypropoxy)-6,6′-diphenyl-1,1′-binaphthalene, 2,2′-bis(2-hydroxypropoxy)-6,6′-di(naphthalene-1-yl)-1,1′-binaphthalene, 2,2′-bis(2-hydroxyethoxy)-6,6′-di(naphthalene-2-yl)-1,1′-binaphthalene, 2,2′-bis(2-hydroxyethoxy)-6,6′-di(9-phenanthryl)-1,1′-binaphthalene, 2-[4-[1-[4-(2-hydroxyethoxy)-3,5-di(naphthalen-1-yl)-phenyl]-1-methyl-ethyl]-2,6-di(naphthalen-1-yl)-phenoxy]ethanol, 2-[4-[1-[4-(2-hydroxyethoxy)-3,5-di(naphthalen-2-yl)-phenyl]-1-methyl-ethyl]-2,6-di(naphthalen-2-yl)-phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol, 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)-phenoxy]ethanol, 2-[4-[1-[4-(2-hydroxyethoxy)-3,5-di(thiantren-1-yl)-phenyl]-1-methyl-ethyl]-2,6-di(thianthren-1-yl)-phenoxy]ethanol, 2-[4-[4-(2-hydroxyethoxy)-3,5-di(naphthalene-1-yl)phenyl]sulfonyl-2,6-di(naphthalene-1-yl)-phenoxy]ethanol, 2-[4-[4-(2-hydroxyethoxy)-3,5-di(naphthalene-2-yl)phenyl]sulfonyl-2,6-di(naphthalene-2-yl)-phenoxy]ethanol, 2-[4-[4-(2-hydroxyethoxy)-3,5-di(phenanthren-9-yl)phenyl]sulfonyl-2,6-di(phenanthren-9-yl)-phenoxy]ethanol, 2-[4-[4-(2-hydroxyethoxy)-3,5-di(thianthrene-1-yl)phenyl]sulfonyl-2,6-di(thianthrene-1-yl)phenoxy]ethanol and 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol and the like.

Among the monomers of the general formula (IV) or of formulae (IV-1) to (IV-8), particular preference is given to the monomers of formulae (IV-1), (IV-2), (IV-3) and (IV-8), even more preference is given to the monomers of formulae (IV-11), (IV-12), (IV-13), (IV-14), (IV-15), (IV-21) and (IV-22), and special preference given to 2,2′-bis(2-hydroxyethoxy)-1,1′-binaphtyl (BNE or BHBNA), 2,2′-bis(2-hydroxyethoxy)-6,6′-diphenyl-1,1′-binaphtyl (DPBHBNA), 9,9-bis(4-(2-hydroxyethoxy)phenyl)fluorene (BPEF), 9,9-bis(6-(2-hydroxyethoxy)-2-naphthyl)fluorene (BNEF), 9,9-bis(4-(2-hydroxyethoxy)-3-phenylphenyl)fluorene (BPPEF), 2-[4-[1-[4-(2-hydroxyethoxy)-3,5-di(phenanthren-9-yl)-phenyl]-1-methylethyl]-2,6-di(phenanthren-9-yl)-phenoxy]ethanol, 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)-phenoxy]ethanol, 2-[4-[1-[4-(2-hydroxyethoxy)-3,5-di(thiantren-1-yl)-phenyl]-1-methyl-ethyl]-2,6-di(thianthren-1-yl)-phenoxy]ethanol, 2-[4-[4-(2-hydroxyethoxy)-3,5-di(phenanthren-9-yl)phenyl]sulfonyl-2,6-di(phenanthren-9-yl)-phenoxy]ethanol, 2-[4-[4-(2-hydroxyethoxy)-3,5-di(thianthrene-1-yl)phenyl]sulfonyl-2,6-di(thianthrene-1-yl)phenoxy]ethanol and 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol.

Accordingly, amongst the structural units of formula (V) that may be comprised in the thermoplastic resin preference is given to structural units of the general formulae (V-1) to (V-8),

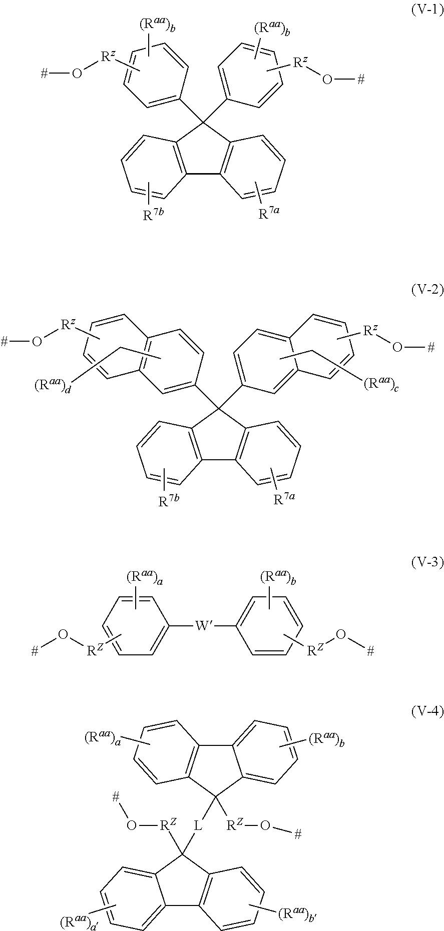

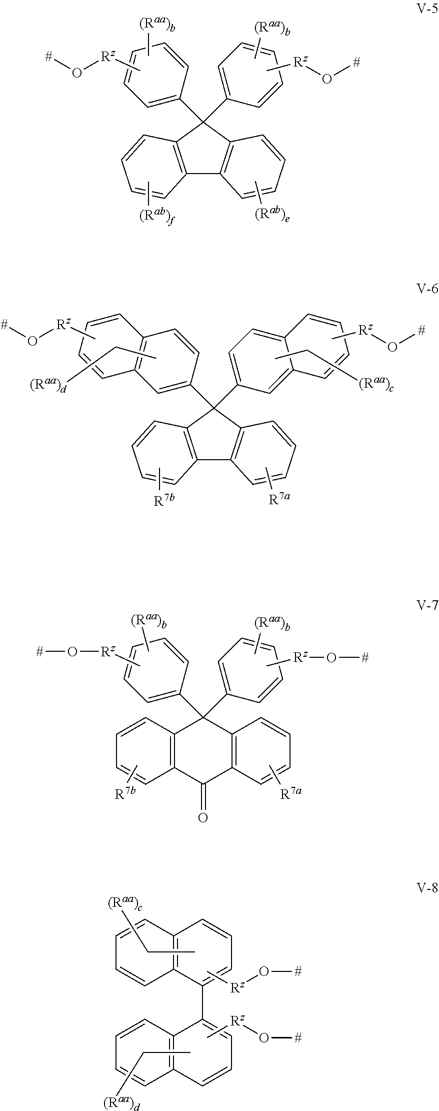

where
a and b are 0, 1, 2 or 3, in particular 0 or 1;
a′ and b′ are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
W′ is S, S(O), SO₂, O, single bond, CH₂, CH(CH₃), C(CH₃)₂, in particular S, S(O), SO₂ or C(CH₃)₂;
and where R^{z}, R^{aa}, R^{ab}, R^{7a}, R^{7b} and L are as defined for formula (V) and where R^{z} is in particular selected from a single bond, CH₂ and OCH₂CH₂.

Particular preference is given to structural units of the general formulae (V-11) to (V-22), where R^{z} and R^{aa} are as defined herein and where R^{z} is in particular selected from a single bond, CH₂ and O—CH₂CH₂, and especially is O—CH₂CH₂:

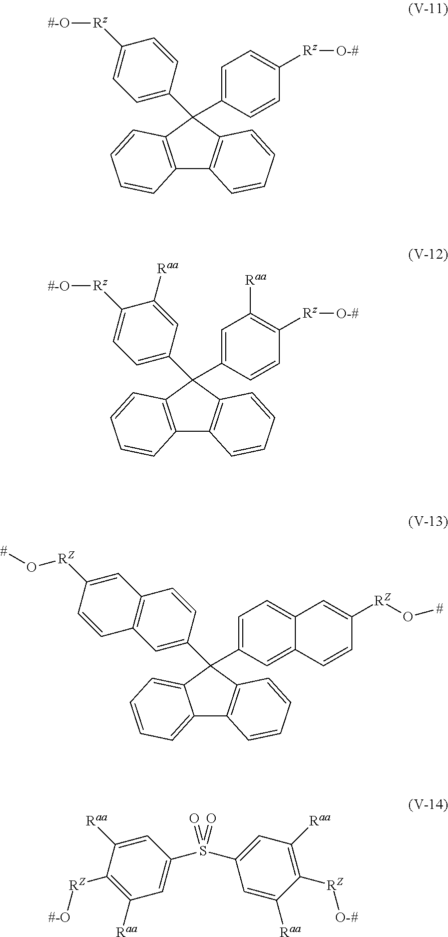

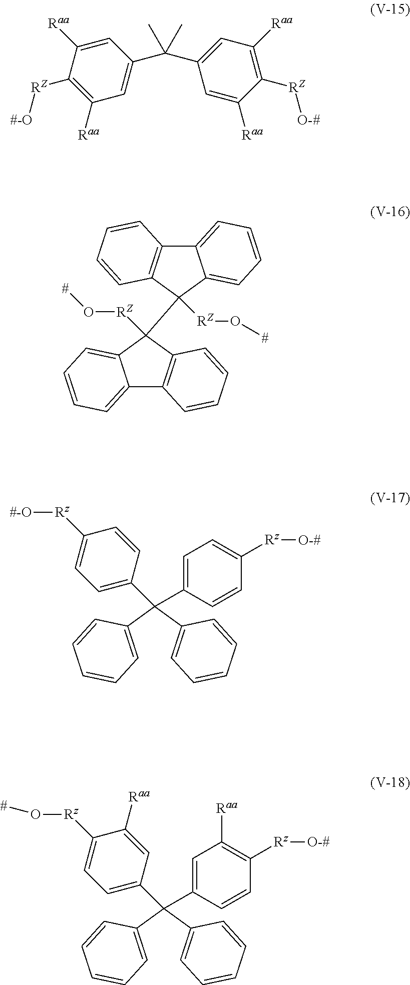

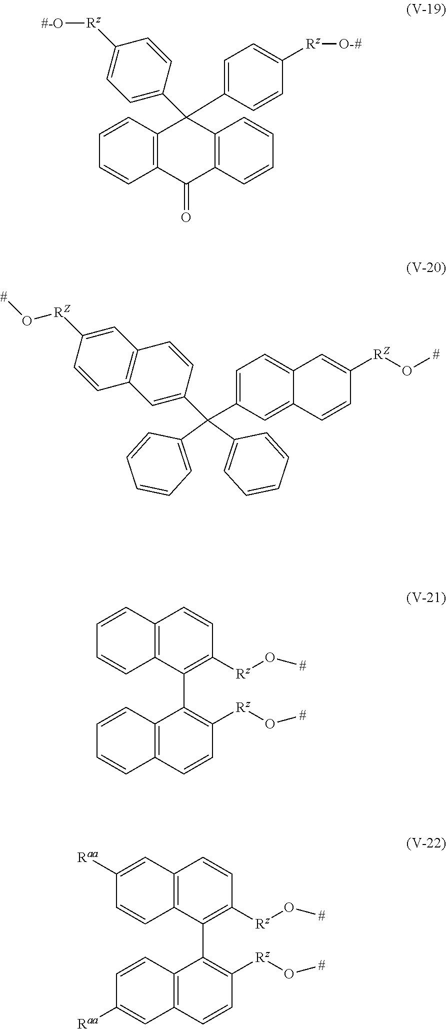

Among the structural units of the formulae (V-1) to (V-8), particular preference is given to the structural units of formulae (V-1), (V-2), (V-3) and (V-8). Among the structural units of the formulae (V-11) to (V-22), particular preference is given to the structural units of formulae (V-11), (V-12), (V-13), (V-14), (V-15), (V-21) and (V-22), and special preference given to structural units derived from 2,2′-bis(2-hydroxyethoxy)-1,1′-binaphtyl (BNE or BHBNA), 2,2′-bis(2-hydroxyethoxy)-6,6′-diphenyl-1,1′-binaphtyl (DPBHBNA), 9,9-bis(4-(2-hydroxyethoxy)phenyl)fluorene (BPEF), 9,9-bis(6-(2-hydroxy-ethoxy)naphthalene-2-yl)fluorene (BNEF), 9,9-bis(4-(2-hydroxyethoxy)-3-phenylphenyl)fluorene (BPPEF), 2-[4-[4-(2-hydroxyethoxy)-3,5-di(thianthrene-1-yl)phenyl]sulfonyl-2,6-di(thianthrene-1-yl)phenoxy]ethanol, 2-[4-[4-(2-hydroxyethoxy)-3,5-di(phenanthren-9-yl)phenyl]sulfonyl-2,6-di(phenanthren-9-yl)-phenoxy]ethanol, 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol, 2-[4-[1-[4-(2-hydroxyethoxy)-3,5-di(1,2-dibenzo[b,d]thien-4-yl)-phenyl]-1-methyl-ethyl]-2,6-di(1,2-dibenzo[b,d]thien-4-yl)-phenoxy]ethanol and 2-[4-[1-[4-(2-hydroxyethoxy)-3,5-di(thiantren-1-yl)-phenyl]-1-methyl-ethyl]-2,6-di(thianthren-1-yl)-phenoxy]ethanol.

In a particular preferred group of embodiments, the thermoplastic resin of the present invention comprises at least one structural unit of the formulae (IIa) or (IIb) and at least one structural unit selected from the group consisting of structural units of the formula (V-11), structural units of the formula (V-12), structural units of the formula (V-13), structural units of the formula (V-14), structural units of the formula (V-15), structural units of the formula (V-21) and structural units of the formula (V-22). In this particular group of embodiments, those thermoplastic resins are preferred, where in the structural units of the formulae (V-11), (V-12), (V-13), (V-14), (V-15), (V-21) and (V-22) the radicals R^{z} are O—CH₂CH₂.

In the thermoplastic resins of this particular preferred group of embodiments, it is preferred that the total molar ratio of the structural units of the formulae (IIa) or (IIb) is in the range from 1 to 99 mol-%, preferably in the range from 10 to 99 mol-%, further preferably in the range from 15 to 97 mol-%, and even further preferably in the range from 25 to 95 mol-% of the total amount of structural units of the formulae (II) and (V).

The compounds of the formulae (IV), (IV-1), (IV-2), (IV-3), (IV-4), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) and (IV-22) are known or can be prepared by analogy to known methods.

For example, the compounds of the formula (IV-8) can be prepared by various synthesis methods, as disclosed e.g. in JP Publication No. 2014-227387, JP Publication No. 2014-227388, JP Publication No. 2015-168658, and JP Publication No. 2015-187098.

For example, 1,1′-binaphthols may be reacted with ethylene glycol monotosylates; alternatively, 1,1′-binaphthols may be reacted with alkylene oxides, halogenoalkanols, or alkylene carbonates; and alternatively, 1,1′-binaphthols may be reacted with ethylene carbonates. Thereby, the compounds of the formula (IV-8) are obtained, where R^{z}—OH is O-Alk⁴-OH or O-Alk⁴-[O-Alk⁴-]_{w}- OH.

For example, the compounds of the formula (IV-2) can be prepared by various synthesis methods, as disclosed e.g. in JP Patent Publication No. 5442800, and JP Publication No. 2014-028806. Examples include:
(a) reacting fluorenes with hydroxy naphthalenes in the presence of hydrochloride gas and mercapto-carboxylic acid;
(b) reacting 9-fluorene with hydroxy naphthalenes in the presence of acid catalyst (and alkyl mercaptan);
(c) reacting fluorenes with hydroxy naphthalenes in the presence of hydrochloride and thiols (such as, mercapto-carboxylic acid);
(d) reacting fluorenes with hydroxy naphthalenes in the presence of sulfuric acid and thiols (such as, mercapto-carboxylic acid) and thereafter to crystallize the product from a crystallization solvent which consists of hydrocarbons and a polar solvent(s) to form bisnaphthol fluorene; and the like.

Thereby, compounds of the formula (IV-2) can be obtained, where R^{z} is a single bond. The compounds of formulae (IV), where R^{Z} is O-Alk⁴- or O-Alk⁴-[O-Alk⁴-]_{w}- can be prepared from compounds of formulae (IV), where R^{z} is a single bond, by reaction with alkylene oxides or haloalkanols. For example, reacting 9,9-bis(hydroxynaphthyl)-fluorenes of the formula (IV-2) where R^{z} is a single bond with alkylene oxides or haloalkanols results in the compounds of the formula (IV-2) where R^{z} is O-Alk⁴- or O-Alk⁴-[O-Alk⁴-]_{w}-. For example, 9,9-bis[6-(2-hydroxyethoxy)naphthyl]fluorene can be prepared by reacting 9,9-bis[6-(2-hydroxynaphthyl]fluorene with 2-chloroethanol under alkaline conditions.

The monomers of formulae (I) and (IV) used for producing the thermoplastic resin may contain certain impurities resulting from their preparation, e.g. the co-monomers (IV) may contain hydroxy compounds, which bear an OH group instead of e.g. a group 0-Alk⁴-OH, or may contain a group O-Alk⁴-[O-Alk⁴]_{w}- instead of a group O-Alk⁴-. The total amount of such impurity compounds is preferably 5000 ppm or lower, more preferably 3000 ppm or lower, still more preferably 2000 ppm or lower, and especially preferably 1000 ppm or lower. The total content of the impurities in the monomers used for preparing the thermoplastic resin is preferably 4000 ppm or lower in particular 1500 ppm or lower, and more preferably 1000 ppm or lower. In particular, the total amount of dihydroxy compounds in which a carbon number of at least one of the radicals R^{z}—OH differs from the formula (IV), is preferably 3000 ppm or lower, more preferably 1500 ppm or lower, still more preferably 1000 ppm or lower, and especially preferably 500 ppm or lower; in the monomer(s) of which the main component is the dihydroxy compound(s) represented by the formula (IV). The total content of the dihydroxy compounds in which a carbon number of at least one of the radicals R^{z}—OH differs from the formula (IV) is further preferably 1000 ppm or lower, and more preferably 500 ppm or lower. Likewise, the amount of impurities in the monomers of formula (I) will be in the range given for the monomers of formula (IV).

Suitable thermoplastic resins for the preparation of optical devices, such as lenses, are in particular polycarbonates, polyestercarbonates and polyesters. Preferred thermoplastic resins for the preparation of optical devices, such as lenses, are in particular polycarbonates.

Said polycarbonates are structurally characterized by having structural units of at least one of the formulae (II), (IIa) and (IIb), respectively, optionally structural units derived from diol monomers, which are different from the monomer compound of the formula (I), e.g. structural units of the formula (V),

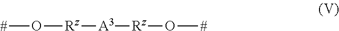

where
#, R^{z} and A³ are as defined herein above;
   and a structural unit of formula (Ill-1) stemming from the carbonate forming component:

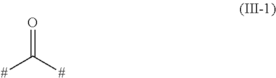

where each #represents a connection point to a neighboring structural unit, i.e. to 0 at the connection point of the structural unit of the formula (II) and, if present, to 0 at the connection point of the structural unit of the formula (V).

Said polyesters are structurally characterized by having structural units of at least one of the formulae (II), (IIa) and (IIb), respectively, optionally structural units derived from diol monomers which are different from the monomer compound of the formula (I), e.g. structural units of the formula V. If X^{1a} and X^{2a} in formula (II) or X^{a} in formulae (IIa) and (IIb) are selected from —CH₂O—, the polyesters may have structural units derived from one or more dicarboxylic acids, e.g. of formula (III-2) in case of a benzene dicarboxylic acid, of formula (III-3) in case of a naphthalene carboxylic acid, of formula (III-4) in case of oxalic acid and of formula (III-5) in case of malonic acid:

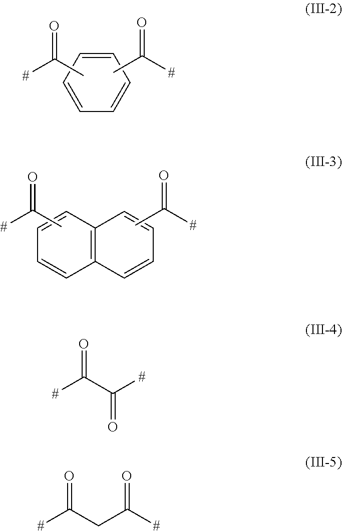

In formula (III-2) to (III-5) each variable #represents a connection point to a neighboring structural unit, i.e. to 0 of the connection point of the structural unit of the formula (II) and, if present, to 0 of the connection point of the structural unit of the formula (V).

Said polyestercarbonates are structurally characterized by having structural units of at least one of the formulae (II), (IIa) and (IIb), respectively, optionally structural units derived from diol monomers which are different from the monomer compound of the formula (I), e.g. structural units of the formula (V), a structural unit of formula (Ill-1) stemming from the carbonate forming component and structural units derived from dicarboxylic acid, e.g. of formula (III-2) in case of a benzene dicarboxylic acid, of formula (III-3) in case of a naphthalene carboxylic acid, of formula (III-4) in case of oxalic acid and of formula (III-5) in case of malonic acid.

A particular group of embodiments relates to thermoplastic copolymer resins, in particular polycarbonates, polyestercarbonates and polyesters, which have both structural units of formula (II) and one or more structural units of formula (V), i.e. resins, in particular polycarbonates, polyestercarbonates and polyesters, which are obtainable by reacting at least one monomer of formula (I) with one or more monomers of formula (IV). In this case the molar ratio of monomers of formula (I) to monomers of formula (IV) and likewise the molar ratio of the structural units of formula (II) to structural units of formula (V) are in the range from 1:99 to 99:1, in particular in the range from 10:90 to 99:1 and especially in the range from 30:70 to 97:3 or in the range from 10:90 to 99:1, in particular in the range from 15:85 to 97:3, more preferably in the range from 20:80 to 96:4 or in the range from 25:75 to 96:4, especially in the range from 27:73 to 96:4 or in the range from 27:73 to 99:1, even more preferably in the range from 27:73 to 90:10 and specifically in the range from 30:70 to 80:20 or in the range from 35:65 to 70:30. Accordingly, the molar ratio of the structural units of the formula (II) is usually from 1 to 99 mol-% in particular from 10 to 99 mol-%, more preferably in the range from 15 to 97 mol-% or in the range from 5 to 99 mol-%, especially in the range from 10 to 97 mol-% or in the range from 17 to 97 mol-%, even more preferably in the range from 17 to 90 mol-% and specifically in range from 20 to 80 mol-% or in the range from 25 to 70 mol-%, based on the total molar amount of structural units of the formulae (II) and (V). Accordingly, the molar ratio of the structural units of the formula (V) is usually from 1 to 99 mol-%, in particular from 1 to 90 mol-%, more preferably in the range from 3 to 85 mol-% or in the range from 1 to 95 mol-%, especially in the range from 3 to 90 mol-% or in the range from 3 to 83 mol-%, even more preferably in the range of 10 to 83 mol-% and specifically in range from 20 to 80 mol-% or in the range from 30 to 75 mol-%, based on the total molar amount of structural units of the formulae (II) and (V).

A specific group of embodiments relates to thermoplastic copolymer resins, in particular polycarbonates, polyestercarbonates and polyesters, which have both structural units of formula (II) and one or more structural units of formulae (V-14) or (V-15), i.e. resins, in particular polycarbonates, polyestercarbonates and polyesters, which are obtainable by reacting at least one monomer of formula (I) with one or more monomers of formulae (IV-14) or (IV-15). In this case the molar ratio of monomers of formula (I) to monomers of formulae (IV-14) and (IV-15) and likewise the molar ratio of the structural units of formula (II) to structural units of formulae (V-14) and (V-15) are in the range from 50:50 to 99:1, in particular in the range from 70:30 to 98:2 and especially in the range from 80:20 to 97:3.

Another specific group of embodiments relates to thermoplastic copolymer resins, in particular polycarbonates, polyestercarbonates and polyesters, which have both structural units of formula (II) and one or more structural units of formulae (V-11), (V-12), (V-13), (V-21) or (V-22), i.e. resins, in particular polycarbonates, polyestercarbonates and polyesters, which are obtainable by reacting at least one monomer of formula (I) with one or more monomers of formulae (IV-11), (IV-12), (IV-13), (IV-21) or (IV-22). In this case the molar ratio of monomers of formula (I) to monomers of formulae (IV-11), (IV-12), (IV-13), (IV-21) and (IV-22) and likewise the molar ratio of the structural units of formula (II) to structural units of formulae (V-11), (V-12), (V-13), (V-21) and (V-22) are in the range from 30:70 to 90:10, in particular in the range from 40:60 to 85:15 and especially in the range from 50:50 to 80:20.

The thermoplastic copolymer resins of the present invention, such as a polycarbonate resin may include either one of a random copolymer structure, a block copolymer structure, and an alternating copolymer structure. The thermoplastic resin according to the present invention does not need to include all of structural units (II) and one or more different structural units (V) in one, same polymer molecule. Namely, the thermoplastic copolymer resin according to the present invention may be a blend resin as long as the above-described structures are each included in any of a plurality of polymer molecules.

For example, the thermoplastic resin including all of structural units (II) and structural units (V) described above may be a copolymer including all of structural units (II) and structural units (V), it may be a mixture of a homopolymer or a copolymer including at least one structural unit (II) and a homopolymer or a copolymer including at least one structural unit (V) or it may be a blend resin of a copolymer including at least one structural unit (II) and a first structural unit (V) and a copolymer including at least one structural unit (II) and at least one other structural unit (V) different from the first structural units (V); etc.

Thermoplastic polycarbonates are obtainable by polycondensation of a diol component and a carbonate forming component. Similarly, thermoplastic polyesters and polyestercarbonates are obtainable by polycondensation of a diol component and a dicarboxylic acid, or an ester forming derivative thereof, and optionally a carbonate forming component.

Specifically, thermoplastic resins (polycarbonate resins) can be prepared by the following methods.

A method for preparing the thermoplastic resin of the present invention, such as a polycarbonate resin, includes a process of melt polycondensation of a dihydroxy component corresponding to the above-mentioned structural units and a diester carbonate. According to the present invention the dihydroxy compound comprises at least one dihydroxy compound represented by the formula (I), in particular by the formulae (la) or (Ib), respectively, as defined herein. In addition to the compound of formula (I), the dihydroxy compound may also comprise one or more dihydroxy compounds represented by the formula (IV), preferably by the formulae (IV-1) to (IV-8), in particular by the formulae (IV-11) to (IV-22), and especially by the formulae (IV-11), (IV-12), (IV-13), (IV-14), (IV-15), (IV-21) or (IV-22).

As is clear from the above, the polycarbonate resin can be formed by reacting a dihydroxy component with a carbonate precursor, such as a diester carbonate, where the dihydroxy component comprises at least one compound represented by the formulae (I), (Ia) and (Ib), respectively, or a combination of at least one compound represented by the formulae (I), (la) and (Ib), respectively, and at least one compound represented by the formulae (IV), (IV-1), (IV-2), (IV-3), (IV-4), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) or (IV-22). Specifically, a polycarbonate resin can be formed by a melt polycondensation process in which the compound represented by the formulae (I), (Ia) and (Ib), respectively, or a combination thereof with at least one compound of the formulae (IV), (IV-1), (IV-2), (IV-3), (IV-4), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) or (IV-22) and a carbonate precursor, such as a diester carbonate, are reacted in the presence of a basic compound catalyst, a transesterification catalyst, or a mixed catalyst thereof, or in the absence of a catalyst.

A thermoplastic resin (or a polymer) other than a polycarbonate resin, such as polyestercarbonates and polyesters is obtained by using the dihydroxy compound represented by the formulae (I), (Ia) and (Ib), respectively, or a combination thereof with at least one compound represented by the formulae (IV), (IV-1), (IV-2), (IV-3), (IV-4-), (IV-5), (IV-6), (IV-7), (IV-8), (IV-11), (IV-12), (IV-13), (IV-14), (IV-15), (IV-16), (IV-17), (IV-18), (IV-19), (IV-20), (IV-21) or (IV-22) as a material (or a monomer).

As mentioned before, the monomers of formula (I) and likewise the co-monomers of formula (IV) used for producing the thermoplastic resin may contain impurities resulting from their preparation.

For example, the monomers of the formulae (IV-1) and (IV-2), where R^{z} is O-Alk⁴- or O-Alk⁴-[O-Alk⁴-]_{w}-, may include a dihydroxy compound in which both R^{z} are a single bond, or a dihydroxy compound in which one of R^{z} is a single bond, instead of O-Alk⁴- or O-Alk⁴-[O-Alk⁴-]_{w}-.

The total amount of such dihydroxy compounds of the formulae (IV-1) or (IV-2) in which at least one of R^{z} differs from O-Alk⁴- or O-Alk⁴-[O-Alk⁴-]_{w}-, is preferably 3000 ppm or lower, more preferably 1500 ppm or lower, still more preferably 1000 ppm or lower, and especially preferably 500 ppm or lower; in the monomer(s) of which main component is the dihydroxy compound(s) represented by the formulae (IV-1) or (IV-2). The total content of the dihydroxy compounds in which at least one of the values of a and b or c and d differs from the formula (IV-1) or (IV-2) is still preferably 300 ppm or lower, and more preferably 200 ppm or lower.

The polycarbonate resins can be obtained by reacting the monomer compounds of the formula (I) or by reacting combination of at least one monomer compound of the formula (I), in particular at least one monomer (I) mentioned herein as preferred, and one or more monomer compounds of the formula (IV), and in particular of the formulae (IV-11), (IV-12), (IV-13), (IV-14), (IV-15), IV-21) or (IV-22), and the like, as dihydroxy components; with carbonate precursors, such as diester carbonates.

However, in a polymerization process for manufacturing the polycarbonate resins, some compounds of the formulae (I) and (IV) may be converted into impurities, where one of or both of the terminal —R^{z}OH radicals are replaced with a different radical, such as a vinyl terminal radical represented by —OCH═CH₂. Because the amount of such impurities is generally small, the products of the formed polymers can be used as polycarbonate resins without a purification process.

The thermoplastic resin of the present invention may also contain minor amount of impurities, for example, as extra contents of thermoplastic resin composition or a part of the polymer skeleton of the thermoplastic resin. The examples of such impurities include phenols formed by a process for forming the thermoplastic resin, unreacted diester carbonates and monomers. The total amount of impurities in the thermoplastic resin may be 5000 ppm or lower, or 2000 ppm or lower. The total amount of impurities in the thermoplastic resin is preferably 1000 ppm or lower, more preferably 500 ppm or lower, still more preferably 200 ppm or lower, and especially preferably 100 ppm or lower.

The total amount of phenols as impurities in the thermoplastic resin may be 3000 ppm or lower, or 2000 ppm or lower. The total amount of phenols as impurities is preferably 1000 ppm or lower, more preferably 800 ppm or lower, still more preferably 500 ppm or lower, and especially preferably 300 ppm or lower.

The total amount of diester carbonates as impurities in the thermoplastic resin is preferably 1000 ppm or lower, more preferably 500 ppm or lower, still more preferably 100 ppm or lower, and especially preferably 50 ppm or lower.

The total amount of unreacted monomers as impurities in the thermoplastic resin is preferably 3000 ppm or lower, more preferably 2000 ppm or lower, still more preferably 1000 ppm or lower, and especially preferably 500 ppm or lower.

The lower limit of the total amount of these impurities is not important, but may be 0.1 ppm, or 1.0 ppm.

The total amount of residual heavy metals, e.g. palladium, as impurity in the thermoplastic resin is preferably 50 ppm or lower, more preferably 10 ppm or lower. The amount of residual palladium can be reduced by standard procedures like treatment with an adsorbent, e.g. active charcoal.

Resins having targeted characteristics can be formed by adjusting the amounts of phenols and diester carbonates. The amounts of phenols, diester carbonates, and monomers can be suitably adjusted by arranging the conditions for polycondensation, the working conditions of devices used for polymerization, or the conditions for extrusion molding after the polycondensation process.

The weight-average molecular weight (Mw), as determined by GPC (gel permeation chromatography), of the thermoplastic resin according to the present invention is preferably in the range from 5000 to 100000 Dalton, more preferably 10000 to 80000 Dalton, especially in the range of 10000 to 50000 Dalton. The GPC measurements may be calibrated by using polystyrene standards. The Mw of a thermoplastic resin of the present invention determined this way is also denoted herein as “polystyrene conversion weight-average molecular weight”. The number-average molecular weight (Mn) of the thermoplastic resin according to the present invention is preferably in the range of 3000 to 30000, more preferably 5000 to 25000, and especially in the range of 7000 to 20000.

The viscosity-average molecular weight (Mv) of the thermoplastic resin according to the present invention is preferably in the range from 8000 to 28000, more preferably 9000 to 22000, and still more preferably 10000 to 18000.

The value of the molecular weight distribution (Mw/Mn) of the thermoplastic resin according to the present invention is preferably 1.5 to 9.0, more preferably 1.8 to 7.0, and still more preferably 2.0 to 4.0.

When a thermoplastic resin has the value of the weight-average molecular weight (Mw) within the above-mentioned suitable range, a molded article made from the thermoplastic resin has high strength. In addition, such a thermoplastic resin with the suitable Mw value is advantageous for molding because of its excellent fluidity.

Preferably, the thermoplastic resin comprises 9% by weight or less, in particular 7% by weight or less and especially 5% by weight or less, e. g. 0.1 to 9% by weight, in particular 0.1 to 7% by weight and especially 0.1 to 5% by weight, of low molecular weight compounds having molecular weight of less than 1000, based on the total weight of the thermoplastic resin. If such low molecular weight compounds are present in the thermoplastic resin in an amount within the above ranges, the mechanical strength of a molded body made from such a thermoplastic resin is commonly increased, especially compared to a molded body made from a thermoplastic resin with a higher amount of the low molecular weight compounds. In addition, a thermoplastic resin according to this embodiment comprising 9% by weight or less, in particular 7% by weight or less and especially 5% by weight of low molecular weight compounds with molecular weights of less than 1000, is not or only slightly prone to precipitation of the low molecular weight compounds, which is also known as bleed-out during a molding process, such as an injection molding process. In contrast, molding of a thermoplastic resin with a higher amount of the low molecular weight compounds may be accompanied by bleed-out to a greater extent.

The thermoplastic resin of the present invention, such as especially the above-mentioned polycarbonate resin, has a high refractive index (n_{D} or n_{d}) and thus is suitable to prepare an optical lens. The values of the refractive index as referred herein are values of a film having a thickness of 0.1 mm may be measured by use of an Abbe refractive index meter by a method of JIS-K-7142. The refractive index of the thermoplastic resin of the present invention, in particular the polycarbonate resin of the present invention, at 23° C. and at a wavelength of 589 nm is, in case the resin includes the structural unit (II), frequently 1.650 or higher, preferably 1.660 or higher, more preferably 1.670 or higher, still more preferably 1.680 or higher, in particular 1.690 or higher, such as 1.700 or higher. For example, the refractive index of the copolycarbonate resin including the structural unit (II) and a structural unit (V) according to the present invention is preferably 1.660 to 1.800, preferably 1.670 to 1.800, still more preferably 1.680 to 1.800.

The Abbe number (v) of the thermoplastic resin of the present invention, in particular the polycarbonate resin of the present invention, is preferably 24 or lower, more preferably 22 or lower, and still more preferably 20 or lower. The Abbe number may be calculated by use of the following equation based on the refractive index at wavelengths of 487 nm, 589 nm and 656 nm at 23° C.:

$$v = ( {n}_{D} - 1 ) / ( {n}_{F} - {n}_{C} )$$

n_{D}: refractive index at a wavelength of 589 nm
n_{C}: refractive index at a wavelength of 656 nm
n_{F}: refractive index at a wavelength of 486 nm

The glass transition temperature (Tg) of the thermoplastic resin of the present invention, in particular the polycarbonate resin of the present invention, is, in consideration of that the polycarbonate is usable for injection molding, frequently in the range of 90 to 185° C., preferably in the range of 90 to 180° C., more preferably in the range of 100 to 170° C., and especially in the range of 110 to 160° C. With regard to the molding fluidity and the molding heat resistance, the lower limit of Tg is preferably 130° C. and more preferably 135° C., and the upper limit of Tg is preferably 180° C. and more preferably 170° C. A glass transition temperature (Tg) in the above given ranges provides a significant range of usable temperature and avoids the risk that the melting temperature of the resin may be too high, and thus the resin may be undesirably decomposed or colored. What is more, it allows for preparing molds having a high surface accuracy. The values given for the glass transition temperature refer to the values measured by differential scanning calorimetry (DSC) using a 10° C./minute heating program according to the protocol of JIS K7121-1987.

The absolute value of the orientation birefringence of the thermoplastic resin of the present invention, in particular the polycarbonate resin of the present invention, is preferably in the range of 0 to 1×10⁻², more preferable in the range of 0 to 5×10⁻³, even more preferable in the range of 0 to 2×10⁻³, in particular in the range of 0 to 1×10⁻³, and specifically in the range of 0 to 0.4×10⁻³.

An optical molded body such as an optical element produced by using a polycarbonate resin of the present invention has a total light transmittance of preferably 85% or higher, more preferably 87% or higher, and especially preferably 88% or higher. A total light transmittance of preferably 85% or higher is as good as that provided by bisphenol A type polycarbonate resin or the like.

The thermoplastic resin according to the present invention has high moisture and heat resistance. The moisture and heat resistance may be evaluated by performing a “PCT test” (pressure cooker test) on a molded body such as an optical element produced by use of the thermoplastic resin and then measuring the total light transmittance of the molded body after the PCT test. In the PCT test, first, an injection molded body having a diameter of 50 mm and a thickness of 3 mm is kept for 20 hours with PC305S III made by HIRAYAMA Corporation under the conditions of 120° C., 0.2 MPa, 100% RH for 20 hours. Then, the sample of the injection molded body is removed from the device and the total light transmittance is measured using the SE2000 type spectroscopic parallax measuring instrument made by Nippon Denshoku Industries Co., Ltd in accordance with the method of JIS-K-7361-1.

The thermoplastic resin according to the present invention has a post-PCT test total light transmittance of 60% or higher, preferably 70% or higher, more preferably 75% or higher, still more preferably 80% or higher, and especially preferably 85% or higher. As long as the total light transmittance is 60% or higher, the thermoplastic resin is considered to have a higher moisture and heat resistance than that of the conventional thermoplastic resin.

The thermoplastic resin according to the present invention has a b value, which represents the hue, of preferably 5 or lower. As the b value is smaller, the color is less yellowish, which is good as a hue.

According to the invention, the diol component, which is used in the preparation of the polycarbonates or polyesters, may additionally comprise one or more diol monomers, which are different from the monomer compound of the formula (I), such as one or more monomers of the formula (IV).

Suitable diol monomers, which are different from the monomer compound of the formula (I), are those, which are conventionally used in the preparation of polycarbonates, e.g.
aliphatic diols such as ethylene glycol, propanediol, butanediol, pentanediol and hexanediol;
alicyclic diols such as tricyclo[5.2.1.02,6]decane dimethanol, cyclohexane-1,4-dimethanol, decalin-2,6-dimethanol, norbornane dimethanol, pentacyclopentadecane dimethanol, cyclopentane-1,3-dimethanol, spiroglycol, 1,4:3,6-dianhydro-D-sorbitol, 1,4:3,6-dianhydro-D-mannitol and 1,4:3,6-dianhydro-L-iditol are also included in examples of the diol; and
aromatic diols, in particular aromatic diols of the formula (IV) such as bis(4-hydroxyphenyl)methane, 1,1-bis(4-hydroxyphenyl)ethane, bis(4-hydroxy-phenyl)ether, bis(4-hydroxyphenyl)sulfoxide, bis(4-hydroxyphenyl)sulfide, bis(4-hydroxyphenyl)sulfone, bis(4-hydroxyphenyl)ketone, 2,2-bis(4-hydroxy-phenyl)propane, 2,2-bis(4-hydroxy-3-t-butylphenyl)propane, 2,2-bis(4-hydroxy-3-methylphenyl)propane, 1,1-bis(4-hydroxyphenyl)cyclopentane, 1,1-bis(4-hydroxy-phenyl)cyclohexane, 2,2-bis(4-hydroxyphenyl)hexafluoropropane, bis(4-hydroxy-phenyl)diphenylmethane, 1,1-bis(4-hydroxyphenyl)-1-phenylethane, α,ω-bis[2-(p-hydroxyphenyl)ethyl]polydimethylsiloxane, α,ω-bis[3-(o-hydroxyphenyl)-propyl]polydimethylsiloxane, 4,4′-[1,3-phenylenebis(1-methylethylidene)hydroxy-phenyl]-1-phenylethane, 9,9-bis(4-hydroxyphenyl)fluorene, 9,9-bis[4-(2-hydroxy-ethoxy)-3-methylphenyl]fluorene, 9,9-bis[4-(2-hydroxyethoxy)-3-tert-butylphenyl]fluorene, 9,9-bis[4-(2-hydroxyethoxy)-3-isopropylphenyl]fluorene, 9,9-bis[4-(2-hydroxyethoxy)-3-cyclohexylphenyl]fluorene, 9,9-bis(4-hydroxy-3-phenyl-phenyl)fluorene, 9,9-bis(4-(2-hydroxyethyl)phenyl)fluorene, 9,9-bis(4-(2-hydroxy-ethyl)-3-phenylphenyl)fluorene, 9,9-bis(6-hydroxy-2-naphthyl)fluorene, 9,9-bis(6-(2-hydroxyethyl)-2-naphthyl)fluorene, 10,10-bis(4-hydroxyphenyl)anthracen-9-on, 10,10-bis(4-(2-hydroxyethyl)phenyl)anthracen-9-on, 2-[4-[4-(2-hydroxyethoxy)-3,5-di(thianthrene-1-yl)phenyl]sulfonyl-2,6-di(thianthrene-1-yl)phenoxy]ethanol, 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol and 2,2′-[1,1′-binaphthalene-2,2′-diylbis(oxy)]diethanol, also termed 2,2′-bis(2-hydroxyethoxy)-1,1′-binaphthyl or 2,2′-bis(2-hydroxyethoxy)-1,1′-binaphthalene (BNE).

Preferably, the diol component comprises at least one monomer of the formula (IV) in addition to the monomer of formula (I). In particular, the total amount of monomers of formulae (I) and (IV) contributes to the diol component by at least 90% by weight, based on the total weight of the diol component or by at least 90 mol-%, based on the total molar amount of the diol monomers of the diol component. In particular, the diol component comprises at least one monomer selected from the monomers of formulae (IV-11) to (IV-22) in addition to the monomer of formula (I). More particularly, the diol component comprises at least one monomer selected from the monomers of formulae (IV-11), (IV-12), (IV-13), (IV-14), (IV-15), (IV-21) and (IV-22) in addition to the monomer of formula (I). Especially, the diol component comprises at least one monomer selected from 2,2′-bis(2-hydroxyethoxy)-1,1′-binaphtyl, 2,2′-bis(2-hydroxyethoxy)-6,6′-diphenyl-1,1′-binaphtyl, 9,9-bis(6-(2-hydroxyethoxy)-2-naphthyl)fluorene, 9,9-bis(4-(2-hydroxyethoxy)phenyl)fluorene, 2-[4-[4-(2-hydroxyethoxy)-3,5-di(thianthrene-1-yl)phenyl]sulfonyl-2,6-di(thianthrene-1-yl)phenoxy]ethanol, 2-[4-[4-(2-hydroxyethoxy)-3,5-di(dibenzo[b,d]thien-4-yl)phenyl]sulfonyl-2,6-dibenzo[b,d]thien-4-yl)phenoxy]ethanol, 2-[4-[1-[4-(2-hydroxyethoxy)-3,5-di(phenanthren-9-yl)-phenyl]-1-methylethyl]-2,6-di(phenanthren-9-yl)-phenoxy]ethanol and 9,9-bis(4-(2-hydroxyethoxy)-3-phenylphenyl)fluorene and combinations thereof in addition to the monomer of formula (I).

Frequently, the relative amount of monomer compound of formula (I), based on the total weight of the diol component, is at least 1% by weight, preferably at least 10% or at least 25% by weight, in particular at least 15% by weight or at least 20% by weight and especially at least 15% by weight or at least 25% by weight, preferably in the range of 1 to 99% by weight or in the range of 10 to 98% by weight, in particular in the range of 15 to 98% by weight or in the range of 20 to 98% by weight or in the range of 25 to 98% by weight or in the range 25 to 97% by weight, especially in the range of of 10 to 96% by weight or in the range of 15 to 95% by weight or in the range 25 to 95% by weight or in the range of 25 to 93% by weight, but may also be as high as 100% by weight.

Frequently, the relative molar amount of monomer compound of formula (I), based on the total molar amount of the diol component, is at least 1 mol-%, preferably at least 10 mol-% or at least 25 mol-%, in particular at least 15 mol-% or at least 20 mol-% and especially at least 15 mol-% or at least 25 mol-%, preferably in the range of 1 to 99 mol-% or in the range of 10 to 98 mol-% or in the range of 15 to 98 mol-% or in the range of 20 to 98 mol-%, in particular in the range of 10 to 96 mol-% or in the range of 15 to 95 mol-% or in the range of 25 to 95 mol-% or in the range of 25 to 93 mol-%, especially in the range of 15 to 90 mol-% or in the range of 20 to 90 mol-% or in the range of 25 to 90 mol-% or in the range of 30 to 90 mol-%, but may also be as high as 100 mol-%.

Consequently, the relative molar amount of monomer compound of formula (IV), based on the total molar amount of the diol component, will not exceed 99 mol-% or 90 mol-% or 75 mol-%, in particular not exceed 85 mol-% or 80 mol-% and especially not exceed 85 mol-% or 75 mol-%, and is preferably in the range of 1 to 99 mol-% or in the range of 2 to 90 mol-% or in the range of 2 to 85 mol-% or in the range of 3 to 75 mol-%, in particular in the range of 4 to 90 mol-% or in the range of 5 to 85 mol-% or in the range of 5 to 75 mol-% or in the range of 7 to 75 mol-%, especially in the range of 10 to 85 mol-% or in the range of 10 to 80 mol-% or in the range of 10 to 75 mol-% or in the range of 10 to 70 mol-%, but may also be as high as 99.9 mol-%.

Frequently, the total molar amount of monomers of formula (I) and monomers of formula (IV) is at least 80 mol-%, in particular at least 90 mol-%, especially at least 95 mol-% or up to 100 mol-%, based on the total molar amount of the diol monomers in the diol component.

Examples of further preferred aromatic dihydroxy compound, which can be used in addition to the monomers of formula (I) and optionally monomers of formula (IV) include, but are not limited to bisphenol A, bisphenol AP, bisphenol AF, bisphenol B, bisphenol BP, bisphenol C, bisphenol E, bisphenol F, bisphenol G, bisphenol M, bisphenol S, bisphenol P, bisphenol PH, bisphenol TMC, bisphenol Z and the like.

In order to adjust the molecular weight and the melt viscosity, the monomers forming the thermoplastic polymer may also include a monofunctional compound, in case of polycarbonates a monofunctional alcohol and in case of polyesters a monofunctional alcohol or a monofunctional carboxylic acid. Suitable monoalcohols are butanol, hexanol and octanol. Suitable monocarboxylic acids include e.g. benzoic acid, propionic acid and butyric acid. In order to increase the molecular weight and the melt viscosity, the monomers forming the thermoplastic polymer may also include a polyfunctional compound, in case of polycarbonates a polyfunctional alcohol having three or more hydroxyl groups and in case of polyesters a polyfunctional alcohol having three or more hydroxyl groups or a polyfunctional carboxylic acid having three or more carboxyl groups. Suitable polyfunctional alcohols are e.g. glycerine, trimethylol propane, pentaerythrit and 1,3,5-trihydroxy pentane. Suitable polyfunctional carboxylic acids having three or more carboxyl groups are e.g. trimellitic acid and pyromellitic acid. The total amount of these compounds, will frequently not exceed 10 mol-%, based on the molar amount of the diol component.

Suitable carbonate forming monomers, are those, which are conventionally used as carbonate forming monomers in the preparation of polycarbonates, include, but are not limited to phosgene, diphosgene and diester carbonates such as diethyl carbonate, diphenyl carbonate, di-p-tolyl carbonate, phenyl-p-tolyl carbonate, di-p-chlorophenyl carbonate and dinaphthyl carbonate. Out of these, diphenyl carbonate is particularly preferred. The carbonate forming monomer is frequently used at a ratio of 0.97 to 1.20 mol, and more preferably 0.98 to 1.10 mol, with respect to 1 mol of the dihydroxy compound(s) in total.

Suitable dicarboxylic acids include, but are not limited to
aliphatic dicarboxylic acids such as oxalic acid, malonic acid, succinic acid, glutaric acid, adipic acid, pimelic acid, suberic acid, azelaic acid;
alicyclic dicarboxylic acids such as tricyclo[5.2.1.02,6]decane dicarboxylic acid, cyclohexane-1,4-dicarboxylic acid, decalin-2,6-dicarboxylic acid, and norbornandicarboxylic acid; and
aromatic dicarboxylic acids, such as benzene dicarboxylic acids, specifically phthalic acid, isophthalic acid, 2-methylterephthalic acid or terephthalic acid, and naphthalene dicarboxylic acids, specifically naphthalene-1,3-dicarboxylic acid, naphthalene-1,4-dicarboxylic acid, naphthalene-1,5-dicarboxylic acid, naphthalene-1,6-dicarboxylic acid, naphthalene-1,7-dicarboxylic acid, naphthalene-2,5-dicarboxylic acid, naphthalene-2,6-dicarboxylic acid, 2-[9-(carboxymethyl)fluoren-9-yl]acetic acid (formula DC1), 2-[9-(carboxymethyl)fluoren-9-yl]propionic acid (formula DC2), 2,2′-bis(carboxymethyloxy)-1,1′-binaphthyl (formula DC3) and naphthalene-2,7-dicarboxylic acid.

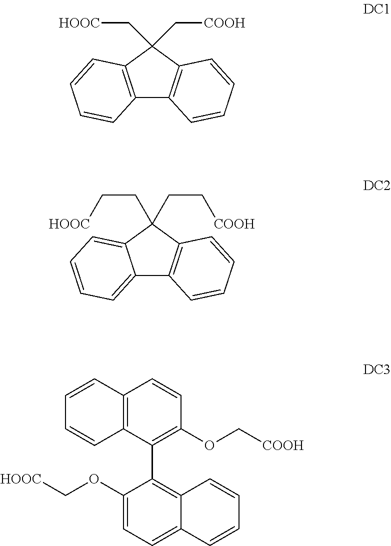 [SMILES: O=C(O)CC1(CC(=O)O)c2ccccc2-c2ccccc21 ; O=C(O)CCC1(CCC(=O)O)c2ccccc2-c2ccccc21 ; O=C(O)COc1ccc2ccccc2c1-c1c(OCC(=O)O)ccc2ccccc12]

Suitable ester forming derivatives of dicarboxylic acids include, but are not limited to the dialkyl esters, the diphenyl esters and the ditolyl esters.

In case of polyesters, the ester forming monomer is frequently used at a ratio of 0.97 to 1.20 mol, and more preferably 0.98 to 1.10 mol, with respect to 1 mol of the dihydroxy compound(s) in total.

The polycarbonates of the present invention can be prepared by reacting a diol component comprising a monomer of formula (I) and optionally a further diol monomer such as a monomer of the formula (IV) and a carbonate forming monomer by analogy to the well known preparation of polycarbonates as described e.g. in U.S. Pat. No. 9,360,593, US 2016/0319069 and US 2017/0276837, to which full reference is made.

The polyesters of the present invention can be prepared by reacting a diol component comprising a monomer of formula (I) and optionally a further diol monomer such as a monomer of the formula (IV) and a dicarboxylic acid or its ester forming derivative by analogy to the well known preparation of polyesters as described e.g. in US 2017/044311 and the references cited therein, to which full reference is made.

The polyestercarbonates of the present invention can be prepared by reacting a diol component comprising a monomer of formula (I) and optionally a further diol monomer such as a monomer of the formula (IV), a carbonate forming monomer and a dicarboxylic acid or its ester forming derivative by analogy to the well known preparation of polyestercarbonates as described in the art.

The polycarbonates, polyesters and polyestercarbonates are usually prepared by reacting the monomers of the diol component with the carbonate forming monomers and/or the ester forming monomers, i.e. the dicarboxylic acids or the ester forming derivatives thereof, in the presence of an esterification catalyst, in particular a transesterification catalyst, in case a carbonate forming monomer or an ester forming derivative of a polycarboxylic acid is used.

Suitable transesterification catalysts are basic compounds, which specifically include but are not limited to alkaline metal compounds, alkaline earth metal compound, nitro-gen-containing compounds, and the like. Likewise, suitable transesterification catalysts are acidic compounds, which specifically include but are not limited to Lewis acid compounds of polyvalent metals, including compounds such as zinc, tin, titanium, zirconium, lead, and the like.

Examples of suitable alkaline metal compound include alkaline metal salts of an organic acid such as acetic acid, stearic acid, benzoic acid, or phenylphorsphoric acid, alkaline metal phenolates, alkaline metal oxides, alkaline metal carbonates, alkaline metal borohydrides, alkaline metal hydrogen carbonates, alkaline metal phosphate, alkaline metal hydrogenphosphate, alkaline metal hydroxides, alkaline metal hydrides, alkaline metal alkoxides, and the like. Specific examples thereof include sodium hydroxide, potassium hydroxide, cesium hydroxide, lithium hydroxide, sodium hydrogen carbonate, sodium carbonate, potassium carbonate, cesium carbonate, lithium carbonate, sodium acetate, potassium acetate, cesium acetate, lithium acetate, sodium stearate, potassium stearate, cesium stearate, lithium stearate, sodium borohydride, sodium borophenoxide, sodium benzoate, potassium benzoate, cesium benzoate, lithium benzoate, disodium hydrogen phosphate, dipotassium hydrogen phosphate, dilithium hydrogen phosphate, and disodium phenylphosphate; and also include disodium salt, dipotassium salt, dicesium salt, dilithium salt of bisphenol A, sodium salt, potassium salt, cesium salt and lithium salt of phenol; and the like.

Examples of the alkaline earth metal compound include alkaline earth metal salts of an organic acid such as acetic acid, stearic acid, benzoic acid, or phenylphorsphoric acid, alkaline earth metal phenolates, alkaline earth metal earth oxides, alkaline earth metal carbonates, alkaline metal borohydrides, alkaline earth metal hydrogen carbonates, alkaline earth metal hydroxides, alkaline earth metal hydrides, alkaline earth metal alkoxides, and the like. Specific examples thereof include magnesium hydroxide, calcium hydroxide, strontium hydroxide, barium hydroxide, magnesium hydrogen carbonate, calcium hydrogen carbonate, strontium hydrogen carbonate, barium hydrogen carbonate, magnesium carbonate, calcium carbonate, strontium carbonate, barium carbonate, magnesium acetate, calcium acetate, strontium acetate, barium acetate, magnesium stearate, calcium stearate, calcium benzoate, magnesium phenylphosphate, and the like.

Examples of the nitrogen-containing compound include quaternary ammoniumhydrox-ide, salt thereof, amines, and the like. Specific examples thereof include quaternary ammoniumhydroxides including an alkyl group, an aryl group or the like, such as tetra-methylammoniumhydroxide, tetraethylammoniumhydroxide, tetrapropylammoniumhy-droxide, tetrabutylammoniumhydroxide, trimethylbenzylammoniumhydroxide, and the like; tertiary amines such as triphenylamine, dimethylbenzylamine, triphenylamine, and the like; secondary amines such as diethylamine, dibutylamine, and the like; primary amines such as propylamine, butylamine, and the like; imidazoles such as 2-methylimidazole, 2-phenylimidazole, benzoimidazole, and the like; bases or basic salts such as ammonia, tetramethylammoniumborohydride, tetrabutylammoniumborohydride, tetrabutylammoniumtetraphenylborate, tetraphenylammoniumtetraphenylborate, and the like.

Preferred examples of the transesterification catalyst include salts of polyvalent metals such as zinc, tin, titanium, zirconium, lead, and the like, in particular the chlorides, alkoxyides, alkanoates, benzoates, acetylacetonates and the like. They may be used independently or in a combination of two or more. Specific examples of such transesterification catalyst include zinc acetate, zinc benzoate, zinc 2-ethylhexanoate, tin chloride (II), tin chloride (IV), tin acetate (II), tin acetate (IV), dibutyltinlaurate, dibu-tyltinoxide, dibutyltinmethoxide, zirconiumacetylacetonate, zirconium oxyacetate, zirco-niumtetrabutoxide, lead acetate (II), lead acetate (IV), and the like.

The transesterification catalyst are frequently used at a ratio of 10⁻⁹ to 10⁻³ mol, preferably 10⁻⁷ to 10⁻⁴ mol, with respect to 1 mol of the dihydroxy compound(s) in total.

Frequently, the polycarbonates, polyesters and polyestercarbonates are prepared by a melt polycondensation method. In the melt polycondensation the monomers are reacted in the absence of an additional inert solvent. While the reaction is performed any byproduct formed in the transesterification reaction is removed by heating the reaction mixture at ambient pressure or reduced pressure.

The melt polycondensation reaction preferably comprises charging the monomers and catalyst into a reactor and subjecting the reaction mixture to conditions, where the reaction between the monomers and the formation of the byproduct takes place. It has been found advantageous, if the byproduct resides for at least a while in the polycondensation reaction. However, in order to drive the polycondensation reaction to the product side, it is beneficial to remove at least a portion of the formed byproduct during or preferably at the end of the polycondensation reaction. In order to allow the byproduct in the reaction mixture, the pressure may be controlled by closing the reactor, or by increasing or decreasing the pressure. The reaction time for this step is 20 minutes or longer and 240 minutes or shorter, preferably 40 minutes or longer and 180 minutes or shorter, and especially preferably 60 minutes or longer and 150 minutes or shorter. In this step, in the case where the byproduct is removed by distillation soon after being generated, the finally obtained thermoplastic resin has a low content of high molecular-weight resin molecules. By contrast, in the case where the byproduct is allowed to re-side in the reactor for a certain time, the finally obtained thermoplastic resin has a high content of high molecular-weight resin molecules.

The melt polycondensation reaction may be performed in a continuous system or in a batch system. The reactor usable for the reaction may be of a vertical type including an anchor-type stirring blade, a Maxblend® stirring blade, a helical ribbon-type stirring blade or the like; of a horizontal type including a paddle blade, a lattice blade, an eye glass-type blade or the like; or an extruder type including a screw. A reactor including a combination of such reactors is preferably usable in consideration of the viscosity of the polymerization product.

According to the method for producing the thermoplastic resin, such as a polycarbonate resin, after the polymerization reaction is finished, the catalyst may be removed or deactivated in order to maintain the thermal stability and the hydrolysis stability. A preferred method for deactivating the catalyst is the addition of an acidic substance. Specific examples of the acidic substance include esters such as butyl benzoate and the like; aromatic sulfonates such as p-toluenesulfonic acid and the like; aromatic sulfonic acid esters such as butyl p-toluenesulfonate, hexyl p-toluenesulfonate, and the like; phosphoric acids such as phosphorous acid, phosphoric acid, phosphonic acid, and the like; phosphorous acid esters such as triphenyl phosphite, monophenyl phosphite, diphenyl phosphite, diethyl phosphite, di-n-propyl phosphite, di-n-butyl phosphite, di-n-hexyl phosphite, dioctyl phosphite, monooctyl phosphite, and the like; phosphoric acid esters such as triphenyl phosphate, diphenyl phosphate, monophenyl phosphate, dibutyl phosphate, dioctyl phosphate, monooctyl phosphate, and the like; phosphonic acids such as diphenyl phosphonic acid, dioctyl phosphonic acid, dibutyl phosphonic acid, and the like; phosphonic acid esters such as diethyl phenylphosphonate, and the like; phosphines such as triphenylphosphine, bis(diphenylphosphino)ethane, and the like; boric acids such as boric acid, phenylboric acid, and the like; aromatic sulfonic acid salts such as tetarabutylphosphonium dodecylbenzensulfonate salt, and the like; organic halides such as chloride stearate, benzoyl chloride, chloride p-toluenesulfonate, and the like; alkylsulfonic acids such as dimethylsulfonic acid, and the like; organic halides such as benzyl chloride, and the like. These deactivators are frequently used at 0.01 to 50 mol, preferably 0.3 to 20 mol, with respect to the catalyst. After the catalyst has been deactivated, there may be a step of removing low boiling point compounds from the polymer by distillation. The distillation is preferably performed at reduced pressure, e.g. at a pressure of 0.1 to 1 mm Hg at a temperature of 200 to 350° C. For this step, a horizontal device including a stirring blade having a high surface renewal capability such as a paddle blade, a lattice blade, an eye glass-type blade or the like, or a thin film evaporator is preferably used.

It is desirable that the thermoplastic resin such as a polycarbonate resin has a very small amount of foreign objects. Therefore, the molten product is preferably filtered to remove any solids from the melt. The mesh of the filter is preferably 5 μm or less, and more preferably 1 μm or less. It is preferred that the generated polymer is filtrated by a polymer filter. The mesh of the polymer filter is preferably 100 μm or less, and more preferably 30 μm or less. A step of sampling a resin pellet needs to be performed in a low dust environment, needless to say. The dust environment is preferably of class 6 or lower, and more preferably of class 5 or lower.

The thermoplastic resin may be molded by any conventional molding procedure for producing optical elements. Suitable molding procedures include but are not limited to injection molding, compression molding, casting, roll processing, extrusion molding, extension and the like.

While it is possible to mold the thermoplastic resin of the invention as such, it is also possible to mold a resin composition, which contains at least one thermoplastic resin of the invention and which further contains at least one additive and/or further resin. Suitable additives include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like. Suitable further resins are e.g. another polycarbonate resin, polyester carbonate resin, polyester resin, polyamide, polyacetal and the like, which does not contain repeating units of the formula (I).

Examples of the antioxidant include but are not limited to triethyleneglycol-bis[3-(3-tert-butyl-5-methyl-4-hydroxyphenyl)propionate], 1,6-hexanediol-bis[3-(3,5-di-tert-butyl-4-hydroxyphenyl)propionate], pentaerythritol-tetrakis[3-(3,5-di-tert-butyl-4-hydroxyphenyl)propionate], octadecyl-3-(3,5-di-tert-butyl-4-hydroxyphenyl)propionate, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2(3H)-one, 5,7-Di-tert-butyl-3-(1,2dimethylphenyl)benzofuran-2(3H)-one, 1,3,5-trimethyl-2,4,6-tris(3,5-di-tert-butyl-4-hydroxybenzyl)benzene, N,N-hexamethylenebis(3,5-di-tert-butyl-4-hydroxy-hydrocinnamide, 3,5-di-tert-butyl-4-hydroxy-benzylphosphonate-diethylester, tris(3,5-di-tert-butyl-4-hydroxybenzyl)isocyanurate, and 3,9-bis{1,1-dimethyl-2-[β-(3-tert-butyl-4-hydroxy-5-methylphenyl)propionyloxy]ethyl}-2,4,8,10-tetraoxaspiro(5,5)undecane, and the like.

Among these examples, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2(3H)-one, and 5,7-Di-tert-butyl-3-(1,2dimethylphenyl)benzofuran-2(3H)-one are more preferred. The content of the antioxidant in the thermoplastic resin is preferably 0.001 to 0.3 parts by weight with respect to 100 parts by weight of the thermoplastic resin.

Examples of the processing stabilizer include but are not limited to phosphorus-based processing stabilizers, sulfur-based processing stabilizers, and the like. Examples of the phosphorus-based processing stabilizer include phosphorous acid, phosphoric acid, phosphonous acid, phosphonic acid, esters thereof, and the like. Specific examples thereof include triphenylphosphite, tris(nonylphenyl)phosphite, tris(2,4-di-tert-butylphenyl)phosphite, tris(2,6-di-tert-butylphenyl)phosphite, tridecylphosphite, trioctylphosphite, trioctadecylphosphite, didecylmonophenylphosphite, dioctylmo-nophenylphosphite, diisopropylmonophenylphosphite, monobutyldiphenylphosphite, monodecyldiphenylphosphite, monooctyldiphenylphosphite, bis(2,6-di-tert-butyl-4-methylphenyl)pentaerythritoldiphosphite, 2,2-methylenebis(4,6-di-tert-butylphenyl)octylphosphite, bis(nonylphenyl)pentaerythritoldiphosphite, bis(2,4-dicumylphenyl)pentaerythritoldiphosphite, bis(2,4-di-tert-butylphenyl)pentaerythritoldiphosphite, distearylpentaerythritoldiphosphite, tributylphosphate, triethylphosphate, trimethylphosphate, triphenylphosphate, diphenylmonoorthoxenylphosphate, dibu-tylphosphate, dioctylphosphate, diisopropylphosphate, dimethyl benzenephosphonate, diethyl benzenephosphonate, dipropyl benzenephosphonate, tetrakis(2,4-di-t-butylphenyl)-4,4′-biphenylenediphosphonite, tetrakis(2,4-di-t-butylphenyl)-4,3′-biphenylenediphosphonite, tetrakis(2,4-di-t-butylphenyl)-3,3′-biphenylenediphosphonite, bis(2,4-di-tert-butylphenyl)-4-phenyl-phenylphosphonite, bis(2,4-di-tert-butylphenyl)-3-phenyl-phenylphosphonite, and the like. The content of the phosphorus-based processing stabilizer in the thermoplastic resin composition is preferably 0.001 to 0.2 parts by weight with respect to 100 parts by weight of the thermoplastic resin.

Examples of the sulfur-based processing stabilizer include but are not limited to pen-taerythritol-tetrakis(3-laurylthiopropionate), pentaerythritol-tetrakis(3-myristylthiopropionate), pentaerythritol-tetrakis(3-stearylthiopropionate), dilauryl-3,3′-thiodipropionate, dimyristyl-3,3′-thiodipropionate, distearyl-3,3′-thiodipropionate, and the like. The content of the sulfur-based processing stabilizer in the thermoplastic resin compositon is preferably 0.001 to 0.2 parts by weight with respect to 100 parts by weight of the thermoplastic resin.

Preferred releasing agents contain at least 90% by weight of an ester of an alcohol and a fatty acid. Specific examples of the ester of an alcohol and a fatty acid include an ester of a monovalent alcohol and a fatty acid, and a partial ester or a total ester of a polyvalent alcohol and a fatty acid. Preferred examples of the above-described ester of an alcohol and a fatty acid include the esters of a monovalent alcohol having a carbon number of 1 to 20 and a saturated fatty acid having a carbon number of 10 to 30. Preferred examples of partial or total esters of a polyvalent alcohol and a fatty acid include the partial or total ester of a polyvalent alcohol having a carbon number of 2 to 25 and a saturated fatty acid having a carbon number of 10 to 30. Specific examples of the ester of a monovalent alcohol and a fatty acid include stearyl stearate, palmityl palmi-tate, butyl stearate, methyl laurate, isopropyl palmitate, and the like. Specific examples of the partial or total ester of a polyvalent alcohol and a fatty acid include monoglyceride stearate, monoglyceride stearate, diglyceride stearate, triglyceride stearate, mono-sorbitate stearate, monoglyceride behenate, monoglyceride caprylate, monoglyceride laurate, pentaerythritol monostearate, pentaerythritol tetrastearate, pentaerythritol tetrapelargonate, propyleneglycol monostearate, biphenyl biphenate, sorbitan monostearate, 2-ethylhexylstearate, total or partial esters of dipentaerythritol such as dipentaerythritol hexastearate and the like, etc. The content of the releasing agent in the resin composition is preferably 0.005 to 2.0 parts by weight, more preferably 0.01 to 0.6 parts by weight, and still more preferably 0.02 to 0.5 parts by weight, with respect to 100 parts by weight of the thermoplastic resin.

Preferred ultraviolet absorbers are selected from the group consisting of benzotriazole-based ultraviolet absorbers, benzophenone-based ultraviolet absorbers, triazine-based ultraviolet absorbers, cyclic iminoester-based ultraviolet absorbers, and cyanoacrylate-based ultraviolet absorbers. Namely, the following ultraviolet absorbers may be used independently or in a combination of two or more.

Examples of benzotriazole-based ultraviolet absorbers include 2-(2-hydroxy-5-methylphenyl)benzotriazole, 2-(2-hydroxy-5-tert-octylphenyl)benzotriazole, 2-(2-hydroxy-3,5-dicumylphenyl)phenylbenzotriazole, 2-(2-hydroxy-3-tert-butyl-5-methylphenyl)-5-chlorobenzotriazole, 2,2′-methylenebis[4-(1,1,3,3-tetramethylbutyl)-6-(2N-benzotriazole-2-yl)phenol)], 2-(2-hydroxy-3,5-di-tert-butylphenyl)benzotriazole, 2-(2-hydroxy-3,5-di-tert-butylphenyl)-5-chlorobenzotriazole, 2-(2-hydroxy-3,5-di-tert-amylphenyl)benzotriazole, 2-(2-hydroxy-5-tert-octylphenyl)benzotriazole, 2-(2-hydroxy-5-tert-butylphenyl)benzotriazole, 2-(2-hydroxy-4-octoxyphenyl)benzotriazole, 2,2′-methylenebis(4-cumyl-6-benzotriazolephenyl), 2,2′-p-phenylenebis(1,3-benzoxazine-4-one), 2-[2-hydroxy-3-(3,4,5,6-tetrahydrophthalimidemethyl)-5-methylphenyl]benzotriazole, and the like.

Examples of benzophenone-based ultraviolet absorbers include 2,4-dihydroxybenzophenone, 2-hydroxy-4-methoxybenzophenone, 2-hydroxy-4-octoxybenzophenone, 2-hydroxy-4-benzyloxybenzophenone, 2-hydroxy-4-methoxy-5-sulfoxybenzophenone, 2-hydroxy-4-methoxybenzophenone-5-sulfonic acid hydrate, 2,2′-dihydroxy-4-methoxybenzophenone, 2,2′,4,4′-tetrahydroxybenzophenone, 2,2′-dihydroxy-4,4′-dimethoxybenzophenone, 2,2′-dihydroxy-4,4′-dimethoxy-5-sodiumsulfoxybenzophenone, bis(5-benzoyl-4-hydroxy-2-methoxyphenyl)methane, 2-hydroxy-4-n-dodecyloxybenzophenone, 2-hydroxy-4-methoxy-2′-carboxybenzophenone, and the like.

Examples of triazine-based ultraviolet absorbers include 2-(4,6-diphenyl-1,3,5-triazine-2-yl)-5-([(hexyl)oxy]-phenol, 2-(4,6-bis(2,4-dimethylphenyl)-1,3,5-triazine-2-yl)-5-([(octyl)oxy]-phenol, and the like.

Examples of cyclic iminoester-based ultraviolet absorbers include 2,2′-bis(3,1-benzoxazine-4-one), 2,2′-p-phenylenebis(3,1-benzoxazine-4-one), 2,2′-m-phenylenebis(3,1-benzoxazine-4-one), 2,2′-(4,4′diphenylene)bis(3,1-benzoxazine-4-one), 2,2′-(2,6-naphthalene)bis(3,1-benzoxazine-4-one), 2,2′-(1,5-naphthalene)bis(3,1-benzoxazine-4-one), 2,2′-(2-methyl-p-phenylene)bis(3,1-benzoxazine-4-one), 2,2′-(2-nitro-p-phenylene)bis(3,1-benzoxazine-4-one), 2,2′-(2-chloro-p-phenylene)bis(3,1-benzoxazine-4-one), and the like.

Examples of cyanoacrylate-based ultraviolet absorbers include 1,3-bis-[(2′-cyano-3′,3′-diphenylacryloyl)oxy]-2,2-bis(((2-cyano-3,3-diphenylacryloyl)oxy)methyl)propane, 1,3-bis-[(2-cyano-3,3-diphenylacryloyl)oxy]benzene, and the like.

The content of the ultraviolet absorber in the resin composition is preferably 0.01 to 3.0 parts by weight, more preferably 0.02 to 1.0 parts by weight, and still more preferably 0.05 to 0.8 parts by weight, with respect to 100 parts by weight of the thermoplastic resin. The ultraviolet absorber contained in such a range of content in accordance with the use may provide a sufficient climate resistance to the thermoplastic resin.

As mentioned above, the thermoplastic polymer resins, in particular the polycarbonate resins, comprising repeating units of formulae (II), (IIa) and (IIb), respectively, as described herein, provide high transparency and high refractive index to thermoplastic resins, which therefore are suitable for preparing optical devices, where high transparency and high refractive index is required. More precisely, the thermoplastic polycarbonates having structural units of formulae (II), (IIa) and (IIb), respectively, are characterized by having a high refractive index, which is preferably at least 1.640, more preferably at least 1.660, in particular at least 1.670.

The contribution of the monomer of the formulae (I), (Ia) and (Ib), respectively, to the refractive index of the thermoplastic resin, in particular a polycarbonate resin, will de-pend from the refractive index of said monomer and the relative amount of said monomer in the thermoplastic resin. In general, a higher refractive index of the monomer contained in the thermoplastic resin will result in a higher refractive index of the resulting thermoplastic resin. Apart from that, the refractive index of a thermoplastic resin comprising structural units of the formula (II) can be calculated from the refractive indices of the monomers used for preparing the thermoplastic resin, which in turn can be determined by measurement with a refractometer or by ab initio calculation, e.g. using the computer software ACD/ChemSketch 2012 (Advanced Chemistry Development, Inc.).

In case of thermoplastic copolymer resins, the refractive index of the thermoplastic resin, in particular a polycarbonate resin, can be calculated from the refractive indices of the homopolymers of the respective monomers, which form the copolymer resin, by the following so called “Fox equation”:

$$1 / {n}_{D} = {x}_{1} / {n}_{D 1} + {x}_{2} / {n}_{D 2} + … {x}_{n} / {n}_{Dn} ,$$

where no is the refractive index of the copolymer, x₁, x₂, . . . xₙ are the mass fractions of the monomers 1, 2, . . . n in the copolymer and n_{D1}, n_{D2}, . . . n_{Dn} are the refractive indices of the homopolymers synthesized from only one of the monomers 1, 2, . . . n at a time. In case of polycarbonates, x₁, x₂, . . . xₙ are the mass fractions of the OH monomers 1, 2, . . . n, based on the total amount of OH monomer. It is apparent that a higher refractive index of a homopolymer will result in a higher refractive index of the copolymer.

The refractive indices of the thermoplastic resins can be determined directly or indirectly. For direct determination, the refractive indices no of the thermoplastic resins are measured at wavelength of 589 nm in accordance with the protocol JIS-K-7142 using an Abbe refractometer and applying a 0.1 mm film of the thermoplastic resin. In case of the refractive indices of the homopolycarbonates of the compounds of formula (I), the refractive indices can also be determined indirectly. For this, a co-polycarbonate of the respective monomer of formula (I) with 9,9-bis(4-(2-hydroxyethoxy)phenyl)fluorene and diphenyl carbonate is prepared according to the protocol of example 1 in column 48 of U.S. Pat. No. 9,360,593 and the refractive indices no of the co-polycarbonate is measured at wavelength of 589 nm in accordance with the protocol JIS-K-7142 using an Abbe refractometer and applying a 0.1 mm film of the co-polycarbonate. From the thus measured refractive indices n_{D}, the refractive index of the homopolycarbonate of the respective monomer can be calculated by applying the Fox equation and the known refractive index of 9,9-bis(4-(2-hydroxyethoxy)phenyl)fluorene (no(589 nm)=1.639).

The compounds of formula (I) can be obtained in a purity, which provides for a low yellowness index Y.I., as determined in accordance with ASTM E313, which may also be important for the use in the preparation of optical resins.

More precisely, the yellowness index Y.I., as determined in accordance with ASTM E313, of the compounds of formula (I) preferably does not exceed 100, more preferably 50, even more preferably 20, in particular 10 or 5.

The thermoplastic resin according to the present invention has a high refractive index and a low Abbe number. The thermoplastic resin of the present invention can be used for producing a transparent conductive substrate usable for a liquid crystal display, an organic EL display, a solar cell and the like. Also, the thermoplastic resin of the present invention can be used as a structural material for optical parts, such as, optical disks, liquid crystal panels, optical cards, optical sheets, optical fibers, connectors, evaporated plastic reflecting mirrors, displays, and the like; or used as optical devices suitable for functional material purpose.

Accordingly, molded articles, such as optical devices can be formed using the thermoplastic resins of the present invention. The optical devices include optical lenses, and optical films. The specific examples of the optical devices include lenses, films, mirrors, filters, prisms, and so on. These optical devices can be formed by arbitrary production process, for example, by injection molding, compression molding, injection compression molding, extrusion molding, or solution casting.

Because of an excellent moldability and a high heat resistance, the thermoplastic resins of the present invention are very suitable for production of optical lenses which re-quires injection molding. For molding, the thermoplastic resins of the present invention, such as the polycarbonate resin, can be used with other thermoplastic resins, for example, different polycarbonate resin, polyestercarbonate resin, polyester resin, and other resins, as a mixture.

In addition, the thermoplastic resins of the present invention can be mixed with additives for forming the optical devices. As the additives for forming the optical devices, above-mentioned ones can be used. The additives may include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like.

As is clear from the above, another aspect of the present invention relates to an optical device made of a thermoplastic resin as defined above, where the thermoplastic resin comprising a structural unit represented by the formula (II) and optionally of formula (V). As regards to the preferred meanings and preferred embodiments of the structural units of the formulae (II) and (V), reference is made to the statements given above.

An optical device made of an optical resin comprising the repeating units of the formula (II) and optionally repeating units of the formula (V) as defined herein are usually optical molded articles such as optical lenses, for example car head lamp lenses, Fresnel lenses, fθ lenses for laser printers, camera lenses, lenses for glasses and projection lenses for rear projection TV's, CD-ROM pick-up lenses, but also optical disks, optical elements for image display media, optical films, film substrates, optical filters or prisms, liquid crystal panels, optical cards, optical sheets, optical fibers, optical connectors, eposition plastic reflective mirrors, and the like. Here particular preference is given to optical lenses and optical films. Optical resins comprising repeating units of the formula (II) and optionally repeating units of the formula (V) are also useful for producing a transparent conductive substrate usable for an optical device suitable as a structural member or a functional member of a transparent conductive substrate for a liquid crystal display, an organic EL display, a solar cell and the like.

The optical lens produced from the thermoplastic resin according to the present invention has a high refractive index, a low Abbe number and a low degree of birefringence, and is highly moisture and heat resistant. Therefore, the optical lens can be used in the field in which a costly glass lens having a high refractive index is conventionally used, such as for a telescope, binoculars, a TV projector and the like. It is preferred that the optical lens is used in the form of an aspherical lens. Merely one aspherical lens may make the spherical aberration substantially zero. Therefore, it is not necessary to use a plurality of spherical lenses to remove the spherical aberration. Thereby the weight and the production cost of a device including the spherical aberration is decreased. An aspherical lens is useful especially as a camera lens among various types of optical lenses. The present invention easily provides an aspherical lens having a high refractive index and a low level of birefringence, which is technologically difficult to produce by processing glass.

An optical lens of the present invention may be formed, for example, by injection molding, compression molding, injection compression molding or casting the resin the repeating units of the formula (II) and optionally repeating units of the formula (V) as defined herein.

The optical lens of the present invention is characterized by a small optical distortion. An optical lens comprising a conventional optical resin has a large optical distortion. Although it is not impossible to reduce the value of an optical distortion by molding conditions, the condition widths are very small, thereby making molding extremely difficult. Since the resin having repeating units of the formula (II) and optionally repeating units of the formula (V) as defined herein has an extremely small optical distortion caused by the orientation of the resin and a small molding distortion, an excellent optical element can be obtained without setting molding conditions strictly.

To manufacture the optical lens of the present invention by injection molding, it is preferred that the lens should be molded at a cylinder temperature of 260° C. to 320° C. and a mold temperature of 100° C. to 140° C.

The optical lens of the present invention is advantageously used as an aspherical lens as required. Since spherical aberration can be substantially nullified with a single aspherical lens, spherical aberration does not need to be removed with a combination of spherical lenses, thereby making it possible to reduce the weight and the production cost. Therefore, out of optical lenses, the aspherical lens is particularly useful as a camera lens.

Since resins having repeating units of the formula (II) and optionally repeating units of the formula (V) as defined herein have a high moldability, they are particularly useful as the material of an optical lens, which is thin and small in size and has a complex shape.

As a lens size, the thickness of the center part of the lens is 0.05 to 3.0 mm, preferably 0.05 to 2.0 mm, more preferably 0.1 to 2.0 mm. The diameter of the lens is 1.0 to 20.0 mm, preferably 1.0 to 10.0 mm, more preferably 3.0 to 10.0 mm. It is preferably a meniscus lens, which is convex on one side and concave on the other side.

The surface of the optical lens of the present invention may have a coating layer such as an antireflection layer or a hard coat layer as required. The antireflection layer may be a single layer or a multi-layer and composed of an organic material or inorganic material but preferably an inorganic material. Examples of the inorganic material include oxides and fluorides such as silicon oxide, aluminum oxide, zirconium oxide, titanium oxide, cerium oxide, magnesium oxide and magnesium fluoride.

The optical lens of the present invention may be formed by an arbitrary method such as metal molding, cutting, polishing, laser machining, discharge machining or edging. Metal molding is preferred.

An optical film produced by the use of the thermoplastic resin according to the present invention is high in transparency and heat resistance, and therefore is preferably usable for a liquid crystal substrate film, an optical memory card or the like. In order to avoid foreign objects from being incorporated into the optical film as much as possible, the molding needs to be performed in a low dust environment, needless to say. The dust environment is preferably of class 6 or lower, and more preferably of class 5 or lower.

The following examples serve as further illustration of the invention.

### 1. Abbreviations

m.p.: melting point
eq.: molar equivalent(s)
MEK: 2-butanone (methylethyl ketone)
TH F: tetrahydrofuran
TBME: tert-butyl methyl ether
MeOH: methanol
2-Me-THF: 2-methyl tetrahydrofuran
K₂CO₃: potassium carbonate
KI: potassium iodide
Na₂CO₃: sodium carbonate
NaHCO₃: sodium bicarbonate
NaOH: sodium hydroxide
NaBH₄: sodium borohydride
NH₄Cl: ammonium chloride
Na₂SO₄: sodium sulfate
HCl: hydrochloric acid
Oxone®: 2KHSO₅—KHSO₄—K₂SO₄ (oxidizing agent)
TLC: thin layer chromatography
n_{D}: refractive index
h: hour(s)

### 2. Preparation of Monomers of Formula (I)

### 2.1 Analytics Relating to Monomers of Formula (I)

¹H-NMR spectra were determined at 23° C. using an 80 MHz NMR-spectrometer (Magritek Spinsolve 80).

Melting points of the compounds were determined by Buchi Melting Point B-545.

### 2.2 Preparation of Starting Materials

### Example A: Synthesis of 6,6′-bis(naphthalen-2-yl)[1,1′-binaphthalene]-2,2′-diol [D2NBNA]

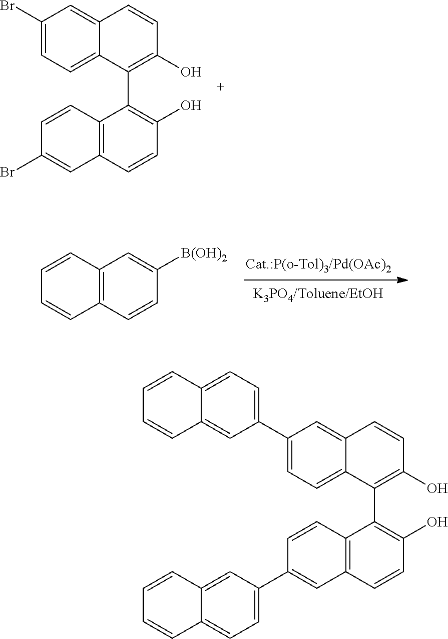 [SMILES: Oc1ccc2cc(Br)ccc2c1-c1c(O)ccc2cc(Br)ccc12 ; OB(O)c1ccc2ccccc2c1 ; Oc1ccc2cc(-c3ccc4ccccc4c3)ccc2c1-c1c(O)ccc2cc(-c3ccc4ccccc4c3)ccc12]

A mixture of 136.7 g (300 mmol) of 6,6′-dibromo-1,1′-bi-2-naphthol, 900 mL of toluene, 900 mL of ethanol, 2-naphthylboronic acid (108.35 g; 630 mmol), K₃PO₄ (140.1 g; 660 mmol), 312 g of water and 365 mg of tris-(o-tolyl)phosphin [P(o-Tol)₃] was heated to 60° C. and 67 mg (0.1 mol-%) of Pd(OCOCH₃)₂(palladium(II)acetate) were carefully added. The mixture was heated to reflux for 2.5 h until TLC control (MeOH/H₂O 3:1) showed complete conversion. The mixture was cooled to 70° C. The water phase was separated. The water phase was extract once with MEK (100 mL). The combined organic phase was washed subsequently with a saturated aqueous solution of Na₂CO₃ (400 mL), 125 mL of 2 M aqueous HCl and brine (350 mL). The organic phase was treated at room temperature with activated charcoal for 1 h, dried over Na₂SO₄ (40 g) and filtered over Celite. The solvent was removed under reduced pressure and the residue was taken up in hot MEK (700 g). Then, 200 g of toluene were added and the mixture was concentrated under reduced pressure to a mass of 240 g. Then, toluene was added (120 g) and the mixture was again concentrated under reduced pressure to a mass of 240 g. Toluene was added (240 g) and the mixture was concentrated under reduced pressure to a mass of 440 g. The resulting mixture was cooled to room temperature and stirred overnight. The formed crystals were collected by filtration, washed with toluene and pentane and dried at 60° C. to yield 172 g of the title compound D2NBNA (chemical purity UPLC: 97.6%).

¹H NMR (80 MHz, CDCl₃) δ=7.7-7.6 (m, 2M) 7.6-7.0 (m, 12H), 7.0-6.5 (m, 10H), 4.53 (bs, 2H, OH) ppm;
m. p. [DSC]=143.0° C.

### 2.3 Preparation Examples

### Example 1: {[1,1′-binaphthalene]-2,2′-diylbis(oxy-4,1-phenylene)}dimethanol (BNAD4PODMO) (compound of formula (Ia), with X=—CH₂OH and A=1,4-phenylene; compound 1 of table A)

### Step 1: Synthesis of 4,4′-[[1,1′-binaphthalene]-2,2′-diylbis(oxy)]dibenzaldehyde (BNAD4PODA)

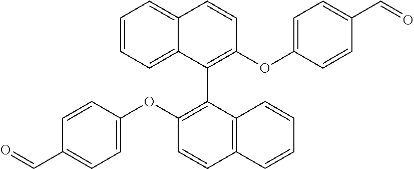 [SMILES: O=Cc1ccc(Oc2ccc3ccccc3c2-c2c(Oc3ccc(C=O)cc3)ccc3ccccc23)cc1]

### 4,4′-[[1,1′-binaphthalene]-2,2′-diylbis(oxy)]dibenzaldehyde

A mixture of rac-1,1′-bi-2-naphthol (144.61 g, 500 mmol, 1 eq) and K₂CO₃ (172.76 g, 1.25 mol, 2.5 eq) in N,N-dimethylformamide (530 mL) was stirred at 60° C. for 1 h. To this mixture was added 4-fluorobenzaldehyde (155.14 g, 1.25 mol, 2.5 eq) and it was stirred under reflux for approx. 2 h until TLC control (cyclohexane/ethyl acetate 1:1) showed complete conversion. The mixture was cooled to room temperature. Then, the mixture was slowly added to a mixture of 200 mL of concentrated aqueous HCl (37%) and 4 L of water. The resulting suspension was stirred for 1 h. The solid product was collected by filtration. The filter cake was washed 3 times with water (3×500 ml) and once with isopropanol (500 ml) and dried at 60° C. to yield 247.6 g of the title compound (BNAD4PODA), which can be used without purification for the next step (step 2).
m.p. [DSC]=235.9 C

¹H NMR (80 MHz, CDCl₃) δ=9.77 (s, 2H), 8.11-7.73 (m, 4H, 7.73-7.08 (m, 12H, 6.81 (d, J=8.6 Hz, 4H) ppm.

### Step 2: Synthesis of {[1,1′-binaphthalene]-2,2′-diylbis(oxy-4,1-phenylene)}dimethanol (BNAD4PODMO)

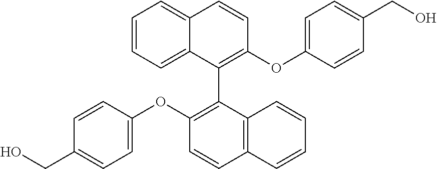 [SMILES: OCc1ccc(Oc2ccc3ccccc3c2-c2c(Oc3ccc(CO)cc3)ccc3ccccc23)cc1]

### [[1,1′-binaphthalene]-2,2′-diylbis(oxy-4,1-phenylene)]dimethanol

To a mixture of BNAD4PODA (100.93 g, 200 mmol, 1.00 eq; obtained in step 1) in THF (1000 g) was added portion wise NaBH₄ (15.1 g, 400 mmol, 2.00 eq). The reaction mixture was stirred under reflux for 1 h until TLC control (MTBE) showed complete conversion. The reaction mixture was cooled to 0° C. (ice bath) and 10% (w/w) hydrochloric acid was carefully added (approx. 100 ml) to adjust the pH to <3. The solvent was removed under reduced pressure. The remaining residue was taken up in 2-MeTHF (350 ml) and water (250 ml). The water phase was removed. The organic phase was washed with an aqueous solution of NaOH (1M; 250 ml), with 10% (w/w) hydrochloric acid (250 ml), with an aqueous saturated solution of Na₂CO₃ (250 ml) and brine (250 ml). The organic phase was dried over Na₂SO₄ and treated with 5 g of activated charcoal (Norit DX Ultra). The charcoal and the sodium sulphate was removed by filtration over celite and the filtrate was concentrated under reduced pressure to yield 103.6 g of crude product. This material was dissolved in hot toluene (333 g). The mixture was cooled to 50° C., seeded and cooled slowly to room temperature. The solid was collected by filtration, washed with toluene (3×30 g) and n-pentane (200 ml) and dried at 60° C. to yield the title compound as a white solid (83.9 g, chemical purity: 99.1%).

The material was dissolved again in 400 g of 2-MeTHF, washed with aqueous NaOH (10% w/w; 133 ml) and two times with brine (2×100 ml). The organic phase was dried over Na₂SO₄ and treated with 5 g of activated charcoal (Norit DX Ultra). The charcoal and the sodium sulphate was removed by filtration over celite and the filtrate was concentrated under reduced pressure to yield 97.5 g of crude product. This material was dissolved in hot toluene (273 g). The mixture was cooled to 50° C., seeded and cooled slowly to room temperature. The solid was collected by filtration, washed with toluene (3×30 g) and dried at 60° C. to yield the title compound as a white solid (75.6 g, chemical purity UPLC: 99.34%).
m.p. [DSC]=144.3° C.

¹H NMR (80 MHz, CDCl₃) δ=7.83 (d, J=9.0 Hz, 4H), 7.52-7.07 (m, 8H), 7.02-6.82 (m, 4H), 6.79-6.47 (m, 4H), 4.43 (s, 4H) 2.45 (bs, 2H) ppm.

### Example 2: {[1,1′-binaphthalene]-2,2′-diylbis(oxynaphthalene-4,1-diyl)}dimethanol (BNAD4NODMO) (compound of formula (Ia), with X=—CH₂OH, A=1,4-naphthylene and R=H; compound 4 of table A)

### Step 1: Synthesis of 4,4′-[[1,1′-binaphthalene]-2,2′-diylbis(oxy)]di(naphthalene-1-carbaldehyde) (BNAD4NODA)

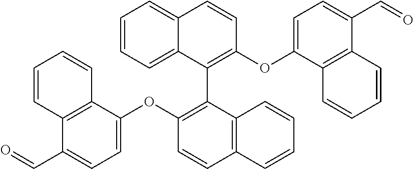 [SMILES: O=Cc1ccc(Oc2ccc3ccccc3c2-c2c(Oc3ccc(C=O)c4ccccc34)ccc3ccccc23)c2ccccc12]

### 4,4′-[[1,1′-binaphthalene]-2,2′-diylbis(oxy)]di(naphthalene-1-carbaldehyde)

A mixture of rac. 1,1′-bi-2-naphthol (98.3 g, 340 mmol, 1 eq) and K₂CO₃ (117.5 g, 850 mmol, 2.5 eq) in dimethylformamide (360 mL) was stirred at 60° C. for 1 h. To this mixture was added 4-fluoronaphthylaldehyd (141.7 g, 850 mmol, 2.5 eq) at 50° C. and it was stirred under reflux for approx. 2 h until TLC control (methanol/H₂O 3:1) showed complete conversion. The mixture was cooled to 70° C. Then, the mixture was slowly added to a mixture of 136 mL of concentrated aqueous HCl (37%) and 3100 L of water. The resulting suspension was stirred for 3 h. The solid product was collected by filtration. The filter cake was washed 3 times with water (3×500 ml), 3 times with isopropanol (3×230 ml) and 3 times with methanol (3×230 ml) and dried at 60° C. for 2 days to yield 208.4 g of the title compound (BNAD4NODA), which can be used without purification for the next step (step 2).

¹H NMR (80 MHz, CDCl₃) δ=10.13 (s, 2H), 9.3 (d, J=8.08 Hz, 2H), 8.22-7.27 (m, 20H), 6.60 (d, J=8.0 Hz, 2H) ppm.

### Step 2: Synthesis of {[1,1′-binaphthalene]-2,2′-diylbis(oxynaphthalene-4,1-diyl)}dimethanol (BNAD4NODMO)

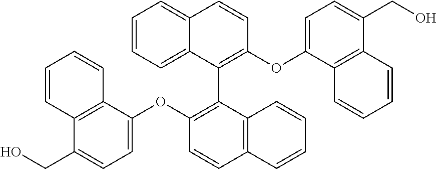 [SMILES: OCc1ccc(Oc2ccc3ccccc3c2-c2c(Oc3ccc(CO)c4ccccc34)ccc3ccccc23)c2ccccc12]

### [[1,1′-binaphthalene]-2,2′-diylbis(oxynaphthalene-4,1-diyl)]dimethanol

To a mixture of BNAD4NODA (124.53 g, 200 mmol, 1.00 eq; obtained in step 1) in 2-MeTHF (1000 g) was added portion wise NaBH₄ (15.2 g, 402 mmol, 2.00 eq). The reaction mixture was stirred under reflux for 10 h. An additional portion of NaBH₄ (15.2 g, 402 mmol, 2.00 eq) was added and the mixture was stirred for 1 h until TLC control (MTBE) showed complete conversion. The reaction mixture was cooled to room temperature and washed three times with water (3×250 ml). Note: the water phase was quenched with 10% (w/w) hydrochloric acid and discarded. The organic phase was washed 3 times with an aqueous saturated solution of citric acid (10% w/w; 3×250 ml) and brine (250 ml). The organic phase was dried over Na₂SO₄ and treated with 5 g of activated charcoal (Norit DX Ultra). The charcoal and the sodium sulphate was removed by filtration over celite. To the filtrate was added toluene (240 g) and the mixture was concentrated under reduced pressure to 316 g (removal of 2-MeTHF). Toluene (360 g) was added and the mixture was stirred overnight at room temperature. The formed crystals were collected by filtration, washed with toluene (3×30 g) and n-pentane (200 ml) and dried at 60° C. to yield the title compound as a off-white solid (103.2 g, chemical purity: 96.7%).

The material was dissolved in 500 g of THE and was treated with 5 g of activated charcoal (Norit DX Ultra) at 40° C. for 2 h. The charcoal was removed by filtration over celite and to the filtrate was added toluene (200 g). Then the mixture was concentrated under reduced pressure to remove THE (residue: 234 g of product dissolved in toluene). The remaining solution was stirred overnight at room temperature. The formed crystals were collected by filtration, washed with toluene (3×30 g) and n-pentane (200 ml) and dried at 60° C. to yield the title compound as a white solid (82.6 g, chemical purity UPLC: 97.7%).

The recrystallization procedure described above was repeated. By this the title compound was obtained as a white solid (73.6 g, chemical purity UPLC: 97.9%).

¹H NMR (80 MHz, Acetone-d₆) δ=7.82-7.32 (m, 8H), 7.26-6.50 (m, 14H), 6.34 (d, J=7.8 Hz, 2H), 4.56 (d, J=5.3 Hz, 4H), 3.78 (t, J=5.5 Hz, 2H) ppm.
m.p. [DSC]=201.3° C.

### Example 3: [(6,6′-Bis(naphthalen-2-yl)[1,1′-binaphthalene]-2,2′-diyl)bis(oxy-4,1-phenylene)]dimethanol (D2NBNAD4PODMO) (compound of formula (Ia), with X=—CH₂OH, A=1,4-phenylene and R=2-naphthyl; compound 148 of table A)

### Step 1: 4,4′-[(6,6′-bis(naphthalen-2-yl)[1,1′-binaphthalene]-2,2′-diyl)bis(oxy)]dibenzaldehyde (D2NBNAD4PODA)

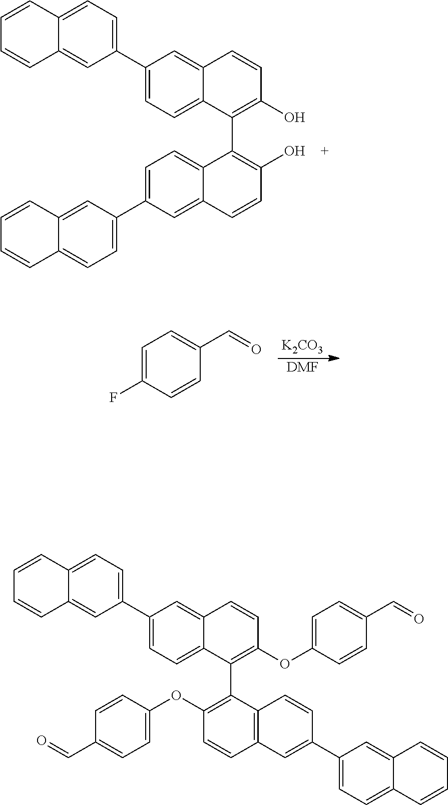

A mixture of D2NBNA (108.81 g, 200 mmol, 1 eq., obtained in Example A) and K₂CO₃ (69.1 g, 500 mmol, 2.5 eq.) in N,N-dimethylformamide (200 g) was stirred at 60° C. for 1 h. To this mixture was added 4-fluorobenzaldehyde (62.1 g, 500 mmol, 2.5 eq.) and it was stirred under reflux for approx. 36 h until TLC control (cyclohexane/ethyl acetate 1:1) showed complete conversion. The mixture was cooled to room temperature and was then slowly added to a mixture of 80 mL of concentrated aqueous HCl (37%) and 1.6 L of water. The resulting suspension was stirred for 1 h. The solid product was collected by filtration. The filter cake was washed with water (3×150 mL) and with isopropanol (2×100 mL) and once with n-pentane (100 mL) and dried at 60° C. to yield 150 g of the crude title compound D2NBNAD4PODA. The material was dissolved in THF (450 g). Then, toluene (600 g) was added and the mixture was concentrated under reduced pressure to a total mass of 700 g. The resulting suspension was stirred at room temperature for 1 h. The solid product was collected by filtration. The filter cake was washed with toluene (2×100 mL) and with n-pentane (100 mL) and dried at 60° C. to yield 95.8 g of the title compound D2NBNAD4PODA with a chemical purity (UPLC) of >98%.

¹H NMR (80 MHz, DMSO-d₆) J=9.80 (s, 2H, CHO), 8.60-6.85 (m, 32H) ppm.

### Step 2: Synthesis of [(6,6′-bis(naphthalen-2-yl)[1,1′-binaphthalene]-2,2′-diyl)bis(oxy-4,1-phenylene)]dimethanol (D2NBNAD4PODMO) via reduction of D2NBNAD4PODA

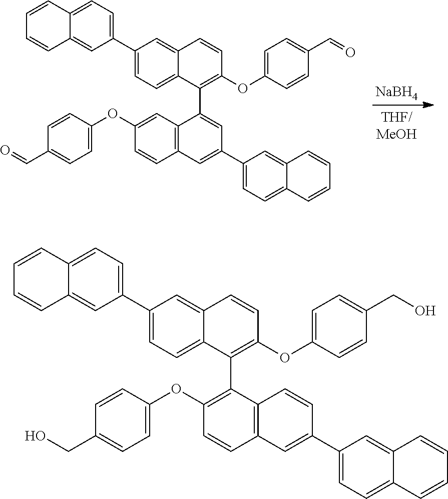 [SMILES: O=Cc1ccc(Oc2ccc3cc(-c4ccc5ccccc5c4)cc(-c4c(Oc5ccc(C=O)cc5)ccc5cc(-c6ccc7ccccc7c6)ccc45)c3c2)cc1 ; "NaBH4" ; OCc1ccc(Oc2ccc3cc(-c4ccc5ccccc5c4)ccc3c2-c2c(Oc3ccc(CO)cc3)ccc3cc(-c4ccc5ccccc5c4)ccc23)cc1]

To a mixture of D2NBNAD4PODA (95.8 g, 125 mmol, 1.00 eq., obtained in step 1) and THF (640 g) was added portionwise NaBH₄ (10 g, 264 mmol, 2.1 eq.) and 8.5 g of methanol. The reaction mixture was stirred under reflux for 2 h until TLC control (TBME) showed complete conversion. The reaction mixture was cooled to 0° C. (ice bath) and an aqueous solution of HCl (10% by weight) was carefully added (approx. 100 mL) to adjust the pH to <3. To this mixture was added 2-Me-THF (250 mL) and water (100 mL). The mixture was concentrated under reduced pressure to remove THF. The phases were separated at 60° C. and the organic phase was washed with an aqueous solution of NaOH (1 M; 200 mL), with an aqueous solution of HCl (10% by weight, 200 mL) and with brine (200 mL). The organic phase was dried over Na₂SO₄ and treated with 5 g of activated charcoal (Norit DX Ultra). The charcoal and Na₂SO₄ were removed by filtration over celite and the filtrate was concentrated under reduced pressure. Then, toluene (500 g) was added and the suspension was stirred at room temperature for 1 h. The solid was collected by filtration, washed with toluene (3×30 g) and n-pentane (200 mL) and dried at 60° C. to yield 63.5 g of the title compound D2NBNAD4PODMO with a chemical purity of 98.9%.

¹H NMR (80 MHz, DMSO-d₆) J=8.51 (s, 2H) 8.4-7.8 (m, 14H), 7.7-7.1 (m, 12H), 6.86 (d, J=8.5 Hz, 4H), 5.19 (t, J=5.6 Hz, 2H, OH), 4.83 (d, J=5.6 Hz, 4H, CH₂) ppm.

### Example 4: [(6,6′-Bis(naphthalen-2-yl)[1,1′-binaphthalene]-2,2′-diyl)bis(oxynaphthalene-4,1-diyl)]dimethanol (D2NBNAD4NODMO); (compound of formula (la), with X=—CH₂OH, A=1,4-naphthylene and R=2-naphthyl; compound 150 of table A)

### Step 1: Synthesis of 4,4′-[(6,6′-bis(naphthalen-2-yl)[1,1′-binaphthalene]-2,2′-diyl)bis(oxy)]di(naphthalene-1-carbaldehyde) (D2NBNAD4NODA)

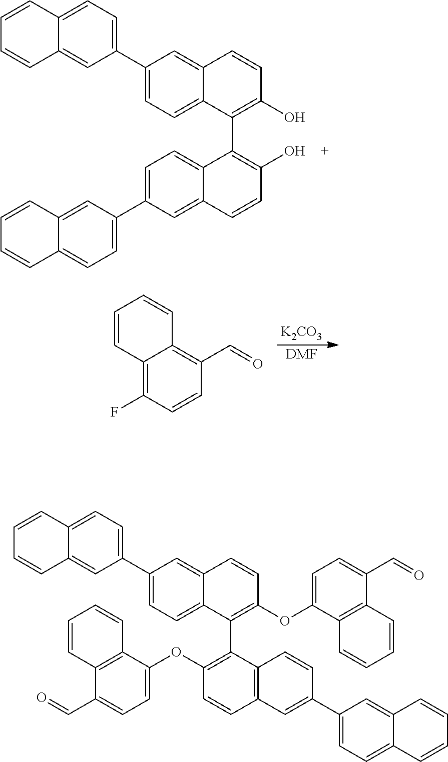

A mixture of D2NBNA (98 g, 180 mmol, 1 eq., obtained in Example A) and K₂CO₃ (62.2 g, 450 mmol, 2.5 eq.) in dimethylformamide (280 mL) was stirred at 60° C. for 1 h. To this mixture was added 4-fluoronaphthylaldehyd (80 g, 450 mmol, 2.5 eq.) at 50° C. and it was stirred under reflux for approx. 4 h until TLC control (cyclohexane/ethyl acetate 3:1) showed complete conversion. The mixture was cooled to 70° C. Then, the mixture was slowly added to a mixture of 100 mL of concentrated aqueous HCl (37%) and 2000 mL of water. The resulting suspension was stirred for 3 h. The solid product was collected by filtration. The filter cake was washed with water (3×250 mL), with ethanol (2×200 mL) and with pentane (200 mL) and dried at 60° C. to yield 151.7 g of the crude title compound D2NBNAD4NODA. The crude material was recrystallized from THF (750 g) to yield 95.5 g of the title compound D2NBNAD4NODA, which was used in the following step 2.

### Step 2: Synthesis of [(6,6′-bis(naphthalen-2-yl) [1,1′-binaphthalene]-2,2′-diyl)bis(oxynaphthalene-4,1-diyl)]dimethanol (D2NBNAD4NODMO) via reduction of D2NBNAD4NODA

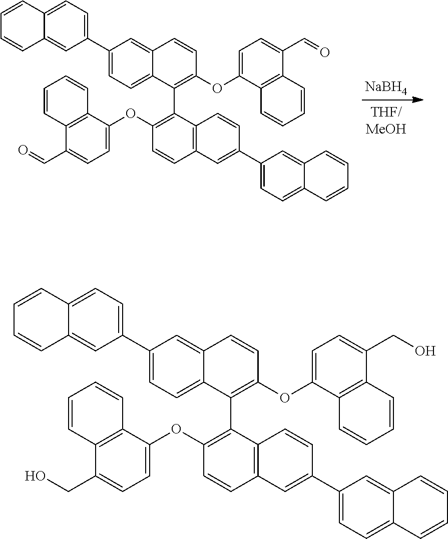 [SMILES: O=Cc1ccc(Oc2ccc3cc(-c4ccc5ccccc5c4)ccc3c2-c2c(Oc3ccc(C=O)c4ccccc34)ccc3cc(-c4ccc5ccccc5c4)ccc23)c2ccccc12 ; "NaBH4" ; OCc1ccc(Oc2ccc3cc(-c4ccc5ccccc5c4)ccc3c2-c2c(Oc3ccc(CO)c4ccccc34)ccc3cc(-c4ccc5ccccc5c4)ccc23)c2ccccc12]

To a mixture of D2NBNAD4NODA (95 g, 109.9 mmol, 1.00 eq.; obtained in step 1) in 2-MeTHF (600 g) was added portionwise NaBH₄ (8.7 g, 231 mmol, 2.1 eq.). To the reaction mixture methanol (7 g) was slowly added and the mixture was stirred under reflux for 3 h. An additional portion of NaBH₄ (2 g) was added and the mixture was stirred for further 2 h under reflux until TLC control (cyclohexane/ethyl acetate 1:1) showed complete conversion. The reaction mixture was cooled to 0° C. (ice bath) and an aqueous solution of HCl (10% by weight) was carefully added (approx. 100 mL) to adjust the pH to <3. To this mixture was added 2-MeTHF (500 mL) and water (200 mL). The phases were separated and the organic phase was concentrated under reduced pressure. The residue was taken up in 2-MeTHF (800 mL) and water (200 mL). The organic phase was washed with an aqueous solution of NaOH (1 M; 200 mL), with an aqueous solution of HCl (10% by weight, 200 mL) and with brine (200 mL). The organic phase was dried over Na₂SO₄. Afterwards, Na₂SO₄ was removed by filtration over celite and the filtrate was concentrated under reduced pressure. Then, toluene (300 g) was added and the suspension was stirred at room temperature for 1 h. The solid was collected by filtration, washed with toluene (3×30 g) and n-pentane (200 mL) and dried at 60° C. to yield the title compound as an off-white solid (73.7 g). This material was recrystallized from hot THE (1.7 L) and dried at 60° C. to yield the title compound D2NBNAD4NODMO as a white solid (41 g) with a chemical purity of 99%.

¹H NMR (80 MHz, DMSO-d₆) J=8.33 (d, J=10.4 Hz, 4H), 8.2-7.7 (m, 16H), 7.7-7.0 (m, 14H), 6.85 (d, J=7.2 Hz, 2H), 5.19 (t, J=5.2 Hz, 2H, OH), 4.82 (d, J=5.2 Hz, 4H, CH₂) ppm;
m.p. [DSC]=232° C.

### Example 5: 4,4′-[[1,1′-binaphthalene]-2,2′-diylbis(oxy)]dibenzoic acid (BNAD4PODCA) (compound of formula (Ia), with X=—COOH, A=1,4-phenylene and R=H; compound 34 of table A)

### Step 1: Synthesis of 4,4′-[[1,1′-binaphthalene]-2,2′-diylbis(oxy)]dibenzonitrile (BNAD4PODCN)

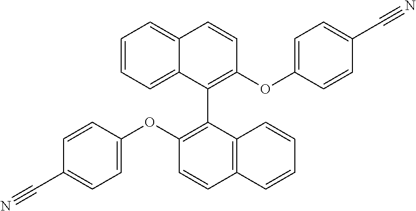 [SMILES: N#Cc1ccc(Oc2ccc3ccccc3c2-c2c(Oc3ccc(C#N)cc3)ccc3ccccc23)cc1]

### 4,4′-[[1,1′-binaphthalene]-2,2′-diylbis(oxy)]dibenzonitrile

A mixture of rac-1,1′-bi-2-naphthol (101.23 g, 350 mmol, 1 eq) and K₂CO₃ (120.93 g, 1.25 mol, 2.5 eq) in N,N-dimethylformamide (350 mL) was stirred at 60° C. for 1 h. To this mixture was added 4-fluorobenzonitrile (105.97 g, 1.25 mol, 2.5 eq) and it was stirred under reflux for approx. 2 h until TLC control (methanol/H₂O=3:1) showed complete conversion. The mixture was cooled to room temperature. Then, the mixture was slowly added to a mixture of 20 mL of concentrated aqueous HCl (37%) and 400 mL of water. The resulting suspension was stirred for 1 h. The solid product was collected by filtration. The filter cake was washed 3 times with water (3×250 mL) and once with isopropanol (250 mL) and dried at 60° C. to yield 170 g of the title compound (BNAD4PODCN). The crude product was recrystallized from a THE/toluene mixture to give pure BNAD4PODCN (120.6 g) with chemical purity of 97.8%.

¹H NMR (80 MHz, CDCl₃)=δ 8.03 (dd, J=8.1, 4.2 Hz, 4H), 7.78-7.17 (m, 12H), 6.88 (d, J=6.78 Hz, 4H) ppm.
m.p. [DSC]=215.3° C.

### Step 2: Synthesis of 4,4′-[[1,1′-binaphthalene]-2,2′-diylbis(oxy)]dibenzoic acid (BNAD4PODCA)

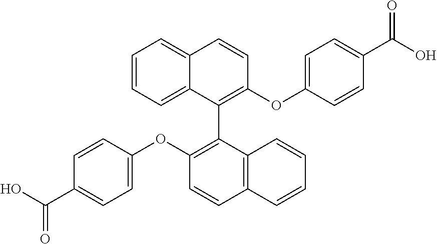 [SMILES: O=C(O)c1ccc(Oc2ccc3ccccc3c2-c2c(Oc3ccc(C(=O)O)cc3)ccc3ccccc23)cc1]

### 4,4′-[[1,1′-binaphthalene]-2,2′-diylbis(oxy)]dibenzoic acid

A mixture of BNAD4PODCN (10.0 g, 20.47 mmol; obtained in step 1) in 38 g of ethanol and a solution of KOH (34.5 g) in water (75 mL) was stirred at reflux for 24 h until TLC control (methanol/H₂O=3:1, acidified with AcOH) showed complete conversion. The reaction mixture was cooled to room temperature and a part of ethanol was distilled off. Then, the mixture was quenched with concentrated aqueous HCl (37%) until pH=1 is reached. The resulting suspension was stirred for 30 min. The solid product was collected by filtration. The filter cake was washed 3 times with water (3×25 mL) and once with isopropanol (25 mL) and dried at 60° C. to yield 10.75 g (99.74%) of the title compound (BNAD4PODCA) with chemical purity of 94.4%.

¹H NMR (80 MHz, DMSO-de)=δ 7.99 (t, J=7.4 Hz, 4H), 7.69 (d, J=7.69 Hz, 4H), 7.50-7.04 (m, 8H), 6.78 (d, J=6.77 Hz, 4) ppm.
m.p. [DSC]=314.2° C.

### Example 6: 4,4′-[[1,1′-binaphthalene]-2,2′-diylbis(oxy)]dibenzoate (BNAD4PODCAMe) (compound of formula (Ia), with X=—COOMe, A=1,4-phenylene and R=H; compound 67 of table A)

### Example 6a: Synthesis of BNAD4PODCAMe Via Oxidation of BNAD4PODA (See Example 1, Step 1) in Methanol

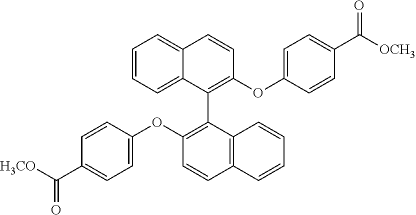 [SMILES: COC(=O)c1ccc(Oc2ccc3ccccc3c2-c2c(Oc3ccc(C(=O)OC)cc3)ccc3ccccc23)cc1]

### dimethyl 4,4′-[[1,1′-binaphthalene]-2,2′-diylbis(oxy)]dibenzoate

A suspension of BNAD4PODA (68.6 g, 137.33 mmol; synthesized according to step 1 of example 1) and Oxone® (83.6 g) in 690 g methanol was stirred at 60° C. for 60 h until TLC control (cyclohexane/ethyl acetate=3:1) showed complete conversion. The reaction mixture was cooled to room temperature and the solid was filtered off and washed 3 times with methanol (3×50 mL). The solid was suspended in 500 mL of water and the suspension was stirred at room temperature for 1 h. Finally, the crude product BNAD4PODCAMe was collected by filtration. The filter cake was washed 3 times with water (3×50 mL) and once with isopropanol (25 mL) and dried at 60° C. to yield 11.93 g (16%) of the title compound BNAD4PODCAMe with a chemical purity of 96.2%.

¹H NMR (80 MHz, CDCl₃)=δ 8.18-7.54 (m, 8H), 7.50-7.15 (m, 8H, 6.79 (d, J=6.76 Hz, 4H), 3.93 (s, 6H, OCH₃) ppm.
m.p. [DSC]=225.4° C.

### Example 6b: Synthesis of BNAD4PODCAMe by Esterification of BNAD4PODCA (See Example 5)

A suspension of BNAD4PODCA (5.0 g, 9.3 mmol; synthesized according to example 5) in 35 mL of thionyl chloride (SOCl₂) was stirred at reflux for 4 h until the solid was completely dissolved. The excess of thionyl chloride was completely removed under reduced pressure and the obtained solid was dried in vacuum at 60° C. for 1 h. To the dried solid 40 mL of methanol was slowly added, and the obtained suspension was stirred at reflux for 6 h. The suspension was cooled to room temperature and the crude product was collected by filtration. The filter cake was washed 3 times with methanol (3×10 mL) and dried in vacuum at 60° C. to yield 4.83 g (93.9%) of a title compound BNAD4PODCAMe.

¹H NMR (80 MHz, CDCl₃)=δ 8.18-7.54 (m, 8H), 7.50-7.15 (m, 8H), 6.79 (d, J=6.76 Hz, 4H), 3.93 (s, 6H, OCH₃) ppm.

### 2.4 Refractive Indices No of Monomers of Formula (I)

The following table C lists refractive indices of some monomers of formula (I) that were calculated using the software ACD/ChemSketch 2012 (Advanced Chemistry Development, Inc.). The individual monomers are identified in table C by their entry numbers in tables A and B, respectively. In addition, it has been verified by quantum chemical calculations for all monomers included in table C that they do not, or only to a negligible extent, absorb in the visible light range and are therefore basically colorless.

TABLE C

| Entry # | from Table | n_{D} (calc.) monomer |
|---|---|---|
| 1 | A | 1.71 |
| 2 | A | 1.71 |
| 3 | A | 1.71 |
| 4 | A | 1.75 |
| 5 | A | 1.75 |
| 6 | A | 1.75 |
| 7 | A | 1.70 |
| 8 | A | 1.70 |
| 9 | A | 1.70 |
| 10 | A | 1.75 |
| 11 | A | 1.75 |
| 12 | A | 1.73 |
| 13 | A | 1.82 |
| 14 | A | 1.82 |
| 15 | A | 1.82 |
| 16 | A | 1.82 |
| 17 | A | 1.82 |
| 18 | A | 1.82 |
| 19 | A | 1.79 |
| 20 | A | 1.79 |
| 21 | A | 1.79 |
| 22 | A | 1.79 |
| 23 | A | 1.79 |
| 24 | A | 1.79 |
| 25 | A | 1.79 |
| 26 | A | 1.79 |
| 27 | A | 1.79 |
| 28 | A | 1.79 |
| 29 | A | 1.79 |
| 30 | A | 1.79 |
| 31 | A | 1.79 |
| 32 | A | 1.79 |
| 33 | A | 1.79 |
| 34 | A | 1.72 |
| 35 | A | 1.72 |
| 36 | A | 1.72 |
| 37 | A | 1.76 |
| 38 | A | 1.76 |
| 39 | A | 1.76 |
| 40 | A | 1.71 |
| 41 | A | 1.71 |
| 42 | A | 1.71 |
| 43 | A | 1.76 |
| 44 | A | 1.76 |
| 45 | A | 1.75 |
| 46 | A | 1.83 |
| 47 | A | 1.83 |
| 48 | A | 1.83 |
| 49 | A | 1.83 |
| 50 | A | 1.83 |
| 51 | A | 1.83 |
| 52 | A | 1.80 |
| 53 | A | 1.80 |
| 54 | A | 1.80 |
| 55 | A | 1.80 |
| 56 | A | 1.80 |
| 57 | A | 1.80 |
| 58 | A | 1.80 |
| 59 | A | 1.80 |
| 60 | A | 1.80 |
| 61 | A | 1.80 |
| 62 | A | 1.80 |
| 63 | A | 1.80 |
| 64 | A | 1.80 |
| 65 | A | 1.80 |
| 66 | A | 1.80 |
| 67 | A | 1.66 |
| 68 | A | 1.66 |
| 69 | A | 1.66 |
| 70 | A | 1.71 |
| 71 | A | 1.71 |
| 72 | A | 1.71 |
| 73 | A | 1.67 |
| 74 | A | 1.67 |
| 75 | A | 1.67 |
| 76 | A | 1.71 |
| 77 | A | 1.71 |
| 78 | A | 1.70 |
| 79 | A | 1.77 |
| 80 | A | 1.77 |
| 81 | A | 1.77 |
| 82 | A | 1.77 |
| 83 | A | 1.77 |
| 84 | A | 1.77 |
| 85 | A | 1.74 |
| 86 | A | 1.74 |
| 87 | A | 1.74 |
| 88 | A | 1.74 |
| 89 | A | 1.74 |
| 90 | A | 1.74 |
| 91 | A | 1.75 |
| 92 | A | 1.75 |
| 93 | A | 1.75 |
| 94 | A | 1.75 |
| 95 | A | 1.75 |
| 96 | A | 1.75 |
| 97 | A | 1.75 |
| 98 | A | 1.75 |
| 99 | A | 1.75 |
| 100 | A | 1.70 |
| 101 | A | 1.70 |
| 102 | A | 1.74 |
| 103 | A | 1.74 |
| 104 | A | 1.74 |
| 105 | A | 1.70 |
| 106 | A | 1.70 |
| 107 | A | 1.73 |
| 108 | A | 1.71 |
| 109 | A | 1.71 |
| 110 | A | 1.74 |
| 111 | A | 1.74 |
| 112 | A | 1.74 |
| 113 | A | 1.70 |
| 114 | A | 1.70 |
| 115 | A | 1.74 |
| 116 | A | 1.67 |
| 117 | A | 1.67 |
| 118 | A | 1.70 |
| 119 | A | 1.70 |
| 120 | A | 1.70 |
| 121 | A | 1.67 |
| 122 | A | 1.67 |
| 123 | A | 1.70 |
| 124 | A | 1.74 |
| 125 | A | 1.74 |
| 126 | A | 1.77 |
| 127 | A | 1.77 |
| 128 | A | 1.77 |
| 129 | A | 1.73 |
| 130 | A | 1.73 |
| 131 | A | 1.76 |
| 132 | A | 1.74 |
| 133 | A | 1.74 |
| 134 | A | 1.77 |
| 135 | A | 1.77 |
| 136 | A | 1.77 |
| 137 | A | 1.73 |
| 138 | A | 1.73 |
| 139 | A | 1.77 |
| 140 | A | 1.70 |
| 141 | A | 1.70 |
| 142 | A | 1.73 |
| 143 | A | 1.73 |
| 144 | A | 1.73 |
| 145 | A | 1.70 |
| 146 | A | 1.73 |
| 147 | A | 1.73 |
| 148 | A | 1.74 |
| 149 | A | 1.74 |
| 150 | A | 1.77 |
| 151 | A | 1.77 |
| 152 | A | 1.77 |
| 153 | A | 1.73 |
| 154 | A | 1.73 |
| 155 | A | 1.76 |
| 156 | A | 1.74 |
| 157 | A | 1.74 |
| 158 | A | 1.77 |
| 159 | A | 1.77 |
| 160 | A | 1.77 |
| 161 | A | 1.73 |
| 162 | A | 1.73 |
| 163 | A | 1.77 |
| 164 | A | 1.70 |
| 165 | A | 1.70 |
| 166 | A | 1.73 |
| 167 | A | 1.73 |
| 168 | A | 1.73 |
| 169 | A | 1.70 |
| 170 | A | 1.70 |
| 171 | A | 1.73 |
| 172 | A | 1.77 |
| 173 | A | 1.77 |
| 174 | A | 1.79 |
| 175 | A | 1.79 |
| 176 | A | 1.79 |
| 177 | A | 1.75 |
| 178 | A | 1.75 |
| 179 | A | 1.79 |
| 180 | A | 1.77 |
| 181 | A | 1.77 |
| 182 | A | 1.80 |
| 183 | A | 1.80 |
| 184 | A | 1.80 |
| 185 | A | 1.76 |
| 186 | A | 1.76 |
| 187 | A | 1.79 |
| 188 | A | 1.73 |
| 189 | A | 1.73 |
| 190 | A | 1.76 |
| 191 | A | 1.76 |
| 192 | A | 1.76 |
| 193 | A | 1.73 |
| 194 | A | 1.73 |
| 195 | A | 1.73 |
| 196 | A | 1.79 |
| 197 | A | 1.79 |
| 198 | A | 1.81 |
| 199 | A | 1.81 |
| 200 | A | 1.81 |
| 201 | A | 1.77 |
| 202 | A | 1.77 |
| 203 | A | 1.81 |
| 204 | A | 1.80 |
| 205 | A | 1.80 |
| 206 | A | 1.82 |
| 207 | A | 1.82 |
| 208 | A | 1.82 |
| 209 | A | 1.78 |
| 210 | A | 1.78 |
| 211 | A | 1.81 |
| 212 | A | 1.75 |
| 213 | A | 1.75 |
| 214 | A | 1.78 |
| 215 | A | 1.78 |
| 216 | A | 1.78 |
| 217 | A | 1.74 |
| 218 | A | 1.74 |
| 219 | A | 1.77 |
| 220 | A | 1.77 |
| 221 | A | 1.77 |
| 222 | A | 1.80 |
| 223 | A | 1.80 |
| 224 | A | 1.80 |
| 225 | A | 1.75 |
| 226 | A | 1.75 |
| 227 | A | 1.79 |
| 228 | A | 1.78 |
| 229 | A | 1.78 |
| 230 | A | 1.80 |
| 231 | A | 1.80 |
| 232 | A | 1.80 |
| 233 | A | 1.76 |
| 234 | A | 1.76 |
| 235 | A | 1.80 |
| 236 | A | 1.74 |
| 237 | A | 1.74 |
| 238 | A | 1.76 |
| 239 | A | 1.76 |
| 240 | A | 1.76 |
| 241 | A | 1.73 |
| 242 | A | 1.73 |
| 243 | A | 1.76 |
| 1 | B | 1.70 |
| 2 | B | 1.71 |
| 3 | B | 1.67 |
| 4 | B | 1.74 |
| 5 | B | 1.74 |
| 6 | B | 1.70 |
| 7 | B | 1.74 |
| 8 | B | 1.74 |
| 9 | B | 1.70 |
| 10 | B | 1.77 |
| 11 | B | 1.77 |
| 12 | B | 1.73 |
| 13 | B | 1.79 |
| 14 | B | 1.80 |
| 15 | B | 1.75 |
| 16 | B | 1.77 |
| 17 | B | 1.78 |
| 18 | B | 1.74 |
| 19 | B | 1.70 |
| 20 | B | 1.71 |
| 21 | B | 1.67 |
| 22 | B | 1.74 |
| 23 | B | 1.74 |
| 24 | B | 1.70 |
| 25 | B | 1.74 |
| 26 | B | 1.74 |
| 27 | B | 1.70 |
| 28 | B | 1.77 |
| 29 | B | 1.77 |
| 30 | B | 1.73 |
| 31 | B | 1.79 |
| 32 | B | 1.80 |
| 33 | B | 1.75 |
| 34 | B | 1.77 |
| 35 | B | 1.78 |
| 36 | B | 1.74 |
| 37 | B | 1.74 |
| 38 | B | 1.74 |
| 39 | B | 1.70 |
| 40 | B | 1.77 |
| 41 | B | 1.77 |
| 42 | B | 1.73 |
| 43 | B | 1.77 |
| 44 | B | 1.77 |
| 45 | B | 1.73 |
| 46 | B | 1.79 |
| 47 | B | 1.80 |
| 48 | B | 1.76 |
| 49 | B | 1.81 |
| 50 | B | 1.82 |
| 51 | B | 1.78 |
| 52 | B | 1.80 |
| 53 | B | 1.80 |
| 54 | B | 1.76 |
| 55 | B | 1.74 |
| 56 | B | 1.74 |
| 57 | B | 1.70 |
| 58 | B | 1.77 |
| 59 | B | 1.77 |
| 60 | B | 1.73 |
| 61 | B | 1.77 |
| 62 | B | 1.77 |
| 63 | B | 1.73 |
| 64 | B | 1.79 |
| 65 | B | 1.80 |
| 66 | B | 1.76 |
| 67 | B | 1.81 |
| 68 | B | 1.82 |
| 69 | B | 1.78 |
| 70 | B | 1.80 |
| 71 | B | 1.80 |
| 72 | B | 1.76 |
| 73 | B | 1.69 |
| 74 | B | 1.70 |
| 75 | B | 1.67 |
| 76 | B | 1.73 |
| 77 | B | 1.73 |
| 78 | B | 1.70 |
| 79 | B | 1.73 |
| 80 | B | 1.73 |
| 81 | B | 1.70 |
| 82 | B | 1.75 |
| 83 | B | 1.76 |
| 84 | B | 1.73 |
| 85 | B | 1.77 |
| 86 | B | 1.78 |
| 87 | B | 1.74 |
| 88 | B | 1.75 |
| 89 | B | 1.76 |
| 90 | B | 1.73 |
| 91 | B | 1.73 |
| 92 | B | 1.73 |
| 93 | B | 1.70 |
| 94 | B | 1.75 |
| 95 | B | 1.76 |
| 96 | B | 1.73 |
| 97 | B | 1.75 |
| 98 | B | 1.76 |
| 99 | B | 1.73 |
| 100 | B | 1.77 |
| 101 | B | 1.78 |
| 102 | B | 1.75 |
| 103 | B | 1.79 |
| 104 | B | 1.80 |
| 105 | B | 1.76 |
| 106 | B | 1.78 |
| 107 | B | 1.78 |
| 108 | B | 1.75 |
| 109 | B | 1.73 |
| 110 | B | 1.73 |
| 111 | B | 1.70 |
| 112 | B | 1.75 |
| 113 | B | 1.76 |
| 114 | B | 1.73 |
| 115 | B | 1.75 |
| 116 | B | 1.76 |
| 117 | B | 1.73 |
| 118 | B | 1.77 |
| 119 | B | 1.78 |
| 120 | B | 1.75 |
| 121 | B | 1.79 |
| 122 | B | 1.80 |
| 123 | B | 1.76 |
| 124 | B | 1.78 |
| 125 | B | 1.78 |
| 126 | B | 1.75 |
| 127 | B | 1.75 |
| 128 | B | 1.76 |
| 129 | B | 1.73 |
| 130 | B | 1.77 |
| 131 | B | 1.78 |
| 132 | B | 1.75 |
| 133 | B | 1.77 |
| 134 | B | 1.78 |
| 135 | B | 1.75 |
| 136 | B | 1.79 |
| 137 | B | 1.80 |
| 138 | B | 1.77 |
| 139 | B | 1.81 |
| 140 | B | 1.82 |
| 141 | B | 1.78 |
| 142 | B | 1.80 |
| 143 | B | 1.80 |
| 144 | B | 1.77 |
| 145 | B | 1.77 |
| 146 | B | 1.78 |
| 147 | B | 1.74 |
| 148 | B | 1.79 |
| 149 | B | 1.80 |
| 150 | B | 1.76 |
| 151 | B | 1.79 |
| 152 | B | 1.80 |
| 153 | B | 1.76 |
| 154 | B | 1.81 |
| 155 | B | 1.82 |
| 156 | B | 1.78 |
| 157 | B | 1.83 |
| 158 | B | 1.83 |
| 159 | B | 1.80 |
| 160 | B | 1.81 |
| 161 | B | 1.82 |
| 162 | B | 1.79 |
| 163 | B | 1.75 |
| 164 | B | 1.76 |
| 165 | B | 1.73 |
| 166 | B | 1.78 |
| 167 | B | 1.78 |
| 168 | B | 1.75 |
| 169 | B | 1.78 |
| 170 | B | 1.78 |
| 171 | B | 1.75 |
| 172 | B | 1.80 |
| 173 | B | 1.80 |
| 174 | B | 1.77 |
| 175 | B | 1.81 |
| 176 | B | 1.82 |
| 177 | B | 1.79 |
| 178 | B | 1.80 |
| 179 | B | 1.80 |
| 180 | B | 1.77 |
| 181 | B | 1.69 |
| 182 | B | 1.70 |
| 183 | B | 1.67 |
| 184 | B | 1.73 |
| 185 | B | 1.73 |
| 186 | B | 1.70 |
| 187 | B | 1.73 |
| 188 | B | 1.73 |
| 189 | B | 1.70 |
| 190 | B | 1.75 |
| 191 | B | 1.76 |
| 192 | B | 1.73 |
| 193 | B | 1.77 |
| 194 | B | 1.78 |
| 195 | B | 1.74 |
| 196 | B | 1.75 |
| 197 | B | 1.76 |
| 198 | B | 1.73 |
| 199 | B | 1.73 |
| 200 | B | 1.73 |
| 201 | B | 1.70 |
| 202 | B | 1.75 |
| 203 | B | 1.76 |
| 204 | B | 1.73 |
| 205 | B | 1.75 |
| 206 | B | 1.76 |
| 207 | B | 1.73 |
| 208 | B | 1.77 |
| 209 | B | 1.78 |
| 210 | B | 1.75 |
| 211 | B | 1.79 |
| 212 | B | 1.80 |
| 213 | B | 1.76 |
| 214 | B | 1.78 |
| 215 | B | 1.78 |
| 216 | B | 1.75 |
| 217 | B | 1.73 |
| 218 | B | 1.73 |
| 219 | B | 1.70 |
| 220 | B | 1.75 |
| 221 | B | 1.76 |
| 222 | B | 1.73 |
| 223 | B | 1.75 |
| 224 | B | 1.76 |
| 225 | B | 1.73 |
| 226 | B | 1.77 |
| 227 | B | 1.78 |
| 228 | B | 1.75 |
| 229 | B | 1.79 |
| 230 | B | 1.80 |
| 231 | B | 1.76 |
| 232 | B | 1.78 |
| 233 | B | 1.78 |
| 234 | B | 1.75 |
| 235 | B | 1.75 |
| 236 | B | 1.76 |
| 237 | B | 1.73 |
| 238 | B | 1.77 |
| 239 | B | 1.78 |
| 240 | B | 1.75 |
| 241 | B | 1.77 |
| 242 | B | 1.78 |
| 243 | B | 1.75 |
| 244 | B | 1.79 |
| 245 | B | 1.80 |
| 246 | B | 1.77 |
| 247 | B | 1.81 |
| 248 | B | 1.82 |
| 249 | B | 1.78 |
| 250 | B | 1.80 |
| 251 | B | 1.80 |
| 252 | B | 1.77 |
| 253 | B | 1.77 |
| 254 | B | 1.78 |
| 255 | B | 1.74 |
| 256 | B | 1.79 |
| 257 | B | 1.80 |
| 258 | B | 1.76 |
| 259 | B | 1.79 |
| 260 | B | 1.80 |
| 261 | B | 1.76 |
| 262 | B | 1.81 |
| 263 | B | 1.82 |
| 264 | B | 1.78 |
| 265 | B | 1.83 |
| 266 | B | 1.83 |
| 267 | B | 1.80 |
| 268 | B | 1.81 |
| 269 | B | 1.82 |
| 270 | B | 1.79 |
| 271 | B | 1.75 |
| 272 | B | 1.76 |
| 273 | B | 1.73 |
| 274 | B | 1.78 |
| 275 | B | 1.78 |
| 276 | B | 1.75 |
| 277 | B | 1.78 |
| 278 | B | 1.78 |
| 279 | B | 1.75 |
| 280 | B | 1.80 |
| 281 | B | 1.80 |
| 282 | B | 1.77 |
| 283 | B | 1.81 |
| 284 | B | 1.82 |
| 285 | B | 1.79 |
| 286 | B | 1.80 |
| 287 | B | 1.80 |
| 288 | B | 1.77 |
| 289 | B | 1.73 |
| 290 | B | 1.73 |
| 291 | B | 1.70 |
| 292 | B | 1.75 |
| 293 | B | 1.76 |
| 294 | B | 1.73 |
| 295 | B | 1.75 |
| 296 | B | 1.76 |
| 297 | B | 1.73 |
| 298 | B | 1.77 |
| 299 | B | 1.78 |
| 300 | B | 1.75 |
| 301 | B | 1.79 |
| 302 | B | 1.80 |
| 303 | B | 1.76 |
| 304 | B | 1.78 |
| 305 | B | 1.78 |
| 306 | B | 1.75 |
| 307 | B | 1.75 |
| 308 | B | 1.76 |
| 309 | B | 1.73 |
| 310 | B | 1.77 |
| 311 | B | 1.78 |
| 312 | B | 1.75 |
| 313 | B | 1.77 |
| 314 | B | 1.78 |
| 315 | B | 1.75 |
| 316 | B | 1.79 |
| 317 | B | 1.80 |
| 318 | B | 1.77 |
| 319 | B | 1.81 |
| 320 | B | 1.82 |
| 321 | B | 1.78 |
| 322 | B | 1.80 |
| 323 | B | 1.80 |
| 324 | B | 1.77 |
| 325 | B | 1.75 |
| 326 | B | 1.76 |
| 327 | B | 1.73 |
| 328 | B | 1.77 |
| 329 | B | 1.78 |
| 330 | B | 1.75 |
| 331 | B | 1.77 |
| 332 | B | 1.78 |
| 333 | B | 1.75 |
| 334 | B | 1.79 |
| 335 | B | 1.80 |
| 336 | B | 1.77 |
| 337 | B | 1.81 |
| 338 | B | 1.82 |
| 339 | B | 1.78 |
| 340 | B | 1.80 |
| 341 | B | 1.80 |
| 342 | B | 1.77 |
| 343 | B | 1.77 |
| 344 | B | 1.78 |
| 345 | B | 1.75 |
| 346 | B | 1.79 |
| 347 | B | 1.80 |
| 348 | B | 1.77 |
| 349 | B | 1.79 |
| 350 | B | 1.80 |
| 351 | B | 1.77 |
| 352 | B | 1.81 |
| 353 | B | 1.82 |
| 354 | B | 1.79 |
| 355 | B | 1.81 |
| 356 | B | 1.83 |
| 357 | B | 1.80 |
| 358 | B | 1.81 |
| 359 | B | 1.82 |
| 360 | B | 1.79 |
| 361 | B | 1.79 |
| 362 | B | 1.80 |
| 363 | B | 1.76 |
| 364 | B | 1.81 |
| 365 | B | 1.82 |
| 266 | B | 1.78 |
| 367 | B | 1.81 |
| 368 | B | 1.82 |
| 369 | B | 1.78 |
| 370 | B | 1.83 |
| 371 | B | 1.83 |
| 372 | B | 1.80 |
| 373 | B | 1.84 |
| 374 | B | 1.85 |
| 375 | B | 1.81 |
| 376 | B | 1.83 |
| 377 | B | 1.83 |
| 378 | B | 1.80 |
| 379 | B | 1.78 |
| 380 | B | 1.78 |
| 381 | B | 1.75 |
| 382 | B | 1.80 |
| 383 | B | 1.80 |
| 384 | B | 1.77 |
| 385 | B | 1.80 |
| 386 | B | 1.80 |
| 387 | B | 1.77 |
| 388 | B | 1.81 |
| 389 | B | 1.82 |
| 390 | B | 1.79 |
| 391 | B | 1.83 |
| 392 | B | 1.83 |
| 393 | B | 1.80 |
| 394 | B | 1.81 |
| 395 | B | 1.82 |
| 396 | B | 1.79 |
| 397 | B | 1.73 |
| 398 | B | 1.73 |
| 399 | B | 1.70 |
| 400 | B | 1.75 |
| 401 | B | 1.76 |
| 402 | B | 1.73 |
| 403 | B | 1.75 |
| 404 | B | 1.76 |
| 405 | B | 1.73 |
| 406 | B | 1.77 |
| 407 | B | 1.78 |
| 408 | B | 1.75 |
| 409 | B | 1.79 |
| 410 | B | 1.80 |
| 411 | B | 1.76 |
| 412 | B | 1.78 |
| 413 | B | 1.78 |
| 414 | B | 1.75 |
| 415 | B | 1.75 |
| 416 | B | 1.76 |
| 417 | B | 1.73 |
| 418 | B | 1.77 |
| 419 | B | 1.78 |
| 420 | B | 1.75 |
| 421 | B | 1.77 |
| 422 | B | 1.78 |
| 423 | B | 1.75 |
| 424 | B | 1.79 |
| 425 | B | 1.80 |
| 426 | B | 1.77 |
| 427 | B | 1.81 |
| 428 | B | 1.82 |
| 429 | B | 1.78 |
| 430 | B | 1.80 |
| 431 | B | 1.80 |
| 432 | B | 1.77 |
| 433 | B | 1.75 |
| 434 | B | 1.76 |
| 435 | B | 1.73 |
| 436 | B | 1.77 |
| 437 | B | 1.78 |
| 438 | B | 1.75 |
| 439 | B | 1.77 |
| 440 | B | 1.78 |
| 441 | B | 1.75 |
| 442 | B | 1.79 |
| 443 | B | 1.80 |
| 444 | B | 1.77 |
| 445 | B | 1.81 |
| 446 | B | 1.82 |
| 447 | B | 1.78 |
| 448 | B | 1.80 |
| 449 | B | 1.80 |
| 450 | B | 1.77 |
| 451 | B | 1.77 |
| 452 | B | 1.78 |
| 453 | B | 1.75 |
| 454 | B | 1.79 |
| 455 | B | 1.80 |
| 456 | B | 1.77 |
| 457 | B | 1.79 |
| 458 | B | 1.80 |
| 459 | B | 1.77 |
| 460 | B | 1.81 |
| 461 | B | 1.82 |
| 462 | B | 1.79 |
| 463 | B | 1.81 |
| 464 | B | 1.83 |
| 465 | B | 1.80 |
| 466 | B | 1.81 |
| 467 | B | 1.82 |
| 468 | B | 1.79 |
| 469 | B | 1.79 |
| 470 | B | 1.80 |
| 471 | B | 1.76 |
| 472 | B | 1.81 |
| 473 | B | 1.82 |
| 474 | B | 1.78 |
| 475 | B | 1.81 |
| 476 | B | 1.82 |
| 477 | B | 1.78 |
| 478 | B | 1.83 |
| 479 | B | 1.83 |
| 480 | B | 1.80 |
| 481 | B | 1.84 |
| 482 | B | 1.85 |
| 483 | B | 1.81 |
| 484 | B | 1.83 |
| 485 | B | 1.83 |
| 486 | B | 1.80 |
| 487 | B | 1.78 |
| 488 | B | 1.78 |
| 489 | B | 1.75 |
| 490 | B | 1.80 |
| 491 | B | 1.80 |
| 492 | B | 1.77 |
| 493 | B | 1.80 |
| 494 | B | 1.80 |
| 495 | B | 1.77 |
| 496 | B | 1.81 |
| 497 | B | 1.82 |
| 498 | B | 1.79 |
| 499 | B | 1.83 |
| 500 | B | 1.83 |
| 501 | B | 1.80 |
| 502 | B | 1.81 |
| 503 | B | 1.82 |
| 504 | B | 1.79 |

### 3. Preparation of Polycarbonate Resins from Monomers of Formula (I)

### 3.1 Analytics Relating to Resins Prepared from Monomers of Formula (I)

### Refractive Index (n_{D}):

Refractive indexes were measured using the test pieces obtained by the general procedure for preparing homopolycarbonates described in section 3.2 below. The measurements were conducted at a temperature of 23° C. and at a wavelength of 589 nm using the Rudolph Instruments J257 Automatic refractometer.

Abbe number (ν):

Abbe numbers were determined using samples with a thickness of approx. 3 mm, which were the same as those used in the method for measuring the refractive indexes described above. The refractive index values were measured using the Metricon 2010M Prism Coupler at a temperature of 23° C. and at wavelengths of 486 nm, 589 nm and 656 nm. The Abbe number was then calculated using the following formula:

$$v = ( {n}_{D} - 1 ) / ( {n}_{F} - {n}_{C} )$$

n_{D}: refractive index at a wavelength of 589 nm
n_{C}: refractive index at a wavelength of 656 nm
n_{F}: refractive index at a wavelength of 486 nm

### 3.2 Examples for the Preparation of Homopolycarbonates

### General Procedure

1.0 mmol of a monomer of formula (I), 214 mg (1.0 eq.) of diphenyl carbonate and 11 μl of a 0.01 mM aqueous solution of NaHCO₃ were mixed thoroughly and were afterwards dried for 30 minutes at 30° C. and 500 mbar. Half of this mixture was then transferred into a test tube (diameter: 10 mm, length: 80 mm) and heated in an oil bath at 180° C. to 200° C. for 3 hours under a gentle stream of argon. For mixing an overhead stirrer with a speed of about 35 rpm was used. Afterwards the heating was switched off and the formed polymer was allowed to cool slowly in the oil bath to room temperature. The test tube is cut off with the tube cutter just above the polymer surface, and the test piece lens obtained is freed by hitting the test tube section with a rubber mallet. The homopolycarbonates prepared by this procedure, together with the refractive indexes and Abbe numbers measured for them, are listed in table D below.

TABLE D

| Entry | from | Monomer | | |
|---|---|---|---|---|
| # | Table | of Example | n_{D} | ν |
| 1 | A | 1 | 1.705 | 18.7 |
| 4 | A | 2 | 1.732 | 15.9 |
| 148 | A | 3 | 1.774 | 16.5 |
| 150 | A | 4 | 1.798 | — |

We claim:

1. A use of a compound of the formula (I) 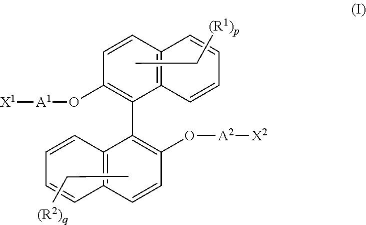
where
X¹ and X² are independently selected from —CH₂OH and —C(O)OR^{x}, where R^{X} is selected from the group consisting of hydrogen, phenyl, benzyl and C₁-C₄-alkyl;
A¹ and A² are independently selected from the group consisting of a mono- or polycyclic arylene having from 6 to 18 carbon atoms as ring members and a mono- or polycyclic hetarylene having a total of 5 to 18 atoms, which are ring members, where 1, 2, 3 or 4 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1, 2, 3 or 4 radicals R^{Ar1};
R¹ and R² are independently selected from the group consisting of halogen, C₂-C₃-alkynyl, CN, R, S(O)ₖR, NHR, OR, CH₅R′₃₋₅, NR₂, C(O)R and CH═CHR″, it being possible that R¹ and R² are identical or different if p+q>1, where s on each occurrence is 0, 1 or 2 and where k on each occurrence is 0, 1 or 2;
p and q are independently 0, 1 or 2;
R is selected from the group consisting of C₁-C₄-alkyl and a radical Q¹;
Q¹ is selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R^{Ar1} is selected from the group consisting of R¹¹, S(O)ₖR¹¹, NHR¹¹, OR¹¹, CN, halogen, CHₜR′₃-t, NR¹¹₂ and CH═CHR″, where R^{Ar1} may be identical or different if more than one is present on the same (het)aryl or (het)arylene group, where t on each occurrence is 0, 1 or 2 and where k on each occurrence is 0, 1 or 2;
R¹¹ is selected from the group consisting of C₁-C₄-alkyl and a radical Q²;
Q² is selected from the group consisting of mono- or polycyclic aryl having from 6 to 18 carbon atoms as ring members and mono- or polycyclic hetaryl having a total of 5 to 18 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R′ is selected from the group consisting of mono- or polycyclic aryl having from 6 to 18 carbon atoms as ring members and mono- or polycyclic hetaryl having a total of 5 to 18 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R″ is selected from hydrogen, methyl, phenyl and naphthyl, where phenyl and naphthyl are unsubstituted or substituted by 1, 2, 3 or 4 identical or different radicals R″;
R′″ is selected from the group consisting of phenyl, halogen, OCH₃, CH₃, N(CH₃)₂ and C(O)CH₃;
as a monomer for producing a thermoplastic resin, selected from polyesters, polycarbonates and polyestercarbonates.

2. A compound of the formula (I) 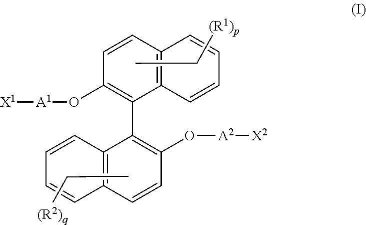
where
X¹ and X² are independently selected from —CH₂OH and —C(O)OR^{x}, where R^{x} is selected from the group consisting of hydrogen, phenyl, benzyl and C₁-C₄-alkyl;
A¹ and A² are independently selected from the group consisting of a mono- or polycyclic arylene having from 6 to 18 carbon atoms as ring members and a mono- or polycyclic hetarylene having a total of 5 to 18 atoms, which are ring members, where 1, 2, 3 or 4 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1, 2, 3 or 4 radicals R^{Ar1};
R¹ and R² are independently selected from the group consisting of halogen, C₂-C₃-alkynyl, CN, R, S(O)ₖR, NHR, OR, CH₅R′₃₋₅, NR₂, C(O)R and CH═CHR″, it being possible that R¹ and R² are identical or different if p+q>1, where s on each occurrence is 0, 1 or 2 and where k on each occurrence is 0, 1 or 2;
p and q are independently 0, 1 or 2;
R is selected from the group consisting of C₁-C₄-alkyl and a radical Q¹;
Q¹ is selected from the group consisting of mono- or polycyclic aryl having from 6 to 26 carbon atoms as ring members and mono- or polycyclic hetaryl having a total of 5 to 26 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R^{Ar1} is selected from the group consisting of R¹¹, S(O)ₖR¹¹, NHR¹¹, OR¹¹, CN, halogen, CHₜR′₃-t, NR¹¹₂ and CH═CHR″, where R^{Ar1} may be identical or different if more than one is present on the same (het)aryl or (het)arylene group, where t on each occurrence is 0, 1 or 2 and where k on each occurrence is 0, 1 or 2;
R¹¹ is selected from the group consisting of C₁-C₄-alkyl and a radical Q²;
Q² is selected from the group consisting of mono- or polycyclic aryl having from 6 to 18 carbon atoms as ring members and mono- or polycyclic hetaryl having a total of 5 to 18 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R′ is selected from the group consisting of mono- or polycyclic aryl having from 6 to 18 carbon atoms as ring members and mono- or polycyclic hetaryl having a total of 5 to 18 atoms, which are ring members, where 1, 2, 3 or 4 of these ring member atoms of hetaryl are selected from nitrogen, sulfur and oxygen, while the remainder of these ring member atoms of hetarylene are carbon atoms, where mono- or polycyclic aryl and mono- or polycyclic hetaryl are unsubstituted or carry 1, 2, 3 or 4 radicals R′″;
R″ is selected from hydrogen, methyl, phenyl and naphthyl, where phenyl and naphthyl are unsubstituted or substituted by 1, 2, 3 or 4 identical or different radicals R′″;
R′″ is selected from the group consisting of phenyl, halogen, OCH₃, CH₃, N(CH₃)₂ and C(O)CH₃;
except for compounds of formula (I), where A¹ and A² are both unsubstituted 1,4-phenylene, p and q are both 0, and X¹ and X² are both —CH₂OH or —C(O)OH and further except for the compound of formula (I), where A¹ and A² are both unsubstituted 2,3-quinolinylene, p and q are both 0, and X¹ and X² are both —CH₂OH.

3. The use of claim 1 or the compound of claim 2, where X¹ and X² are both —CH₂OH.

4. The use of claim 1 or the compound of claim 2, where X¹ and X² are both —C(O)OR^{x}, where R^{x} is selected from the group consisting of hydrogen, phenyl, benzyl and C₁-C₄-alkyl, especially from hydrogen, methyl and ethyl, and in particular from hydrogen and methyl.

5. The use or the compound of any one of the preceding claims, where A¹ and A² are independently selected from the group consisting of a mono- or polycyclic arylene having from 6 to 14 carbon atoms as ring members and mono- or polycyclic hetarylene having a total of 5 to 14 atoms, which are ring members, where 1 or 2 of these ring member atoms of hetarylene are selected from nitrogen, sulfur and oxygen, while the remainder of these ring member atoms of hetarylene are carbon atoms, where mono- or polycyclic arylene and mono- or polycyclic hetarylene are unsubstituted or carry 1 or 2 radicals R^{A}r.

6. The use or the compound of any one of the preceding claims, where A¹ and A² are independently selected from the group consisting of a polycyclic arylene having from 6 to 18 carbon atoms as ring members, where polycyclic arylene is unsubstituted or carry 1 or 2 radicals R^{Ar1}.

7. The use or the compound of any one of claims 1 to 4, where A¹ and A² are independently selected from a the group consisting of phenylene, naphthylene, 1,2-dihydroacenaphthylene, biphenylylene, 1,1′-oxydiphenylene, 1,1′-thiodiphenylene, 9H-fluorenylene, 11H-benzo[a]fluorenylene, 11H-benzo[b]fluorenylene, 7H-benzo[c]fluorenylene, anthracylene, phenanthrylene, benzo[c]phenanthrylene, pyrenylene, chrysenylene, picenylene, triphenylenylene, furanylene, benzo[b]furanylene, dibenzo[b,d]furanylene, naphtho[1,2-b]furanylene, naphtho[2,3-b]furanylene, naphtho[2,1-b]furanylene, benzo[b]naphtho[1,2-a]furanylene, benzo[b]naphtho[2,3-d]furanylene, benzo[b]naphtho[2,1-d]furanylene, benzo[1,2-b:4,3-b′]difuranylene, benzo[1.2-b:6,5-b′]difuranylene, benzo[1,2-b:5,4-b′]difuranylene, benzo[1,2-b:4,5-b′]difuranylene, 9H-xanthylene, tribenzo[b,d,f]oxepinylene, oxanthrenylene, 2H-naphtho[1,8-d,e][1,3]dioxinylene, phenoxathiinylene, dinaphtho[2,3-b:2′,3′-d]furanylene, oxanthrenylene, benzo[a]oxanthrenylene, benzo[b]oxanthrenylene, thienylene, benzo[b]thienylene, dibenzo[b,a]thienylene, naphtho[1,2-b]thienylene, naphtho[2,3-b]thienylene, naphtho[2,1-b]thienylene, benzo[b]naphtho[1,2-d]thienylene, benzo[b]naphtho[2,3-d]thienylene, benzo[b]naphtho[2,1-d]thienylene, benzo[1,2-b-4,3-b′]dithienylene, benzo[1,2-b-6,5-b′]dithienylene, benzo[1,2-b:5,4-b′]dithienylene, benzo[1,2-b:4,5-b′]dithienylene, 9H-thioxanthylene, 6H-dibenzo[b,a]thiopyranylene, 1,4-benzodithiinylene, naphtho[1,2-b][1,4]dithiinylene, naphtho[2,3-b][1,4]dithiinylene, 9H-10-thia-anthracylene, thianthrenylene, benzo[a]thianthrenylene, benzo[b]thianthrenylene, dibenzo[a,c]thianthrenylene, dibenzo[a,h]thianthrenylene, dibenzo[a,d]thianthrenylene, dibenzo[a,j]thianthrenylene, dibenzo[b,d]thianthrenylene, 2H-naphtho[1,8-b,c]thienylene, dibenzo[b,d]thiepinylene, dibenzo[b,f]thiepinylene, 5H-phenanthro[4,5-b,c]thiopyranylene, tribenzo[b,d,f]thiepinylene, 2,5-dihydronaphtho[1,8-b,c.4,5-b′c′]dithienylene, 2,6-dihydronaphtho[1,8-b,c-.5,4-b′,c′]dithienylene, tribenzo[a,c,d]thianthrenylene, benzo[b]naphtho[1,8-e,f][1,4]dithiepinylene, dinaphtho[2,3-b-2′,3′-d]thienylene, 5H-phenanthro[1,10-b,c]thienylene, 7H-phenanthro[1,10-c,b]thienylene, dibenzo[d,d′]benzo[1,2-b-4,5-b′]dithienylene and dibenzo[d,d′]benzo[1,2-b:5,4-b′]dithienylene, where the aforementioned radicals are unsubstituted or carry 1 radical R^{Ar1}.

8. The use or the compound of claim 7, where A¹ and A² are independently selected from the group consisting of phenylene, naphthylene, biphenylylenebenzo[b]furanylene, dibenzo[b,d]furanylene, benzo[b]thienylene, dibenzo[b,d]thienylene, 9H-fluorenylene, and thianthrenylene, where phenylene, naphthylene, biphenylylenebenzo[b]furanylene, dibenzo[b,d]furanylene, benzo[b]thienylene, dibenzo[b,d]thienylene, 9H-fluorenylene, and thianthrenylene are unsubstituted or substituted by 1 radical Q², which is in particular selected from the group consisting of phenyl, naphthyl, phenanthryl, thianthrenyl and dibenzo[b,d]thienyl.

9. The use or the compound of claim 8, where A¹ and A² are independently selected from the group consisting of 1,4-phenylene, 1,3-phenylene, 1,2-phenylene, 1,4-naphthylene, 1,2-naphthylene, 2,1-naphthylene, 2,6-naphthylene, 4,4′-biphenylylene, 3,3′-biphenylylene, 2,2′-biphenylylene, 2,4-dibenzo[b,d]thienylene, 2,6-dibenzo[b,d]thienylene, 4,2-dibenzo[b,d]thienylene, 2,8-dibenzo[b,a]thienylene, 4,6-dibenzo[b,d]thienylene, 6,2-dibenzo[b,d]thienylene, 2,4-dibenzo[b,d]furanylene, 2,6-dibenzo[b,d]furanylene, 4,2-dibenzo[b,d]furanylene, 2,8-dibenzo[b,d]furanylene, 4,6-dibenzo[b,d]furanylene, 6,2-dibenzo[b,d]furanylene, 1,2-thianthrenylene, 2,1-thianthrenylene, 3,2-thianthrenylene, 1,3-thianthrenylene, 1,4-thianthrenylene, 1,6-thianthrenylene, 2,7-thianthrenylene, 2,8-thianthrenylene, 1,9-thianthrenylene, 2,7-9H-fluroenylene, 9,2-9H-fluroenylene, 2,9-9H-fluroenylene, 2-(phenyl)-1,4-phenylene, 3-(phenyl)-1,4-phenylene, 2-(1-naphthyl)-1,4-phenylene, 3-(1-naphthyl)-1,4-phenylene, 2-(2-naphthyl)-1,4-phenylene, 3-(2-naphthyl)-1,4-phenylene, 2-(9-phenanthryl)-1,4-phenylene, 3-(9-phenanthryl)-1,4-phenylene, 2-(dibenzo[b,d]thien-4-yl)-1,4-phenylene, 3-(dibenzo[b,d]thien-4-yl)-1,4-phenylene, 2-(thianthren-1-yl)-1,4-phenylene, 3-(thianthren-1-yl)-1,4-phenylene, 2-phenyl-1,4-naphthylene, 2-(1-naphthyl)-1,4-naphthylene, 2-(2-naphthyl)-1,4-naphthylene, 2-(9-phenanthryl)-1,4-naphthylene, 2-(dibenzo[b,]thien-4-yl)-1,4-naphthylene, 2-(thianthren-1-yl)-1,4-naphthylene, 8-phenyl-2,6-naphthylene, 8-(1-naphthyl)-2,6-naphthylene, 8-(2-naphthyl)-2,6-naphthylene, 8-(9-phenanthryl)-2,6-naphthylene, 8-(dibenzo[b,d]thien-4-yl)-2,6-naphthylene and 8-(thianthren-1-yl)-2,6-naphthylene.

10. The use or the compound of any one of the preceding claims, where A¹ and A² have the same meaning.

11. The use or the compound of any one of the preceding claims, where p and q are both 1 and where R¹ and R² are bound to the positions 6 and 6′, respectively, of the binapthyl moiety of formula (I).

12. The use or the compound of any one of the preceding claims, where R¹ and R² are independently selected from the group consisting of fluorine, CN, methyl, methoxy and the radical Q¹, and where R¹ and R² are in particular a radical Q¹ which is mono- or polycyclic aryl having from 6 to 14 carbon atoms as ring members and mono- or polycyclic hetaryl having a total of 5 to 14 atoms, which are ring members, where 1 or 2 of these ring member atoms of hetaryl are selected from nitrogen, sulfur and oxygen, while the remainder of these ring member atoms of hetaryl are carbon atoms, and where R¹ and R² are specifically from the group consisting of phenyl, naphthyl, phenanthryl, thianthrenyl and dibenzo[b,a]thienyl.

13. The use or the compound of any one of the preceding claims, where R¹ and R² have the same meaning.

14. The use or the compound of any one of the preceding claims, where p and q are both 0.

15. The use or the compound of any one of the preceding claims, where formula (I) is represented by formula (Ia), where X, R⁰ and A are as defined in one row of table A: 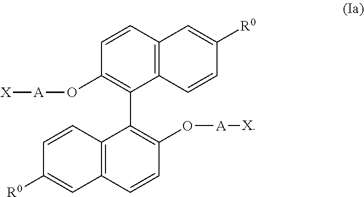
TABLE A

| # | X | A *) | R⁰ |
|---|---|---|---|
| 1 | —CH₂OH | 1,4-phenylene | H |
| 2 | —CH₂OH | 1,3-phenylene | H |
| 3 | —CH₂OH | 1,2-phenylene | H |
| 4 | —CH₂OH | 1,4-naphthylene | H |
| 5 | —CH₂OH | 2,1-naphthylene | H |
| 6 | —CH₂OH | 2,6-naphthylene | H |
| 7 | —CH₂OH | 4,4′-biphenylylene | H |
| 8 | —CH₂OH | 3,3′-biphenylylene | H |
| 9 | —CH₂OH | 2,2′-biphenylylene | H |
| 10 | —CH₂OH | 2,7-9H-fluorenylene | H |
| 11 | —CH₂OH | 2,9-9H-fluorenylene | H |
| 12 | —CH₂OH | 9,2-9H-fluorenylene | H |
| 13 | —CH₂OH | 2,4-dibenzo[b,d]thienylene | H |
| 14 | —CH₂OH | 2,6-dibenzo[b,d]thienylene | H |
| 15 | —CH₂OH | 4,2-dibenzo[b,d]thienylene | H |
| 16 | —CH₂OH | 6,2-dibenzo[b,d]thienylene | H |
| 17 | —CH₂OH | 4,6-dibenzo[b,d]thienylene | H |
| 18 | —CH₂OH | 2,8-dibenzo[b,d]thienylene | H |
| 19 | —CH₂OH | 2,4-dibenzo[b,d]furanylene | H |
| 20 | —CH₂OH | 2,6-dibenzo[b,d]furanylene | H |
| 21 | —CH₂OH | 4,2-dibenzo[b,d]furanylene | H |
| 22 | —CH₂OH | 6,2-dibenzo[b,d]furanylene | H |
| 23 | —CH₂OH | 4,6-dibenzo[b,d]furanylene | H |
| 24 | —CH₂OH | 2,8-dibenzo[b,d]furanylene | H |
| 25 | —CH₂OH | 1,2-thianthrenylene | H |
| 26 | —CH₂OH | 2,1-thianthrenylene | H |
| 27 | —CH₂OH | 1,3-thianthrenylene | H |
| 28 | —CH₂OH | 2,3-thianthrenylene | H |
| 29 | —CH₂OH | 1,4-thianthrenylene | H |
| 30 | —CH₂OH | 1,6-thianthrenylene | H |
| 31 | —CH₂OH | 2,7-thianthrenylene | H |
| 32 | —CH₂OH | 2,8-thianthrenylene | H |
| 33 | —CH₂OH | 1,9-thianthrenylene | H |
| 34 | —C(O)OH | 1,3-phenylene | H |
| 35 | —C(O)OH | 1,2-phenylene | H |
| 36 | —C(O)OH | 1,4-naphthylene | H |
| 37 | —C(O)OH | 2,1-naphthylene | H |
| 38 | —C(O)OH | 2,6-naphthylene | H |
| 39 | —C(O)OH | 4,4′-biphenylylene | H |
| 40 | —C(O)OH | 3,3′-biphenylylene | H |
| 41 | —C(O)OH | 2,2′-biphenylylene | H |
| 42 | —C(O)OH | 2,7-9H-fluorenylene | H |
| 43 | —C(O)OH | 2,9-9H-fluorenylene | H |
| 44 | —C(O)OH | 9,2-9H-fluorenylene | H |
| 45 | —C(O)OH | 2,4-dibenzo[b,d]thienylene | H |
| 46 | —C(O)OH | 2,6-dibenzo[b,d]thienylene | H |
| 47 | —C(O)OH | 4,2-dibenzo[b,d]thienylene | H |
| 48 | —C(O)OH | 6,2-dibenzo[b,d]thienylene | H |
| 49 | —C(O)OH | 4,6-dibenzo[b,d]thienylene | H |
| 50 | —C(O)OH | 2,8-dibenzo[b,d]thienylene | H |
| 51 | —C(O)OH | 2,4-dibenzo[b,d]furanylene | H |
| 52 | —C(O)OH | 2,6-dibenzo[b,d]furanylene | H |
| 53 | —C(O)OH | 4,2-dibenzo[b,d]furanylene | H |
| 54 | —C(O)OH | 6,2-dibenzo[b,d]furanylene | H |
| 55 | —C(O)OH | 4,6-dibenzo[b,d]furanylene | H |
| 56 | —C(O)OH | 2,8-dibenzo[b,d]furanylene | H |
| 57 | —C(O)OH | 1,2-thianthrenylene | H |
| 58 | —C(O)OH | 2,1-thianthrenylene | H |
| 59 | —C(O)OH | 1,3-thianthrenylene | H |
| 60 | —C(O)OH | 2,3-thianthrenylene | H |
| 61 | —C(O)OH | 1,4-thianthrenylene | H |
| 62 | —C(O)OH | 1,6-thianthrenylene | H |
| 63 | —C(O)OH | 2,7-thianthrenylene | H |
| 64 | —C(O)OH | 2,8-thianthrenylene | H |
| 65 | —C(O)OH | 1,9-thianthrenylene | H |
| 66 | —C(O)OCH₃ | 1,3-phenylene | H |
| 67 | —C(O)OCH₃ | 1,2-phenylene | H |
| 68 | —C(O)OCH₃ | 1,4-naphthylene | H |
| 69 | —C(O)OCH₃ | 2,1-naphthylene | H |
| 70 | —C(O)OCH₃ | 2,6-naphthylene | H |
| 71 | —C(O)OCH₃ | 4,4′-biphenylylene | H |
| 72 | —C(O)OCH₃ | 3,3′-biphenylylene | H |
| 73 | —C(O)OCH₃ | 2,2′-biphenylylene | H |
| 74 | —C(O)OCH₃ | 2,7-9H-fluorenylene | H |
| 75 | —C(O)OCH₃ | 2,9-9H-fluorenylene | H |
| 76 | —C(O)OCH₃ | 9,2-9H-fluorenylene | H |
| 77 | —C(O)OCH₃ | 2,4-dibenzo[b,d]thienylene | H |
| 78 | —C(O)OCH₃ | 2,6-dibenzo[b,d]thienylene | H |
| 79 | —C(O)OCH₃ | 4,2-dibenzo[b,d]thienylene | H |
| 80 | —C(O)OCH₃ | 6,2-dibenzo[b,d]thienylene | H |
| 81 | —C(O)OCH₃ | 4,6-dibenzo[b,d]thienylene | H |
| 82 | —C(O)OCH₃ | 2,8-dibenzo[b,d]thienylene | H |
| 83 | —C(O)OCH₃ | 2,4-dibenzo[b,d]furanylene | H |
| 84 | —C(O)OCH₃ | 2,6-dibenzo[b,d]furanylene | H |
| 85 | —C(O)OCH₃ | 4,2-dibenzo[b,d]furanylene | H |
| 86 | —C(O)OCH₃ | 6,2-dibenzo[b,d]furanylene | H |
| 87 | —C(O)OCH₃ | 4,6-dibenzo[b,d]furanylene | H |
| 88 | —C(O)OCH₃ | 2,8-dibenzo[b,d]furanylene | H |
| 89 | —C(O)OCH₃ | 1,2-thianthrenylene | H |
| 90 | —C(O)OCH₃ | 2,1-thianthrenylene | H |
| 91 | —C(O)OCH₃ | 1,3-thianthrenylene | H |
| 92 | —C(O)OCH₃ | 2,3-thianthrenylene | H |
| 93 | —C(O)OCH₃ | 1,4-thianthrenylene | H |
| 94 | —C(O)OCH₃ | 1,6-thianthrenylene | H |
| 95 | —C(O)OCH₃ | 2,7-thianthrenylene | H |
| 96 | —C(O)OCH₃ | 2,8-thianthrenylene | H |
| 97 | —C(O)OCH₃ | 1,9-thianthrenylene | H |
| 98 | —CH₂OH | 1,4-phenylene | phenyl |
| 99 | —CH₂OH | 1,2-phenylene | phenyl |
| 100 | —CH₂OH | 1,4-naphthylene | phenyl |
| 101 | —CH₂OH | 2,1-naphthylene | phenyl |
| 102 | —CH₂OH | 2,6-naphthylene | phenyl |
| 103 | —CH₂OH | 4,4′-biphenylylene | phenyl |
| 104 | —CH₂OH | 2,2′-biphenylylene | phenyl |
| 105 | —CH₂OH | 2,7-9H-fluorenylene | phenyl |
| 106 | —C(O)OH | 1,4-phenylene | phenyl |
| 107 | —C(O)OH | 1,2-phenylene | phenyl |
| 108 | —C(O)OH | 1,4-naphthylene | phenyl |
| 109 | —C(O)OH | 2,1-naphthylene | phenyl |
| 110 | —C(O)OH | 2,6-naphthylene | phenyl |
| 111 | —C(O)OH | 4,4′-biphenylylene | phenyl |
| 112 | —C(O)OH | 2,2′-biphenylylene | phenyl |
| 113 | —C(O)OH | 2,7-9H-fluorenylene | phenyl |
| 114 | —C(O)OCH₃ | 1,4-phenylene | phenyl |
| 115 | —C(O)OCH₃ | 1,2-phenylene | phenyl |
| 116 | —C(O)OCH₃ | 1,4-naphthylene | phenyl |
| 117 | —C(O)OCH₃ | 2,1-naphthylene | phenyl |
| 118 | —C(O)OCH₃ | 2,6-naphthylene | phenyl |
| 119 | —C(O)OCH₃ | 4,4′-biphenylylene | phenyl |
| 120 | —C(O)OCH₃ | 2,2′-biphenylylene | phenyl |
| 121 | —C(O)OCH₃ | 2,7-9H-fluorenylene | phenyl |
| 122 | —CH₂OH | 1,4-phenylene | 1-naphthyl |
| 123 | —CH₂OH | 1,2-phenylene | 1-naphthyl |
| 124 | —CH₂OH | 1,4-naphthylene | 1-naphthyl |
| 125 | —CH₂OH | 2,1-naphthylene | 1-naphthyl |
| 126 | —CH₂OH | 2,6-naphthylene | 1-naphthyl |
| 127 | —CH₂OH | 4,4′-biphenylylene | 1-naphthyl |
| 128 | —CH₂OH | 2,2′-biphenylylene | 1-naphthyl |
| 129 | —CH₂OH | 2,7-9H-fluorenylene | 1-naphthyl |
| 130 | —C(O)OH | 1,4-phenylene | 1-naphthyl |
| 131 | —C(O)OH | 1,2-phenylene | 1-naphthyl |
| 132 | —C(O)OH | 1,4-naphthylene | 1-naphthyl |
| 133 | —C(O)OH | 2,1-naphthylene | 1-naphthyl |
| 134 | —C(O)OH | 2,6-naphthylene | 1-naphthyl |
| 135 | —C(O)OH | 4,4′-biphenylylene | 1-naphthyl |
| 136 | —C(O)OH | 2,2′-biphenylylene | 1-naphthyl |
| 137 | —C(O)OH | 2,7-9H-fluorenylene | 1-naphthyl |
| 138 | —C(O)OCH₃ | 1,4-phenylene | 1-naphthyl |
| 139 | —C(O)OCH₃ | 1,2-phenylene | 1-naphthyl |
| 140 | —C(O)OCH₃ | 1,4-naphthylene | 1-naphthyl |
| 141 | —C(O)OCH₃ | 2,1-naphthylene | 1-naphthyl |
| 142 | —C(O)OCH₃ | 2,6-naphthylene | 1-naphthyl |
| 143 | —C(O)OCH₃ | 4,4′-biphenylylene | 1-naphthyl |
| 144 | —C(O)OCH₃ | 2,2′-biphenylylene | 1-naphthyl |
| 145 | —C(O)OCH₃ | 2,7-9H-fluorenylene | 1-naphthyl |
| 146 | —CH₂OH | 1,4-phenylene | 2-naphthyl |
| 147 | —CH₂OH | 1,2-phenylene | 2-naphthyl |
| 148 | —CH₂OH | 1,4-naphthylene | 2-naphthyl |
| 149 | —CH₂OH | 2,1-naphthylene | 2-naphthyl |
| 150 | —CH₂OH | 2,6-naphthylene | 2-naphthyl |
| 151 | —CH₂OH | 4,4′-biphenylylene | 2-naphthyl |
| 152 | —CH₂OH | 2,2′-biphenylylene | 2-naphthyl |
| 153 | —CH₂OH | 2,7-9H-fluorenylene | 2-naphthyl |
| 154 | —C(O)OH | 1,4-phenylene | 2-naphthyl |
| 155 | —C(O)OH | 1,2-phenylene | 2-naphthyl |
| 156 | —C(O)OH | 1,4-naphthylene | 2-naphthyl |
| 157 | —C(O)OH | 2,1-naphthylene | 2-naphthyl |
| 158 | —C(O)OH | 2,6-naphthylene | 2-naphthyl |
| 159 | —C(O)OH | 4,4′-biphenylylene | 2-naphthyl |
| 160 | —C(O)OH | 2,2′-biphenylylene | 2-naphthyl |
| 161 | —C(O)OH | 2,7-9H-fluorenylene | 2-naphthyl |
| 162 | —C(O)OCH₃ | 1,4-phenylene | 2-naphthyl |
| 163 | —C(O)OCH₃ | 1,2-phenylene | 2-naphthyl |
| 164 | —C(O)OCH₃ | 1,4-naphthylene | 2-naphthyl |
| 165 | —C(O)OCH₃ | 2,1-naphthylene | 2-naphthyl |
| 166 | —C(O)OCH₃ | 2,6-naphthylene | 2-naphthyl |
| 167 | —C(O)OCH₃ | 4,4′-biphenylylene | 2-naphthyl |
| 168 | —C(O)OCH₃ | 2,2′-biphenylylene | 2-naphthyl |
| 169 | —C(O)OCH₃ | 2,7-9H-fluorenylene | 2-naphthyl |
| 170 | —CH₂OH | 1,4-phenylene | 9-phenanthryl |
| 171 | —CH₂OH | 1,2-phenylene | 9-phenanthryl |
| 172 | —CH₂OH | 1,4-naphthylene | 9-phenanthryl |
| 173 | —CH₂OH | 2,1-naphthylene | 9-phenanthryl |
| 174 | —CH₂OH | 2,6-naphthylene | 9-phenanthryl |
| 175 | —CH₂OH | 4,4′-biphenylylene | 9-phenanthryl |
| 176 | —CH₂OH | 2,2′-biphenylylene | 9-phenanthryl |
| 177 | —CH₂OH | 2,7-9H-fluorenylene | 9-phenanthryl |
| 178 | —C(O)OH | 1,4-phenylene | 9-phenanthryl |
| 179 | —C(O)OH | 1,2-phenylene | 9-phenanthryl |
| 180 | —C(O)OH | 1,4-naphthylene | 9-phenanthryl |
| 181 | —C(O)OH | 2,1-naphthylene | 9-phenanthryl |
| 182 | —C(O)OH | 2,6-naphthylene | 9-phenanthryl |
| 183 | —C(O)OH | 4,4′-biphenylylene | 9-phenanthryl |
| 184 | —C(O)OH | 2,2′-biphenylylene | 9-phenanthryl |
| 185 | —C(O)OH | 2,7-9H-fluorenylene | 9-phenanthryl |
| 186 | —C(O)OCH₃ | 1,4-phenylene | 9-phenanthryl |
| 187 | —C(O)OCH₃ | 1,2-phenylene | 9-phenanthryl |
| 188 | —C(O)OCH₃ | 1,4-naphthylene | 9-phenanthryl |
| 189 | —C(O)OCH₃ | 2,1-naphthylene | 9-phenanthryl |
| 190 | —C(O)OCH₃ | 2,6-naphthylene | 9-phenanthryl |
| 191 | —C(O)OCH₃ | 4,4′-biphenylylene | 9-phenanthryl |
| 192 | —C(O)OCH₃ | 2,2′-biphenylylene | 9-phenanthryl |
| 193 | —C(O)OCH₃ | 2,7-9H-fluorenylene | 9-phenanthryl |
| 194 | —CH₂OH | 1,4-phenylene | dibenzo[b,d]thien-4- |
| | | | yl |
| 195 | —CH₂OH | 1,2-phenylene | dibenzo[b,d]thien-4- |
| | | | yl |
| 196 | —CH₂OH | 1,4-naphthylene | dibenzo[b,d]thien-4- |
| | | | yl |
| 197 | —CH₂OH | 2,1-naphthylene | dibenzo[b,d]thien-4- |
| | | | yl |
| 198 | —CH₂OH | 2,6-naphthylene | dibenzo[b,d]thien-4- |
| | | | yl |
| 199 | —CH₂OH | 4,4′-biphenylylene | dibenzo[b,d]thien-4- |
| | | | yl |
| 200 | —CH₂OH | 2,2′-biphenylylene | dibenzo[b,d]thien-4- |
| | | | yl |
| 201 | —CH₂OH | 2,7-9H-fluorenylene | dibenzo[b,d]thien-4- |
| | | | yl |
| 202 | —C(O)OH | 1,4-phenylene | dibenzo[b,d]thien-4- |
| | | | yl |
| 203 | —C(O)OH | 1,2-phenylene | dibenzo[b,d]thien-4- |
| | | | yl |
| 204 | —C(O)OH | 1,4-naphthylene | dibenzo[b,d]thien-4- |
| | | | yl |
| 205 | —C(O)OH | 2,1-naphthylene | dibenzo[b,d]thien-4- |
| | | | yl |
| 206 | —C(O)OH | 2,6-naphthylene | dibenzo[b,d]thien-4- |
| | | | yl |
| 207 | —C(O)OH | 4,4′-biphenylylene | dibenzo[b,d]thien-4- |
| | | | yl |
| 208 | —C(O)OH | 2,2′-biphenylylene | dibenzo[b,d]thien-4- |
| | | | yl |
| 209 | —C(O)OH | 2,7-9H-fluorenylene | dibenzo[b,d]thien-4- |
| | | | yl |
| 210 | —C(O)OCH₃ | 1,4-phenylene | dibenzo[b,d]thien-4- |
| | | | yl |
| 211 | —C(O)OCH₃ | 1,2-phenylene | dibenzo[b,d]thien-4- |
| | | | yl |
| 212 | —C(O)OCH₃ | 1,4-naphthylene | dibenzo[b,d]thien-4- |
| | | | yl |
| 213 | —C(O)OCH₃ | 2,1-naphthylene | dibenzo[b,d]thien-4- |
| | | | yl |
| 214 | —C(O)OCH₃ | 2,6-naphthylene | dibenzo[b,d]thien-4- |
| | | | yl |
| 215 | —C(O)OCH₃ | 4,4′-biphenylylene | dibenzo[b,d]thien-4- |
| | | | yl |
| 216 | —C(O)OCH₃ | 2,2′-biphenylylene | dibenzo[b,d]thien-4- |
| | | | yl |
| 217 | —C(O)OCH₃ | 2,7-9H-fluorenylene | dibenzo[b,d]thien-4- |
| | | | yl |
| 218 | —CH₂OH | 1,4-phenylene | thianthren-1-yl |
| 219 | —CH₂OH | 1,2-phenylene | thianthren-1-yl |
| 220 | —CH₂OH | 1,4-naphthylene | thianthren-1-yl |
| 221 | —CH₂OH | 2,1-naphthylene | thianthren-1-yl |
| 222 | —CH₂OH | 2,6-naphthylene | thianthren-1-yl |
| 223 | —CH₂OH | 4,4′-biphenylylene | thianthren-1-yl |
| 224 | —CH₂OH | 2,2′-biphenylylene | thianthren-1-yl |
| 225 | —CH₂OH | 2,7-9H-fluorenylene | thianthren-1-yl |
| 226 | —C(O)OH | 1,4-phenylene | thianthren-1-yl |
| 227 | —C(O)OH | 1,2-phenylene | thianthren-1-yl |
| 228 | —C(O)OH | 1,4-naphthylene | thianthren-1-yl |
| 229 | —C(O)OH | 2,1-naphthylene | thianthren-1-yl |
| 230 | —C(O)OH | 2,6-naphthylene | thianthren-1-yl |
| 231 | —C(O)OH | 4,4′-biphenylylene | thianthren-1-yl |
| 232 | —C(O)OH | 2,2′-biphenylylene | thianthren-1-yl |
| 233 | —C(O)OH | 2,7-9H-fluorenylene | thianthren-1-yl |
| 234 | —C(O)OCH₃ | 1,4-phenylene | thianthren-1-yl |
| 235 | —C(O)OCH₃ | 1,2-phenylene | thianthren-1-yl |
| 236 | —C(O)OCH₃ | 1,4-naphthylene | thianthren-1-yl |
| 237 | —C(O)OCH₃ | 2,1-naphthylene | thianthren-1-yl |
| 238 | —C(O)OCH₃ | 2,6-naphthylene | thianthren-1-yl |
| 239 | —C(O)OCH₃ | 4,4′-biphenylylene | thianthren-1-yl |
| 240 | —C(O)OCH₃ | 2,2′-biphenylylene | thianthren-1-yl |
| 241 | —C(O)OCH₃ | 2,7-9H-fluorenylene | thianthren-1-yl |

*) the linkage positions “n, m-” included in the names of the moieties A are to be understood such that the first one, i.e. n, indicates the position of the carbon atom linked to X, and the second one, i.e. m, indicates the position of the carbon atom linked to the O atom.

16. The use or the compound of any one of the preceding claims, where formula (I) is represented by one of the formulae (Ia.1), (Ia.2), (Ia.3) or (Ia.4), where X, R⁰ and Q⁰ are as defined in one row of table B: 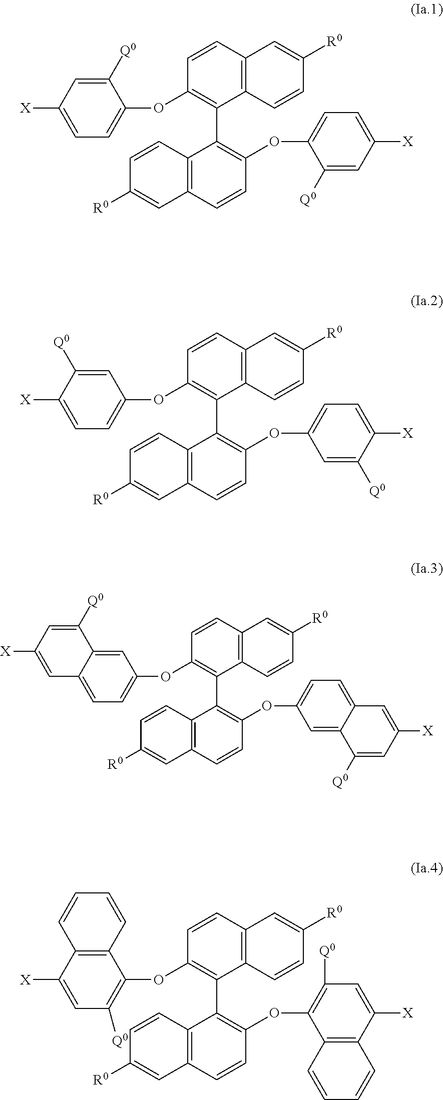
TABLE B

| # | Formula | X | Q⁰ | R⁰ |
|---|---|---|---|---|
| 1 | Ia.1 | —CH₂OH | phenyl | H |
| 2 | Ia.1 | —C(O)OH | phenyl | H |
| 3 | Ia.1 | —C(O)OCH₃ | phenyl | H |
| 4 | Ia.1 | —CH₂OH | 1-naphthyl | H |
| 5 | Ia.1 | —C(O)OH | 1-naphthyl | H |
| 6 | Ia.1 | —C(O)OCH₃ | 1-naphthyl | H |
| 7 | Ia.1 | —CH₂OH | 2-naphthyl | H |
| 8 | Ia.1 | —C(O)OH | 2-naphthyl | H |
| 9 | Ia.1 | —C(O)OCH₃ | 2-naphthyl | H |
| 10 | Ia.1 | —CH₂OH | 9-phenanthryl | H |
| 11 | Ia.1 | —C(O)OH | 9-phenanthryl | H |
| 12 | Ia.1 | —C(O)OCH₃ | 9-phenanthryl | H |
| 13 | Ia.1 | —CH₂OH | dibenzo[b,d]thien-4-yl | H |
| 14 | Ia.1 | —C(O)OH | dibenzo[b,d]thien-4-yl | H |
| 15 | Ia.1 | —C(O)OCH₃ | dibenzo[b,d]thien-4-yl | H |
| 16 | Ia.1 | —CH₂OH | thianthren-1-yl | H |
| 17 | Ia.1 | —C(O)OH | thianthren-1-yl | H |
| 18 | Ia.1 | —C(O)OCH₃ | thianthren-1-yl | H |
| 19 | Ia.2 | —CH₂OH | phenyl | H |
| 20 | Ia.2 | —C(O)OH | phenyl | H |
| 21 | Ia.2 | —C(O)OCH₃ | phenyl | H |
| 22 | Ia.2 | —CH₂OH | 1-naphthyl | H |
| 23 | Ia.2 | —C(O)OH | 1-naphthyl | H |
| 24 | Ia.2 | —C(O)OCH₃ | 1-naphthyl | H |
| 25 | Ia.2 | —CH₂OH | 2-naphthyl | H |
| 26 | Ia.2 | —C(O)OH | 2-naphthyl | H |
| 27 | Ia.2 | —C(O)OCH₃ | 2-naphthyl | H |
| 28 | Ia.2 | —CH₂OH | 9-phenanthryl | H |
| 29 | Ia.2 | —C(O)OH | 9-phenanthryl | H |
| 30 | Ia.2 | —C(O)OCH₃ | 9-phenanthryl | H |
| 31 | Ia.2 | —CH₂OH | dibenzo[b,d]thien-4-yl | H |
| 32 | Ia.2 | —C(O)OH | dibenzo[b,d]thien-4-yl | H |
| 33 | Ia.2 | —C(O)OCH₃ | dibenzo[b,d]thien-4-yl | H |
| 34 | Ia.2 | —CH₂OH | thianthren-1-yl | H |
| 35 | Ia.2 | —C(O)OH | thianthren-1-yl | H |
| 36 | Ia.2 | —C(O)OCH₃ | thianthren-1-yl | H |
| 37 | Ia.3 | —CH₂OH | phenyl | H |
| 38 | Ia.3 | —C(O)OH | phenyl | H |
| 39 | Ia.3 | —C(O)OCH₃ | phenyl | H |
| 40 | Ia.3 | —CH₂OH | 1-naphthyl | H |
| 41 | Ia.3 | —C(O)OH | 1-naphthyl | H |
| 42 | Ia.3 | —C(O)OCH₃ | 1-naphthyl | H |
| 43 | Ia.3 | —CH₂OH | 2-naphthyl | H |
| 44 | Ia.3 | —C(O)OH | 2-naphthyl | H |
| 45 | Ia.3 | —C(O)OCH₃ | 2-naphthyl | H |
| 46 | Ia.3 | —CH₂OH | 9-phenanthryl | H |
| 47 | Ia.3 | —C(O)OH | 9-phenanthryl | H |
| 48 | Ia.3 | —C(O)OCH₃ | 9-phenanthryl | H |
| 49 | Ia.3 | —CH₂OH | dibenzo[b,d]thien-4-yl | H |
| 50 | Ia.3 | —C(O)OH | dibenzo[b,d]thien-4-yl | H |
| 51 | Ia.3 | —C(O)OCH₃ | dibenzo[b,d]thien-4-yl | H |
| 52 | Ia.3 | —CH₂OH | thianthren-1-yl | H |
| 53 | Ia.3 | —C(O)OH | thianthren-1-yl | H |
| 54 | Ia.3 | —C(O)OCH₃ | thianthren-1-yl | H |
| 55 | Ia.4 | —CH₂OH | phenyl | H |
| 56 | Ia.4 | —C(O)OH | phenyl | H |
| 57 | Ia.4 | —C(O)OCH₃ | phenyl | H |
| 58 | Ia.4 | —CH₂OH | 1-naphthyl | H |
| 59 | Ia.4 | —C(O)OH | 1-naphthyl | H |
| 60 | Ia.4 | —C(O)OCH₃ | 1-naphthyl | H |
| 61 | Ia.4 | —CH₂OH | 2-naphthyl | H |
| 62 | Ia.4 | —C(O)OH | 2-naphthyl | H |
| 63 | Ia.4 | —C(O)OCH₃ | 2-naphthyl | H |
| 64 | Ia.4 | —CH₂OH | 9-phenanthryl | H |
| 65 | Ia.4 | —C(O)OH | 9-phenanthryl | H |
| 66 | Ia.4 | —C(O)OCH₃ | 9-phenanthryl | H |
| 67 | Ia.4 | —CH₂OH | dibenzo[b,d]thien-4-yl | H |
| 68 | Ia.4 | —C(O)OH | dibenzo[b,d]thien-4-yl | H |
| 69 | Ia.4 | —C(O)OCH₃ | dibenzo[b,d]thien-4-yl | H |
| 70 | Ia.4 | —CH₂OH | thianthren-1-yl | H |
| 71 | Ia.4 | —C(O)OH | thianthren-1-yl | H |
| 72 | Ia.4 | —C(O)OCH₃ | thianthren-1-yl | H |
| 73 | Ia.1 | —CH₂OH | phenyl | phenyl |
| 74 | Ia.1 | —C(O)OH | phenyl | phenyl |
| 75 | Ia.1 | —C(O)OCH₃ | phenyl | phenyl |
| 76 | Ia.1 | —CH₂OH | 1-naphthyl | phenyl |
| 77 | Ia.1 | —C(O)OH | 1-naphthyl | phenyl |
| 78 | Ia.1 | —C(O)OCH₃ | 1-naphthyl | phenyl |
| 79 | Ia.1 | —CH₂OH | 2-naphthyl | phenyl |
| 80 | Ia.1 | —C(O)OH | 2-naphthyl | phenyl |
| 81 | Ia.1 | —C(O)OCH₃ | 2-naphthyl | phenyl |
| 82 | Ia.1 | —CH₂OH | 9-phenanthryl | phenyl |
| 83 | Ia.1 | —C(O)OH | 9-phenanthryl | phenyl |
| 84 | Ia.1 | —C(O)OCH₃ | 9-phenanthryl | phenyl |
| 85 | Ia.1 | —CH₂OH | dibenzo[b,d]thien-4-yl | phenyl |
| 86 | Ia.1 | —C(O)OH | dibenzo[b,d]thien-4-yl | phenyl |
| 87 | Ia.1 | —C(O)OCH₃ | dibenzo[b,d]thien-4-yl | phenyl |
| 88 | Ia.1 | —CH₂OH | thianthren-1-yl | phenyl |
| 89 | Ia.1 | —C(O)OH | thianthren-1-yl | phenyl |
| 90 | Ia.1 | —C(O)OCH₃ | thianthren-1-yl | phenyl |
| 91 | Ia.1 | —CH₂OH | phenyl | 1-naphthyl |
| 92 | Ia.1 | —C(O)OH | phenyl | 1-naphthyl |
| 93 | Ia.1 | —C(O)OCH₃ | phenyl | 1-naphthyl |
| 94 | Ia.1 | —CH₂OH | 1-naphthyl | 1-naphthyl |
| 95 | Ia.1 | —C(O)OH | 1-naphthyl | 1-naphthyl |
| 96 | Ia.1 | —C(O)OCH₃ | 1-naphthyl | 1-naphthyl |
| 97 | Ia.1 | —CH₂OH | 2-naphthyl | 1-naphthyl |
| 98 | Ia.1 | —C(O)OH | 2-naphthyl | 1-naphthyl |
| 99 | Ia.1 | —C(O)OCH₃ | 2-naphthyl | 1-naphthyl |
| 100 | Ia.1 | —CH₂OH | 9-phenanthryl | 1-naphthyl |
| 101 | Ia.1 | —C(O)OH | 9-phenanthryl | 1-naphthyl |
| 102 | Ia.1 | —C(O)OCH₃ | 9-phenanthryl | 1-naphthyl |
| 103 | Ia.1 | —CH₂OH | dibenzo[b,d]thien-4-yl | 1-naphthyl |
| 104 | Ia.1 | —C(O)OH | dibenzo[b,d]thien-4-yl | 1-naphthyl |
| 105 | Ia.1 | —C(O)OCH₃ | dibenzo[b,d]thien-4-yl | 1-naphthyl |
| 106 | Ia.1 | —CH₂OH | thianthren-1-yl | 1-naphthyl |
| 107 | Ia.1 | —C(O)OH | thianthren-1-yl | 1-naphthyl |
| 108 | Ia.1 | —C(O)OCH₃ | thianthren-1-yl | 1-naphthyl |
| 109 | Ia.1 | —CH₂OH | phenyl | 2-naphthyl |
| 110 | Ia.1 | —C(O)OH | phenyl | 2-naphthyl |
| 111 | Ia.1 | —C(O)OCH₃ | phenyl | 2-naphthyl |
| 112 | Ia.1 | —CH₂OH | 1-naphthyl | 2-naphthyl |
| 113 | Ia.1 | —C(O)OH | 1-naphthyl | 2-naphthyl |
| 114 | Ia.1 | —C(O)OCH₃ | 1-naphthyl | 2-naphthyl |
| 115 | Ia.1 | —CH₂OH | 2-naphthyl | 2-naphthyl |
| 116 | Ia.1 | —C(O)OH | 2-naphthyl | 2-naphthyl |
| 117 | Ia.1 | —C(O)OCH₃ | 2-naphthyl | 2-naphthyl |
| 118 | Ia.1 | —CH₂OH | 9-phenanthryl | 2-naphthyl |
| 119 | Ia.1 | —C(O)OH | 9-phenanthryl | 2-naphthyl |
| 120 | Ia.1 | —C(O)OCH₃ | 9-phenanthryl | 2-naphthyl |
| 121 | Ia.1 | —CH₂OH | dibenzo[b,d]thien-4-yl | 2-naphthyl |
| 122 | Ia.1 | —C(O)OH | dibenzo[b,d]thien-4-yl | 2-naphthyl |
| 123 | Ia.1 | —C(O)OCH₃ | dibenzo[b,d]thien-4-yl | 2-naphthyl |
| 124 | Ia.1 | —CH₂OH | thianthren-1-yl | 2-naphthyl |
| 125 | Ia.1 | —C(O)OH | thianthren-1-yl | 2-naphthyl |
| 126 | Ia.1 | —C(O)OCH₃ | thianthren-1-yl | 2-naphthyl |
| 127 | Ia.1 | —CH₂OH | phenyl | 9-phenanthryl |
| 128 | Ia.1 | —C(O)OH | phenyl | 9-phenanthryl |
| 129 | Ia.1 | —C(O)OCH₃ | phenyl | 9-phenanthryl |
| 130 | Ia.1 | —CH₂OH | 1-naphthyl | 9-phenanthryl |
| 131 | Ia.1 | —C(O)OH | 1-naphthyl | 9-phenanthryl |
| 132 | Ia.1 | —C(O)OCH₃ | 1-naphthyl | 9-phenanthryl |
| 133 | Ia.1 | —CH₂OH | 2-naphthyl | 9-phenanthryl |
| 134 | Ia.1 | —C(O)OH | 2-naphthyl | 9-phenanthryl |
| 135 | Ia.1 | —C(O)OCH₃ | 2-naphthyl | 9-phenanthryl |
| 136 | Ia.1 | —CH₂OH | 9-phenanthryl | 9-phenanthryl |
| 137 | Ia.1 | —C(O)OH | 9-phenanthryl | 9-phenanthryl |
| 138 | Ia.1 | —C(O)OCH₃ | 9-phenanthryl | 9-phenanthryl |
| 139 | Ia.1 | —CH₂OH | dibenzo[b,d]thien-4-yl | 9-phenanthryl |
| 140 | Ia.1 | —C(O)OH | dibenzo[b,d]thien-4-yl | 9-phenanthryl |
| 141 | Ia.1 | —C(O)OCH₃ | dibenzo[b,d]thien-4-yl | 9-phenanthryl |
| 142 | Ia.1 | —CH₂OH | thianthren-1-yl | 9-phenanthryl |
| 143 | Ia.1 | —C(O)OH | thianthren-1-yl | 9-phenanthryl |
| 144 | Ia.1 | —C(O)OCH₃ | thianthren-1-yl | 9-phenanthryl |
| 145 | Ia.1 | —CH₂OH | phenyl | dibenzo[b,d]thien-4-yl |
| 146 | Ia.1 | —C(O)OH | phenyl | dibenzo[b,d]thien-4-yl |
| 147 | Ia.1 | —C(O)OCH₃ | phenyl | dibenzo[b,d]thien-4-yl |
| 148 | Ia.1 | —CH₂OH | 1-naphthyl | dibenzo[b,d]thien-4-yl |
| 149 | Ia.1 | —C(O)OH | 1-naphthyl | dibenzo[b,d]thien-4-yl |
| 150 | Ia.1 | —C(O)OCH₃ | 1-naphthyl | dibenzo[b,d]thien-4-yl |
| 151 | Ia.1 | —CH₂OH | 2-naphthyl | dibenzo[b,d]thien-4-yl |
| 152 | Ia.1 | —C(O)OH | 2-naphthyl | dibenzo[b,d]thien-4-yl |
| 153 | Ia.1 | —C(O)OCH₃ | 2-naphthyl | dibenzo[b,d]thien-4-yl |
| 154 | Ia.1 | —CH₂OH | 9-phenanthryl | dibenzo[b,d]thien-4-yl |
| 155 | Ia.1 | —C(O)OH | 9-phenanthryl | dibenzo[b,d]thien-4-yl |
| 156 | Ia.1 | —C(O)OCH₃ | 9-phenanthryl | dibenzo[b,d]thien-4-yl |
| 157 | Ia.1 | —CH₂OH | dibenzo[b,d]thien-4-yl | dibenzo[b,d]thien-4-yl |
| 158 | Ia.1 | —C(O)OH | dibenzo[b,d]thien-4-yl | dibenzo[b,d]thien-4-yl |
| 159 | Ia.1 | —C(O)OCH₃ | dibenzo[b,d]thien-4-yl | dibenzo[b,d]thien-4-yl |
| 160 | Ia.1 | —CH₂OH | thianthren-1-yl | dibenzo[b,d]thien-4-yl |
| 161 | Ia.1 | —C(O)OH | thianthren-1-yl | dibenzo[b,d]thien-4-yl |
| 162 | Ia.1 | —C(O)OCH₃ | thianthren-1-yl | dibenzo[b,d]thien-4-yl |
| 163 | Ia.1 | —CH₂OH | phenyl | thianthren-1-yl |
| 164 | Ia.1 | —C(O)OH | phenyl | thianthren-1-yl |
| 165 | Ia.1 | —C(O)OCH₃ | phenyl | thianthren-1-yl |
| 166 | Ia.1 | —CH₂OH | 1-naphthyl | thianthren-1-yl |
| 167 | Ia.1 | —C(O)OH | 1-naphthyl | thianthren-1-yl |
| 168 | Ia.1 | —C(O)OCH₃ | 1-naphthyl | thianthren-1-yl |
| 169 | Ia.1 | —CH₂OH | 2-naphthyl | thianthren-1-yl |
| 170 | Ia.1 | —C(O)OH | 2-naphthyl | thianthren-1-yl |
| 171 | Ia.1 | —C(O)OCH₃ | 2-naphthyl | thianthren-1-yl |
| 172 | Ia.1 | —CH₂OH | 9-phenanthryl | thianthren-1-yl |
| 173 | Ia.1 | —C(O)OH | 9-phenanthryl | thianthren-1-yl |
| 174 | Ia.1 | —C(O)OCH₃ | 9-phenanthryl | thianthren-1-yl |
| 175 | Ia.1 | —CH₂OH | dibenzo[b,d]thien-4-yl | thianthren-1-yl |
| 176 | Ia.1 | —C(O)OH | dibenzo[b,d]thien-4-yl | thianthren-1-yl |
| 177 | Ia.1 | —C(O)OCH₃ | dibenzo[b,d]thien-4-yl | thianthren-1-yl |
| 178 | Ia.1 | —CH₂OH | thianthren-1-yl | thianthren-1-yl |
| 179 | Ia.1 | —C(O)OH | thianthren-1-yl | thianthren-1-yl |
| 180 | Ia.1 | —C(O)OCH₃ | thianthren-1-yl | thianthren-1-yl |
| 181 | Ia.2 | —CH₂OH | phenyl | phenyl |
| 182 | Ia.2 | —C(O)OH | phenyl | phenyl |
| 183 | Ia.2 | —C(O)OCH₃ | phenyl | phenyl |
| 184 | Ia.2 | —CH₂OH | 1-naphthyl | phenyl |
| 185 | Ia.2 | —C(O)OH | 1-naphthyl | phenyl |
| 186 | Ia.2 | —C(O)OCH₃ | 1-naphthyl | phenyl |
| 187 | Ia.2 | —CH₂OH | 2-naphthyl | phenyl |
| 188 | Ia.2 | —C(O)OH | 2-naphthyl | phenyl |
| 189 | Ia.2 | —C(O)OCH₃ | 2-naphthyl | phenyl |
| 190 | Ia.2 | —CH₂OH | 9-phenanthryl | phenyl |
| 191 | Ia.2 | —C(O)OH | 9-phenanthryl | phenyl |
| 192 | Ia.2 | —C(O)OCH₃ | 9-phenanthryl | phenyl |
| 193 | Ia.2 | —CH₂OH | dibenzo[b,d]thien-4-yl | phenyl |
| 194 | Ia.2 | —C(O)OH | dibenzo[b,d]thien-4-yl | phenyl |
| 195 | Ia.2 | —C(O)OCH₃ | dibenzo[b,d]thien-4-yl | phenyl |
| 196 | Ia.2 | —CH₂OH | thianthren-1-yl | phenyl |
| 197 | Ia.2 | —C(O)OH | thianthren-1-yl | phenyl |
| 198 | Ia.2 | —C(O)OCH₃ | thianthren-1-yl | phenyl |
| 199 | Ia.2 | —CH₂OH | phenyl | 1-naphthyl |
| 200 | Ia.2 | —C(O)OH | phenyl | 1-naphthyl |
| 201 | Ia.2 | —C(O)OCH₃ | phenyl | 1-naphthyl |
| 202 | Ia.2 | —CH₂OH | 1-naphthyl | 1-naphthyl |
| 203 | Ia.2 | —C(O)OH | 1-naphthyl | 1-naphthyl |
| 204 | Ia.2 | —C(O)OCH₃ | 1-naphthyl | 1-naphthyl |
| 205 | Ia.2 | —CH₂OH | 2-naphthyl | 1-naphthyl |
| 206 | Ia.2 | —C(O)OH | 2-naphthyl | 1-naphthyl |
| 207 | Ia.2 | —C(O)OCH₃ | 2-naphthyl | 1-naphthyl |
| 208 | Ia.2 | —CH₂OH | 9-phenanthryl | 1-naphthyl |
| 209 | Ia.2 | —C(O)OH | 9-phenanthryl | 1-naphthyl |
| 210 | Ia.2 | —C(O)OCH₃ | 9-phenanthryl | 1-naphthyl |
| 211 | Ia.2 | —CH₂OH | dibenzo[b,d]thien-4-yl | 1-naphthyl |
| 212 | Ia.2 | —C(O)OH | dibenzo[b,d]thien-4-yl | 1-naphthyl |
| 213 | Ia.2 | —C(O)OCH₃ | dibenzo[b,d]thien-4-yl | 1-naphthyl |
| 214 | Ia.2 | —CH₂OH | thianthren-1-yl | 1-naphthyl |
| 215 | Ia.2 | —C(O)OH | thianthren-1-yl | 1-naphthyl |
| 216 | Ia.2 | —C(O)OCH₃ | thianthren-1-yl | 1-naphthyl |
| 217 | Ia.2 | —CH₂OH | phenyl | 2-naphthyl |
| 218 | Ia.2 | —C(O)OH | phenyl | 2-naphthyl |
| 219 | Ia.2 | —C(O)OCH₃ | phenyl | 2-naphthyl |
| 220 | Ia.2 | —CH₂OH | 1-naphthyl | 2-naphthyl |
| 221 | Ia.2 | —C(O)OH | 1-naphthyl | 2-naphthyl |
| 222 | Ia.2 | —C(O)OCH₃ | 1-naphthyl | 2-naphthyl |
| 223 | Ia.2 | —CH₂OH | 2-naphthyl | 2-naphthyl |
| 224 | Ia.2 | —C(O)OH | 2-naphthyl | 2-naphthyl |
| 225 | Ia.2 | —C(O)OCH₃ | 2-naphthyl | 2-naphthyl |
| 226 | Ia.2 | —CH₂OH | 9-phenanthryl | 2-naphthyl |
| 227 | Ia.2 | —C(O)OH | 9-phenanthryl | 2-naphthyl |
| 228 | Ia.2 | —C(O)OCH₃ | 9-phenanthryl | 2-naphthyl |
| 229 | Ia.2 | —CH₂OH | dibenzo[b,d]thien-4-yl | 2-naphthyl |
| 230 | Ia.2 | —C(O)OH | dibenzo[b,d]thien-4-yl | 2-naphthyl |
| 231 | Ia.2 | —C(O)OCH₃ | dibenzo[b,d]thien-4-yl | 2-naphthyl |
| 232 | Ia.2 | —CH₂OH | thianthren-1-yl | 2-naphthyl |
| 233 | Ia.2 | —C(O)OH | thianthren-1-yl | 2-naphthyl |
| 234 | Ia.2 | —C(O)OCH₃ | thianthren-1-yl | 2-naphthyl |
| 235 | Ia.2 | —CH₂OH | phenyl | 9-phenanthryl |
| 236 | Ia.2 | —C(O)OH | phenyl | 9-phenanthryl |
| 237 | Ia.2 | —C(O)OCH₃ | phenyl | 9-phenanthryl |
| 238 | Ia.2 | —CH₂OH | 1-naphthyl | 9-phenanthryl |
| 239 | Ia.2 | —C(O)OH | 1-naphthyl | 9-phenanthryl |
| 240 | Ia.2 | —C(O)OCH₃ | 1-naphthyl | 9-phenanthryl |
| 241 | Ia.2 | —CH₂OH | 2-naphthyl | 9-phenanthryl |
| 242 | Ia.2 | —C(O)OH | 2-naphthyl | 9-phenanthryl |
| 243 | Ia.2 | —C(O)OCH₃ | 2-naphthyl | 9-phenanthryl |
| 244 | Ia.2 | —CH₂OH | 9-phenanthryl | 9-phenanthryl |
| 245 | Ia.2 | —C(O)OH | 9-phenanthryl | 9-phenanthryl |
| 246 | Ia.2 | —C(O)OCH₃ | 9-phenanthryl | 9-phenanthryl |
| 247 | Ia.2 | —CH₂OH | dibenzo[b,d]thien-4-yl | 9-phenanthryl |
| 248 | Ia.2 | —C(O)OH | dibenzo[b,d]thien-4-yl | 9-phenanthryl |
| 249 | Ia.2 | —C(O)OCH₃ | dibenzo[b,d]thien-4-yl | 9-phenanthryl |
| 250 | Ia.2 | —CH₂OH | thianthren-1-yl | 9-phenanthryl |
| 251 | Ia.2 | —C(O)OH | thianthren-1-yl | 9-phenanthryl |
| 252 | Ia.2 | —C(O)OCH₃ | thianthren-1-yl | 9-phenanthryl |
| 253 | Ia.2 | —CH₂OH | phenyl | dibenzo[b,d]thien-4-yl |
| 254 | Ia.2 | —C(O)OH | phenyl | dibenzo[b,d]thien-4-yl |
| 255 | Ia.2 | —C(O)OCH₃ | phenyl | dibenzo[b,d]thien-4-yl |
| 256 | Ia.2 | —CH₂OH | 1-naphthyl | dibenzo[b,d]thien-4-yl |
| 257 | Ia.2 | —C(O)OH | 1-naphthyl | dibenzo[b,d]thien-4-yl |
| 258 | Ia.2 | —C(O)OCH₃ | 1-naphthyl | dibenzo[b,d]thien-4-yl |
| 259 | Ia.2 | —CH₂OH | 2-naphthyl | dibenzo[b,d]thien-4-yl |
| 260 | Ia.2 | —C(O)OH | 2-naphthyl | dibenzo[b,d]thien-4-yl |
| 261 | Ia.2 | —C(O)OCH₃ | 2-naphthyl | dibenzo[b,d]thien-4-yl |
| 262 | Ia.2 | —CH₂OH | 9-phenanthryl | dibenzo[b,d]thien-4-yl |
| 263 | Ia.2 | —C(O)OH | 9-phenanthryl | dibenzo[b,d]thien-4-yl |
| 264 | Ia.2 | —C(O)OCH₃ | 9-phenanthryl | dibenzo[b,d]thien-4-yl |
| 265 | Ia.2 | —CH₂OH | dibenzo[b,d]thien-4-yl | dibenzo[b,d]thien-4-yl |
| 266 | Ia.2 | —C(O)OH | dibenzo[b,d]thien-4-yl | dibenzo[b,d]thien-4-yl |
| 267 | Ia.2 | —C(O)OCH₃ | dibenzo[b,d]thien-4-yl | dibenzo[b,d]thien-4-yl |
| 268 | Ia.2 | —CH₂OH | thianthren-1-yl | dibenzo[b,d]thien-4-yl |
| 269 | Ia.2 | —C(O)OH | thianthren-1-yl | dibenzo[b,d]thien-4-yl |
| 270 | Ia.2 | —C(O)OCH₃ | thianthren-1-yl | dibenzo[b,d]thien-4-yl |
| 271 | Ia.2 | —CH₂OH | phenyl | thianthren-1-yl |
| 272 | Ia.2 | —C(O)OH | phenyl | thianthren-1-yl |
| 273 | Ia.2 | —C(O)OCH₃ | phenyl | thianthren-1-yl |
| 274 | Ia.2 | —CH₂OH | 1-naphthyl | thianthren-1-yl |
| 275 | Ia.2 | —C(O)OH | 1-naphthyl | thianthren-1-yl |
| 276 | Ia.2 | —C(O)OCH₃ | 1-naphthyl | thianthren-1-yl |
| 277 | Ia.2 | —CH₂OH | 2-naphthyl | thianthren-1-yl |
| 278 | Ia.2 | —C(O)OH | 2-naphthyl | thianthren-1-yl |
| 279 | Ia.2 | —C(O)OCH₃ | 2-naphthyl | thianthren-1-yl |
| 280 | Ia.2 | —CH₂OH | 9-phenanthryl | thianthren-1-yl |
| 281 | Ia.2 | —C(O)OH | 9-phenanthryl | thianthren-1-yl |
| 282 | Ia.2 | —C(O)OCH₃ | 9-phenanthryl | thianthren-1-yl |
| 283 | Ia.2 | —CH₂OH | dibenzo[b,d]thien-4-yl | thianthren-1-yl |
| 284 | Ia.2 | —C(O)OH | dibenzo[b,d]thien-4-yl | thianthren-1-yl |
| 285 | Ia.2 | —C(O)OCH₃ | dibenzo[b,d]thien-4-yl | thianthren-1-yl |
| 286 | Ia.2 | —CH₂OH | thianthren-1-yl | thianthren-1-yl |
| 287 | Ia.2 | —C(O)OH | thianthren-1-yl | thianthren-1-yl |
| 288 | Ia.2 | —C(O)OCH₃ | thianthren-1-yl | thianthren-1-yl |
| 289 | Ia.3 | —CH₂OH | phenyl | phenyl |
| 290 | Ia.3 | —C(O)OH | phenyl | phenyl |
| 291 | Ia.3 | —C(O)OCH₃ | phenyl | phenyl |
| 292 | Ia.3 | —CH₂OH | 1-naphthyl | phenyl |
| 293 | Ia.3 | —C(O)OH | 1-naphthyl | phenyl |
| 294 | Ia.3 | —C(O)OCH₃ | 1-naphthyl | phenyl |
| 295 | Ia.3 | —CH₂OH | 2-naphthyl | phenyl |
| 296 | Ia.3 | —C(O)OH | 2-naphthyl | phenyl |
| 297 | Ia.3 | —C(O)OCH₃ | 2-naphthyl | phenyl |
| 298 | Ia.3 | —CH₂OH | 9-phenanthryl | phenyl |
| 299 | Ia.3 | —C(O)OH | 9-phenanthryl | phenyl |
| 300 | Ia.3 | —C(O)OCH₃ | 9-phenanthryl | phenyl |
| 301 | Ia.3 | —CH₂OH | dibenzo[b,d]thien-4-yl | phenyl |
| 302 | Ia.3 | —C(O)OH | dibenzo[b,d]thien-4-yl | phenyl |
| 303 | Ia.3 | —C(O)OCH₃ | dibenzo[b,d]thien-4-yl | phenyl |
| 304 | Ia.3 | —CH₂OH | thianthren-1-yl | phenyl |
| 305 | Ia.3 | —C(O)OH | thianthren-1-yl | phenyl |
| 306 | Ia.3 | —C(O)OCH₃ | thianthren-1-yl | phenyl |
| 307 | Ia.3 | —CH₂OH | phenyl | 1-naphthyl |
| 308 | Ia.3 | —C(O)OH | phenyl | 1-naphthyl |
| 309 | Ia.3 | —C(O)OCH₃ | phenyl | 1-naphthyl |
| 310 | Ia.3 | —CH₂OH | 1-naphthyl | 1-naphthyl |
| 311 | Ia.3 | —C(O)OH | 1-naphthyl | 1-naphthyl |
| 312 | Ia.3 | —C(O)OCH₃ | 1-naphthyl | 1-naphthyl |
| 313 | Ia.3 | —CH₂OH | 2-naphthyl | 1-naphthyl |
| 314 | Ia.3 | —C(O)OH | 2-naphthyl | 1-naphthyl |
| 315 | Ia.3 | —C(O)OCH₃ | 2-naphthyl | 1-naphthyl |
| 316 | Ia.3 | —CH₂OH | 9-phenanthryl | 1-naphthyl |
| 317 | Ia.3 | —C(O)OH | 9-phenanthryl | 1-naphthyl |
| 318 | Ia.3 | —C(O)OCH₃ | 9-phenanthryl | 1-naphthyl |
| 319 | Ia.3 | —CH₂OH | dibenzo[b,d]thien-4-yl | 1-naphthyl |
| 320 | Ia.3 | —C(O)OH | dibenzo[b,d]thien-4-yl | 1-naphthyl |
| 321 | Ia.3 | —C(O)OCH₃ | dibenzo[b,d]thien-4-yl | 1-naphthyl |
| 322 | Ia.3 | —CH₂OH | thianthren-1-yl | 1-naphthyl |
| 323 | Ia.3 | —C(O)OH | thianthren-1-yl | 1-naphthyl |
| 324 | Ia.3 | —C(O)OCH₃ | thianthren-1-yl | 1-naphthyl |
| 325 | Ia.3 | —CH₂OH | phenyl | 2-naphthyl |
| 326 | Ia.3 | —C(O)OH | phenyl | 2-naphthyl |
| 327 | Ia.3 | —C(O)OCH₃ | phenyl | 2-naphthyl |
| 328 | Ia.3 | —CH₂OH | 1-naphthyl | 2-naphthyl |
| 329 | Ia.3 | —C(O)OH | 1-naphthyl | 2-naphthyl |
| 330 | Ia.3 | —C(O)OCH₃ | 1-naphthyl | 2-naphthyl |
| 331 | Ia.3 | —CH₂OH | 2-naphthyl | 2-naphthyl |
| 332 | Ia.3 | —C(O)OH | 2-naphthyl | 2-naphthyl |
| 333 | Ia.3 | —C(O)OCH₃ | 2-naphthyl | 2-naphthyl |
| 334 | Ia.3 | —CH₂OH | 9-phenanthryl | 2-naphthyl |
| 335 | Ia.3 | —C(O)OH | 9-phenanthryl | 2-naphthyl |
| 336 | Ia.3 | —C(O)OCH₃ | 9-phenanthryl | 2-naphthyl |
| 337 | Ia.3 | —CH₂OH | dibenzo[b,d]thien-4-yl | 2-naphthyl |
| 338 | Ia.3 | —C(O)OH | dibenzo[b,d]thien-4-yl | 2-naphthyl |
| 339 | Ia.3 | —C(O)OCH₃ | dibenzo[b,d]thien-4-yl | 2-naphthyl |
| 340 | Ia.3 | —CH₂OH | thianthren-1-yl | 2-naphthyl |
| 341 | Ia.3 | —C(O)OH | thianthren-1-yl | 2-naphthyl |
| 342 | Ia.3 | —C(O)OCH₃ | thianthren-1-yl | 2-naphthyl |
| 343 | Ia.3 | —CH₂OH | phenyl | 9-phenanthryl |
| 344 | Ia.3 | —C(O)OH | phenyl | 9-phenanthryl |
| 345 | Ia.3 | —C(O)OCH₃ | phenyl | 9-phenanthryl |
| 346 | Ia.3 | —CH₂OH | 1-naphthyl | 9-phenanthryl |
| 347 | Ia.3 | —C(O)OH | 1-naphthyl | 9-phenanthryl |
| 348 | Ia.3 | —C(O)OCH₃ | 1-naphthyl | 9-phenanthryl |
| 349 | Ia.3 | —CH₂OH | 2-naphthyl | 9-phenanthryl |
| 350 | Ia.3 | —C(O)OH | 2-naphthyl | 9-phenanthryl |
| 351 | Ia.3 | —C(O)OCH₃ | 2-naphthyl | 9-phenanthryl |
| 352 | Ia.3 | —CH₂OH | 9-phenanthryl | 9-phenanthryl |
| 353 | Ia.3 | —C(O)OH | 9-phenanthryl | 9-phenanthryl |
| 354 | Ia.3 | —C(O)OCH₃ | 9-phenanthryl | 9-phenanthryl |
| 355 | Ia.3 | —CH₂OH | dibenzo[b,d]thien-4-yl | 9-phenanthryl |
| 356 | Ia.3 | —C(O)OH | dibenzo[b,d]thien-4-yl | 9-phenanthryl |
| 357 | Ia.3 | —C(O)OCH₃ | dibenzo[b,d]thien-4-yl | 9-phenanthryl |
| 358 | Ia.3 | —CH₂OH | thianthren-1-yl | 9-phenanthryl |
| 359 | Ia.3 | —C(O)OH | thianthren-1-yl | 9-phenanthryl |
| 360 | Ia.3 | —C(O)OCH₃ | thianthren-1-yl | 9-phenanthryl |
| 361 | Ia.3 | —CH₂OH | phenyl | dibenzo[b,d]thien-4-yl |
| 362 | Ia.3 | —C(O)OH | phenyl | dibenzo[b,d]thien-4-yl |
| 363 | Ia.3 | —C(O)OCH₃ | phenyl | dibenzo[b,d]thien-4-yl |
| 364 | Ia.3 | —CH₂OH | 1-naphthyl | dibenzo[b,d]thien-4-yl |
| 365 | Ia.3 | —C(O)OH | 1-naphthyl | dibenzo[b,d]thien-4-yl |
| 366 | Ia.3 | —C(O)OCH₃ | 1-naphthyl | dibenzo[b,d]thien-4-yl |
| 367 | Ia.3 | —CH₂OH | 2-naphthyl | dibenzo[b,d]thien-4-yl |
| 368 | Ia.3 | —C(O)OH | 2-naphthyl | dibenzo[b,d]thien-4-yl |
| 369 | Ia.3 | —C(O)OCH₃ | 2-naphthyl | dibenzo[b,d]thien-4-yl |
| 370 | Ia.3 | —CH₂OH | 9-phenanthryl | dibenzo[b,d]thien-4-yl |
| 371 | Ia.3 | —C(O)OH | 9-phenanthryl | dibenzo[b,d]thien-4-yl |
| 372 | Ia.3 | —C(O)OCH₃ | 9-phenanthryl | dibenzo[b,d]thien-4-yl |
| 373 | Ia.3 | —CH₂OH | dibenzo[b,d]thien-4-yl | dibenzo[b,d]thien-4-yl |
| 374 | Ia.3 | —C(O)OH | dibenzo[b,d]thien-4-yl | dibenzo[b,d]thien-4-yl |
| 375 | Ia.3 | —C(O)OCH₃ | dibenzo[b,d]thien-4-yl | dibenzo[b,d]thien-4-yl |
| 376 | Ia.3 | —CH₂OH | thianthren-1-yl | dibenzo[b,d]thien-4-yl |
| 377 | Ia.3 | —C(O)OH | thianthren-1-yl | dibenzo[b,d]thien-4-yl |
| 378 | Ia.3 | —C(O)OCH₃ | thianthren-1-yl | dibenzo[b,d]thien-4-yl |
| 379 | Ia.3 | —CH₂OH | phenyl | thianthren-1-yl |
| 380 | Ia.3 | —C(O)OH | phenyl | thianthren-1-yl |
| 381 | Ia.3 | —C(O)OCH₃ | phenyl | thianthren-1-yl |
| 382 | Ia.3 | —CH₂OH | 1-naphthyl | thianthren-1-yl |
| 383 | Ia.3 | —C(O)OH | 1-naphthyl | thianthren-1-yl |
| 384 | Ia.3 | —C(O)OCH₃ | 1-naphthyl | thianthren-1-yl |
| 385 | Ia.3 | —CH₂OH | 2-naphthyl | thianthren-1-yl |
| 386 | Ia.3 | —C(O)OH | 2-naphthyl | thianthren-1-yl |
| 387 | Ia.3 | —C(O)OCH₃ | 2-naphthyl | thianthren-1-yl |
| 388 | Ia.3 | —CH₂OH | 9-phenanthryl | thianthren-1-yl |
| 389 | Ia.3 | —C(O)OH | 9-phenanthryl | thianthren-1-yl |
| 390 | Ia.3 | —C(O)OCH₃ | 9-phenanthryl | thianthren-1-yl |
| 391 | Ia.3 | —CH₂OH | dibenzo[b,d]thien-4-yl | thianthren-1-yl |
| 392 | Ia.3 | —C(O)OH | dibenzo[b,d]thien-4-yl | thianthren-1-yl |
| 393 | Ia.3 | —C(O)OCH₃ | dibenzo[b,d]thien-4-yl | thianthren-1-yl |
| 394 | Ia.3 | —CH₂OH | thianthren-1-yl | thianthren-1-yl |
| 395 | Ia.3 | —C(O)OH | thianthren-1-yl | thianthren-1-yl |
| 396 | Ia.3 | —C(O)OCH₃ | thianthren-1-yl | thianthren-1-yl |
| 397 | Ia.4 | —CH₂OH | phenyl | phenyl |
| 398 | Ia.4 | —C(O)OH | phenyl | phenyl |
| 399 | Ia.4 | —C(O)OCH₃ | phenyl | phenyl |
| 400 | Ia.4 | —CH₂OH | 1-naphthyl | phenyl |
| 401 | Ia.4 | —C(O)OH | 1-naphthyl | phenyl |
| 402 | Ia.4 | —C(O)OCH₃ | 1-naphthyl | phenyl |
| 403 | Ia.4 | —CH₂OH | 2-naphthyl | phenyl |
| 404 | Ia.4 | —C(O)OH | 2-naphthyl | phenyl |
| 405 | Ia.4 | —C(O)OCH₃ | 2-naphthyl | phenyl |
| 406 | Ia.4 | —CH₂OH | 9-phenanthryl | phenyl |
| 407 | Ia.4 | —C(O)OH | 9-phenanthryl | phenyl |
| 408 | Ia.4 | —C(O)OCH₃ | 9-phenanthryl | phenyl |
| 409 | Ia.4 | —CH₂OH | dibenzo[b,d]thien-4-yl | phenyl |
| 410 | Ia.4 | —C(O)OH | dibenzo[b,d]thien-4-yl | phenyl |
| 411 | Ia.4 | —C(O)OCH₃ | dibenzo[b,d]thien-4-yl | phenyl |
| 412 | Ia.4 | —CH₂OH | thianthren-1-yl | phenyl |
| 413 | Ia.4 | —C(O)OH | thianthren-1-yl | phenyl |
| 414 | Ia.4 | —C(O)OCH₃ | thianthren-1-yl | phenyl |
| 415 | Ia.4 | —CH₂OH | phenyl | 1-naphthyl |
| 416 | Ia.4 | —C(O)OH | phenyl | 1-naphthyl |
| 417 | Ia.4 | —C(O)OCH₃ | phenyl | 1-naphthyl |
| 418 | Ia.4 | —CH₂OH | 1-naphthyl | 1-naphthyl |
| 419 | Ia.4 | —C(O)OH | 1-naphthyl | 1-naphthyl |
| 420 | Ia.4 | —C(O)OCH₃ | 1-naphthyl | 1-naphthyl |
| 421 | Ia.4 | —CH₂OH | 2-naphthyl | 1-naphthyl |
| 422 | Ia.4 | —C(O)OH | 2-naphthyl | 1-naphthyl |
| 423 | Ia.4 | —C(O)OCH₃ | 2-naphthyl | 1-naphthyl |
| 424 | Ia.4 | —CH₂OH | 9-phenanthryl | 1-naphthyl |
| 425 | Ia.4 | —C(O)OH | 9-phenanthryl | 1-naphthyl |
| 426 | Ia.4 | —C(O)OCH₃ | 9-phenanthryl | 1-naphthyl |
| 427 | Ia.4 | —CH₂OH | dibenzo[b,d]thien-4-yl | 1-naphthyl |
| 428 | Ia.4 | —C(O)OH | dibenzo[b,d]thien-4-yl | 1-naphthyl |
| 429 | Ia.4 | —C(O)OCH₃ | dibenzo[b,d]thien-4-yl | 1-naphthyl |
| 430 | Ia.4 | —CH₂OH | thianthren-1-yl | 1-naphthyl |
| 431 | Ia.4 | —C(O)OH | thianthren-1-yl | 1-naphthyl |
| 432 | Ia.4 | —C(O)OCH₃ | thianthren-1-yl | 1-naphthyl |
| 433 | Ia.4 | —CH₂OH | phenyl | 2-naphthyl |
| 434 | Ia.4 | —C(O)OH | phenyl | 2-naphthyl |
| 435 | Ia.4 | —C(O)OCH₃ | phenyl | 2-naphthyl |
| 436 | Ia.4 | —CH₂OH | 1-naphthyl | 2-naphthyl |
| 437 | Ia.4 | —C(O)OH | 1-naphthyl | 2-naphthyl |
| 438 | Ia.4 | —C(O)OCH₃ | 1-naphthyl | 2-naphthyl |
| 439 | Ia.4 | —CH₂OH | 2-naphthyl | 2-naphthyl |
| 440 | Ia.4 | —C(O)OH | 2-naphthyl | 2-naphthyl |
| 441 | Ia.4 | —C(O)OCH₃ | 2-naphthyl | 2-naphthyl |
| 442 | Ia.4 | —CH₂OH | 9-phenanthryl | 2-naphthyl |
| 443 | Ia.4 | —C(O)OH | 9-phenanthryl | 2-naphthyl |
| 444 | Ia.4 | —C(O)OCH₃ | 9-phenanthryl | 2-naphthyl |
| 445 | Ia.4 | —CH₂OH | dibenzo[b,d]thien-4-yl | 2-naphthyl |
| 446 | Ia.4 | —C(O)OH | dibenzo[b,d]thien-4-yl | 2-naphthyl |
| 447 | Ia.4 | —C(O)OCH₃ | dibenzo[b,d]thien-4-yl | 2-naphthyl |
| 448 | Ia.4 | —CH₂OH | thianthren-1-yl | 2-naphthyl |
| 449 | Ia.4 | —C(O)OH | thianthren-1-yl | 2-naphthyl |
| 450 | Ia.4 | —C(O)OCH₃ | thianthren-1-yl | 2-naphthyl |
| 451 | Ia.4 | —CH₂OH | phenyl | 9-phenanthryl |
| 452 | Ia.4 | —C(O)OH | phenyl | 9-phenanthryl |
| 453 | Ia.4 | —C(O)OCH₃ | phenyl | 9-phenanthryl |
| 454 | Ia.4 | —CH₂OH | 1-naphthyl | 9-phenanthryl |
| 455 | Ia.4 | —C(O)OH | 1-naphthyl | 9-phenanthryl |
| 456 | Ia.4 | —C(O)OCH₃ | 1-naphthyl | 9-phenanthryl |
| 457 | Ia.4 | —CH₂OH | 2-naphthyl | 9-phenanthryl |
| 458 | Ia.4 | —C(O)OH | 2-naphthyl | 9-phenanthryl |
| 459 | Ia.4 | —C(O)OCH₃ | 2-naphthyl | 9-phenanthryl |
| 460 | Ia.4 | —CH₂OH | 9-phenanthryl | 9-phenanthryl |
| 461 | Ia.4 | —C(O)OH | 9-phenanthryl | 9-phenanthryl |
| 462 | Ia.4 | —C(O)OCH₃ | 9-phenanthryl | 9-phenanthryl |
| 463 | Ia.4 | —CH₂OH | dibenzo[b,d]thien-4-yl | 9-phenanthryl |
| 464 | Ia.4 | —C(O)OH | dibenzo[b,d]thien-4-yl | 9-phenanthryl |
| 465 | Ia.4 | —C(O)OCH₃ | dibenzo[b,d]thien-4-yl | 9-phenanthryl |
| 466 | Ia.4 | —CH₂OH | thianthren-1-yl | 9-phenanthryl |
| 467 | Ia.4 | —C(O)OH | thianthren-1-yl | 9-phenanthryl |
| 468 | Ia.4 | —C(O)OCH₃ | thianthren-1-yl | 9-phenanthryl |
| 469 | Ia.4 | —CH₂OH | phenyl | dibenzo[b,d]thien-4-yl |
| 470 | Ia.4 | —C(O)OH | phenyl | dibenzo[b,d]thien-4-yl |
| 471 | Ia.4 | —C(O)OCH₃ | phenyl | dibenzo[b,d]thien-4-yl |
| 472 | Ia.4 | —CH₂OH | 1-naphthyl | dibenzo[b,d]thien-4-yl |
| 473 | Ia.4 | —C(O)OH | 1-naphthyl | dibenzo[b,d]thien-4-yl |
| 474 | Ia.4 | —C(O)OCH₃ | 1-naphthyl | dibenzo[b,d]thien-4-yl |
| 475 | Ia.4 | —CH₂OH | 2-naphthyl | dibenzo[b,d]thien-4-yl |
| 476 | Ia.4 | —C(O)OH | 2-naphthyl | dibenzo[b,d]thien-4-yl |
| 477 | Ia.4 | —C(O)OCH₃ | 2-naphthyl | dibenzo[b,d]thien-4-yl |
| 478 | Ia.4 | —CH₂OH | 9-phenanthryl | dibenzo[b,d]thien-4-yl |
| 479 | Ia.4 | —C(O)OH | 9-phenanthryl | dibenzo[b,d]thien-4-yl |
| 480 | Ia.4 | —C(O)OCH₃ | 9-phenanthryl | dibenzo[b,d]thien-4-yl |
| 481 | Ia.4 | —CH₂OH | dibenzo[b,d]thien-4-yl | dibenzo[b,d]thien-4-yl |
| 482 | Ia.4 | —C(O)OH | dibenzo[b,d]thien-4-yl | dibenzo[b,d]thien-4-yl |
| 483 | Ia.4 | —C(O)OCH₃ | dibenzo[b,d]thien-4-yl | dibenzo[b,d]thien-4-yl |
| 484 | Ia.4 | —CH₂OH | thianthren-1-yl | dibenzo[b,d]thien-4-yl |
| 485 | Ia.4 | —C(O)OH | thianthren-1-yl | dibenzo[b,d]thien-4-yl |
| 486 | Ia.4 | —C(O)OCH₃ | thianthren-1-yl | dibenzo[b,d]thien-4-yl |
| 487 | Ia.4 | —CH₂OH | phenyl | thianthren-1-yl |
| 488 | Ia.4 | —C(O)OH | phenyl | thianthren-1-yl |
| 489 | Ia.4 | —C(O)OCH₃ | phenyl | thianthren-1-yl |
| 490 | Ia.4 | —CH₂OH | 1-naphthyl | thianthren-1-yl |
| 491 | Ia.4 | —C(O)OH | 1-naphthyl | thianthren-1-yl |
| 492 | Ia.4 | —C(O)OCH₃ | 1-naphthyl | thianthren-1-yl |
| 493 | Ia.4 | —CH₂OH | 2-naphthyl | thianthren-1-yl |
| 494 | Ia.4 | —C(O)OH | 2-naphthyl | thianthren-1-yl |
| 495 | Ia.4 | —C(O)OCH₃ | 2-naphthyl | thianthren-1-yl |
| 496 | Ia.4 | —CH₂OH | 9-phenanthryl | thianthren-1-yl |
| 497 | Ia.4 | —C(O)OH | 9-phenanthryl | thianthren-1-yl |
| 498 | Ia.4 | —C(O)OCH₃ | 9-phenanthryl | thianthren-1-yl |
| 499 | Ia.4 | —CH₂OH | dibenzo[b,d]thien-4-yl | thianthren-1-yl |
| 500 | Ia.4 | —C(O)OH | dibenzo[b,d]thien-4-yl | thianthren-1-yl |
| 501 | Ia.4 | —C(O)OCH₃ | dibenzo[b,d]thien-4-yl | thianthren-1-yl |
| 502 | Ia.4 | —CH₂OH | thianthren-1-yl | thianthren-1-yl |
| 503 | Ia.4 | —C(O)OH | thianthren-1-yl | thianthren-1-yl |
| 504 | Ia.4 | —C(O)OCH₃ | thianthren-1-yl | thianthren-1-yl |
